(19) United States
(12) Patent Application Publication
(10) Pub. No.: US 2025/0302954 A1
(43) Pub. Date: Oct. 2, 2025

(54) METHODS TO OVERCOME DRUG RESISTANCE BY RE-SENSITIZING CANCER CELLS TO TREATMENT WITH A PRIOR THERAPY VIA TREATMENT WITH A T CELL THERAPY

(71) Applicant: Celgene Corporation, Summit, NJ (US)

(72) Inventors: Nathan MARTIN, Seattle, WA (US); Nicholas STONG, Summit, NJ (US); Timothy CAMPBELL, San Francisco, CA (US); Jennifer Erin FLYNT, Summit, NJ (US); Ethan THOMPSON, Seattle, WA (US); Shari KAISER, Seattle, WA (US)

(73) Assignee: Celgene Corporation, Summit, NJ (US)

(21) Appl. No.: 18/864,515

(22) PCT Filed: May 10, 2023
(86) PCT No.: PCT/US2023/066849

§ 371 Date: Nov. 8, 2024

Related U.S. Application Data
(60) Provisional application No. 63/340,794, filed on May 11, 2022.
(60) Provisional application No. 63/350,152, filed on Jun. 8, 2022.

(51) Int. Cl. A61K 40/11; A61K 31/4985; A61K 31/506; A61K 31/52; A61K 35/17; A61K 40/31; A61K 40/32; A61P 35/00

(52) U.S. Cl. CPC A61K 40/11; A61K 31/4985; A61K 31/506; A61K 31/52; A61K 35/17; A61K 40/31; A61K 40/32; A61P 35/00; A61K 2239/21; A61K 2239/22

(57) ABSTRACT

Provided herein are methods to overcome drug resistance by re-sensitizing cancer cells to treatment with a prior therapy via treatment with a T cell therapy.

### CROSS-REFERENCE TO RELATED APPLICATIONS

This application claims priority from U.S. provisional application No. 63/340,794, filed May 11, 2022, entitled “METHODS OF TREATMENT WITH T CELL THERAPIES,” and U.S. provisional application No. 63/350,152, filed Jun. 8, 2022, entitled “METHODS OF TREATMENT WITH T CELL THERAPIES,” the contents of each are incorporated by reference in their entirety.

### FIELD

The present disclosure relates in some aspects to methods, uses, compositions, and kits of T cell therapies for treating subjects with a cancer, including those who have relapsed following treatment with, or are refractory to, a prior therapy for treating the cancer. The T cell therapy includes cells that express recombinant receptors such as chimeric antigen receptors (CARs), as well as T cell engagers (TCEs). In some embodiments, the cancer is a B cell malignancy, such as multiple myeloma.

### INCORPORATION BY REFERENCE OF SEQUENCES LISTING

The present application is being filed along with a Sequence Listing in electronic format. The Sequence Listing is provided as a file entitled 683772002540SeqList.xml, created May 10, 2023, which is 398,315 bytes in size. The information in the electronic format of the Sequence Listing is incorporated by reference in its entirety.

### BACKGROUND

Various strategies are available for treating cancers, including those involving the administration of small molecules, antibodies, or both. In some cases, a subject relapses after treatment with, or becomes refractory to, a prior therapy, such as by the development of resistance-conferring mutations. Improved strategies are needed to overcome such resistance, such as by re-sensitizing cancer cells to treatment with a prior therapy via treatment with a T cell therapy. Provided are methods, uses, compositions, and kits that meet such needs.

### SUMMARY

Provided herein is a method of treating a cancer, comprising: (a) selecting a subject having a cancer for treatment with a subsequent therapy for treating the cancer, wherein the subject was previously administered a T cell therapy for treating the cancer and a prior therapy for treating the cancer, and wherein: (i) the subject was administered the T cell therapy at a time when the subject had relapsed following treatment with, or was refractory to, the prior therapy; (ii) following administration of the T cell therapy, the subject achieves minimum residual disease (MRD) negative status; and (iii) after the subject achieving MRD negative status, the cancer progresses in the subject; and (b) administering the subsequent therapy to the subject, wherein the prior therapy and the subsequent therapy are of the same class of therapy.

Also provided herein is a method of selecting a subject having a cancer in which the cancer is re-sensitized to a class of therapy, comprising: (a) administering a T cell therapy to a subject having a cancer at a time when the subject has relapsed following treatment with, or is refractory to, a prior therapy for treating the cancer; and (b) selecting the subject for treatment with a subsequent therapy for treating the cancer, wherein the subject is selected for treatment with the subsequent therapy if: (i) following administration of the T cell therapy, the subject achieves minimum residual disease (MRD) negative status; and (ii) after the subject achieves MRD negative status, the cancer progresses in the subject, wherein the prior therapy and the subsequent therapy are of the same class of therapy. In some embodiments, the method further comprises (c) administering the subsequent therapy to the subject.

Also provided herein is a method of treating a cancer, comprising: (a) selecting a subject having a cancer for treatment with a subsequent therapy for treating the cancer, wherein the subject was previously administered a T cell therapy for treating the cancer and a prior therapy for treating the cancer, and wherein: (i) the subject was administered the T cell therapy at a time when the subject had relapsed following treatment with, or was refractory to, the prior therapy; (ii) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and (iii) following administration of the T cell therapy, cells of the cancer do not comprise at least one of the high risk features that the cells of the cancer comprised prior to administration of the T cell therapy; and (b) administering the subsequent therapy to the subject, wherein the prior therapy and the subsequent therapy are of the same class of therapy.

Also provided herein is a method of selecting a subject having a cancer in which the cancer is re-sensitized to a class of therapy, comprising: (a) administering a T cell therapy to a subject having a cancer at a time when the subject has relapsed following treatment with, or is refractory to, a prior therapy for treating the cancer; and (b) selecting the subject for treatment with a subsequent therapy for treating the cancer, wherein the subject is selected for treatment with the subsequent therapy if: (i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and (ii) following administration of the T cell therapy, cells of the cancer do not comprise at least one of the high risk features that the cells of the cancer comprised prior to administration of the T cell therapy, wherein the prior therapy and the subsequent therapy are of the same class of therapy. In some embodiments, the method further comprises (c) administering the subsequent therapy to the subject.

Also provided herein is a method of treating a cancer, comprising: (a) administering to a subject having a cancer a T cell therapy for treating the cancer at a time when the subject has relapsed following treatment with, or is refractory to, a prior therapy for treating the cancer; and (b) administering a subsequent therapy for treating the cancer to the subject, wherein the prior therapy and the subsequent therapy are of the same class of therapy.

Also provided herein is a method of re-sensitizing a cancer in a subject, comprising: (a) administering to a subject having a cancer a T cell therapy for treating the cancer at a time when the subject has relapsed following treatment with, or is refractory to, a prior therapy for treating the cancer; and (b) administering a subsequent therapy for treating the cancer to the subject, wherein the prior therapy and the subsequent therapy are of the same class of therapy.

In some embodiments, the method further comprises, prior to (b), selecting the subject for treatment with the subsequent therapy, wherein the subject is selected for treatment with the subsequent therapy if: (i) following administration of the T cell therapy, the subject achieves minimum residual disease (MRD) negative status; and (ii) subsequent to the subject achieving MRD negative status, the cancer progresses in the subject.

In some embodiments, within about 1 month, about 2 months, about 3 months, about 6 months, or about 12 months of administration of the T cell therapy, the subject achieves MRD negative status.

In some embodiments, (i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and (ii) following administration of the T cell therapy, cells of the cancer do not comprise at least one of the high risk feature(s) that the cells of the cancer comprised prior to administration of the T cell therapy.

In some embodiments, within about 1 month, about 2 months, about 3 months, about 6 months, or about 12 months of administration of the T cell therapy, the cells of the cancer do not comprise at least one of the high risk feature(s) that the cells of the cancer comprised prior to administration of the T cell therapy.

In some embodiments, prior to administration of the T cell therapy, cells of the cancer comprise a CRBN mutation. In some embodiments, within about 1 month, about 2 months, about 3 months, about 6 months, or about 12 months of administration of the T cell therapy, cells of the cancer do not comprise a CRBN mutation. In some embodiments, the CRBN mutation is in exon 10 of the CRBN gene. In some embodiments, the CRBN mutation reduces or inhibits binding of thalidomide to the CRBN protein.

In some embodiments, the cancer is a B cell malignancy. In some embodiments, the cancer is a multiple myeloma (MM). In some embodiments, the MM is a relapsed/refractory (R/R) MM. In some embodiments, the cancer is a leukemia or a lymphoma. In some embodiments, the cancer is a leukemia. In some embodiments, the cancer is a lymphoma. In some embodiments, the leukemia or the lymphoma is selected from the group consisting of: acute lymphoblastic leukemia (ALL), adult ALL, chronic lymphoblastic leukemia (CLL), small lymphocytic lymphoma (SLL), non-Hodgkin lymphoma (NHL), and large B cell lymphoma (LBCL).

In some embodiments, the class of therapy is immunomodulatory drugs. In some embodiments, the prior therapy and the subsequent therapy both bind the cereblon (CRBN) protein. In some embodiments, the prior therapy and the subsequent therapy both induce degradation of Ailos and/or Ikaros. In some embodiments, the prior therapy and the subsequent therapy both induce degradation of Ailos. In some embodiments, the prior therapy and the subsequent therapy both induce degradation of Ikaros. In some embodiments, the prior therapy and the subsequent therapy both induce degradation of Ailos and Ikaros.

In some embodiments, the prior therapy is selected from among the group consisting of: thalidomide, lenalidomide, pomalidomide, iberdomide, CC-92480, CC-99282, CC-91633, and CC-90009. In some embodiments, the prior therapy is thalidomide. In some embodiments, the prior therapy is lenalidomide. In some embodiments, the prior therapy is pomalidomide. In some embodiments, the prior therapy is iberdomide. In some embodiments, the prior therapy is CC-92480. In some embodiments, the prior therapy is CC-99282. In some embodiments, the prior therapy is CC-91633. In some embodiments, the prior therapy is CC-90009. In some embodiments, the subsequent therapy is selected from among the group consisting of: thalidomide, lenalidomide, pomalidomide, iberdomide, CC-92480, CC-99282, CC-91633, and CC-90009. In some embodiments, the subsequent therapy is thalidomide. In some embodiments, the subsequent therapy is lenalidomide. In some embodiments, the subsequent therapy is pomalidomide. In some embodiments, the subsequent therapy is iberdomide. In some embodiments, the subsequent therapy is CC-92480. In some embodiments, the subsequent therapy is CC-99282. In some embodiments, the subsequent therapy is CC-91633. In some embodiments, the subsequent therapy is CC-90009.

In some embodiments, the class of therapy is proteasome inhibitors. In some embodiments, the prior therapy is selected from among the group consisting of: bortezomib, carfilzomib and ixazomib. In some embodiments, the prior therapy is bortezomib. In some embodiments, the prior therapy is carfilzomib. In some embodiments, the prior therapy is ixazomib. In some embodiments, the subsequent therapy is selected from among the group consisting of: bortezomib, carfilzomib and ixazomib. In some embodiments, the subsequent therapy is bortezomib. In some embodiments, the subsequent therapy is carfilzomib. In some embodiments, the subsequent therapy is ixazomib.

In some embodiments, the class of therapy is anti-CD38 antibodies. In some embodiments, the prior therapy is daratumumab or isatuximab. In some embodiments, the prior therapy is daratumumab. In some embodiments, the prior therapy is isatuximab. In some embodiments, the subsequent therapy is daratumumab or isatuximab. In some embodiments, the subsequent therapy is daratumumab. In some embodiments, the subsequent therapy is isatuximab.

In some embodiments, the class of therapy is inhibitors of Bruton's tyrosine kinase (BTK). In some embodiments, the prior therapy is selected from among the group consisting of: ibrutinib, acalabrutinib, zanubrutinib, evobrutinib, tirabrutinib, and SNS-062. In some embodiments, the prior therapy is ibrutinib. In some embodiments, the prior therapy is acalabrutinib. In some embodiments, the prior therapy is zanubrutinib. In some embodiments, the prior therapy is evobrutinib. In some embodiments, the prior therapy is tirabrutinib. In some embodiments, the prior therapy is SNS-062. In some embodiments, the subsequent therapy is selected from among the group consisting of: ibrutinib, acalabrutinib, zanubrutinib, evobrutinib, tirabrutinib, and SNS-062. In some embodiments, the subsequent therapy is ibrutinib. In some embodiments, the subsequent therapy is acalabrutinib. In some embodiments, the subsequent therapy is zanubrutinib. In some embodiments, the subsequent therapy is evobrutinib. In some embodiments, the subsequent therapy is tirabrutinib. In some embodiments, the subsequent therapy is SNS-062.

In some embodiments, the class of therapy is inhibitors of BCL-2. In some embodiments, the prior therapy is selected from among the group consisting of: venetoclax, navitoclax, ABT737, maritoclax, obatoclax, and clitocine. In some embodiments, the prior therapy is venetoclax. In some embodiments, the prior therapy is navitoclax. In some embodiments, the prior therapy is ABT737. In some embodiments, the prior therapy is maritoclax. In some embodiments, the prior therapy is obatoclax. In some embodiments, the prior therapy is clitocine. In some embodiments, the subsequent therapy is venetoclax. In some embodiments, the subsequent therapy is navitoclax. In some embodiments, the subsequent therapy is ABT737. In some embodiments, the subsequent therapy is maritoclax. In some embodiments, the subsequent therapy is obatoclax. In some embodiments, the subsequent therapy is clitocine.

In some embodiments, the subsequent therapy is a maintenance therapy.

In some embodiments, the T cell therapy comprises a dose of T cells expressing a recombinant receptor. In some embodiments, the recombinant receptor is a T cell receptor (TCR). In some embodiments, the recombinant receptor is a chimeric antigen receptor (CAR).

In some embodiments, the CAR comprises an extracellular antigen binding domain that binds to the antigen, a transmembrane domain, and an intracellular signaling region. In some embodiments, the intracellular signaling region comprises an intracellular signaling domain of a CD3-zeta (CD3ζ) chain and a costimulatory signaling region. In some embodiments, the costimulatory signaling region comprises an intracellular signaling domain of CD28, 4-1BB, or ICOS. In some embodiments, the costimulatory signaling region comprises an intracellular signaling domain of CD28. In some embodiments, the costimulatory signaling region comprises an intracellular signaling domain of 4-1BB. In some embodiments, the costimulatory signaling region comprises an intracellular signaling domain of ICOS.

In some embodiments, the transmembrane domain is or comprises a transmembrane domain from CD28 or CD8. In some embodiments, the transmembrane domain is or comprises a transmembrane domain from CD28. In some embodiments, the transmembrane domain is or comprises a transmembrane domain from CD8. In some embodiments, the transmembrane domain is or comprises a transmembrane domain from human CD28 or CD8. In some embodiments, the transmembrane domain is or comprises a transmembrane domain from human CD28. In some embodiments, the transmembrane domain is or comprises a transmembrane domain from human CD8. In some embodiments, the CAR further comprises an extracellular spacer between the extracellular antigen binding domain and the transmembrane domain. In some embodiments, the spacer is from CD8. In some embodiments, the spacer is a CD8α hinge. In some embodiments, the transmembrane domain and the spacer are from CD8.

In some embodiments, the extracellular antigen binding domain binds to B cell maturation antigen (BCMA). In some embodiments, the extracellular antigen-binding domain comprises a variable heavy chain (V_{H}) region. In some embodiments, the extracellular antigen-binding domain comprises a variable heavy chain (V_{H}) region and a variable light chain (V_{L}) region. In some embodiments, the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively; or the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively. In some embodiments, the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively. In some embodiments, the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively.

In some embodiments, the V_{H} region comprises an amino acid sequence set forth in SEQ ID NO: 18 and the V_{L} region comprises the amino acid sequence set forth in SEQ ID NO: 19; or the V_{H} region comprises an amino acid sequence set forth in SEQ ID NO: 24, and the V_{L} region comprises the amino acid sequence set forth in SEQ ID NO: 25. In some embodiments, the V_{H} region comprises an amino acid sequence set forth in SEQ ID NO: 18 and the V_{L} region comprises the amino acid sequence set forth in SEQ ID NO: 19. In some embodiments, the V_{H} region comprises an amino acid sequence set forth in SEQ ID NO: 24, and the V_{L} region comprises the amino acid sequence set forth in SEQ ID NO: 25. In some embodiments, the extracellular antigen-binding domain is a single chain variable fragment (scFv). In some embodiments, the scFv comprises the amino acid sequence set forth in SEQ ID NO: 213 or SEQ ID NO: 188. In some embodiments, the scFv comprises the amino acid sequence set forth in SEQ ID NO: 213. In some embodiments, the scFv comprises the amino acid sequence set forth in SEQ ID NO: 188. In some embodiments, the CAR comprises the amino acid sequence set forth in SEQ ID NO: 116 or SEQ ID NO: 124. In some embodiments, the CAR comprises the amino acid sequence set forth in SEQ ID NO: 116. In some embodiments, the CAR comprises the amino acid sequence set forth in SEQ ID NO: 124. In some embodiments, the CAR is encoded by the polynucleotide sequence set forth in SEQ ID NO: 214.

In some embodiments, the dose of T cells comprises: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells. In some embodiments, the dose of T cells comprises idecabtagene vicleucel cells.

In some embodiments, the extracellular antigen binding domain binds to G protein-coupled receptor, class C group 5 member D (GPRC5D).

In some embodiments, the extracellular antigen binding domain binds to CD19. In some embodiments, the extracellular antigen-binding domain comprises a variable heavy chain (V_{H}) region. In some embodiments, the extracellular antigen-binding domain comprises a variable heavy chain (V_{H}) region and a variable light chain (V_{L}) region. In some embodiments, the V_{H} region comprises a CDR-H1, a CDR-H2, and a CDR-H3 comprising the amino acid sequences set forth in SEQ ID NOS: 251, 252, and 253, respectively; and the V_{L} region comprises a CDR-L1, a CDR-L2, and a CDR-L3 comprising the amino acid sequences set forth in SEQ ID NOS: 248, 249, and 250, respectively; or the V_{H} region comprises a CDR-H1, a CDR-H2, and a CDR-H3 comprising the amino acid sequences set forth in SEQ ID NOS: 260, 261, and 262, respectively; and the V_{L} region comprises a CDR-L1, a CDR-L2, and a CDR-L3 comprising the amino acid sequences set forth in SEQ ID NOS: 257, 258, and 259, respectively. In some embodiments, the V_{H} region comprises a CDR-H1, a CDR-H2, and a CDR-H3 comprising the amino acid sequences set forth in SEQ ID NOS: 251, 252, and 253, respectively; and the V_{L} region comprises a CDR-L1, a CDR-L2, and a CDR-L3 comprising the amino acid sequences set forth in SEQ ID NOS: 248, 249, and 250, respectively. In some embodiments, the V_{H} region comprises a CDR-H1, a CDR-H2, and a CDR-H3 comprising the amino acid sequences set forth in SEQ ID NOS: 260, 261, and 262, respectively; and the V_{L} region comprises a CDR-L1, a CDR-L2, and a CDR-L3 comprising the amino acid sequences set forth in SEQ ID NOS: 257, 258, and 259, respectively. In some embodiments, the V_{H} region comprises an amino acid sequence set forth in SEQ ID NO: 254 and the V_{L} region comprises the amino acid sequence set forth in SEQ ID NO: 255; or the V_{H} region comprises an amino acid sequence set forth in SEQ ID NO: 263 and the V_{L} region comprises the amino acid sequence set forth in SEQ ID NO: 264. In some embodiments, the V_{H} region comprises an amino acid sequence set forth in SEQ ID NO: 254 and the V_{L} region comprises the amino acid sequence set forth in SEQ ID NO: 255. In some embodiments, the V_{H} region comprises an amino acid sequence set forth in SEQ ID NO: 263 and the V_{L} region comprises the amino acid sequence set forth in SEQ ID NO: 264. In some embodiments, the extracellular antigen-binding domain is a single chain variable fragment (scFv). In some embodiments, the scFv comprises the amino acid sequence set forth in SEQ ID NO: 256 or SEQ ID NO: 265. In some embodiments, the scFv comprises the amino acid sequence set forth in SEQ ID NO: 256. In some embodiments, the scFv comprises the amino acid sequence set forth in SEQ ID NO: 265.

In some embodiments, the dose of T cells comprises: lisocabtagene maraleucel cells; tisagenlecleucel cells; axicabtagene ciloleucel cells; or brexucabtagene autoleucel cells. In some embodiments, the dose of T cells comprises lisocabtagene maraleucel cells.

In some embodiments, the dose of T cells comprises CD3⁺ CAR-expressing T cells. In some embodiments, the dose of T cells comprises a combination of CD4⁺ CAR-expressing T cells and CD8+ CAR-expressing T cells. In some embodiments, the ratio of CD4⁺ CAR-expressing T cells to CD8⁺ CAR-expressing T cells in the dose of T cells is approximately 1:1 or is between approximately 1:3 and approximately 3:1. In some embodiments, the ratio of CD4⁺ CAR-expressing T cells to CD8⁺ CAR-expressing T cells in the dose of T cells is approximately 1:1. In some embodiments, the ratio of CD4⁺ CAR-expressing T cells to CD8⁺ CAR-expressing T cells in the dose of T cells is between approximately 1:3 and approximately 3:1.

In some embodiments, in the dose of T cells, the percentage of naive-like T cells and/or central memory T cells is greater than or greater than about 60% of the total T cells in the dose. In some embodiments, in the dose of T cells, the percentage of naive-like T cells and/or central memory T cells is greater than or greater than about 65%, 70%, 80%, 90% or 95% of the total T cells in the dose. In some embodiments, in the dose of T cells, the percentage of naive-like T cells and/or central memory T cells is greater than or greater than about 40% of the total CD4+ T cells in the dose. In some embodiments, in the dose of T cells, the percentage of naive-like T cells and/or central memory T cells is greater than or greater than about 50%, 60%, 70%, 80%, 90% or 95% of the total CD4+ T cells in the dose. In some embodiments, in the dose of T cells, the percentage of naive-like T cells and/or central memory T cells is greater than or greater than about 40% of the total CD8+ T cells in the dose. In some embodiments, in the dose of T cells, the percentage of naive-like T cells and/or central memory T cells is greater than or greater than about 50%, 60%, 70%, 80%, 90% or 95% of total CD8+ T cells in the dose. In some embodiments, the naive-like T cells are CCR7+CD45RA+, CD27+CCR7+, or CD62L−CCR7+. In some embodiments, the naive-like T cells are CCR7+CD45RA+. In some embodiments, the naive-like T cells are CD27+CCR7+. In some embodiments, the naive-like T cells are CD62L−CCR7+.

In some embodiments, the dose of T cells comprises between about 0.5×10⁶ and about 6×10⁸ CAR-positive T cells. In some embodiments, the dose of T cells comprises between about 1×10⁸ and about 6×10⁸ CAR-positive T cells. In some embodiments, the dose of T cells comprises between about 1.5×10⁸ and about 4.5×10⁸ CAR-positive T cells. In some embodiments, the dose of T cells comprises about 1.5×10⁸, 3×10⁸, or about 4.5×10⁸ CAR-positive T cells. In some embodiments, the dose of T cells comprises between about 0.5×10⁶ and about 10×10⁶ CAR-positive T cells.

In some embodiments, the cells of the dose of T cells were obtained from the subject. In some embodiments, the cells of the dose of T cells are autologous to the subject. In some embodiments, the cells of the dose of T cells are allogeneic to the subject.

In some embodiments, the T cell therapy comprises a T cell engager (TCE). In some embodiments, the TCE is selected from among the group consisting of: a bispecific T cell engager (BiTE), a checkpoint-inhibitory T cell engager (CiTE), a simultaneous multiple interaction T cell engagers (SMITE), and BiTE-expressing CAR T cells (CART.BiTE cells). In some embodiments, the TCE is a bispecific T cell engager (BiTE). In some embodiments, the TCE is a checkpoint-inhibitory T cell engager (CiTE). In some embodiments, the TCE is a simultaneous multiple interaction T cell engagers (SMITE). In some embodiments, the TCE is BiTE-expressing CAR T cells (CART.BiTE cells).

In some embodiments, the method comprises, prior to administration of the T cell therapy, administering a lymphodepleting therapy to the subject. In some embodiments, the lymphodepleting therapy is completed between 2 and 7 days before the initiation of administration of the T cell therapy. In some embodiments, the lymphodepleting therapy comprises the administration of fludarabine and/or cyclophosphamide. In some embodiments, the lymphodepleting therapy comprises the administration of fludarabine. In some embodiments, the lymphodepleting therapy comprises the administration of cyclophosphamide. In some embodiments, the lymphodepleting therapy comprises the administration of fludarabine and cyclophosphamide. In some embodiments, the lymphodepleting therapy comprises administration of cyclophosphamide at about 200-400 mg/m². In some embodiments, the lymphodepleting therapy comprises administration of cyclophosphamide at or about 300 mg/m². In some embodiments, the lymphodepleting therapy comprises administration of fludarabine at about 20-40 mg/m². In some embodiments, the lymphodepleting therapy comprises administration of fludarabine at about 30 mg/m². In some embodiments, the lymphodepleting therapy comprises administration of fludarabine at about 20-40 mg/m² and fludarabine at about 30 mg/m². In some embodiments, cyclophosphamide is administered daily for 2-4 days. In some embodiments, cyclophosphamide is administered daily for 3 days. In some embodiments, fludarabine is administered daily for 2-4 days. In some embodiments, fludarabine is administered daily for 3 days. In some embodiments, cyclophosphamide and fludarabine are administered daily for 2-4 days. In some embodiments, cyclophosphamide and fludarabine are administered daily for 3 days. In some embodiments, the lymphodepleting therapy comprises administration of cyclophosphamide at about 500 mg/m². In some embodiments, the lymphodepleting therapy comprises administration of cyclophosphamide at or about 300 mg/m² and fludarabine at about 30 mg/m² daily for 3 days. In some embodiments, the lymphodepleting therapy comprises administration of cyclophosphamide at or about 500 mg/m² and fludarabine at about 30 mg/m² daily for 3 days.

### BRIEF DESCRIPTION OF THE FIGURES

FIG. 1A shows the pre-treatment prevalence of high risk (HR) tumor features in subjects with relapsed/refractory multiple myeloma who were treated with an exemplary anti-BCMA CAR T cell therapy. Minimum residual disease at 3 months: NE: Not evaluated; I: Indeterminate; P: Positive; N: Negative.

FIG. 1B shows the post-treatment prevalence of HR tumor features in subjects with relapsed/refractory multiple myeloma who were treated with an exemplary anti-BCMA CAR T cell therapy. Minimum residual disease at 3 months: NE: Not evaluated; I: Indeterminate; P: Positive; N: Negative.

### DETAILED DESCRIPTION

Provided herein are therapies involving administration of a T cell therapy to a subject having a cancer. In some aspects, the subject has relapsed following treatment with, or is refractory to, a prior therapy for treating the cancer. In some aspects, following administration of the T cell therapy (e.g. CAR T cells or a TCE), the subject is administered a subsequent therapy for treating the cancer, wherein the prior therapy and the subsequent therapy are of the same class of therapy. In some aspects, the prior therapy and the subsequent therapy are both an immunomodulatory drug, such as a structural or functional analog or derivative of thalidomide and/or an inhibitor of E3-ubiquitin ligase. In some aspects, prior to administration of the T cell, the cancer is resistant to treatment with the class of therapy (e.g., immunomodulatory drugs), and administration of the T cell therapy re-sensitizes the cancer to treatment with the class of therapy. In some cases, the cancer is resistant to treatment with immunomodulatory drugs due to a genetic mutation. For example, the cancer may be resistant to treatment with immunomodulatory drugs (e.g., IMiDs® or CELMoDs®) prior to administration of the T cell therapy, such as by acquisition of a resistance-conferring mutation (e.g., in the cereblon [CRBN] gene), and following administration of the T cell therapy, the cancer is sensitive to treatment with immunomodulatory drugs. Thus, in some aspects, the provided methods allow a subject to be treated with a class of therapy to which the cancer was previously resistant. In some embodiments, the therapy involves administration of the T cell therapy, such as a composition including cells for adoptive cell therapy, e.g., such as a T cell therapy (e.g. CAR-expressing T cells), and administration of a subsequent therapy comprising an immunomodulatory drug, such as a structural or functional analog of thalidomide and/or an inhibitor of E3-ubiquitin ligase.

In some aspects, available approaches for treatment of cancer, such as multiple myeloma (e.g. relapsed and refractory MM) are complex and may not always be entirely satisfactory. Patients with relapsed or refractory MM have poor outcomes with currently available therapies. Relapsed and refractory MM often does not respond to further treatments and usually progresses within 2 to 4 months. (Chari et al., N Engl J Med (2019) 381:727-38 and Lonial et al., Lancet Oncol (2020) 21:207-21). In some aspects, choosing a treatment regimen can depend on numerous factors including drug availability, response to prior therapy, aggressiveness of the relapse, eligibility for autologous stem cell transplantation (ASCT), and whether the relapse occurred on or off therapy. In some aspects, MM results in relapses and remissions, and existing regimens in some cases can result in relapse and/or toxicity from the treatment. In some cases, subjects with particularly aggressive disease, such as subjects that have persistent or relapsed disease after various therapies, subjects with a high disease burden, such as a high tumor burden, high risk tumor features, and/or subjects with high risk disease (i.e. high risk cytogenetics), can be particularly difficult to treat, and responses to certain therapies in these subjects can be poor or have a short duration. In some cases, subjects who have been heavily pre-treated, e.g., subjects who have relapsed after several different prior lines of therapy, can exhibit a low response rate and/or high incidence of adverse events.

In particular, outcomes for patients with relapsed and/or refractory multiple myeloma (R/R MM) with previous exposure to immunomodulatory agents, proteasome inhibitors (PIs), and anti-CD38 antibodies are poor. (Chari et al., N Engl J Med (2019) 381:727-38; Lonial et al., Poster presentation at the European Hematology Association (EHA) Virtual Meeting 2021: Abstract EP970; and Richardson et al., J Clin Oncol (2021) 39:757-67). Multiple myeloma patients relapse and become refractory to treatment regimens, commonly due to drug resistance. In particular, multiple myeloma tumors can exhibit increasing prevalence of high-risk or resistance (HR) features with each successive relapse, leading to poorer outcomes in late-line patients.

Resistance to agents such as proteasome inhibitors and IMiDs® (i.e., thalidomide and its derivatives such as pomalidomide and lenalidomide) has been observed, and patients who become resistant to first generation IMiDs® and proteasome inhibitors have significantly worse outcomes (Pinto et al., Cancers (Basel) (2020) 12(2):407). In this way, acquired drug resistance has limited the clinical application of treatments such as proteasome inhibitors and IMiDs®. A primary mechanism underlying resistance to IMiDs® and their newer derivatives CRBN E3 ligase modulators (CELMoDs®) is genetic mutations in the CRBN gene locus. High expression of CRBN has been reported to correlate with improved clinical responses to IMiDs® in multiple myeloma patients, whereas patients resistant to IMiDs® frequently exhibit CRBN mutations. Approximately one-third of relapsed/refractory multiple myeloma patients treated with IMiDs® are reported to have direct CRBN genetic alternations, making it the single most clinically significant contributor to clinical resistance. Such mutations include point mutations, copy loss/structural variations, and an exon 10 splice variant transcript (Wang et al., Biomarker Res (2021) 9:43; Gooding et al. Blood (2021) 137(2):232-37). Thalidomide and its derivatives such as lenalidomide and pomalidomide bind to the CRBN protein at the site of a hydrophobic binding pocket comprised of three tryptophan residues (W380, W386, and W400), which map to CRBN C-terminus exons 10-11 (Neri et al., Blood (2016) 128(22):120). A splice variant of CRBN lacking exon 10, which deletes the thalidomide-binding region, was found to be significantly increased in pomalidomide-refractory patients, and correlates with significantly reduced progression free survival (PFS) (Gooding et al. Blood (2021) 137(2):232-37).

The provided methods are based on observations that the types and numbers of high risk (HR) tumor features, including CRBN mutation(s), are altered following administration of a T cell therapy (e.g., anti-BCMA CAR T cells) to subjects having relapsed/refractory multiple myeloma. For instance, it is observed herein that among samples from subjects having a HR CRBN feature at pretreatment, 33% had no HR feature at disease progression (PD), 17% retained a HR CRBN feature at PD, and 50% lost a HR CRBN feature. Thus, the findings herein indicate that treatment with a T cell therapy (e.g. CAR T cells or a TCE) may change the HR tumor feature landscape of multiple myeloma cells, such that a subject who was previously resistant to treatment with a class of therapy (e.g., immunomodulatory drugs such as IMiDs® or CELMoDs®) may be sensitive to treatment with such drugs following administration of a T cell therapy, including in subjects who achieve minimum residual disease (MRD) negative status following administration of the T cell therapy.

Other HR tumor features include amplification of the long arm of chromosome 1 (amp1q), the MDMS8 gene signature, t(4;14); biallelic p53 inactivation, and high cancer clonal fraction del17p. Copy number gain of 1q21 is among the most common chromosomal aberrations in multiple myeloma, observed in 28-44% of patients at diagnosis. Genes on chromosome 1q, the expression of which can be upregulated with amp1q, are associated with aggressive MM phenotypes. Retrospective analyses show that patients with amp1q have shorter durations of PFS and OS than those without (Bisht et al., Expert Rev. Hematol. (2021) 14(12):1099-14). The MDMS8 gene signature represents a broad genomic loss driving dysregulation of various transcription programs affecting DNA repair and cell cycle/mitotic processes, and has been associated with poor clinical outcomes (Ortiz-Estevez et al., BMC Medical Genomics (2021) 14:295). t(4;14) leads to deregulation of fibroblast growth factor receptor 3 (FGFR3) and multiple myeloma SET domain (MMSET), and is associated with impaired PFS and overall survival (OS) (Sonneveld et al., Blood (2016) 127(24):2955-62). Biallelic p53 inactivation has been observed in 2-4% of newly diagnosed multiple myeloma patients, and is associated with median survival of less than two years (Munawar et al., Blood (2019) 134(10):836-40). Alterations to tumor protein 53 (tp53), including deletions in chromosome 17p (del17p) are associated with poor outcomes in patients with multiple myeloma. Further, increases in the cancer clonal fraction of del17p are observed to associate with shorter survival in newly diagnosed multiple myeloma patients (Thakurta et al., Blood (2019) 133(11):1217-21). For example, clonal content of del17p of greater than or equal to 55% is considered to identify patients with worse PFS and OS outcomes.

In some embodiments, treatment with a T cell therapy, (e.g., CAR T cells) allows for a subject to be subsequently treated with a class of therapy upon disease progression (PD), despite that the subject had previously relapsed following, or was refractory to, the same class of therapy prior to treatment with the T cell therapy. In some embodiments, the ability to effectively treat a subject with the subsequent therapy is due to the loss of one or more HR tumor features following treatment with the T cell therapy. In some embodiments, the class of therapy is proteasome inhibitors. In some embodiments, the class of therapy is immunomodulatory drugs (e.g., IMiDs® and CELMoDs®). In some embodiments, the class of therapy is anti-CD38 antibodies. In some embodiments, the class of therapy is inhibitors of Bruton's tyrosine kinase (BTK). In some embodiments, the class of therapy is inhibitors of BCL-2.

In some embodiments, the methods can be used for treating a cancer, e.g. a B cell malignancy or hematological malignancy, and in particular such diseases, conditions or malignancies in which responses, e.g. complete response, to treatment with the subsequent therapy alone is relatively low compared to treatment also including a T cell therapy (e.g. CAR-expressing T cells). In some embodiments, the cancer is a myeloma, leukemia or lymphoma. In some embodiments, the cancer is multiple myeloma. In some embodiments, the cancer is relapsed/refractory multiple myeloma.

In some embodiments, the methods provided herein are for use in a subject having a cancer in which prior to initiation of administration of the T cell therapy, the subject has relapsed following treatment with a prior therapy, and following administration of the T cell therapy, the subject is treated with a subsequent therapy, wherein the prior therapy and the subsequent therapy are of the same class of therapy (e.g., immunomodulatory drugs). In some embodiments, subjects that have previously relapsed following treatment with, or become refractory to, an immunomodulatory drug, such as a structural or functional analog or derivative of thalidomide and/or an inhibitor of E3 ubiquitin ligase, e.g. lenalidomide, and treated with a subsequent therapy that is also an immunomodulatory drug. In some embodiments, the methods provided herein are for use in a subject having a cancer, in which the immunomodulatory drug administered without prior T cell therapy is insufficient to ameliorate, reduce or prevent the disease or condition in the subject or a symptom or outcome thereof.

Also provided are methods for engineering, preparing, and producing the T cell therapy, compositions containing the T cell therapy and/or subsequent therapy (e.g., immunomodulatory drug), and kits and devices containing and for using, producing and administering the T cell therapy and/or subsequent therapy, such as in accord with the provided methods.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### I. Methods of Treatment and Uses

Provided are methods and uses of a T cell therapy, such as CAR T cells or T cell engagers (TCEs). Also provided are methods and uses of (a) a T cell therapy, such as CAR T cells or T cell engagers (TCEs); and (b) a subsequent therapy, for the treatment of subjects with a cancer.

In some embodiments, the subject has relapsed following treatment with, or is refractory to, a prior therapy for treating the cancer. In some embodiments, the subject is selected for treatment with the subsequent therapy if, following administration of the T cell therapy, the subject achieves minimum residual disease (MRD) negative status, and subsequent to the patient achieving MRD negative status, the cancer progresses in the subject. In some embodiments, the subject is selected for treatment with the subsequent therapy if, prior to administration of the T cell therapy, cells of the cancer comprise one or more high risk feature(s) selected from among the group consisting of amplification of the long arm of chromosome 1, MDMS8 gene signature, a cereblon (CRBN) mutation, biallelic p53 inactivation, high cancer clonal fraction del17p, and t(4,14), and following administration of the T cell therapy, cells of the cancer do not comprise at least one of the high risk features that the cancer comprised prior to administration of the T cell therapy. In some embodiments, prior to administration of the T cell therapy, cells of the cancer comprise a CRBN mutation. In some embodiments, following administration of the T cell therapy, cells of the cancer do not comprises a CRBN mutation.

In some embodiments, the cancer is a B cell malignancy, such as multiple myeloma. In some embodiments, the cancer is multiple myeloma. In some embodiments, the multiple myeloma is a relapsed or refractory multiple myeloma (R/R MM). In some embodiments, the cancer is a leukemia or a lymphoma. In some embodiments, the cancer is a leukemia. In some embodiments, the cancer is a lymphoma.

In some embodiments, the prior therapy and the subsequent therapy are of the same class of therapy (e.g., immunomodulatory drugs, proteasome inhibitors, anti-CD38 antibodies, BTK inhibitors, or BCL-2 inhibitors). In some embodiments, the class of therapy is immunomodulatory drugs. In some embodiments, the class of therapy is proteasome inhibitors. In some embodiments, the class of therapy is anti-CD38 antibodies. In some embodiments, the class of therapy is BTK inhibitors. In some embodiments, the class of therapy is BCL-2 inhibitors.

In some embodiments, the prior therapy and the subsequent therapy bind the CRBN protein. In some embodiments, the class of therapy is immunomodulatory drugs, such as IMIDs™ or CELMoDs™. In some embodiments, the prior therapy is an IMiD™. In some embodiments, the prior therapy is a CELMoD™. In some embodiments, the prior therapy is selected from among the group consisting of thalidomide, lenalidomide, pomalidomide, iberdomide, CC-92480, CC-99282, CC-91633, and CC-90009. In some embodiments, the subsequent therapy is an IMiD™ or a CELMoD™. In some embodiments, the subsequent therapy is selected from among the group consisting of thalidomide, lenalidomide, pomalidomide, iberdomide, CC-92480, CC-99282, CC-91633, and CC-90009. In some embodiments, regimens for administering the immunomodulatory drug can include any as described Section I.B.1.

In some embodiments, the class of therapy is proteasome inhibitors. In some embodiments, the prior therapy is selected from among the group consisting of: bortezomib, carfilzomib and ixazomib. In some embodiments, the subsequent therapy is selected from among the group consisting of: bortezomib, carfilzomib and ixazomib. In some embodiments, the dosage regimens for administering the proteasome inhibitor can include any known in the art.

In some embodiments, the class of therapy is anti-CD38 antibodies. In some embodiments, the prior therapy is daratumumab or isatuximab. In some embodiments, the subsequent therapy is daratumumab or isatuximab. In some embodiments, the dosage regimens for administering the anti-CD38 antibody can include any known in the art.

In some embodiments, the class of therapy is inhibitors of Bruton's tyrosine kinase (BTK). In some embodiments, the prior therapy is selected from among the group consisting of ibrutinib, acalabrutinib, zanubrutinib, evobrutinib, tirabrutinib, and SNS-062. In some embodiments, the subsequent therapy is selected from among the group consisting of ibrutinib, acalabrutinib, zanubrutinib, evobrutinib, tirabrutinib, and SNS-062. In some embodiments, the dosage regimens for administering the BTK inhibitor can include any known in the art.

In some embodiments, the class of therapy is inhibitors of BCL-2. In some embodiments, the prior therapy is selected from among the group consisting of venetoclax, navitoclax, ABT737, maritoclax, obatoclax, and clitocine. In some embodiments, the subsequent therapy is selected from among the group consisting of venetoclax, navitoclax, ABT737, maritoclax, obatoclax, and clitocine. In some embodiments, the dosage regimens for administering the BCL-2 inhibitor can include any known in the art.

In some embodiments, the subsequent therapy is maintenance therapy of any of the subsequent therapy classes or therapies described herein. Appropriate doses for maintenance therapies are known in the art, including for multiple myeloma maintenance therapies (Ludwig et al. Blood (2012) 119(13):3003-15).

In some embodiments, the subsequent therapy is maintenance therapy of lenalidomide. In some embodiments, a maintenance therapy of thalidomide comprises administration of a dose of between about 50 mg and about 400 mg mg per day until disease progression (PD). In some embodiments, a maintenance therapy of thalidomide comprises administration of a dose of about 50 mg per day until PD. In some embodiments, a maintenance therapy of thalidomide comprises administration of a dose of about 100 mg per day until PD. In some embodiments, a maintenance therapy of thalidomide comprises administration of a dose of about 200 mg per day until PD. In some embodiments, a maintenance therapy of thalidomide comprises administration of a dose of about 400 mg per day until PD.

In some embodiments, the subsequent therapy is maintenance therapy of thalidomide. In some embodiments, a maintenance therapy of lenalidomide comprises administration of a dose of 10 mg per day for a cycle of 28 days, with repeated 28-day cycles. In some embodiments, if the 10 mg per day dose is tolerated by the subject, the dose can be increased to 15 mg per day after three 28-day cycles.

In some embodiments, the subsequent therapy is maintenance therapy of bortezomib. In some embodiments, a maintenance therapy of bortezomib comprises administration of a dose of 1.3 mg/m² twice per week, optionally administered subcutaneously alone or with dexamethasone. In some embodiments, a maintenance therapy of bortezomib comprises administration of a dose of 1.3 mg/m² once per week.

In some embodiments, the maintenance therapy is administered to the subject for between about one year and about three years following administration of the T cell therapy. In some embodiments, the maintenance therapy is administered to the subject for between about two years and about three years following administration of the T cell therapy.

In some embodiments, the T cell therapy is adoptive cell therapy. In some embodiments, the T cell therapy is or comprises a tumor infiltrating lymphocytic (TIL) therapy, a transgenic TCR therapy or a recombinant-receptor expressing cell therapy (optionally T cell therapy), which optionally is a chimeric antigen receptor (CAR)-expressing cell therapy. In some embodiments, the T cell therapy is a B cell targeted therapy. In some embodiments, the therapy targets B cell maturation antigen (BCMA). In some embodiments, the cells and dosage regimens for administering the T cell therapy can include any as described in Section IC.1.d.

In some embodiments, the T cell therapy is T cell engaging (TCE) therapy. In some embodiments, the TCE therapy is or comprises a bispecific T cell engager (BiTE), a checkpoint-inhibitory T cell engager (CiTE), a simultaneous multiple interaction T cell engagers (SMITE), or a BiTE-expressing CAR T cells (CART.BiTE cells). In some embodiments, the TCE engaging therapy targets an antigen expressed by a T cell (e.g., CD3) and an antigen expressed by cancer cells (e.g., BCMA).

In some embodiments, any of the prior therapy, the T cell therapy (e.g. CAR-expressing T cells), and the subsequent therapy are provided as pharmaceutical compositions for administration to the subject. In some embodiments, the pharmaceutical compositions contain therapeutically effective amounts of one of the prior therapy, the T cell therapy, and the subsequent therapy.

In some embodiments, the T cell therapy, including engineered cells, such as CAR-T cell therapy, is administered to a subject or patient having a disease or condition to be treated (e.g. cancer) or at risk for having the disease or condition (e.g. cancer). In some aspects, the methods treat, e.g., ameliorate one or more symptom of, the disease or condition, such as by lessening tumor burden in a cancer expressing an antigen recognized by the immunotherapy or immunotherapeutic agent, e.g. recognized by an engineered T cell. In some embodiments, the T cell therapy re-sensitizes cells of the cancer to the class of therapy of the prior therapy. In some embodiments, following treatment with the T cell therapy, cells of the cancer are sensitized to a subsequent therapy, which is of the same class of therapy of the prior therapy.

In some embodiments, the disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein. In particular embodiments, the recombinant receptor expressed on engineered cells of a combination therapy, including a chimeric antigen receptor or transgenic TCR, specifically binds to an antigen associated with the disease or condition.

In some embodiments the cancer or proliferative disease expresses BCMA. In some embodiments, the provided methods employ a recombinant receptor-expressing T cell (e.g. CAR-T cell) that targets BCMA. In some embodiments, the provided methods employ a recombinant receptor-expressing T cell (e.g. CAR-T cell) that targets GPRC5D.

In some embodiments, the methods and uses include 1) administering to the subject a T cell therapy involving T cells expressing genetically engineered cell surface receptors (e.g., recombinant antigen receptor), which generally are chimeric receptors such as chimeric antigen receptors (CARs), directed against or targeting BCMA, at a time when the subject has relapsed following treatment with, or is refractor to a prior therapy, and 2) administering to the subject a subsequent therapy, wherein the prior therapy and the subsequent therapy are of the same class of therapy (e.g., immunomodulatory drugs).

The T cell therapy, e.g., including engineered cells expressing a recombinant receptor, such as a chimeric antigen receptor (CAR), and the subsequent therapy (e.g., an IMiD™ or CELMoD™) or compositions comprising the T cell therapy or the subsequent compound described herein are useful in a variety of therapeutic, diagnostic and prophylactic indications. For example, the combinations are useful in treating a variety of cancers in a subject. Such methods and uses include therapeutic methods and uses, for example, involving administration of the T cell therapy and subsequent therapy (e.g., immunomodulatory drugs) or compositions containing one or both, to a subject having a cancer. In some embodiments, the T cell therapy and the subsequent therapy and/or compositions containing one or both are administered in an effective amount to effect treatment of the cancer. Uses include uses of the T cell therapy and the subsequent therapy and/or compositions containing one or both in such methods and treatments, and in the preparation of a medicament in order to carry out such therapeutic methods. In some embodiments, the methods are carried out by administering the T cell therapy and/or the subsequent therapy, and/or compositions containing one or both, to the subject having or suspected of having the cancer. In some embodiments, the methods thereby treat the cancer in the subject.

Among the diseases to be treated is any cancer in which BCMA is specifically expressed and/or in which BCMA has been targeted for treatment. Cancers associated with BCMA expression include hematologic malignancies such as multiple myeloma, Waldenstrom macroglobulinemia, as well as both Hodgkin's and non-Hodgkin's lymphomas. See Coquery et al., Crit Rev Immunol., 2012, 32(4):287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19(8):2048-2060. In some embodiments, the disease or disorder associated with BCMA is a B cell-related disorder. In some embodiments, the disease or disorder associated with BCMA is one or more diseases or conditions from among glioblastoma, lymphomatoid granulomatosis, post-transplant lymphoproliferative disorder, an immunoregulatory disorder, heavy-chain disease, primary or immunocyte-associated amyloidosis, or monoclonal gammopathy of undetermined significance. In some embodiments, the disease or disorder associated with BCMA is an autoimmune disease or disorder. Such autoimmune diseases or disorder include, but are not limited to, systemic lupus erythematosus (SLE), lupus nephritis, inflammatory bowel disease, rheumatoid arthritis (e.g., juvenile rheumatoid arthritis), ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, polyarteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

Among the diseases, disorders or conditions associated with BCMA are cancers (e.g., a BCMA-expressing cancer), Cancers, e.g. BCMA-expressing cancers, that can be treated include, but are not limited to, neuroblastoma, renal cell carcinoma, colon cancer, colorectal cancer, breast cancer, epithelial squamous cell cancer, melanoma, myeloma (e.g., multiple myeloma), stomach cancer, brain cancer, lung cancer, pancreatic cancer, cervical cancer, ovarian cancer, liver cancer, bladder cancer, prostate cancer, testicular cancer, thyroid cancer, uterine cancer, adrenal cancer and head and neck cancer.

In certain diseases and conditions, BCMA is expressed on malignant cells and cancers. In some embodiments, the cancer (e.g., a BCMA-expressing cancer) is a B cell malignancy. In some embodiments, the cancer (e.g., a BCMA-expressing cancer) is a lymphoma, a leukemia, or a plasma cell malignancy. Lymphomas contemplated herein include, but are not limited to, Burkitt lymphoma (e.g., endemic Burkitt lymphoma or sporadic Burkitt lymphoma), non-Hodgkin's lymphoma (NHL), Hodgkin's lymphoma, Waldenstrom macroglobulinemia, follicular lymphoma, small non-cleaved cell lymphoma, mucosa-associated lymphatic tissue lymphoma (MALT), marginal zone lymphoma, splenic lymphoma, nodal monocytoid B cell lymphoma, immunoblastic lymphoma, large cell lymphoma, diffuse mixed cell lymphoma, pulmonary B cell angiocentric lymphoma, small lymphocytic lymphoma, primary mediastinal B cell lymphoma, lymphoplasmacytic lymphoma (LPL), or mantle cell lymphoma (MCL). Leukemias contemplated here, include, but are not limited to, chronic lymphocytic leukemia (CLL), plasma cell leukemia or acute lymphocytic leukemia (ALL). Also contemplated herein are plasma cell malignancies including, but not limited to, multiple myeloma (e.g., non-secretory multiple myeloma, smoldering multiple myeloma) or plasmacytoma. In some embodiments, the disease or condition is a plasmacytoma, such as extramedullary plasmacytoma. In some embodiments, the subject does not have a plasmacytoma, such as extramedullary plasmacytoma. In some embodiments the disease or condition is multiple myeloma (MM), such as relapsed and/or refractory multiple myeloma (R/R MM).

In some embodiments, the methods may identify a subject who has, is suspected to have, or is at risk for developing a BCMA-associated disease or disorder. Hence, provided are methods for identifying subjects with diseases or disorders associated with elevated BCMA expression and selecting them for treatment with a BCMA-directed T cell therapy (e.g. anti-BCMA CAR T cells).

In some aspects, for example, a subject may be screened for the presence of a disease or disorder associated with elevated BCMA expression, such as a BCMA-expressing cancer. In some embodiments, the methods include screening for or detecting the presence of a BCMA-associated disease, e.g. a tumor or a cancer, such as multiple myeloma. Thus, in some aspects, a sample may be obtained from a patient suspected of having a disease or disorder associated with elevated BCMA expression and assayed for the expression level of BCMA. In some aspects, a subject who tests positive for a BCMA-associated disease or disorder may be selected for treatment by the present methods, and may be administered a therapeutically effective amount of a BCMA-directed T cell therapy (e.g. anti-BCMA CAR T cells) or a pharmaceutical composition thereof as described herein.

In some aspects, a subject may be screened for the level of soluble BCMA (sBCMA), e.g., from a biological sample from the subject, such as the blood or serum. In some aspects, a subject may be screened for the level of sBCMA prior to treatment with the cell therapy. In some aspects, the methods include screening for or detecting the level or amount of sBCMA in a subject that has a disease or disorder associated with BCMA expression, e.g., a tumor or a cancer, such as multiple myeloma. In some aspects, a sample may be obtained from a patient suspected of having a disease or disorder associated with BCMA and assayed for the level or amount of sBCMA, for example, using an assay to detect soluble protein levels, such as an enzyme-linked immunosorbent assay (ELISA). In some aspects, in subjects having a multiple myeloma (MM), sBCMA levels can correlate with the proportion of plasma cells in bone marrow biopsies. In some aspects, in subjects having a multiple myeloma (MM), sBCMA levels can correlate with reduced response to treatment or shorter overall survival or progression free survival (see, e.g., Ghermezi et al., Haematologica 2017, 102(4): 785-795). In some aspects, a subject who exhibits low sBCMA levels may be selected for treatment by the present methods, and may be administered a therapeutically effective amount of a BCMA-directed T cell therapy (e.g. anti-BCMA CAR T cells) or a pharmaceutical composition thereof as described herein.

In some embodiments, the disease or condition associated with BCMA is one that has relapsed in the subject to one or more prior therapies for treating the disease and/or is one in which a subject has not responded to one or more other prior therapies for treating the disease and thus is refractory to treatment with the one or more prior therapies. In particular embodiments, the disease or condition is multiple myeloma that is a relapsed or refractory disease (hereinafter also called relapsed or refractory multiple myeloma or R/R multiple myeloma). In some embodiments, the subject has persistent or relapsed disease, e.g., following treatment with another BCMA-specific antibody and/or cells expressing a BCMA-targeting chimeric receptor and/or other therapy, including chemotherapy, radiation, and/or hematopoietic stem cell transplantation (HSCT), e.g., allogeneic HSCT or autologous HSCT. In some embodiments, the subject is resistant to or refractory to treatment, i.e. does not respond following treatment, with another BCMA-specific antibody and/or cells expressing a BCMA-targeting chimeric receptor and/or other therapy, In some embodiments, the administration of the T cell therapy (e.g. anti-BCMA CAR T cells) in the provided methods effectively treats the subject despite the subject having become resistant or refractory to another BCMA-targeted therapy.

In some embodiments, prior to the initiation of administration of the T cell therapy, the subject has received one or more prior therapies for treating the cancer, e.g. multiple myeloma. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has relapsed or is refractory to treatment with two or more prior therapies. In some embodiments, the subject has relapsed or is refractory to treatment with three or more prior therapies. In some embodiments, the subject has relapsed or is refractory to treatment with four or more prior therapies. In some embodiment, the one more prior therapy may include an autologous stem cell transplant (ASCT), an anti-CD38 antibody, such as daratumumab; an immunomodulatory agent or compounds that as thalidomide, lenalidomide or pomalidomide; a proteasome inhibitor such as bortezomib, carfilzomib or ixazomib; or two or more of any of the above. In some aspects, the subject has relapsed or has been refractory to the one or more prior therapies. For example, the subject has R/R multiple myeloma.

In some aspects, the prior therapies include treatment with autologous stem cell transplant (ASCT); an immunomodulatory agent; a proteasome inhibitor; and an anti-CD38 antibody; unless the subject was not a candidate for or was contraindicated for one or more of the therapies. In some aspects, the subject has relapsed or has been refractory to three or more prior therapies, including treatment with three or more therapies selected from (1) an autologous stem cell transplantation, (2) a proteasome inhibitor and an immunomodulatory agent, either alone or in combination, and (3) an anti-CD38 monoclonal antibody, as a part of a combination therapy or a monotherapy; unless the subject was not a candidate for or was contraindicated for one or more of the therapies. In some embodiments, the immunomodulatory agent is selected from among thalidomide, lenalidomide or pomalidomide. In some embodiments, the proteasome inhibitor is selected from among bortezomib, carfilzomib or ixazomib. In some embodiments, the anti-CD38 antibody is or comprises daratumumab. In some embodiments, the subject must have undergone at least 2 consecutive cycles of treatment for each regimen unless progressive disease was the best response to the regimen.

In some embodiments, the subject has relapsed following treatment with, or is refractory to, a prior therapy. In some embodiments, the prior therapy comprises an immunomodulatory drug. In some embodiments, the prior therapy comprises a proteasome inhibitor. In some embodiments, the prior therapy comprises an anti-CD38 antibody. In some embodiments, the prior therapy comprises an inhibitor of Bruton's tyrosine kinase (BTK). In some embodiments, the prior therapy comprises an inhibitor of BCL-2.

For the prevention or treatment of disease, the appropriate dosage of immunomodulatory compound (e.g., Compound A or Compound B) and/or immunotherapy, such as a T cell therapy (e.g. CAR-expressing T cells), may depend on the type of disease to be treated, the particular immunomodulatory compound, cells and/or recombinant receptors expressed on the cells, the severity and course of the disease, route of administration, whether the immunomodulatory compound and/or the T cell therapy are administered for preventive or therapeutic purposes, previous therapy, frequency of administration, the subject's clinical history and response to the cells, and the discretion of the attending physician. The compositions and cells are in some embodiments suitably administered to the subject at one time or over a series of treatments. Exemplary dosage regimens and schedules for the provided combination therapy are described.

In some embodiments, the T cell therapy and the immunomodulatory compound are administered as part of a further combination treatment, which can be administered simultaneously with or sequentially to, in any order, another therapeutic intervention. In some contexts, the T cell therapy, e.g. engineered T cells, such as CAR-expressing T cells, are co-administered with another therapy sufficiently close in time such that the T cell therapy enhances the effect of one or more additional therapeutic agents, or vice versa. In some embodiments, the cells are administered prior to the one or more additional therapeutic agents. In some embodiments, the T cell therapy, e.g. engineered T cells, such as CAR-expressing T cells, are administered after the one or more additional therapeutic agents. In some embodiments, the combination therapy methods further include a lymphodepleting therapy, such as administration of a chemotherapeutic agent. In some embodiments, the combination therapy further comprises administering another therapeutic agent, such as an anti-cancer agent, a checkpoint inhibitor, or another immune modulating agent. Uses include uses of the combination therapies in such methods and treatments, and uses of such compositions in the preparation of a medicament in order to carry out such combination therapy methods. In some embodiments, the methods and uses thereby treat the disease or condition or disorder, such as a cancer or proliferative disease, in the subject.

Prior to, during or following administration of the immunotherapy (e.g. T cell therapy, such as CAR-T cell therapy) and/or an immunomodulatory compound, the biological activity of the T cell therapy, e.g. the biological activity of the engineered cell populations, in some embodiments is measured, e.g., by any of a number of known methods. Parameters to assess include the ability of the engineered cells to destroy target cells, persistence and other measures of T cell activity, such as measured using any suitable method known in the art, such as assays described further below in Section III. In some embodiments, the biological activity of the cells, e.g., T cells administered for the T cell based therapy, is measured by assaying cytotoxic cell killing, expression and/or secretion of one or more cytokines, proliferation or expansion, such as upon restimulation with antigen. In some aspects the biological activity is measured by assessing the disease burden and/or clinical outcome, such as reduction in tumor burden or load. In some embodiments, administration of one or both agents of the combination therapy and/or any repeated administration of the therapy, can be determined based on the results of the assays before, during, during the course of or after administration of one or both agents of the combination therapy.

In some embodiments, the combined effect of the immunomodulatory compound in combination with the cell therapy can be synergistic compared to treatments involving only the immunomodulatory compound or monotherapy with the cell therapy. For example, in some embodiments, the methods provided herein result in an increase or an improvement in a desired therapeutic effect, such as an increased or an improvement in the reduction or inhibition of one or more symptoms associated with cancer.

In some embodiments, the immunomodulatory compound increases the expansion or proliferation of the engineered T cells, such as CAR T-Cells. In some embodiments, the increase in expansion or proliferation is observed in vivo upon administration to a subject. In some embodiments, the increase in the number of engineered T cells, e.g. CAR-T cells, is increased by greater than or greater than about 1.2-fold, 1.5-fold, 2.0-fold, 3.0-fold, 4.0-fold, 5.0-fold, 6.0-fold, 7.0-fold, 8.0-fold, 9.0-fold, 10.0 fold or more.

### A. Subjects

Also provided are methods of administering and uses, such as therapeutic uses, of a T cell therapy (e.g., CAR T cells) and/or compositions comprising the same. Such methods and uses include therapeutic methods and uses, for example, involving administration of the T cell therapy, and optionally, a subsequent therapy, to a subject having a cancer. In some embodiments, the subject is relapsed following treatment with, or refractory to, a prior therapy for treating the cancer. In some embodiments, the prior therapy and the subsequent therapy are of the same class of therapy (e.g., immunomodulatory drugs). In some embodiments, the T cell therapy, subsequent therapy, and/or composition(s) thereof is/are administered in an effective amount to effect treatment of the cancer. Provided herein are uses of the T cell therapy, and optionally, the subsequent therapy, in such methods and treatments, and in the preparation of a medicament in order to carry out such therapeutic methods. In some embodiments, the methods are carried out by administering the T cell therapy, and optionally the subsequent therapy, or compositions comprising the same, to the subject having the cancer. In some embodiments, the methods thereby treat the cancer in the subject. Also provided herein are of use of any of the compositions, such as pharmaceutical compositions provided herein, for the treatment of a cancer, such as use in a treatment regimen.

In some embodiments, the T cell therapy comprises genetically engineered T cells expressing a recombinant receptor (e.g., a TCR or a CAR). Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

Among the diseases to be treated is a B cell malignancy, such as multiple myeloma (MM). MM is associated with BCMA expression. See Coquery et al., Crit Rev Immunol. (2012) 32(4):287-305 for a review of BCMA. Since BCMA has been implicated in mediating tumor cell survival, it is a potential target for cancer therapy. Chimeric antigen receptors containing mouse anti-human BCMA antibodies and cells expressing such chimeric receptors have been previously described. See Carpenter et al., Clin Cancer Res., 2013, 19(8):2048-2060. In some embodiments, the multiple myeloma (MM) is associated with GPRC5D expression. See Atamaniuk et al., Eur. J. Clin. Invest. (2012) 42(9):953-60; Cohen et al., Hematol. (2013) 18(6):348-51; Frigyesi et al., Blood (2014) 123(9):1336-40. Accordingly, GPRC5D is also a potential target for cancer therapy. Chimeric antigen receptors containing anti-GPRC5D antibodies and cells expressing such chimeric receptors have been previously described. See WO 2016/090312 and Smith et al., Sci. Transl. Med. (2019) 11(485): eaau7746.

In some embodiments the multiple myeloma (MM) is a high risk MM or a relapsed and/or refractory multiple myeloma.

In some embodiments, at the time of administration of the T cell therapy, the multiple myeloma (MM) is a high risk MM. In some embodiments, at the time of administration of the T cell therapy, cells of the multiple myeloma (MM) comprise one or more high risk (HR) tumor features. In some embodiments, a HR tumor feature includes amplification of the long arm of chromosome 1 (amp1q), a cereblon (CRBN) mutation, t(4;14), high cancer clonal fraction del17p, biallelic p53 inactivation, the MDMS8 gene signature, or a combination thereof.

In some embodiments, at the time of administration of the T cell therapy, cells of the multiple myeloma comprise amp1q. In some embodiments, at the time of administration of the T cell therapy, cells of the multiple myeloma comprise a CRBN mutation. In some embodiments, at the time of administration of the T cell therapy, cells of the multiple myeloma comprise t(4;14). In some embodiments, at the time of administration of the T cell therapy, cells of the multiple myeloma comprise high cancer clonal fraction del17p. In some embodiments, at the time of administration of the T cell therapy, cells of the multiple myeloma comprise biallelic p53 inactivation. In some embodiments, at the time of administration of the T cell therapy, cells of the multiple myeloma comprise the MDMS8 gene signature.

In some embodiments, prior to administration of the T cell therapy, cells of the cancer comprise one or more HR feature(s) selected from among the group consisting of amp1q, the MDMS8 gene signature, a CRBN mutation, biallelic p53 inactivation, high cancer clonal fraction del17p, and t(4,14); and following administration of the T cell therapy in, cells of the cancer do not comprise at least one of the HR features that the cells of the cancer comprised prior to administration of the T cell therapy. In some embodiments, a subject is selected from treatment with the subsequent therapy if prior to administration of the T cell therapy, cells of the cancer comprise one or more HR feature(s) selected from among the group consisting of amp1q, the MDMS8 gene signature, a CRBN mutation, biallelic p53 inactivation, high cancer clonal fraction del17p, and t(4,14); and following administration of the T cell therapy, cells of the cancer do not comprise at least one of the high risk features that the cells of the cancer comprised prior to administration of the T cell therapy. In some embodiments, prior to administration of the T cell therapy, cells of the cancer comprise a CRBN mutation. In some embodiments, following administration of the T cell therapy, cells of the cancer do not comprise a CRBN mutation. In some embodiments, the subject is selected from treatment with the subsequent therapy if prior to administration of the T cell therapy, cells of the cancer comprise a CRBN mutation, and following administration of the T cell therapy, cells of the cancer do not comprise a CRBN mutation.

In some embodiments the multiple myeloma (MM) is a relapsed and/or refractory multiple myeloma. In some embodiments the multiple myeloma (MM) is a relapsed and refractory multiple myeloma (r/r MM). In some of any embodiments, at the time of administration, the subject has a R/R MM.

In some embodiments, prior to administration of the cell therapy, the subject does not exhibit minimum residual disease (MRD) negative status. In some embodiments, following administration of the T cell therapy, the subject achieves MRD negative status. In some embodiments, subsequent to the patient achieving MRD negative status after administration of the T cell therapy, the cancer progresses in the subject. In some embodiments, the subject is selected from treatment with the subsequent therapy if, following administration of the T cell therapy, the subject achieves minimum residual disease (MRD) negative status; and subsequent to the subject achieving MRD negative status after administration of the T cell therapy, the cancer progresses in the subject.

In some embodiments, the subject has persistent or relapsed disease, e.g., following treatment with a prior therapy. In some embodiments, prior to the administration of the T cell therapy, the subject has received one or more prior therapies. In some embodiments, the subject has received at least 1, 2, 3, 4, 5, 6, 7, 8, 9, 10, 11, 12, 13, 14, 15, 16, 17, 18, 19 or 20 or more prior therapies. In some embodiments, the subject has received at least 3, 4, 5, 6, 7, 8, 9, 10 or more prior therapies. In some embodiments, the subject has received 3 or more prior therapies.

In some embodiments, the subject has relapsed following treatment with, or is refractory to, a prior therapy. In some embodiments, the prior therapy binds the CRBN protein. In some embodiments, the prior therapy is an immunomodulatory drug. In some embodiments, the immunomodulatory drug is thalidomide, lenalidomide, pomalidomide, iberdomide, CC-92480, CC-99282, CC-91633, or CC-90009. In some embodiments, the prior therapy is a proteasome inhibitor. In some embodiments, the proteasome inhibitor is bortezomib, carfilzomib or ixazomib. In some embodiments, the proteasome inhibitor is bortezomib. In some embodiments, the prior therapy is an anti-CD38 antibody. In some embodiments, the anti-CD38 antibody is daratumumab or isatuximab. In some embodiments, the anti-CD38 antibody is daratumumab. In some embodiments, the prior therapy is an inhibitor of Bruton's tyrosine kinase (BTK). In some embodiments, the BTK inhibitor is ibrutinib, acalabrutinib, zanubrutinib, evobrutinib, tirabrutinib, or SNS-06. In some embodiments, the BTK inhibitor is ibrutinib. In some embodiments, the prior therapy is an inhibitor of BCL-2. In some embodiments, the BCL-2 inhibitor is venetoclax, navitoclax, ABT737, maritoclax, obatoclax, and clitocine. In some embodiments, the BCL-2 inhibitor is venetoclax.

In some embodiments, the assessment for the criteria, diagnosis or indication can be performed at the time of screening the subjects for eligibility or suitability of treatment according to the provided methods, at various steps of the treatment regimen, at the time of receiving lymphodepleting therapy, and/or at or immediately prior to the initiation of administration of the engineered cells or composition thereof.

Among the diseases to be treated is a leukemia or lymphoma. In some embodiments, the cancer is chronic lymphocytic leukemia (CLL), small lymphocytic lymphoma (SLL), acute lymphoblastic leukemia (ALL), acute myelogenous leukemia (AML), chronic myelogenous leukemia (CML), myelodysplastic syndrome (MDS), non-Hodgkin lymphoma (NHL), or a subtype of NHL, such as diffuse large B-cell lymphoma (DLBCL). In some embodiments, the cancer is a leukemia. In some embodiments, the cancer is chronic lymphocytic leukemia (CLL). In some embodiments, the cancer is a lymphoma. In some embodiments, the cancer is small lymphocytic lymphoma (SLL). In some embodiments, the leukemia or lymphoma is associated with expression of CD19. Thus, CD19 is a potential target for cancer therapy. Chimeric antigen receptors containing anti-CD19 antibodies and cells expressing such chimeric receptors have been previously described.

Thus, the provided methods and uses include methods and uses for adoptive cell therapy. In some embodiments, the methods include administration of the cells or a composition containing the cells to a subject, tissue, or cell, such as one having, at risk for, or suspected of having a multiple myeloma. In some embodiments, the cells, populations, and compositions are administered to a subject having a multiple myeloma, e.g., via adoptive cell therapy, such as adoptive T cell therapy. In some embodiments, the cells or compositions are administered to the subject, such as a subject having or at risk for a multiple myeloma. In some aspects, the methods thereby treat, e.g., ameliorate one or more symptom of a multiple myeloma, such as by lessening tumor burden.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the T cell therapy, e.g., adoptive cell therapy, e.g., adoptive T cell therapy, is carried out by autologous transfer, in which the cells are isolated and/or otherwise prepared from the subject who is to receive the T cell therapy, or from a sample derived from such a subject. Thus, in some aspects, the cells are derived from a subject, e.g., patient, in need of a treatment and the cells, following isolation and processing are administered to the same subject.

In some embodiments, the T cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The subject can be male or female and can be any suitable age, including infant, juvenile, adolescent, adult, and geriatric subjects. In some embodiments, the subject is an adult (i.e. 18 years of age or older).

In some embodiments, the dose and/or frequency of administration is determined based on efficacy and/or response. In some embodiments, efficacy is determined by evaluating disease status. Exemplary methods for assessing disease status include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, dose and/or frequency of administration is determined by the expansion and persistence of the recombinant receptor or cell in the blood and/or bone marrow. In some embodiments, dose and/or frequency of administration is determined based on the antitumor activity of the recombinant receptor or engineered cell. In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some embodiments, response is evaluated based on the duration of response following administration of the recombinant receptor or cells. In some examples, dose and/or frequency of administration can be based on toxicity. In some embodiments, dose and/or frequency can be determined based on health-related quality of life (HRQoL) of the subject to which the recombinant receptor and/or cells is/are administered. In some embodiments, dose and/or frequency of administration can be changed, i.e., increased or decreased, based on any of the above criteria.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of selfcare; see e.g., Sorensen et al., (1993) Br J Cancer 67(4) 773-775. In some embodiments, the subjects that are to be administered according to the methods or treatment regimen provided herein include those with an ECOG performance status of 0 or 1. In some embodiments, the subject has an Eastern Cooperative Oncology Group (ECOG) performance status of 0 or 1. In some embodiments, the subject has an Eastern Cooperative Oncology Group (ECOG) performance status of 0. In some embodiments, the subject has an Eastern Cooperative Oncology Group (ECOG) performance status of 1.

In some embodiments, the administration can treat the subject despite the subject having become resistant to another therapy. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, when administered to subjects according to the embodiments described herein, the dose or the composition is capable of achieving stringent complete response (sCR) at least 20%, 30%, 40% 50%, 60% or 70% of subjects that were administered. In some embodiments, when administered to subjects according to the embodiments described herein, the dose or the composition is capable of achieving complete response (CR) at least 20%, 30%, 40% 50%, 60% or 70% of subjects that were administered. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346).

In some embodiments, toxicity and/or side effects of treatment can be monitored and used to adjust dose and/or frequency of administration of the recombinant receptor, e.g., CAR, cells, and or compositions. For example, adverse events and laboratory abnormalities can be monitored and used to adjust dose and/or frequency of administration. Adverse events include infusion reactions, cytokine release syndrome (CRS), neurotoxicity, macrophage activation syndrome, and tumor lysis syndrome (TLS). Any of such events can establish dose-limiting toxicities and warrant decrease in dose and/or a termination of treatment. Other side effects or adverse events which can be used as a guideline for establishing dose and/or frequency of administration include non-hematologic adverse events, which include but are not limited to fatigue, fever or febrile neutropenia, increase in transaminases for a set duration (e.g., less than or equal to 2 weeks or less than or equal to 7 days), headache, bone pain, hypotension, hypoxia, chills, diarrhea, nausea/vomiting, neurotoxicity (e.g., confusion, aphasia, seizures, convulsions, lethargy, and/or altered mental status), disseminated intravascular coagulation, other asymptomatic non-hematological clinical laboratory abnormalities, such as electrolyte abnormalities. Other side effects or adverse events which can be used as a guideline for establishing dose and/or frequency of administration include hematologic adverse events, which include but are not limited to neutropenia, leukopenia, thrombocytopenia, animal, and/or B-cell aplasia and hypogammaglobinemia.

In some embodiments, treatment according to the provided methods can result in a lower rate and/or lower degree of toxicity, toxic outcome or symptom, toxicity-promoting profile, factor, or property, such as a symptom or outcome associated with or indicative of cytokine release syndrome (CRS) or neurotoxicity, such as severe CRS or severe neurotoxicity, for example, compared to administration of other therapies.

In some embodiments, the subject may receive abridging therapy after leukapheresis and before lymphodepleting chemotherapy. A treating physician can determine if bridging therapy is necessary, for example for disease control, during manufacturing of the provided composition or cells. In some embodiments, bridging therapies are discontinued prior to initiation of lymphodepletion. In some embodiments, bridging therapies are discontinued 1 day, 2 days 3 days, 4 days, 5 days, 7 days, 10 days, 14 days, 21 days, 28 days, 45 days, or 60 days before lymphodepletion.

Once the cells are administered to a mammal (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR expressed by the cells in some embodiments is conjugated either directly or indirectly through a linker to a targeting moiety. The practice of conjugating compounds, e.g., the CAR, to targeting moieties is known in the art. See, for instance, Wadwa et al., J. Drug Targeting, 3(2):111 (1995), and U.S. Pat. No. 5,087,616

### B. Prior Therapy

Provided herein are methods of treating a subject having a cancer comprising administration of a T cell therapy (e.g., CAR T cells or a TCE), wherein the subject has relapsed following treatment with, or is refractory to, a prior therapy for treating the cancer. In some embodiments, the methods further comprise, following administration of the T cell therapy, administration of a subsequent therapy for treating the cancer to the subject, wherein the prior therapy and the subsequent therapy are of the same class of therapy. In some embodiments, the class of therapy is immunomodulatory drugs, proteasome inhibitors, anti-CD38 antibodies, BTK inhibitors, or BCL-2 inhibitors. In some embodiments, the class of therapy is immunomodulatory drugs. In some embodiments, the class of therapy is proteasome inhibitors. In some embodiments, the class of therapy is anti-CD38 antibodies. In some embodiments, the class of therapy is BTK inhibitors. In some embodiments, the class of therapy is BCL-2 inhibitors.

### 1. Immunomodulatory Drugs

In some embodiments, the prior therapy for treating the cancer is a immunomodulatory drug. In some embodiments, the immunomodulatory drug is a cereblon-modulating compound. In some embodiments, the immunomodulatory drug is a cereblon-binding compound. Cereblon functions as a substrate receptor for a CRL4 ubiquitin E3 ligase, and the binding of cereblon-modulating compounds can induce the recruitment, ubiquitination, and destruction of certain target substrates, such as Ikaros family zinc finger proteins 1 and 3 (IKZF1 and IKZF3, also known as Ikaros and Aiolos, respectively). In some embodiments, administration of the immunomodulatory drug induces ubiquitination of Aiolos and/or Ikaros. In some embodiments, administration of the immunomodulatory drug induces degradation of Aiolos and/or Ikaros. In some aspects, the degree of degradation induced by the immunomodulatory drug is associated with its antitumor effects, for instance with increased degradation associated with greater antitumor effects by the immunomodulatory drug. In some embodiments, the immunomodulatory drug is an IMiD™ or a CELMoD™.

Exemplary immunomodulatory drugs include the substituted 2-(2,6-dioxopiperidin-3-yl)phthalimides and substituted 2-(2,6-dioxopiperidin-3-yl)-1-oxoisoindoles described in U.S. Pat. Nos. 6,281,230 and 6,316,471. Still other exemplary immunomodulatory drugs belong to a class of isoindole-imides disclosed in U.S. Pat. Nos. 6,395,754, 6,555,554, 7,091,353, U.S. Pat. Publication No. 2004/0029832, and International Publication No. WO 98/54170.

In some embodiments, the immunomodulatory drug is selected from among the group consisting of thalidomide, lenalidomide, pomalidomide, iberdomide (CC-220), CC-92480, CC-99282, CC-91633, and CC-90009, an enantiomer or a mixture of enantiomers thereof, or a pharmaceutically acceptable salt, solvate, hydrate, co-crystal, clathrate, or polymorph thereof. In some embodiments, the immunomodulatory drug is selected from among the group consisting of thalidomide, lenalidomide, pomalidomide, iberdomide (CC-220), CC-92480, CC-99282, CC-91633, and CC-90009 or a pharmaceutically acceptable salt thereof. In some embodiments, the immunomodulatory drug is selected from among the group consisting of thalidomide, lenalidomide, pomalidomide, iberdomide (CC-220), CC-92480, CC-99282, and CC-90009 or a pharmaceutically acceptable salt thereof.

In some embodiments, the immunomodulatory drug is administered at a dose of from or from about 0.1 mg to 100 mg, from or from about 0.1 mg to 75 mg, from or from about 0.1 mg to 50 mg, from or from about 0.1 mg to 25 mg, from or from about 0.1 mg to 10 mg, from or from about 0.1 mg to 5 mg, from or from about 0.1 mg to 1 mg, from or from about 1 mg to 100 mg, from or from about 1 mg to 75 mg, from or from about 1 mg to 50 mg, from or from about 1 mg to 25 mg, from or from about 1 mg to 10 mg, from or from about 1 mg to 5 mg, from or from about 5 mg to 100 mg, from or from about 5 mg to 75 mg, from or from about 5 mg to 50 mg, from or from about 5 mg to 25 mg, from or from about 5 mg to 10 mg, from or from about 10 mg to 100 mg, from or from about 10 mg to 75 mg, from or from about 10 mg to 50 mg, from or from 10 mg to 25 mg, from or from about 25 mg to 100 mg, from or from about 25 mg to 75 mg, from or from about 25 mg to 50 mg, from or from about 50 mg to 100 mg, from or from about 50 mg to 75 mg, or from or from about 75 mg to 100 mg, each inclusive. In some embodiments, the dose is a daily dose. In some embodiments, the dose is a once-daily dose. In some embodiments, the dose is the amount of the immunomodulatory drug that is administered on each of the days on which the immunomodulatory drug is administered.

In some embodiments, the immunomodulatory drug is administered at a dose of from or from about 0.1 mg to about 1.0 mg, from or from about 0.1 mg to 0.9 mg, from or from about 0.1 mg to 0.8 mg, from or from about 0.1 mg to 0.7 mg, from or from about 0.1 mg to 0.6 mg, from or from about 0.1 mg to 0.5 mg, from or from about 0.1 mg to 0.4 mg, from or from about 0.1 mg to 0.3 mg, from or from about 0.1 mg to 0.2 mg, from or from about 0.2 mg to 1.0 mg, from or from about 0.2 mg to 0.9 mg, from or from about 0.2 mg to 0.8 mg, from or from about 0.2 mg to 0.7 mg, from or from about 0.2 mg to 0.6 mg, from or from about 0.2 mg to 0.5 mg, from or from about 0.2 mg to 0.4 mg, from or from about 0.2 mg to 0.3 mg, from or from about 0.3 mg to 1.0 mg, from or from about 0.3 mg to 0.9 mg, from or from about 0.3 mg to 0.8 mg, from or from about 0.3 mg to 0.7 mg, from or from about 0.3 mg to 0.6 mg, from or from about 0.3 mg to 0.5 mg, from or from about 0.3 mg to 0.4 mg, from or from about 0.4 mg to 1.0 mg, from or from about 0.4 mg to 0.9 mg, from or from about 0.4 mg to 0.8 mg, from or from about 0.4 mg to 0.7 mg, from or from about 0.4 mg to 0.6 mg, from or from about 0.4 mg to 0.5 mg, from or from about 0.5 mg to 1.0 mg, from or from about 0.5 mg to 0.9 mg, from or from about 0.5 mg to 0.8 mg, from or from about 0.5 mg to 0.7 mg, from or from about 0.5 mg to 0.6 mg, from or from about 0.6 mg to 1.0 mg, from or from about 0.6 mg to 0.9 mg, from or from about 0.6 mg to 0.8 mg, from or from about 0.6 mg to 0.7 mg, from or from about 0.7 mg to 1.0 mg, from or from about 0.7 mg to 0.9 mg, from or from about 0.7 mg to 0.8 mg, from or from about 0.8 mg to 1.0 mg, from or from about 0.8 mg to 0.9 mg, or from or from about 0.8 mg to 1.0 mg, each inclusive. In some embodiments, the dose is a daily dose. In some embodiments, the dose is a once-daily dose. In some embodiments, the dose is the amount of the immunomodulatory drug that is administered on each of the days on which the immunomodulatory drug is administered.

In some embodiments, the immunomodulatory drug is administered several times a day, twice a day, daily, every other day, three times a week, twice a week, or once a week. In some embodiments, the immunomodulatory drug is administered daily. In some embodiments, the immunomodulatory drug is administered daily for a plurality of consecutive days. In some embodiments, the immunomodulatory drug is administered daily for up to about 7, 8, 9, 10, 11, 12, 13, 14, 15, 16, 17, 18, 19, 20, 21, 22, 23, 24, 25, 26, 27, 28, 29, 30, or more than 30 consecutive days.

In some embodiments, the immunomodulatory drug is administered in a cycle. In some embodiments, the cycle includes an administration period in which the immunomodulatory drug is administered followed by a rest period during which the immunomodulatory drug is not administered. In some embodiments, the rest period is greater than about 1 day, greater than about 3 consecutive days, greater than about 5 consecutive days, greater than about 7 consecutive days, greater than about 8 consecutive days, greater than about 9 consecutive days, greater than about 10 consecutive days, greater than about 11 consecutive days, greater than about 12 consecutive days, greater than about 13 consecutive days, greater than about 14 consecutive days, greater than about 15 consecutive days, greater than about 16 consecutive days, greater than about 17 consecutive days, greater than about 18 consecutive days, greater than about 19 consecutive days, greater than about 20 consecutive days, greater than about 21 consecutive days, or greater than about 28 or more consecutive days. In some embodiments, the immunomodulatory drug is administered once daily for 14 days over a 21-day treatment cycle. In some embodiments, the immunomodulatory drug is administered once daily for 21 days over a 28-day treatment cycle.

In some embodiments, the immunomodulatory drug is administered for at least 2 cycles, at least 3 cycles, at least 4 cycles, at least 5 cycles, at least 6 cycles, at least 7 cycles, at least 8 cycles, at least 9 cycles, at least 10 cycles, at least 11 cycles, or at least 12 cycles. In some embodiments, the immunomodulatory drug is administered for 1, 2, 3, 4, 5, 6, 7, 8, 9, 10, 11, 12, 13, 14, 15, 16, 17, 18, 19, 20, 21, 22, 23, or 24 cycles.

In some embodiments, the immunomodulatory drug is administered orally. In some embodiments, the immunomodulatory drug is administered as a tablet or capsule. In some embodiments, the immunomodulatory drug is administered intravenously.

In some embodiments, the immunomodulatory drug is thalidomide ((RS)-2-(2,6-dioxopiperidin-3-yl)-1H-isoindole-1,3(2H)-dione) having the structure:

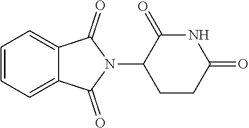 [SMILES: O=C1CCC(N2C(=O)c3ccccc3C2=O)C(=O)N1]

or an enantiomer or a mixture of enantiomers of thalidomide, or a pharmaceutically acceptable salt, solvate, hydrate, co-crystal, clathrate, or polymorph thereof. In some embodiments, the immunomodulatory drug is a pharmaceutically acceptable salt of thalidomide. In some embodiments, the immunomodulatory drug is a solvate of thalidomide. In some embodiments, the immunomodulatory drug is a hydrate of thalidomide. In some embodiments, the immunomodulatory drug is a co-crystal of thalidomide. In some embodiments, the immunomodulatory drug is a clathrate of thalidomide. In some embodiments, the immunomodulatory drug is a polymorph of thalidomide. In some embodiments, the immunomodulatory drug is thalidomide. In some embodiments, the prior therapy is thalidomide. Exemplary dosing regimens for thalidomide administration for treatment of multiple myeloma are described in, e.g., Cavallo et al., Ther Clin Risk Manag (2007) 3(4): 543-552.

In some embodiments, the immunomodulatory drug is lenalidomide (3-(4-amino-1-oxo-1,3-dihydro-2H-isoindol-2-yl)piperidine-2,6-dione) having the structure:

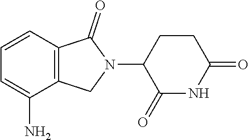 [SMILES: Nc1cccc2c1CN(C1CCC(=O)NC1=O)C2=O]

or an enantiomer or a mixture of enantiomers of lenalidomide, or a pharmaceutically acceptable salt, solvate, hydrate, co-crystal, clathrate, or polymorph thereof. In some embodiments, the immunomodulatory drug is a pharmaceutically acceptable salt of lenalidomide. In some embodiments, the immunomodulatory drug is a solvate of lenalidomide. In some embodiments, the immunomodulatory drug is a hydrate of lenalidomide. In some embodiments, the immunomodulatory drug is a co-crystal of lenalidomide. In some embodiments, the immunomodulatory drug is a clathrate of lenalidomide. In some embodiments, the immunomodulatory drug is a polymorph of lenalidomide. In some embodiments, the immunomodulatory drug is lenalidomide. In some embodiments, the prior therapy is lenalidomide. Exemplary dosing regimens for lenalidomide administration for treatment of multiple myeloma are described in, e.g., Chen et al., Curr Oncol (2013) 20 (2): e136-e149.

In some embodiments, the immunomodulatory drug is pomalidomide (4-amino-2-(2,6-dioxopiperidin-3-yl)isoindole-1,3-dione) having the structure:

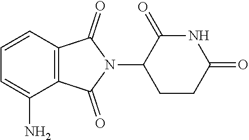 [SMILES: Nc1cccc2c1C(=O)N(C1CCC(=O)NC1=O)C2=O]

or an enantiomer or a mixture of enantiomers of pomalidomide, or a pharmaceutically acceptable salt, solvate, hydrate, co-crystal, clathrate, or polymorph thereof. In some embodiments, the immunomodulatory drug is a pharmaceutically acceptable salt of pomalidomide. In some embodiments, the immunomodulatory drug is a solvate of pomalidomide. In some embodiments, the immunomodulatory drug is a hydrate of pomalidomide. In some embodiments, the immunomodulatory drug is a co-crystal of pomalidomide. In some embodiments, the immunomodulatory drug is a clathrate of pomalidomide. In some embodiments, the immunomodulatory drug is a polymorph of pomalidomide. In some embodiments, the immunomodulatory drug is pomalidomide. In some embodiments, the prior therapy is pomalidomide. Exemplary dosing regimens for pomalidomide administration for treatment of multiple myeloma are described in, e.g., Clark et al., J Adv Pract Oncol (2014) 5(1): 51-56.

In some embodiments, the immunomodulatory drug is iberdomide ((S)-3-[4-(4-morpholin-4-ylmethyl-benzyloxy)-1-oxo-1,3-dihydro-isoindol-2-yl]-piperidine-2,6-dione; also known as CC-220) having the structure:

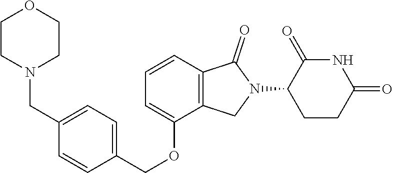 [SMILES: O=C1CC[C@H](N2Cc3c(OCc4ccc(CN5CCOCC5)cc4)cccc3C2=O)C(=O)N1]

or an enantiomer or a mixture of enantiomers of iberdomide, or a pharmaceutically acceptable salt, solvate, hydrate, co-crystal, clathrate, or polymorph thereof. Methods of preparing iberdomide are described in US Pat. Application No. 2011/0196150. In some embodiments, the immunomodulatory drug is a pharmaceutically acceptable salt of iberdomide. In some embodiments, the immunomodulatory drug is a solvate of iberdomide. In some embodiments, the immunomodulatory drug is a hydrate of iberdomide. In some embodiments, the immunomodulatory drug is a co-crystal of iberdomide. In some embodiments, the immunomodulatory drug is a clathrate of iberdomide. In some embodiments, the immunomodulatory drug is a polymorph of iberdomide. In some embodiments, the immunomodulatory drug is iberdomide. In some embodiments, the prior therapy is iberdomide. Exemplary dosing regimens for iberdomide administration for treatment of multiple myeloma are described in, e.g., Lonial et al., Journal of Clinical Oncology 37, no. 15_suppl (May 20, 2019) 8006-8006.

In some embodiments, the immunomodulatory drug is CC-92480 ((S)-4-(4-(4-(((2-(2,6-dioxopiperidin-3-yl)-1-oxoisoindolin-4-yl)oxy)methyl)benzyl)piperazin-1-yl)-3-fluorobenzonitrile) having the structure:

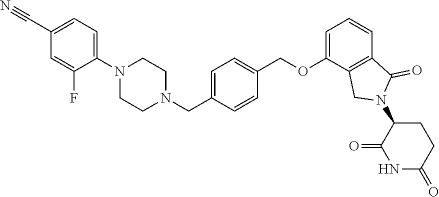 [SMILES: N#Cc1ccc(N2CCN(Cc3ccc(COc4cccc5c4CN([C@H]4CCC(=O)NC4=O)C5=O)cc3)CC2)c(F)c1]

or an enantiomer or a mixture of enantiomers of CC-92480, or a pharmaceutically acceptable salt, solvate, hydrate, co-crystal, clathrate, or polymorph thereof. In some embodiments, the immunomodulatory drug is a pharmaceutically acceptable salt of CC-92480. In some embodiments, the immunomodulatory drug is a solvate of CC-92480. In some embodiments, the immunomodulatory drug is a hydrate of CC-92480. In some embodiments, the immunomodulatory drug is a co-crystal of CC-92480. In some embodiments, the immunomodulatory drug is a clathrate of CC-92480. In some embodiments, the immunomodulatory drug is a polymorph of CC-92480. In some embodiments, the immunomodulatory drug is CC-92480. In some embodiments, the prior therapy is CC-92480. Exemplary dosing regimens for CC-92480 administration for treatment of multiple myeloma are described in, e.g., Richardson et al., Journal of Clinical Oncology 38, no. 15_suppl (May 20, 2020) 8500-8500.

In some embodiments, the immunomodulatory drug is CC-99282 ((S)-2-(2,6-dioxopiperidin-3-yl)-4-((2-fluoro-4-((3-morpholinoazetidin-1-yl)methyl)benzyl)amino)isoindoline-1,3-dione) having the structure:

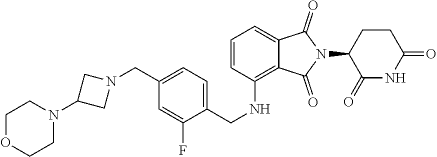 [SMILES: O=C1CC[C@H](N2C(=O)c3cccc(NCc4ccc(CN5CC(N6CCOCC6)C5)cc4F)c3C2=O)C(=O)N1]

or an enantiomer or a mixture of enantiomers of CC-99282, or a pharmaceutically acceptable salt, solvate, hydrate, co-crystal, clathrate, or polymorph thereof. Methods of preparing CC-99282 are described in US Pat. Application No. 2019/0322647. In some embodiments, the immunomodulatory drug is a pharmaceutically acceptable salt of CC-99282. In some embodiments, the immunomodulatory drug is a solvate of CC-99282. In some embodiments, the immunomodulatory drug is a hydrate of CC-99282. In some embodiments, the immunomodulatory drug is a co-crystal of CC-99282. In some embodiments, the immunomodulatory drug is a clathrate of CC-99282. In some embodiments, the immunomodulatory drug is a polymorph of CC-99282. In some embodiments, the immunomodulatory drug is CC-99282. In some embodiments, the prior therapy is CC-99282. Exemplary dosing regimens for CC-99282 administration for treatment of lymphoma are described in, e.g., Michot et al., Blood (2021) 138(Supplement 1): 3574; and Michot et al., Hematological Oncology (2021) 39(S2 Supplement).

In some embodiments, the immunomodulatory drug is CC-91633 or an enantiomer or a mixture of enantiomers of CC-91633, or a pharmaceutically acceptable salt, solvate, hydrate, co-crystal, clathrate, or polymorph thereof. In some embodiments, the immunomodulatory drug is a pharmaceutically acceptable salt of CC-91633. In some embodiments, the immunomodulatory drug is a solvate of CC-91633. In some embodiments, the immunomodulatory drug is a hydrate of CC-91633. In some embodiments, the immunomodulatory drug is a co-crystal of CC-91633. In some embodiments, the immunomodulatory drug is a clathrate of CC-91633. In some embodiments, the immunomodulatory drug is a polymorph of CC-91633. In some embodiments, the immunomodulatory drug is CC-91633. In some embodiments, the prior therapy is CC-91633.

In some embodiments, the immunomodulatory drug is CC-90009 having the structure:

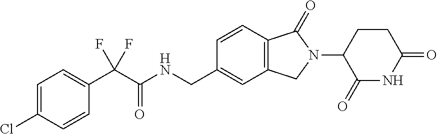 [SMILES: O=C1CCC(N2Cc3cc(CNC(=O)C(F)(F)c4ccc(Cl)cc4)ccc3C2=O)C(=O)N1]

or an enantiomer or a mixture of enantiomers of CC-90009, or a pharmaceutically acceptable salt, solvate, hydrate, co-crystal, clathrate, or polymorph thereof (see, e.g., Surka et al., Blood (2021) 137(5): 661-677). In some embodiments, the immunomodulatory drug is a pharmaceutically acceptable salt of CC-90009. In some embodiments, the immunomodulatory drug is a solvate of CC-90009. In some embodiments, the immunomodulatory drug is a hydrate of CC-90009. In some embodiments, the immunomodulatory drug is a co-crystal of CC-90009. In some embodiments, the immunomodulatory drug is a clathrate of CC-90009. In some embodiments, the immunomodulatory drug is a polymorph of CC-90009. In some embodiments, the immunomodulatory drug is CC-90009. In some embodiments, the prior therapy is CC-90009.

In some embodiments, the term “pharmaceutically acceptable salt” refers to a salt prepared from a pharmaceutically acceptable non-toxic acid or base including an inorganic acid and base and an organic acid and base. Suitable pharmaceutically acceptable base addition salts include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine), and procaine. Suitable non-toxic acids include inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Others are well-known in the art, see for example Remington's Pharmaceutical Sciences, 18th eds., Mack Publishing, Easton PA (1990) or Remington: The Science and Practice of Pharmacy, 19th eds., Mack Publishing, Easton PA (1995).

In some embodiments, the term “stereoisomer” or “stereomerically pure” means one stereoisomer of a drug that is substantially free of other stereoisomers of that drug. For example, a stereomerically pure drug having one chiral center will be substantially free of the opposite enantiomer of the drug. A stereomerically pure drug having two chiral centers will be substantially free of other diastereomers of the drug. A typical stereomerically pure drug comprises greater than about 80% by weight of one stereoisomer of the drug and less than about 20% by weight of other stereoisomers of the drug, greater than about 90% by weight of one stereoisomer of the drug and less than about 10% by weight of the other stereoisomers of the drug, greater than about 95% by weight of one stereoisomer of the drug and less than about 5% by weight of the other stereoisomers of the drug, or greater than about 97% by weight of one stereoisomer of the drug and less than about 3% by weight of the other stereoisomers of the drug. The drugs can have chiral centers and can occur as racemates, individual enantiomers or diastereomers, and mixtures thereof. Methods involving administration of any such isomeric forms of the immunomodulatory drug are included within the embodiments provided herein, including administration of mixtures thereof.

In some embodiments, the immunomodulatory drug contains one chiral center, and can exist as a mixture of enantiomers, e.g., a racemic mixture. This disclosure encompasses the use of stereomerically pure forms of such a drug, as well as the use of mixtures of those forms. For example, mixtures comprising equal or unequal amounts of the enantiomers of the immunomodulatory drug may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E L. Eliel, Ed., Univ. of Notre Dame Press, Notre Dame, IN, 1972).

It is to be understood that the chiral centers of the immunomodulatory drug may undergo epimerization in vivo. As such, one of skill in the art will recognize that in the case of epimerization in vivo, administration of the immunomodulatory drug in its (R) form may be equivalent to administration of the immunomodulatory drug in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

In some embodiments, the term “solvate” means a physical association of a drug with one or more solvent molecules, whether organic or inorganic. This physical association includes hydrogen bonding. In certain instances, the solvate will be capable of isolation, for example when one or more solvent molecules are incorporated in the crystal lattice of the crystalline solid. In some embodiments, “solvate” encompasses both solution-phase and isolable solvates. Exemplary solvates include hydrates, ethanolates, methanolates, isopropanolates, acetonitrile solvates, and ethyl acetate solvates. Methods of solvation are known in the art.

It is understood that, independently of stereomerical or isotopic composition, the immunomodulatory drug can be administered in the form of any of the pharmaceutically acceptable salts described herein. Equally, it is understood that the isotopic composition may vary independently from the stereomerical composition of the immunomodulatory drug. Further, the isotopic composition, while being restricted to those elements present in immunomodulatory drug or salt thereof, may otherwise vary independently from the selection of the pharmaceutically acceptable salt of immunomodulatory drug.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 2. Proteasome Inhibitors

In some embodiments, the prior therapy for treating the cancer is a proteasome inhibitor.

In some embodiments, the proteasome inhibitor inhibits the 26S proteasome. In some embodiments, inhibition of the 26S proteasome inhibits or blocks targeted proteolysis by the proteasome, thereby disrupting cell signaling pathways, which can lead to cell cycle arrest, apoptosis, and inhibition of angiogenesis. In some embodiments, the proteasome inhibitor inhibits nuclear factor kappa B (NFkB).

In some embodiments, the proteasome inhibitor is selected from among the group consisting of bortezomib, carfilzomib, and ixazomib.

In some embodiments, the proteasome inhibitor reversibly inhibits the 26S proteasome. In some embodiments, the proteasome inhibitor is [(1R)-3-methyl-1-[[(2S)-3-phenyl-2-(pyrazine-2-carbonylamino)propanoyl]amino]butyl]boronic acid, also known as bortezomib or Velcade®. In some embodiments, the proteasome inhibitor is bortezomib. In some embodiments, the prior therapy is bortezomib. In some embodiments, the proteasome inhibitor has the following structure

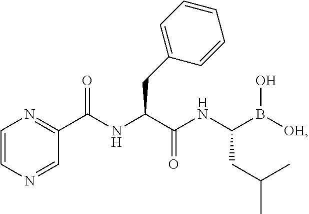 [SMILES: CC(C)C[C@H](NC(=O)[C@H](Cc1ccccc1)NC(=O)c1cnccn1)B(O)O]

or a pharmaceutically acceptable salt, solvate, hydrate, stereoisomer, tautomer or racemic mixtures thereof, including and compositions thereof. In some embodiments, the proteasome inhibitor has the following structure

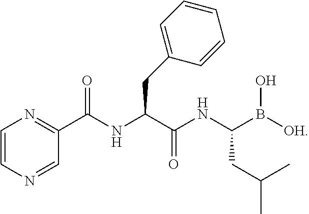 [SMILES: CC(C)C[C@H](NC(=O)[C@H](Cc1ccccc1)NC(=O)c1cnccn1)B(O)O]

Compositions of bortezomib include but are not limited to those described in U.S. Pat. Nos. 5,780,54, 6,083,903, 6,713,446, 6,958,319, 8,962,572, and U.S. Ser. No. 10/314,880; and International Publication Nos. WO 2006/052733 and WO 2016/166653 (each incorporated herein by reference in its entirety).

In some embodiments, the composition comprising bortezomib is a “ready to use” formulation that contains bortezomib in dissolved or solubilized form and is intended to be used as such or upon further dilution in intravenous diluents. In preferred embodiments, pharmaceutical compositions comprising bortezomib are formulated for parenteral administration, e.g. injection or infusion.

Suitable solvents can be selected from aqueous and non-aqueous solvents such as, but are not limited to, glycerin, ethanol, n-propanol, n-butanol, isopropanol, ethyl acetate, dimethyl carbonate, acetonitrile, dichloromethane, methyl ethyl ketone, methyl isobutyl ketone, cyclohexane, dimethylacetamide (DMA), dimethyl sulfoxide (DMSO), N-methyl-2-pyrrolidone (NMP), 1,3-dimethyl-2-imidazolidinone (DMI), acetone, tetrahydrofuran (THF), dimethylformamide (DMF), propylene carbonate (PC), dimethyl isosorbide, water and mixtures thereof. Preferred solvents are ethanol, glycerin and water.

The bortezomib formulation may comprise stabilizers such as sugars and amino acids. Suitable stabilizers include glucose, trehalose, sucrose, mannitol, sorbitol, arginine, glycine, proline, methionine, lysine and the like.

The bortezomib formulation may comprise a chelating agent. Suitable chelating agents include DOTA (1,4,7,10-tetraazacyclododecane-1,4,7,10-tetraacetic acid), DTPA (diethylene triaminepentaacetic acid), EDTA (Ethylenediaminetetraacetic acid), ODDA (1,4,10,13-tetraoxa-7,16-diazacyclooctadecane-7), TTT A (1,7,13-triaza-4, 10,16-trioxacyclooctadecane-N,N′,N″-triacetate), DOTRP (tetraethyleneglycol-1,5,9-triazacyclododecane-N,N′,N″,-tris(methylene phosphonic acid), EGTA (ethylene glycol-bis(P-aminoethyl ether)-tetraacetic acid) and the like.

The bortezomib formulation may also contain one or more antioxidants. Suitable anti-oxidants include, but are not limited to monothioglycerol, ascorbic acid, sodium bisulfite, sodium metabisulfite, L-cysteine, thioglycolic acid, citric acid, tartaric acid, phosphoric acid, gluconic acid, thiodipropionic acid and the like. Most preferred anti-oxidant is monothioglycerol.

The bortezomib formulation for use in the present invention may optionally contain other pharmaceutically acceptable adjuvants such as buffering agents, pH adjusting agents, preservatives, tonicity modifiers and the like. The lists of solvents, stabilizers, chelating agents and antioxidants listed above may also be used in pharmaceutical compositions comprising other cytotoxic agents described herein unless stated otherwise.

In some embodiments, the proteasome inhibitor is a selective proteasome inhibitor. In some embodiments, the proteasome inhibitor is an irreversible proteasome inhibitor. In some embodiments, the proteasome inhibitor is an irreversible and selective proteasome inhibitor. In some embodiments, the proteasome inhibitor is an analog of epoxomicin. In some embodiments, the proteasome inhibitor irreversibly and selectively binds to N-terminal threonine-containing active sites of the 20S proteasome. In some embodiments, the proteasome inhibitor is (2S)-4-methyl-N-[(2S)-1-[[(2S)-4-methyl-1-[(2R)-2-methyloxiran-2-yl]-1-oxopentan-2-yl]amino]-1-oxo-3-phenylpropan-2-yl]-2-[[(2S)-2-[(2-morpholin-4-ylacetyl)amino]-4-phenylbutanoyl]amino]pentanamide, also known as carfilzomib or Kyprolis®. In some embodiments, the proteasome inhibitor is carfilzomib. In some embodiments, the prior therapy is carfilzomib. In some embodiments, the proteasome inhibitor has the following structure

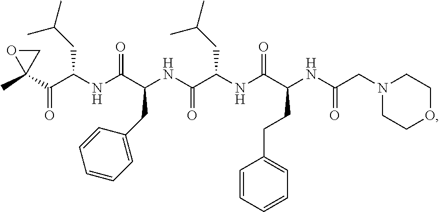 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

or a pharmaceutically acceptable salt, solvate, hydrate, stereoisomer, tautomer or racemic mixtures thereof, including and compositions thereof. In some embodiments, the proteasome inhibitor has the following structure

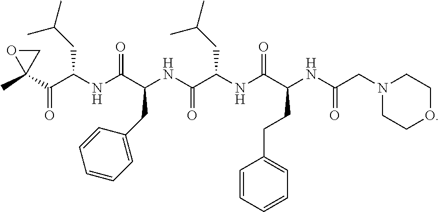 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Compositions of carfilzomib include but are not limited to those described in U.S. Pat. Nos. 7,232,818, 7,417,042, 7,491,704, 7,737,112, 8,129,346, 8,207,127, 8,207,125, 8,207,126, 8,207,297, 9,493,582, 9,511,109, and U.S. Ser. No. 10/098,890; and International Publication Nos. WO2015198257 (each incorporated herein by reference in its entirety).

In some embodiments, the proteasome inhibitor reversibly inhibits the CT-L proteolytic (β5) site of the 20S proteasome. In some embodiments, the proteasome inhibitor is [(1R)-1-[[2-[(2,5-dichlorobenzoyl)amino]acetyl]amino]-3-methylbutyl]boronic acid, also known as ixazomib or Ninlaro®. In some embodiments, the proteasome inhibitor is ixazomib. In some embodiments, the prior therapy is ixazomib. In some embodiments, the proteasome inhibitor has the following structure

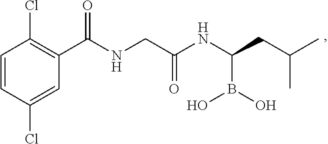 [SMILES: CC(C)C[C@H](NC(=O)CNC(=O)c1cc(Cl)ccc1Cl)B(O)O]

or a pharmaceutically acceptable salt, solvate, hydrate, stereoisomer, tautomer or racemic mixtures thereof, including and compositions thereof. In some embodiments, the proteasome inhibitor has the following structure

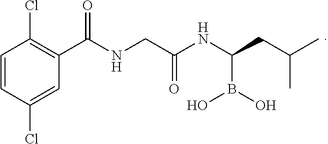 [SMILES: CC(C)C[C@H](NC(=O)CNC(=O)c1cc(Cl)ccc1Cl)B(O)O]

Compositions of carfilzomib include but are not limited to those described in U.S. Pat. Nos. 8,871,745, 8,530,694, 7,442,830, 9,175,017, 8,003,819, 9,233,115, 8,546,608, 7,687,662, and 8,859,504; and International Publication Nos. WO 2016/165677, WO 2017/174064, WO 2017/046815 (each incorporated herein by reference in its entirety).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### 3. Anti-CD38 Antibodies

In some embodiments, the prior therapy for treating the cancer is an anti-CD38 antibody. In some embodiments, the anti-CD38 antibody is a monoclonal antibody. In some embodiments, the anti-CD38 antibody is a fully human antibody or a chimeric antibody.

In some embodiments, the anti-CD38 antibody is a fully human antibody. In some embodiments, the anti-CD38 antibody is selected from among the group consisting of daratumumab, MOR202, and TAK-079. In some embodiments, the anti-CD38 antibody comprises a CDRH-1, a CDRH-2, and a CDR-H3 comprising the amino acid sequences of SEQ ID NOS:270-272, respectively. In some embodiments, the anti-CD38 antibody comprises a CDRL-1, a CDRL-2, and a CDR-L3 comprising the amino acid sequences of SEQ ID NOS:273-275, respectively. In some embodiments, the anti-CD38 antibody comprises a CDRH-1, a CDRH-2, and a CDR-H3 comprising the amino acid sequences of SEQ ID NOS:270-272, respectively; and a CDRL-1, a CDRL-2, and a CDR-L3 comprising the amino acid sequences of SEQ ID NOS:273-275, respectively. In some embodiments, the anti-CD38 antibody comprises the VH region set forth in SEQ ID NO: 276. In some embodiments, the anti-CD38 antibody comprises the VL region set forth in SEQ ID NO: 277. In some embodiments, the anti-CD38 antibody comprises the VH region set forth in SEQ ID NO: 276 and the VL region set forth in SEQ ID NO: 277. In some embodiments, the anti-CD38 antibody is daratumumab. In some embodiments, the prior therapy is daratumumab.

In some embodiments, the antibody is a chimeric antibody. In some embodiments, the anti-CD38 antibody comprises a CDRH-1, a CDRH-2, and a CDR-H3 comprising the amino acid sequences of SEQ ID NOS:278-280, respectively. In some embodiments, the anti-CD38 antibody comprises a CDRL-1, a CDRL-2, and a CDR-L3 comprising the amino acid sequences of SEQ ID NOS:281-283, respectively. In some embodiments, the anti-CD38 antibody comprises a CDRH-1, a CDRH-2, and a CDR-H3 comprising the amino acid sequences of SEQ ID NOS:278-280, respectively; and a CDRL-1, a CDRL-2, and a CDR-L3 comprising the amino acid sequences of SEQ ID NOS:281-283, respectively. In some embodiments, the anti-CD38 antibody comprises the VH region set forth in SEQ ID NO: 284. In some embodiments, the anti-CD38 antibody comprises the VL region set forth in SEQ ID NO: 285. In some embodiments, the anti-CD38 antibody comprises the VH region set forth in SEQ ID NO: 284 and the VL region set forth in SEQ ID NO: 285. In some embodiments, the anti-CD38 antibody is isatuximab. In some embodiments, the prior therapy is isatuximab.

### 4. BTK Inhibitors

In some embodiments, the prior therapy for treating the cancer is an inhibitor of Bruton's tyrosine kinase (BTK).

In some embodiments, the BTK inhibitor is an inhibitor of one or more members of the TEC family of kinases, including Bruton's tyrosine kinase (BTK), IL-2 inducible T-cell kinase (ITK), tec protein tyrosine kinase (TEC), bone marrow tyrosine kinase gene in chromosome X protein (BMX) non-receptor tyrosine kinase (also known as Epithelial and endothelial tyrosine kinase; ETK), and TXK tyrosine kinase (TXK). In some embodiments, the BTK inhibitor inhibits IL-2 inducible T-cell kinase (ITK). In some embodiments, the BTK inhibitor inhibits both BTK and ITK.

In some embodiments, the BTK inhibitor is an irreversible inhibitor of one or more TEC family kinases. In some embodiments, the BTK inhibitor is an irreversible inhibitor of BTK. In some embodiments, the BTK inhibitor is an irreversible inhibitor of ITK.

In some embodiments, the BTK inhibitor inhibits BTK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the BTK inhibitor binds to BTK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the BTK inhibitor for BTK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the BTK inhibitor inhibits ITK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the BTK inhibitor binds to ITK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the BTK inhibitor for ITK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the BTK inhibitor inhibits both BTK and ITK. In some embodiments, the BTK inhibitor inhibits both BTK and ITK with a half-maximal inhibitory concentration (IC₅₀) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the BTK inhibitor binds to both BTK and ITK with an equilibrium dissociation constant (Kd) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the inhibition constant (Ki) of the BTK inhibitor for both BTK and ITK is less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, or less than or less than about 0.1 nM.

In some embodiments, the IC₅₀, Kd and/or Ki is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17.) In some embodiments, inhibitors can be assessed for their affinity for a particular kinase or kinases, such as by using competition ligand binding assays (Ma et al., Expert Opin Drug Discov. 2008 June; 3(6):607-621) From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the equilibrium dissociation constant (Kd) and/or the inhibition constant (Ki values) can be determined additionally or alternatively. IC₅₀ and Kd can be calculated by any number of means known in the art. The inhibition constant (Ki values) can be calculated from the IC₅₀ and Kd values according to the Cheng-Prusoff equation: Ki=IC₅₀/(1+L/Kd), where L is the concentration of the BTK inhibitor (Biochem Pharmacol 22: 3099-3108, 1973). Ki is the concentration of unlabeled inhibitor that would cause occupancy of 50% of the binding sites present in the absence of ligand or other competitors.

In some embodiments, the BTK inhibitor is a small molecule.

In some embodiments, the BTK inhibitor is an inhibitor of a tyrosine protein kinase that has an accessible cysteine residue near the active site of the tyrosine kinase. In some embodiments, the BTK inhibitor of one or more TEC family kinases forms a covalent bond with a cysteine residue on the protein tyrosine kinase. In some embodiments, the cysteine residue is a Cys 481 residue. In some embodiments, the cysteine residue is a Cys 442 residue. In some embodiments, the BTK inhibitor is an irreversible BTK inhibitor that binds to Cys 481. In some embodiments, the BTK inhibitor is an ITK inhibitor that binds to Cys 442. In some embodiments, the BTK inhibitor comprises a Michael acceptor moiety that forms a covalent bond with the appropriate cysteine residue of the tyrosine kinase. In some embodiments, the Michael acceptor moiety preferentially binds with the appropriate cysteine side chain of the tyrosine kinase protein relative to other biological molecules that also contain an assessable —SH moiety.

In some embodiments, the BTK inhibitor is an ITK inhibitor compound described in PCT Application Numbers WO2002/0500071, WO2005/070420, WO2005/079791, WO2007/076228, WO2007/058832, WO2004/016610, WO2004/016611, WO2004/016600, WO2004/016615, WO2005/026175, WO2006/065946, WO2007/027594, WO2007/017455, WO2008/025820, WO2008/025821, WO2008/025822, WO2011/017219, WO2011/090760, WO2009/158571, WO2009/051822, WO2014/082085, WO2014/093383, WO2014/105958, and WO2014/145403, which are each incorporated by reference in their entireties. In some embodiments, the BTK inhibitor is an ITK inhibitor compound described in U.S. Application Numbers US20110281850, US2014/0256704, US20140315909, and US20140303161, which are each incorporated by reference in their entireties. In some embodiments, the BTK inhibitor is an ITK inhibitor compound described in U.S. Pat. No. 8,759,358, which is incorporated by reference in its entirety.

In some embodiments, the BTK inhibitor, such as a BTK/ITK inhibitor, has a structure selected from

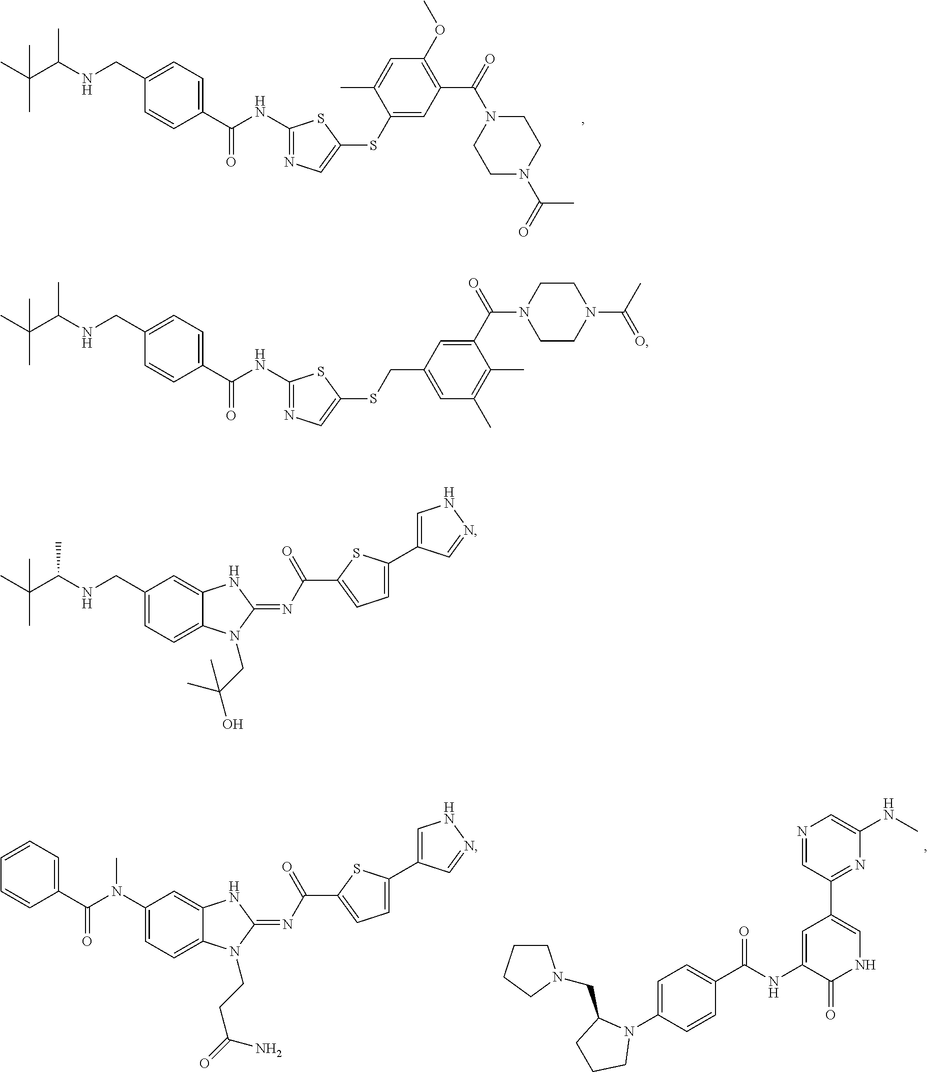

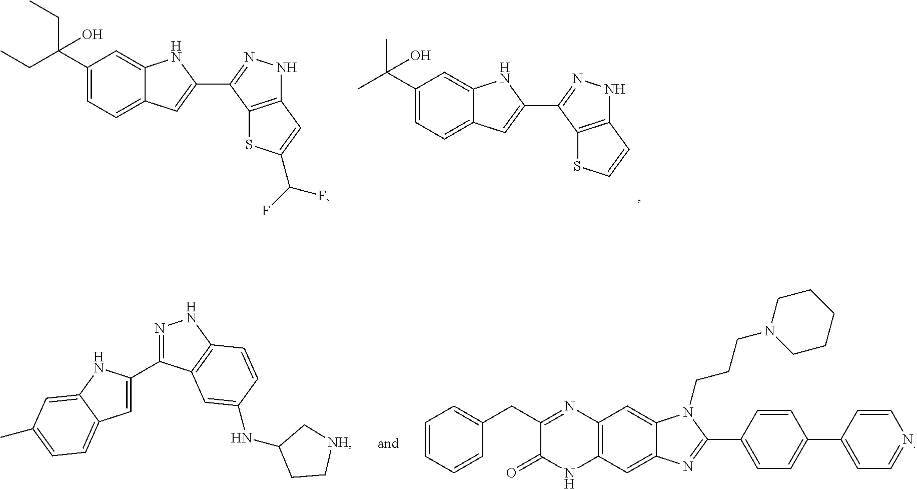 [SMILES: CCC(O)(CC)c1ccc2cc(-c3n[nH]c4cc(C(F)F)sc34)[nH]c2c1 ; CC(C)(O)c1ccc2cc(-c3n[nH]c4ccsc34)[nH]c2c1 ; Cc1ccc2cc(-c3n[nH]c4ccc(NC5CCNC5)cc34)[nH]c2c1 ; O=c1[nH]c2cc3nc(-c4ccc(-c5ccncc5)cc4)n(CCCN4CCCCC4)c3cc2nc1Cc1ccccc1]

Exemplary inhibitors of BTK and/or ITK are known in the art. In some embodiments, the BTK inhibitor is an inhibitor as described in Byrd et al., N Engl J Med. 2016; 374(4):323-32; Cho et al., J Immunol. 2015, doi:10.4049/jimmunol.1501828; Zhong et. al., J Biol. Chem., 2015, 290(10): 5960-78; Hendriks et al., Nature, 2014, 14: 219-232; Akinleye et al., Journal of Hematology & Oncology 2013, 6:59; Wang et al., ACS Med Chem Lett. 2012 Jul. 26; 3(9): 705-9; Howard et al., J Med Chem. 2009 Jan. 22; 52(2):379-88; Anastassiasdis et al., Nat Biotechnol. 2011 Oct. 30; 29(11): 1039-45; Davis, et al., Nat Biotechnol, 2011; 29:1046-51; Bamborough et al., J Med Chem. 2008 Dec. 25; 51(24):7898-914; Roth et al., J Med Chem. 2015; 58:1053-63; Galkin et al., Proc Natl Acad Sci USA. 2007; 104:270-5; Singh et al., J Med Chem. 2012; 55:3614-43; Hall et al., J Med Chem. 2009 May 28; 52(10):3191-204; Zhou et al., Nature. 2009 Dec. 24; 462(7276):1070-4; Zapf et al., J Med Chem. 2012; 55:10047-63; Shi et al., Bioorg Med Chem Lett, 2014; 24:2206-11; Illig, et al., J Med Chem. 2011; 54:7860-83; and U.S. Patent Application Publication No: 20140371241.

Non-limiting examples of BTK inhibitors, such as a BTK/ITK inhibitor include Ibrutinib (PL-32765); PRN694; Spebrutinib (CC-292 or AVL-292); PCI-45292; RN-486; Compound 2c; AT9283; BML-275; Dovitinib (TKI158); Foretinib (GSK1363089); Gö6976; GSK-3 Inhibitor IX; GSK-3 Inhibitor XIII; Hesperadin; IDR E804; K-252a; Lestaurtinib (CEP701); Nintedanib (BIBF 1120); NVP-TAE684; R406; SB218078; Staurosporine (AM-2282); Sunitinib (SU11248); Syk Inhibitor; WZ3146; WZ4002; BDBM50399459 (CHEMBL2179805); BDBM50399460 (CHEMBL2179804); BDBM50399458 (CHEMBL2179806); BDBM50399461 (CHEMBL2179790); BDBM50012060 (CHEMBL3263640); BDBM50355504 (CHEMBL1908393); BDBM50355499 (CHEMBL1908395::CHEMBL1908842).

In some embodiments, the BTK inhibitor is selected from among the group consisting of ibrutinib, acalabrutinib, zanubrutinib, evobrutinib, tirabrutinib, and SNS-062.

In some embodiments, the BTK inhibitor, such as a BTK/ITK inhibitor is or comprises ibrutinib. In some embodiments, the BTK inhibitor is ibrutinib. In some embodiments, the prior therapy is ibrutinib. In some embodiments, the BTK inhibitor is has or comprises the following structure:

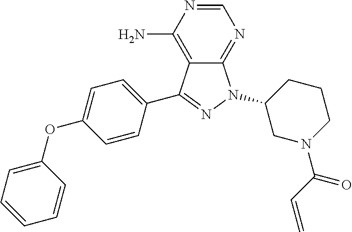 [SMILES: C=CC(=O)N1CCC[C@@H](n2nc(-c3ccc(Oc4ccccc4)cc3)c3c(N)ncnc32)C1]

or an enantiomer or mixture of enantiomers thereof, or a pharmaceutically acceptable salt, solvate, hydrate, co-crystal, clathrate, or polymorph thereof. In some embodiments, the BTK inhibitor is ibrutinib and has or comprises the following structure:

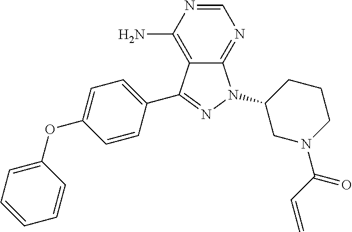 [SMILES: C=CC(=O)N1CCC[C@@H](n2nc(-c3ccc(Oc4ccccc4)cc3)c3c(N)ncnc32)C1]

or an enantiomer or mixture of enantiomers thereof, or a pharmaceutically acceptable salt, solvate, hydrate, co-crystal, clathrate, or polymorph thereof.

In some embodiments, the BTK inhibitor is an inhibitor as described in U.S. Patent No. US 2014/0371241; US 2015/0140085; US 2015/0238490; US 2015/0352116; US 2015/0361504; US 2016/0022683; US 2016/0022684; US 2016/0038495; US 2016/0038496; US 2016/0287592; US 2017/0002009; US 2017/0079981; US 2017/0128448; US 2017/0209462; US 2017/0226108; US 2017/0226114; US 2017/0305914; US 2017/0360796; US 2017/0368173; US 2018/0009814; US 2018/0028537; US 2018/0051026; US 2018/0071293; US 2018/0071295; US 2018/0072737; U.S. Pat. Nos. 7,514,444; 8,008,309; 8,476,284; 8,497,277; 8,697,711; 8,703,780; 8,735,403; 8,754,090; 8,754,091; 8,957,079; 8,999,999; 9,125,889; 9,181,257; 9,296,753; 9,545,407; 9,655,857; 9,717,731; 9,725,455; 9,730,938; 9,751,889; and 9,884,869. In some embodiments, the BTK inhibitor is or comprises ibrutinib. In some aspects, the BTK inhibitor is or comprises ibrutinib or 1-[(3R)-3-[4-amino-3-(4-phenoxyphenyl)pyrazolo[3,4-d]pyrimidin-1-yl]piperidin-1-yl]prop-2-en-1-one (also known as 1-[(3R)-3-[4-Amino-3-(4-phenoxyphenyl)-1H-pyrazolo[3,4-d]pyrimidin-1-yl]-1-piperidinyl]-2-propen-1-one; 1-[(3R)-3-[4-amino-3-(4-phenoxyphenyl)pyrazolo[3,4-d]pyrimidin-1-yl]piperidin-1-yl]prop-2-en-1-one; 1-((3R)-3-(4-amino-3-(4-phenoxyphenyl)-1H-pyrazolo(3,4-d)pyrimidin-1-yl)-1-piperidinyl)-2-Propen-1-one; 936563-96-1; PCI-32765; IMBRUVICA; UNII-1X70OSD4VX; PCI32765; CRA-032765; 1X70OSD4VX; or CHEBI:76612). In some aspects, the BTK inhibitor is or comprises 1-[(3R)-3-[4-amino-3-(4-phenoxyphenyl)pyrazolo[3,4-d]pyrimidin-1-yl]piperidin-1-yl]prop-2-en-1-one (also known as 1-[(3R)-3-[4-Amino-3-(4-phenoxyphenyl)-1H-pyrazolo[3,4-d]pyrimidin-1-yl]-1-piperidinyl]-2-propen-1-one; 1-[(3R)-3-[4-amino-3-(4-phenoxyphenyl)pyrazolo[3,4-d]pyrimidin-1-yl]piperidin-1-yl]prop-2-en-1-one; 1-((3R)-3-(4-amino-3-(4-phenoxyphenyl)-1H-pyrazolo(3,4-d)pyrimidin-1-yl)-1-piperidinyl)-2-Propen-1-one; 936563-96-1; PCI-32765; IMBRUVICA; UNII-1X70OSD4VX; PCI32765; CRA-032765; 1X70OSD4VX; or CHEBI.76612).

In some embodiments, the BTK inhibitor, such as a BTK/ITK inhibitor, is an enantiomer or a mixture of enantiomers of 1-[(3R)-3-[4-amino-3-(4-phenoxyphenyl)pyrazolo[3,4-d]pyrimidin-1-yl]piperidin-1-yl]prop-2-en-1-one, or a pharmaceutically acceptable salt, solvate, hydrate, co-crystal, clathrate, or polymorph of 1-[(3R)-3-[4-amino-3-(4-phenoxyphenyl)pyrazolo[3,4-d]pyrimidin-1-yl]piperidin-1-yl]prop-2-en-1-one. In some embodiments, the BTK inhibitor, such as a BTK/ITK inhibitor, is a solvate of 1-[(3R)-3-[4-amino-3-(4-phenoxyphenyl)pyrazolo[3,4-d]pyrimidin-1-yl]piperidin-1-yl]prop-2-en-1-one. In some embodiments, the BTK inhibitor, such as a BTK/ITK inhibitor, is a hydrate of 1-[(3R)-3-[4-amino-3-(4-phenoxyphenyl)pyrazolo[3,4-d]pyrimidin-1-yl]piperidin-1-yl]prop-2-en-1-one. In some embodiments, the BTK inhibitor, such as a BTK/ITK inhibitor, is a pharmaceutically acceptable sale of 1-[(3R)-3-[4-amino-3-(4-phenoxyphenyl)pyrazolo[3,4-d]pyrimidin-1-yl]piperidin-1-yl]prop-2-en-1-one. In some embodiments, the BTK inhibitor, such as a BTK/ITK inhibitor, is 1-[(3R)-3-[4-amino-3-(4-phenoxyphenyl)pyrazolo[3,4-d]pyrimidin-1-yl]piperidin-1-yl]prop-2-en-1-one. In certain embodiments, a BTK inhibitor, e.g., ibrutinib, is a solid. In certain embodiments, a BTK inhibitor, e.g., ibrutinib, is hydrated. In certain embodiments, a BTK inhibitor, e.g., ibrutinib, is solvated. In certain embodiments, a BTK inhibitor, e.g., ibrutinib, is anhydrous. In certain embodiments, a BTK inhibitor, e.g., ibrutinib, is nonhygroscopic.

In certain embodiments, a BTK inhibitor, e.g., ibrutinib, is amorphous. In certain embodiments, a BTK inhibitor, e.g., ibrutinib, is crystalline. In certain embodiments, the solid form of a BTK inhibitor, e.g., ibrutinib, is in a crystalline form described in U.S. Pat. No. 9,751,889, which is incorporated herein by reference in its entirety.

The solid forms of a BTK inhibitor, e.g., ibrutinib, can be prepared according to the methods described in the disclosure of WO 2016/151438, U.S. Pat. No. 9,884,869, US 2017/0226108; WO 2016/151438; WO 2017/134684; WO 2015/145415; WO 2017/137446; WO 2016/088074; WO 2017/134684; WO 2015/145415; WO 2017/085628; and WO 2017/134588 or any one or combined available method(s).

In some embodiments, a BTK inhibitor, e.g., ibrutinib, provided herein contains one chiral center, and can exist as a mixture of enantiomers, e.g., a racemic mixture. This disclosure encompasses the use of stereomerically pure forms of such a compound, as well as the use of mixtures of those forms. For example, mixtures comprising equal or unequal amounts of the enantiomers of a BTK inhibitor, e.g., ibrutinib, provided herein may be used in methods and compositions disclosed herein. These isomers may be asymmetrically synthesized or resolved using standard techniques such as chiral columns or chiral resolving agents. See, e.g., Jacques, J., et al, Enantiomers, Racemates and Resolutions (Wiley-Interscience, New York, 1981); Wilen, S. H., et al, Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E L. Eliel, Ed., Univ. of Notre Dame Press, Notre Dame, IN, 1972).

In some embodiments, the BTK inhibitor is acalabrutinib. In some embodiments, the BTK inhibitor is SNS-062. In some embodiments, the prior therapy is SNS-062. In some embodiments, the prior therapy is acalabrutinib. In some embodiments, the BTK inhibitor is evobrutinib. In some embodiments, the prior therapy is evobrutinib. In some embodiments, the BTK inhibitor is tirabrutinib. In some embodiments, the Prior therapy is tirabrutinib. In some embodiments, the prior therapy is zanubrutinib. In some embodiments, the BTK inhibitor is zanubrutinib.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### 5. BCL-2 Inhibitors

In some embodiments, the prior therapy for treating the cancer is a BCL-2 inhibitor.

In some embodiments, the BCL-2 inhibitor is a selective BCL-2 inhibitor. In some embodiments, a selective BCL-2 inhibitor is a compound or agent, such as an inhibitor of a prosurvival BCL-2 family protein, that is capable of being provided at a dosing regimen (e.g. dose and/or duration) that reduces or blocks BCL-2 activity and/or signaling to a greater extent than that of other prosurvival BCL-2 family proteins (e.g. BCLXL, BCLW, BCLB, MCL1). In some cases, a selective BCL-2 inhibitor reduces or blocks the activity of BCL-2 signaling and/or activity when provided at a dosing regimen, but does not reduce or block the signaling and/or activity of other prosurvival BCL-2 family proteins when provided at the same dosing regimen. In some cases, selective BCL-2 inhibitors exert minimal or no effects on the activity and/or signaling of other prosurvival BCL-2 family proteins, when provided at a dosing regimen.

In some embodiments, the BCL-2 inhibitor is a nonselective BCL-2 inhibitor. In some embodiments, a nonselective BCL-2 inhibitor is a compound or agent, such as an inhibitor of a prosurvival BCL-2 family protein, that reduces or blocks the activity of more than one prosurvival BCL-2 family protein. In some cases, a nonselective BCL-2 inhibitor is a compound or agent, such as an inhibitor of a prosurvival BCL-2 family protein, that is capable of being provided at a dosing regimen (e.g. dose and/or duration) that reduces or blocks the activity and/or signaling of a prosurvival BCL-2 family protein, e.g. BCL-2 and additionally reduces or blocks the activity and/or signaling of one or more other prosurvival BCL-2 family proteins (e.g. BCLXL, BCLW, BCLB, MCL1). In some cases, a nonselective BCL-2 inhibitor reduces or blocks the activity and/or signaling of a prosurvival BCL-2 family protein (e.g. BCL-2) when provided at a dosing regimen, and also reduces or blocks the signaling and/or activity of one or more other prosurvival BCL-2 family proteins (e.g. BCLXL, BCLW, BCLB, MCL1) when provided at the same dosing regimen.

In some embodiments, the BCL-2 inhibitor inhibits BCL-2 with a half-maximal inhibitory concentration (IC50) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, less than or less than about 0.1 nM, or less than or less than about 0.01 nM. In some embodiments, the BCL-2 inhibitor inhibits one or more other prosurvival BCL-2 family proteins, such as BCLXL, BCLW, BCLB, and/or MCL1 with a half-maximal inhibitory concentration (IC50) of less than or less than about 1000 nM, less than or less than about 900 nM, less than or less than about 800 nM, less than or less than about 700 nM, less than or less than about 600 nM, less than or less than about 500 nM, less than or less than about 400 nM, less than or less than about 300 nM, less than or less than about 200 nM, less than or less than about 100 nM, less than or less than about 90 nM, less than or less than about 80 nM, less than or less than about 70 nM, less than or less than about 60 nM, less than or less than about 50 nM, less than or less than about 40 nM, less than or less than about 30 nM, less than or less than about 20 nM, less than or less than about 10 nM, less than or less than about 9 nM, less than or less than about 8 nM, less than or less than about 7 nM, less than or less than about 6 nM, less than or less than about 5 nM, less than or less than about 4 nM, less than or less than about 3 nM, less than or less than about 2 nM, less than or less than about 1 nM, less than or less than about 0.9 nM, less than or less than about 0.8 nM, less than or less than about 0.7 nM, less than or less than about 0.6 nM, less than or less than about 0.5 nM, less than or less than about 0.4 nM, less than or less than about 0.3 nM, less than or less than about 0.2 nM, less than or less than about 0.1 nM, or less than or less than about 0.01 nM.

In some embodiments, the inhibition constant (Ki) of the BCL-2 inhibitor for BCL-2 is lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 10 times lower, at least 100 times lower, at least 1,000 times lower, at least 5,000 times lower, at least 10,000 times lower, or at least 20,000 times lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 1,000 times lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 5,000 times lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 10,000 times lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 20,000 times lower than the inhibition constant (Ki) of the BCL-2 inhibitor for one or more other prosurvival BCL-2 family proteins, such as BCLXL, BCLW, BCLB, and/or MCL1. In some embodiments, the inhibition constant (Ki) of the BCL-2 inhibitor for BCL-2 is at least 1,000 times lower than the inhibition constant (Ki) of the BCL-2 inhibitor for BCLXL. In some embodiments, the inhibition constant (Ki) of the BCL-2 inhibitor for BCL-2 is at least 4,000 times lower than the inhibition constant (Ki) of the BCL-2 inhibitor for BCLXL. In some embodiments, the inhibition constant (Ki) of the BCL-2 inhibitor for BCL-2 is at least 20,000 times lower than the inhibition constant (Ki) of the BCL-2 inhibitor for BCLW.

In some embodiments, the inhibition constant (Ki) of the BCL-2 inhibitor for BCL-2 is less than about 10 μM. In some embodiments, the inhibition constant (Ki) of the BCL-2 inhibitor for BCL-2 is less than about 1 μM. In some embodiments, the inhibition constant (Ki) of the BCL-2 inhibitor for BCL-2 is less than about 0.1 μM. In some embodiments, the inhibition constant (Ki) of the BCL-2 inhibitor for BCL-2 is less than about 10 nM. In some embodiments, the inhibition constant (Ki) of the BCL-2 inhibitor for BCL-2 is less than about 1.0 nM. In some embodiments, the inhibition constant (Ki) of the BCL-2 inhibitor for BCL-2 is less than about 0.1 nM. In some embodiments, the inhibition constant (Ki) of the BCL-2 inhibitor for BCL-2 is less than about 0.01 nM. In some embodiments, the inhibition constant (Ki) of the BCL-2 inhibitor for one or more other prosurvival BCL-2 family proteins, such as BCLXL, BCLW, BCLB, and/or MCL, is less than about 10 μM. In In some embodiments, the inhibition constant (Ki) of the BCL-2 inhibitor for one or more other prosurvival BCL-2 family proteins, such as BCLXL, BCLW, BCLB, and/or MCL1, is less than about 1 μM. In some embodiments, the inhibition constant (Ki) of the BCL-2 inhibitor one or more other prosurvival BCL-2 family proteins, such as BCLXL, BCLW, BCLB, and/or MCL1, is less than about 0.1 μM. In some embodiments, the inhibition constant (Ki) of the BCL-2 inhibitor for one or more other prosurvival BCL-2 family proteins, such as BCLXL, BCLW, BCLB, and/or MCL1, is less than about 10 nM. In some embodiments, the inhibition constant (Ki) of the BCL-2 inhibitor for one or more other prosurvival BCL-2 family proteins, such as BCLXL, BCLW, BCLB, and/or MCL1, is less than about 1.0 nM. In some embodiments, the inhibition constant (Ki) of the BCL-2 inhibitor for one or more other prosurvival BCL-2 family proteins, such as BCLXL, BCLW, BCLB, and/or MCL1, is less than about 0.1 nM. In some embodiments, the inhibition constant (Ki) of the BCL-2 inhibitor for one or more other prosurvival BCL-2 family proteins, such as BCLXL, BCLW, BCLB, and/or MCL1, is less than about 0.01 nM.

In some embodiments, the IC₅₀, Kd and/or Ki is measured or determined using an in vitro assay. Assays to assess or quantitate or measure activity of protein tyrosine kinase inhibitors as described are known in the art. Such assays can be conducted in vitro and include assays to assess the ability of an agent to inhibit a specific biological or biochemical function. In some embodiments, kinase activity studies can be performed. Protein tyrosine kinases catalyze the transfer of the terminal phosphate group from adenosine triphosphate (ATP) to the hydroxyl group of a tyrosine residue of the kinase itself or another protein substrate. In some embodiments, kinase activity can be measured by incubating the kinase with the substrate (e.g., inhibitor) in the presence of ATP. In some embodiments, measurement of the phosphorylated substrate by a specific kinase can be assessed by several reporter systems including colorimetric, radioactive, and fluorometric detection. (Johnson, S. A. & T. Hunter (2005) Nat. Methods 2:17). In some embodiments, inhibitors can be assessed for their affinity for a particular kinase or kinases, such as by using competition ligand binding assays (Ma et al., Expert Opin Drug Discov. 2008 June; 3(6):607-621). From these assays, the half-maximal inhibitory concentration (IC₅₀) can be calculated. IC₅₀ is the concentration that reduces a biological or biochemical response or function by 50% of its maximum. In some cases, such as in kinase activity studies, IC₅₀ is the concentration of the compound that is required to inhibit the target kinase activity by 50%. In some cases, the dissociation constant (Kd) and/or the inhibition constant (Ki values) can be determined additionally or alternatively. IC₅₀ and Kd can be calculated by any number of means known in the art. The inhibition constant (Ki values) can be calculated from the IC50 and Kd values according to the Cheng-Prusoff equation: Ki=IC50/(1+L/Kd), where L is the concentration of the inhibitor (Biochem Pharmacol 22: 3099-3108, 1973). Ki is the concentration of unlabeled inhibitor that would cause occupancy of 50% of the binding sites present in the absence of ligand or other competitors.

In some embodiments, the inhibitor is a small molecule.

In some embodiments, the BCL-2 inhibitor is an inhibitor of BCL-2, including but not limited to those described in U.S. Pat. Nos. 9,174,982, 8,546,399, 7,030,115, 7,390,799, 7,709,467, 8,624,027, 7,906,505, 6,720,338, published PCT application WO 13/096060, published PCT application WO 02/097053, published US application US 2016/0220573, U.S. Pat. No. 7,354,928, published US application 2015/0056186, and published PCT application WO 05/049594, which are each incorporated by reference in their entireties.

In some embodiments, the BCL-2 inhibitor is selected from among the group consisting of venetoclax, navitoclax, ABT737, maritoclax, obatoclax, and clitocine. In some embodiments, the BCL-2 inhibitor inhibits MCL1, such as maritoclax. In some embodiments, the BCL-2 inhibitor inhibits BCL-2, BCLXL, and BCLW, such as navitoclax. In some embodiments, the BCL-2 inhibitor inhibits BCL-2, such as venetoclax.

In some embodiments, the BCL-2 inhibitor inhibits or reduces the activity of BCL-2, BCLXL, BCLW, BCLB, BFL1, and/or MCL1. In some cases, the BCL-2 inhibitor inhibits or reduces the activity of MCL1, such as maritoclax. In some cases, the BCL-2 inhibitor induces proteasomal degradation of MCL1. In some cases, the BCL-2 inhibitor induces accumulation of MCL1. In some cases the BCL-2 inhibitor is maritoclax. In some embodiments, the prior therapy is maritoclax. In some cases, the BCL-2 inhibitor has the structure

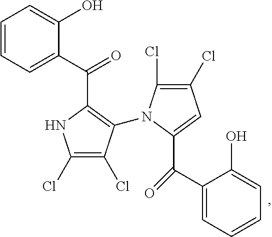 [SMILES: O=C(c1ccccc1O)c1[nH]c(Cl)c(Cl)c1-n1c(C(=O)c2ccccc2O)cc(Cl)c1Cl]

or a pharmaceutically acceptable salt, solvate, hydrate, stereoisomer, tautomer or racemic mixtures thereof, including and compositions thereof.

In some embodiments, the BCL-2 inhibitor inhibits or reduces the activity of BCL-2, BCLXL, and BCLW, such as navitoclax. In some cases, the BCL-2 inhibitor is navitoclax. In some embodiments, the prior therapy is navitoclax. In some cases, the BCL-2 inhibitor has the structure

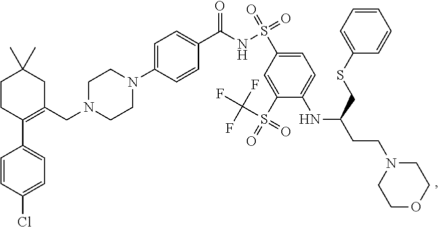 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

or a pharmaceutically acceptable salt, solvate, hydrate, stereoisomer, tautomer or racemic mixtures thereof, including and compositions thereof, for the treatment of subjects with cancer.

In some embodiments, the BCL-2 inhibitor inhibits or reduces the activity of BCL-2, such as venetoclax. In some cases, the BCL-2 inhibitor is venetoclax. In some embodiments, the prior therapy is venetoclax. In some cases, the BCL-2 inhibitor has the structure

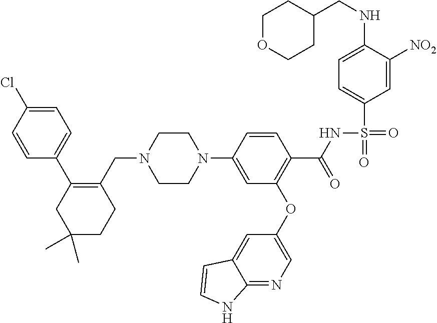 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

or a pharmaceutically acceptable salt, solvate, hydrate, stereoisomer, tautomer or racemic mixtures thereof, including and compositions thereof.

Exemplary BCL-2 inhibitors include, but are not limited to venetoclax (ABT-199), navitoclax (ABT-263), ABT-737, AT-101/GDC-0199 (Gossypol), apogossypol, TW-37, G3139 (Genasense), GX15-070 (obatoclax), sabutoclax, HA14-1, antimycin A, BH3I-1, YC137, maritoclax (marinopyyrole A), clitocine, UMI-77, WEHI-539, and 544563.

In some embodiments, the BCL-2 inhibitor is ABT737. In some embodiments, the prior therapy is ABT737. In some embodiments, the BCL-2 inhibitor is obatoclax. In some embodiments, the prior therapy is obatoclax. In some embodiments, the BCL-2 inhibitor is clitocine. In some embodiments, the prior therapy is clitocine.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of the structure.

### C. T Cell Therapy

Provided herein are methods of treating a subject having a cancer, comprising administration of a T cell therapy (e.g., CAR T cells of a TCE), wherein the subject has relapsed following treatment with, or refractory to, a prior therapy for treating the cancer. In some embodiments, the methods further comprise, following administration of the T cell therapy, administration of a subsequent therapy for treating the cancer to the subject, wherein the prior therapy and the subsequent therapy are of the same class of therapy. In some embodiments, the T cell therapy is selected from the group consisting of a dose of T cells expressing a recombinant receptor (e.g. a chimeric antigen receptor) or a T cell engager (e.g. a bispecific T cell engager).

### 1. Cells Expressing Recombinant Receptors

In some embodiments, the T cell therapy comprises engineered T cells expressing a recombinant receptor (e.g., a chimeric antigen receptor), such as one that contains an extracellular domain including an antigen binding moiety, such as an antibody or fragment as described herein. Also provided are populations of such cells, compositions containing such cells and/or enriched for such cells, such as in which cells expressing an antigen-binding moiety make up at least 50, 60, 70, 80, 90, 91, 92, 93, 94, 95, 96, 97, 98, 99, or more percent of the total cells in the composition or cells of a certain type such as PBMCs, T cells or CD3+, CD8+ or CD4+ cells.

Among the compositions are pharmaceutical compositions and formulations for administration, such as for adoptive cell therapy. Also provided are therapeutic methods for administering the cells and compositions to subjects, e.g., patients.

Thus, also provided are genetically engineered cells expressing the recombinant receptors containing the antibodies, e.g., cells containing the CARs. The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid encoding a recombinant receptor described herein.

In some embodiments, the T cell therapy for use in accord with the provided methods includes administering engineered T cells expressing recombinant receptors designed to recognize and/or specifically bind to molecules associated with a cancer. In some cases, the recombinant receptor binds to an antigen expressed by a cancer such as multiple myeloma, for example relapsed and refractory (R/R) multiple myeloma (MM) (e.g., BCMA). In some cases, the recombinant receptor binds to an antigen expressed by a cancer such as multiple myeloma, for example relapsed and refractory (R/R) multiple myeloma (MM) (e.g., GPRC5D). In some cases, the recombinant receptor binds to an antigen expressed by a cancer such as a leukemia or lymphoma, for example relapsed and refractory (R/R) leukemia or lymphoma (e.g., CD19). In some embodiments, binding to the antigen results in a response, such as an immune response against such molecules upon binding to such molecules. In some embodiments, the cells contain or are engineered to contain an engineered receptor, e.g., an engineered antigen receptor, such as a chimeric antigen receptor (CAR), or a T cell receptor (TCR). The recombinant receptor, such as a CAR, generally includes an extracellular antigen (or ligand) binding domain that is directed against an antigen (e.g., BCMA), linked to one or more intracellular signaling components, in some aspects via linkers and/or transmembrane domain(s). In some aspects, the engineered cells are provided as pharmaceutical compositions and formulations suitable for administration to a subjects, such as for adoptive cell therapy. Also provided are therapeutic methods for administering the cells and compositions to subjects, e.g., patients.

In some embodiments, the cells include one or more nucleic acids introduced via genetic engineering, and thereby express recombinant or genetically engineered products of such nucleic acids. In some embodiments, gene transfer is accomplished by first stimulating the cells, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

a. Recombinant Receptors, e.g. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

In some embodiments of the provided methods and uses, the engineered cells, such as T cells, express a chimeric receptor, such as a chimeric antigen receptor (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding the antigen, single chain antibody fragments, including single chain variable fragments (scFv), and single domain antibodies (e.g., sdAb, sdFv, nanobody, VHH) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific or trispecific, antibodies, diabodies, triabodies, and tetrabodies, tandem di-scFv, tandem tri-scFv. Unless otherwise stated, the term “antibody” should be understood to encompass functional antibody fragments thereof also referred to herein as “antigen-binding fragments.” The term also encompasses intact or full-length antibodies, including antibodies of any class or sub-class, including IgG and sub-classes thereof, IgM, IgE, IgA, and IgD.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (VH and VL, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Among the antigen binding domains included in the CARs are antibody fragments. An “antibody fragment” or “antigen-binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; heavy chain variable (VH) regions, single-chain antibody molecules such as scFvs and single-domain antibodies comprising only the VH region; and multispecific antibodies formed from antibody fragments. In particular embodiments, the antibodies are single-chain antibody fragments comprising a heavy chain variable (VH) region and/or a light chain variable (VL) region, such as scFvs.

Single-domain antibodies (sdAbs) are antibody fragments comprising all or a portion of the heavy chain variable region or all or a portion of the light chain variable region of an antibody. In certain embodiments, a single-domain antibody is a human single-domain antibody. In certain embodiments, a single-domain antibody is a human single-domain antibody. In some embodiments, the CAR comprises an antibody heavy chain domain that specifically binds an antigen expressed by a cancer.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR includes an antigen-binding portion or portions of an antibody molecule, such as a single-chain antibody fragment (scFv) derived from the variable heavy (VH) and variable light (VL) chains of a monoclonal antibody (mAb), or a single domain antibody (sdAb), such as sdFv, nanobody, VHH and VNAR. In some embodiments, an antigen-binding fragment comprises antibody variable regions joined by a flexible linker.

In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the CAR comprises a sdAb. In some embodiments, the CAR comprises two sdAbs. In some embodiments, each of the two sdAbs is a VH domain. In some embodiments, the two sdAbs bind to different epitopes of an antigen (e.g., BCMA). In some embodiments, the two sdAbs bind to the same epitope of an antigen (e.g., BCMA). In some embodiments, the antibody or antigen binding fragment is an scFv comprising a heavy chain variable (VH) region and a light chain variable (VL) region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the antibodies included in the provided CARs are murine antibodies. A “murine antibody” is an antibody with an amino acid sequence corresponding to that of an antibody produced by a murine or a murine cell, or non-murine source that utilizes murine antibody repertoires or other murine antibody-encoding sequences, including murine antibody libraries.

Also among the antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

Among the antibodies included in the provided CARs are those that are monoclonal antibodies, including monoclonal antibody fragments. The term “monoclonal antibody” as used herein refers to an antibody obtained from or within a population of substantially homogeneous antibodies, i.e., the individual antibodies comprising the population are identical, except for possible variants containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. The term is not to be construed as requiring production of the antibody by any particular method. A monoclonal antibody may be made by a variety of techniques, including but not limited to generation from a hybridoma, recombinant DNA methods, phage-display and other antibody display methods.

Thus, in some embodiments, the chimeric antigen receptor, including TCR-like CARS, includes an extracellular portion containing an antibody or antibody fragment. In some embodiments, the antibody or fragment includes an scFv. In some embodiments, the antibody or antigen-binding fragment thereof is a single-chain antibody fragment, such as a single chain variable fragment (scFv) or a diabody or a single domain antibody (sdAb). In some embodiments, the antibody or antigen-binding fragment is a single domain antibody comprising only the VH region. In some embodiments, the antibody or antigen binding fragment is an scFv comprising a heavy chain variable (VH) region and a light chain variable (VL) region.

In some embodiments, the antibody is an antigen-binding fragment, such as a scFv, that includes one or more linkers joining two antibody domains or regions, such as a heavy chain variable (VH) region and a light chain variable (VL) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO:26) or GGGS (3GS; SEQ ID NO:27), such as between 2, 3, 4, and 5 repeats of such a sequence. Exemplary linkers include those having or consisting of an sequence set forth in SEQ ID NO:28 (GGGGSGGGGSGGGGS), SEQ ID NO:29 (GSTSGSGKPGSGEGSTKG), SEQ ID NO: 30 (SRGGGGSGGGGSGGGGSLEMA), or SEQ ID NO:38 (ASGGGGSGGRASGGGGS). In some embodiments, the linker is or comprises the sequence set forth in SEQ ID NO:29.

In some embodiments of the provided methods, chimeric receptors, such as a chimeric antigen receptors, contain one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, WO2016/0046724, WO2016/014789, WO2016/090320, WO2016/094304, WO2017/025038, WO2017/173256, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, 8,479,118, and 9,765,342, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

Exemplary antigen receptors, e.g., CARs, also include any described in Marofi et al., Stem Cell Res Ther 12: 81 (2021); Townsend et al., J Exp Clin Cancer Res 37: 163 (2018); Ma et al., Int J Biol Sci 15(12): 2548-2560 (2019); Zhao and Cao, Front Immunol 10: 2250 (2019); Han et al., J Cancer 12(2): 326-334 (2021); Specht et al., Cancer Res 79: 4 Supplement, Abstract P2-09-13; Byers et al., Journal of Clinical Oncology 37, no. 15_suppl (2019); Panowski et al., Cancer Res 79 (13 Supplement) 2326 (2019); and Sauer et al., Blood 134 (Supplement 1): 1932 (2019); or can contain any of the antibodies or antigen-binding fragments described in U.S. Pat. Nos. 8,153,765; 8,603,477, 8,008,450; U.S. Pub. No. US20120189622 or US20100260748; and International PCT Publication Nos. WO2006099875, WO2009080829, WO2012092612, WO2014210064.

Further exemplary antigen receptors, e.g., CARs, such as anti-BCMA CARs, include the CARs of idecabtagene vicleucel, ABECMA©, BCMA02, JCARH125, JNJ-68284528 (LCAR-B38M; ciltacabtagene autoleucel; CARVYKTI™) (Janssen/Legend), P-BCMA-101 (Poseida), PBCAR269A (Poseida), P-BCMA-Allo1 (Poseida), Allo-715 (Pfizer/Allogene), CT053 (Carsgen), Descartes-08 (Cartesian), PHE885 (Novartis), ARI-002 (Hospital Clinic Barcelona, IDIBAPS), and CTX120 (CRISPR Therapeutics). In a particular embodiment, the CAR is the CAR of idecabtagene vicleucel cells. In a particular embodiment, the CAR is the CAR of ABECMA® cells (cells used in ABECMA® immunotherapy). In a particular embodiment, the CAR is the CAR of ciltacabtagene autoleucel cells. In a particular embodiment, the CAR is the CAR of CARVYKTI™ cells (cells used in CARVYKTI™ immunotherapy).

Exemplary antigen receptors, e.g., CARs, also include the CARs of FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel), ABECMA® (idecabtagene vicleucel), and CARVYKTI™ (ciltacabtagene autoleucel). In some of any of the provided embodiments, the CAR is the CAR of BREYANZI® (lisocabtagene maraleucel), TECARTUS™ (brexucabtagene autoleucel), KYMRIAH™ (tisagenlecleucel), YESCARTA™ (axicabtagene ciloleucel), ABECMA® (idecabtagene vicleucel), or CARVYKTI™ (ciltacabtagene autoleucel). In some of any of the provided embodiments, the CAR is the CAR of BREYANZI® (lisocabtagene maraleucel, see Sehgal et al., 2020, Journal of Clinical Oncology 38:15_suppl, 8040; Teoh et al., 2019, Blood 134(Supplement_1):593; and Abramson et al., 2020, The Lancet 396(10254): 839-852). In some of any of the provided embodiments, the CAR is the CAR of TECARTUS™ (brexucabtagene autoleucel, see Mian and Hill, 2021, Expert Opin Biol Ther; 21(4):435-441; and Wang et al., 2021, Blood 138(Supplement 1):744). In some of any of the provided embodiments, the CAR is the CAR of KYMRIAH™ (tisagenlecleucel, see Bishop et al., 2022, N Engl J Med 386:629:639; Schuster et al., 2019, N Engl J Med 380:45-56; Halford et al., 2021, Ann Pharmacother 55(4):466-479; Mueller et al., 2021, Blood Adv. 5(23):4980-4991; and Fowler et al., 2022, Nature Medicine 28:325-332). In some of any of the provided embodiments, the CAR is the CAR of YESCARTA™ (axicabtagene ciloleucel, see Neelapu et al., 2017, N Engl J Med 377(26):2531-2544; Jacobson et al., 2021, The Lancet 23(1):P91-103; and Locke et al., 2022, N Engl J Med 386:640-654). In some of any of the provided embodiments, the CAR is the CAR of ABECMA® (idecabtagene vicleucel, see Raje et al., 2019, N Engl J Med 380:1726-1737; and Munshi et al., 2021, N Engl J Med 384:705-716). In some of any of the provided embodiments, the CAR is the CAR of CARVYKTI™ (ciltacabtagene autoleucel, see Berdeja et al., Lancet. 2021 Jul. 24; 398(10297):314-324; and Martin, Abstract #549 [Oral], presented at 2021 American Society of Hematology (ASH) Annual Meeting & Exposition)).

In some embodiments, the antigen is BCMA. In some embodiments, the CAR includes a BCMA-binding portion or portions of the antibody molecule, such as a heavy chain variable (VH) region and/or light chain variable (VL) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin. Cancer Res., 2013, 19(8):2048-2060; Feng et al., Scand. J. Immunol. (2020) 92:e12910; U.S. Pat. Nos. 9,034,324 9,765,342; U.S. Patent Publication Nos. US2016/0046724, US20170183418; and International PCT Application Nos. WO 2016090320, WO2016090327, WO2016094304, WO2016014565, WO2016014789, WO2010104949, WO2017025038, WO2017173256, WO2018085690, or WO2021091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO2017025038 or WO2018028647. In some embodiments, the anti-BCMA CAR contains two single-domain anti-BCMA antibodies. In some embodiments, the two single-domain anti-BCMA antibodies are derived from one or more antibodies described in WO2017025038 or WO2018028647. In some embodiments, the BCMA binding domain comprises or consists of A37353-G4S-A37917 (G4S being a linker between the two binding domains), described in WO2017025038 or WO2018028647, and provided, e.g., in SEQ ID NOs: 300, 301 and 302 of WO2017025038 or WO2018028647 (with or without signal peptide). In some embodiments, the anti-BCMA CAR contains an antigen-binding domain that is an scFv containing a variable heavy (VH) and/or a variable light (VL) region. In some embodiments, the scFv containing a variable heavy (VH) and/or a variable light (VL) region is derived from an antibody described in WO2016090320 or WO2016090327. In some embodiments, the scFv containing a variable heavy (VH) and/or a variable light (VL) region is derived from an antibody described in WO 2019/090003. In some embodiments, the scFv containing a variable heavy (VH) and/or a variable light (VL) region is derived from an antibody described in WO2016094304 or WO2021091978. In some embodiments, the scFv containing a variable heavy (VH) and/or a variable light (VL) region is derived from an antibody described in WO2018133877. In some embodiments, the scFv containing a variable heavy (VH) and/or a variable light (VL) region is derived from an antibody described in WO2019149269. In some embodiments, the anti-BCMA CAR is any as described in WO2019173636 or WO2020051374A. In some embodiments, the anti-BCMA CAR is any as described in WO2018102752. In some embodiments, the anti-BCMA CAR is any as described in WO2020112796 or WO2021173630.

In some embodiments, the antibody, e.g., the anti-BCMA antibody or antigen-binding fragment, contains a heavy and/or light chain variable (VH or VL) region sequence as described, or a sufficient antigen-binding portion thereof. In some embodiments, the anti-BCMA antibody, e.g., antigen-binding fragment, contains a VH region sequence or sufficient antigen-binding portion thereof that contains a CDR-H1, CDR-H2 and/or CDR-H3 as described. In some embodiments, the anti-BCMA antibody, e.g., antigen-binding fragment, contains a VL region sequence or sufficient antigen-binding portion that contains a CDR-L1, CDR-L2 and/or CDR-L3 as described. In some embodiments, the anti-BCMA antibody, e.g., antigen-binding fragment, contains a VH region sequence that contains a CDR-H1, CDR-H2 and/or CDR-H3 as described and contains a VL region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a VH region sequence or a sufficient antigen-binding portion thereof, such as any of the above described VH sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a VH region further comprises a light chain or a sufficient antigen binding portion thereof. For example, in some embodiments, the antibody or antigen-binding fragment thereof contains a VH region and a VL region, or a sufficient antigen-binding portion of a VH and VL region. In such embodiments, a VH region sequence can be any of the above described VH sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8):2048-2060, U.S. Pat. No. 9,765,342, WO 2016/090320, WO2016090327, WO2010104949A2, WO2016/0046724, WO2016/014789, WO2016/094304, WO2017/025038, and WO2017173256.

In some embodiments, the anti-BCMA CAR contains an antigen-binding domain, such as an scFv, containing a variable heavy (VH) and/or a variable light (VL) region derived from an antibody described in WO2016094304 or WO2021091978. In some embodiments, the antigen-binding domain is an antibody fragment containing a variable heavy chain (VH) and a variable light chain (VL) region. In some embodiments, the anti-BCMA CAR contains an antigen-binding domain, such as an scFv, containing a variable heavy (VH) and/or a variable light (VL) region derived from an antibody described in WO 2016/090320 or WO2016090327.

In some embodiments, the antigen-binding domain is an antibody fragment containing a variable heavy chain (VH) and a variable light chain (VL) region. In some aspects, the VH region is or includes an amino acid sequence having at least 90%, 91%, 92%, 93%, 94%, 95%, 96%, 97%, 98% or 99% sequence identity to the VH region amino acid sequence set forth in any of SEQ ID NOs: 18, 20, 22, 24, 32, 34, 36, 41, 43, 45, 47, 49, 51, 53, 55, 57, 59, 61, 63, 65, 67, 69, 71, 73, 75, 145, 147, 149 and 151; and/or the VL region is or includes an amino acid sequence having at least 90%, 91%, 92%, 93%, 94%, 95%, 96%, 97%, 98%, or 99% sequence identity to the VL region amino acid sequence set forth in any of SEQ ID NOs: 19, 21, 23, 25, 33, 35, 37, 42, 44, 46, 48, 50, 52, 54, 56, 58, 60, 62, 64, 66, 68, 70, 72, 74, 76, 146, 148, 150 and 152.

In some embodiments, the antigen-binding domain, such as an scFv, contains a VH set forth in SEQ ID NO: 18 and a VL set forth in SEQ ID NO: 19. In some embodiments, the antigen-binding domain, such as an scFv, contains a VH set forth in SEQ ID NO: 20 and a VL set forth in SEQ ID NO:21. In some embodiments, the antigen-binding domain, such as an scFv, contains a VH set forth in SEQ ID NO: 22 and a VL set forth in SEQ ID NO:23. In some embodiments, the antigen-binding domain, such as an scFv, contains a VH set forth in SEQ ID NO: 24 and a VL set forth in SEQ ID NO:25. In some embodiment the antigen-binding domain, such as an scFv, contains a VH set forth in SEQ ID NO: 32 and a VL set forth in SEQ ID NO:33. In some embodiments, the antigen-binding domain, such as an scFv, contains a VH set forth in SEQ ID NO: 34 and a VL set forth in SEQ ID NO:35. In some embodiments, the antigen-binding domain, such as an scFv, contains a VH set forth in SEQ ID NO: 36 and a VL set forth in SEQ ID NO:37. In some embodiments, the antigen-binding domain, such as an scFv, contains a VH set forth in SEQ ID NO: 41 and a VL set forth in SEQ ID NO: 42. In some embodiments, the antigen-binding domain, such as an scFv, contains a VH set forth in SEQ ID NO: 43 and a VL set forth in SEQ ID NO: 44. In some embodiments, the antigen-binding domain, such as an scFv, contains a VH set forth in SEQ ID NO: 45 and a VL set forth in SEQ ID NO: 46. In some embodiments, the antigen-binding domain, such as an scFv, contains a VH set forth in SEQ ID NO: 47 and a VL set forth in SEQ ID NO: 48. In some embodiments, the antigen-binding domain, such as an scFv, contains a VH set forth in SEQ ID NO: 49 and a VL set forth in SEQ ID NO: 50. In some embodiments, the antigen-binding domain, such as an scFv, contains a VH set forth in SEQ ID NO: 51 and a VL set forth in SEQ ID NO: 52. In some embodiments, the antigen-binding domain, such as an scFv, contains a VH set forth in SEQ ID NO: 53 and a VL set forth in SEQ ID NO: 54. In some embodiments, the antigen-binding domain, such as an scFv, contains a VH set forth in SEQ ID NO: 55 and a VL set forth in SEQ ID NO: 56. In some embodiments, the antigen-binding domain, such as an scFv, contains a VH set forth in SEQ ID NO: 57 and a VL set forth in SEQ ID NO: 58. In some embodiments, the antigen-binding domain, such as an scFv, contains a VH set forth in SEQ ID NO: 59 and a VL set forth in SEQ ID NO: 60. In some embodiments, the antigen-binding domain, such as an scFv, contains a VH set forth in SEQ ID NO: 61 and a VL set forth in SEQ ID NO: 62. In some embodiments, the antigen-binding domain, such as an scFv, contains a VH set forth in SEQ ID NO: 63 and a VL set forth in SEQ ID NO: 64. In some embodiments, the antigen-binding domain, such as an scFv, contains a VH set forth in SEQ ID NO: 65 and a VL set forth in SEQ ID NO: 66. In some embodiments, the antigen-binding domain, such as an scFv, contains a VH set forth in SEQ ID NO: 67 and a VL set forth in SEQ ID NO: 68. In some embodiments, the antigen-binding domain, such as an scFv, contains a VH set forth in SEQ ID NO: 69 and a VL set forth in SEQ ID NO: 70. In some embodiments, the antigen-binding domain, such as an scFv, contains a VH set forth in SEQ ID NO: 71 and a VL set forth in SEQ ID NO: 72. In some embodiments, the antigen-binding domain, such as an scFv, contains a VH set forth in SEQ ID NO: 73 and a VL set forth in SEQ ID NO: 74. In some embodiments, the antigen-binding domain, such as an scFv, contains a VH set forth in SEQ ID NO: 75 and a VL set forth in SEQ ID NO: 76. In some embodiments, the antigen-binding domain, such as an scFv, contains a VH set forth in SEQ ID NO: 145 and a VL set forth in SEQ ID NO: 146. In some embodiments, the antigen-binding domain, such as an scFv, contains a VH set forth in SEQ ID NO: 147 and a VL set forth in SEQ ID NO: 148. In some embodiments, the antigen-binding domain, such as an scFv, contains a VH set forth in SEQ ID NO: 149 and a VL set forth in SEQ ID NO: 150. In some embodiments, the antigen-binding domain, such as an scFv, contains a VH set forth in SEQ ID NO: 151 and a VL set forth in SEQ ID NO: 152. In some embodiments, the VH or VL has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing VH or VL sequences, and retains binding to BCMA. In some embodiments, the VH region is amino-terminal to the VL region. In some embodiments, the VH region is carboxy-terminal to the VL region. In some embodiments, the variable heavy and variable light chains are connected by a linker. In some embodiments, the linker is set forth in SEQ ID NO: 28, 29, 30, or 38.

Among a provided anti-BCMA CAR is a CAR in which the antibody or antigen-binding fragment contains a VH region comprising the sequence set forth in SEQ ID NO: 18 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 18; and contains a VL region comprising the sequence set forth in SEQ ID NO: 19 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 19. In some embodiments, the antibody or antigen-binding fragment of the provided CAR contains a VH region that has a CDRH1, a CDRH2 and a CDRH3 comprising the amino acid sequence of SEQ ID NOS: 189, 190, and 191, respectively and a VL region that has a CDRL1, a CDRL2 and a CDRL3 comprising the amino acid sequence of SEQ ID NOS: 192, 193, and 194, respectively. In some embodiments, the antibody or antigen-binding fragment of the provided CAR contains a VH region that has a CDRH1, a CDRH2 and a CDRH3 comprising the amino acid sequence of SEQ ID NOS: 195, 196, and 197, respectively and a VL region that has a CDRL1, a CDRL2 and a CDRL3 comprising the amino acid sequence of SEQ ID NOS: 198, 199, and 200, respectively. In some embodiments, the antibody or antigen-binding fragment of the provided CAR contains a VH region that has a CDRH1, a CDRH2 and a CDRH3 comprising the amino acid sequence of SEQ ID NOS: 201, 202, and 203, respectively and a VL region that has a CDRL1, a CDRL2 and a CDRL3 comprising the amino acid sequence of SEQ ID NOS: 204, 205, and 206, respectively. In some embodiments, the antibody or antigen-binding fragment of the provided CAR contains a VH region that has a CDRH1, a CDRH2 and a CDRH3 comprising the amino acid sequence of SEQ ID NOS: 207, 208, and 209, respectively and a VL region that has a CDRL1, a CDRL2 and a CDRL3 comprising the amino acid sequence of SEQ ID NOS: 210, 211, and 212, respectively. In some embodiments, the VH region comprises the sequence set forth in SEQ ID NO: 18 and the VL region comprises the sequence set forth in SEQ ID NO: 19. In some embodiments, the antibody or antigen-binding fragment is a single-chain antibody fragment, such as an scFv. In some embodiments, the scFv comprises the sequence of amino acids set forth in SEQ ID NO:213 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:213. In some embodiments, the anti-BCMA CAR has the sequence of amino acids set forth in SEQ NO: 116 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 116. In some embodiments, the anti-BCMA CAR is encoded by the polynucleotide sequence set forth in SEQ NO: 214 or a polynucleotide sequence of at least at or about 90%, at or about 91%, at or about 92%, at or about 93%, at or about 94%, at or about 95%, at or about 96%, at or about 97%, at or about 98%, or at or about 99% identity to SEQ ID NO:214.

Among a provided anti-BCMA CAR is a CAR in which the antibody or antigen-binding fragment contains a VH region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:24; and contains a VL region comprising the sequence set forth in SEQ ID NO:25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:25. In some embodiments, the antibody or antigen-binding fragment of the provided CAR contains a VH region that has a CDRH1, a CDRH2 and a CDRH3 comprising the amino acid sequence of SEQ ID NOS: 173, 174 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 176, 177 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 178, 179 and 175, respectively and a VL region that has a CDRL1, a CDRL2 and a CDRL3 comprising the amino acid sequence of SEQ ID NOS: 183, 184 and 185, respectively. In some embodiments, the antibody or antigen-binding fragment of the provided CAR contains a VH region that has a CDRH1, a CDRH2 and a CDRH3 comprising the amino acid sequence of SEQ ID NOS: 180, 181 and 182, respectively and a VL region that has a CDRL1, a CDRL2 and a CDRL3 comprising the amino acid sequence of SEQ ID NOS: 186, 187 and 185, respectively. In some embodiments, the VH region comprises the sequence set forth in SEQ ID NO:24 and the VL region comprises the sequence set forth in SEQ ID NO:25. In some embodiments, the antibody or antigen-binding fragment is a single-chain antibody fragment, such as an scFv. In some embodiments, the scFv comprises the sequence of amino acids set forth in SEQ ID NO: 188 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:188. In some embodiments, the anti-BCMA CAR has the sequence of amino acids set forth in SEQ NO: 124 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 124. In some embodiments, the anti-BCMA CAR has the sequence of amino acids set forth in SEQ NO: 125 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 125.

In some embodiments, the scFv comprises the amino acid sequence set forth in any one of SEQ ID NOS: 216-247, or an amino acid sequence having at least 90, 95, 96, 97, 98, 99, or 100% sequence identity to a sequence set forth in any one of SEQ ID NOS: 216-247.

In some embodiments, the antigen-binding domain comprises an sdAb. In some embodiments, the antigen-binding domain contains the sequence set forth by SEQ ID NO:77. In some embodiments, the antigen-binding domain comprises a sequence at least or about 50, 60, 70, 80, 85, 90, 95, 96, 97, 98, 99, or 100% identical to the sequence set forth by SEQ ID NO:77.

In some embodiments, the CAR comprises the amino acid sequence set forth in any one of SEQ ID NOS: 90-141, or an amino acid sequence having at least 90, 95, 96, 97, 98, or 99% sequence identity to a sequence set forth in any one of SEQ ID NOS: 190-141.

In some embodiments, the antigen targeted by the receptor is CD20, CD19, CD22, ROR1, CD45, CD21, CD5, CD33, Igkappa, Iglambda, CD79a, CD79b or CD30. In particular aspects, the antigen is CD19.

In some embodiments, the antibody or an antigen-binding fragment (e.g. scFv or VH domain) specifically recognizes an antigen, such as CD19. In some embodiments, the antibody or antigen-binding fragment is derived from, or is a variant of, antibodies or antigen-binding fragment that specifically binds to CD19. In some embodiments, the antigen is CD19. In some embodiments, the scFv contains a VH and a V_{L} derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments the antigen-binding domain includes a V_{H} and/or V_{L} derived from FMC63, which, in some aspects, can be an scFv. FMC63 generally refers to a mouse monoclonal IgG1 antibody raised against Nalm-1 and -16 cells expressing CD19 of human origin (Ling, N. R., et al. (1987). Leucocyte typing III. 302). In some embodiments, the FMC63 antibody comprises CDR-H1 and CDR-H2 set forth in SEQ ID NO: 251 and 252, respectively, and CDR-H3 set forth in SEQ ID NO: 253 or 266 and CDR-L1 set forth in SEQ ID NO: 248 and CDR-L2 set forth in SEQ ID NO: 249 or 267 and CDR-L3 sequences set forth in SEQ ID NO: 250 or 268. In some embodiments, the FMC63 antibody comprises the heavy chain variable region (V_{H}) comprising the amino acid sequence of SEQ ID NO: 254 and the light chain variable region (V_{L}) comprising the amino acid sequence of SEQ ID NO: 255.

In some embodiments, the scFv comprises a variable light chain containing the CDR-L1 sequence of SEQ ID NO:248, a CDR-L2 sequence of SEQ ID NO:249, and a CDR-L3 sequence of SEQ ID NO:250 and/or a variable heavy chain containing a CDR-H1 sequence of SEQ ID NO:251, a CDR-H2 sequence of SEQ ID NO:252, and a CDR-H3 sequence of SEQ ID NO:253, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of FMC63 set forth in SEQ ID NO:254 and a variable light chain region of FMC63 set forth in SEQ ID NO:255, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:29. In some embodiments, the scFv comprises, in order, a V_{H}, a linker, and a V_{L}. In some embodiments, the scFv comprises, in order, a V_{L}, a linker, and a V_{H}. In some embodiments, the scFv is encoded by a sequence of nucleotides set forth in SEQ ID NO:269 or a sequence that exhibits at least 85%, 86%, 87%, 88%, 89%, 90%, 91%, 92%, 93%, 94%, 95%, 96%, 97%, 98%, or 99% sequence identity to SEQ ID NO:269. In some embodiments, the scFv comprises the sequence of amino acids set forth in SEQ ID NO:256 or a sequence that exhibits at least 85%, 86%, 87%, 88%, 89%, 90%, 91%, 92%, 93%, 94%, 95%, 96%, 97%, 98%, or 99% sequence identity to SEQ ID NO:256.

In some embodiments the antigen-binding domain includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). In some embodiments, the SJ25C1 antibody comprises CDR-H1, CDR-H2 and CDR-H3 set forth in SEQ ID NOS: 260-262, respectively, and CDR-L1, CDR-L2 and CDR-L3 sequences set forth in SEQ ID NOS: 257-259, respectively. In some embodiments, the SJ25C1 antibody comprises the heavy chain variable region (V_{H}) comprising the amino acid sequence of SEQ ID NO: 263 and the light chain variable region (V_{L}) comprising the amino acid sequence of SEQ ID NO: 264. In some embodiments, the scFv comprises a variable light chain containing a CDR-L1 sequence of SEQ ID NO:257, a CDR-L2 sequence of SEQ ID NO: 258, and a CDR-L3 sequence of SEQ ID NO:259 and/or a variable heavy chain containing a CDR-H1 sequence of SEQ ID NO:260, a CDR-H2 sequence of SEQ ID NO:261, and a CDR-H3 sequence of SEQ ID NO:262, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the scFv comprises a variable heavy chain region of SJ25C1 set forth in SEQ ID NO:263 and a variable light chain region of SJ25C1 set forth in SEQ ID NO:264, or a variant of any of the foregoing having at least 85%, 86%, 87%, 88%, 89%, 90%, 91%, 92%, 93%, 94%, 95%, 96%, 97%, 98%, 99% or more sequence identity thereto. In some embodiments, the variable heavy and variable light chains are connected by a linker. In some embodiments, the linker is set forth in SEQ ID NO:28. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:265 or a sequence that exhibits at least 85%, 86%, 87%, 88%, 89%, 90%, 91%, 92%, 93%, 94%, 95%, 96%, 97%, 98%, or 99% sequence identity to SEQ ID NO:265.

In some embodiments, the antigen is CD20. In some embodiments, the scFv contains a V_{H} and a V_{L} derived from an antibody or an antibody fragment specific to CD20. In some embodiments, the antibody or antibody fragment that binds CD20 is an antibody that is or is derived from rituximab, such as rituximab scFv.

In some embodiments, the antigen is CD22. In some embodiments, the scFv contains a V_{H} and a V_{L} derived from an antibody or an antibody fragment specific to CD22. In some embodiments, the antibody or antibody fragment that binds CD22 is an antibody that is or is derived from m971, such as m971 scFv.

In some embodiments, the antigen or antigen binding domain is GPRC5D. In some embodiments, the scFv contains a V_{H} and a V_{L} derived from an antibody or an antibody fragment specific to GPRC5D. In some embodiments, the antibody or antibody fragment that binds GPRC5D is or contains a V_{H} and a V_{L} from an antibody or antibody fragment set forth in International Publication Nos. WO 2016/090329 and WO 2016/090312.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a CH1/CL, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a CH1/CL, and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to CH2 and CH3 domains, or IgG4 hinge linked to the CH3 domain. Exemplary spacers include IgG4 hinge alone, IgG4 hinge linked to CH2 and CH3 domains, or IgG4 hinge linked to the CH3 domain. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a CH2 and CH3 region. In some embodiments, one of more of the hinge, CH2 and CH3 is derived all or in part from IgG4 or IgG2. In some cases, the hinge, CH2 and CH3 is derived from IgG4. In some aspects, one or more of the hinge, CH2 and CH3 is chimeric and contains sequence derived from IgG4 and IgG2. In some examples, the spacer contains an IgG4/2 chimeric hinge, an IgG2/4 CH2, and an IgG4 CH3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the CH2 region, of the full-length IgG4 Fc sequence or an N176Q at position 176, in the CH2 region, of the full-length IgG4 Fc sequence.

In some embodiments, the spacer has the sequence ESKYGPPCPPCP (set forth in SEQ ID NO: 1), and is encoded by the sequence set forth in SEQ ID NO: 2. In some embodiments, the spacer has the sequence set forth in SEQ ID NO: 3. In some embodiments, the spacer has the sequence set forth in SEQ ID NO: 4. In some embodiments, the encoded spacer is or contains the sequence set forth in SEQ ID NO: 31. In some embodiments, the constant region or portion is of IgD. In some embodiments, the spacer has the sequence set forth in SEQ ID NO: 5. In some embodiments, the spacer has the sequence set forth in SEQ ID NO: 89.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA) or RAAA (SEQ ID NO: 144), is present and forms a linkage between the scFv and the spacer region of the CAR. In some embodiments, the spacer has the sequence set forth in SEQ ID NO: 78. In some embodiments, the spacer has the sequence set forth in SEQ ID NO: 80. In some embodiments, the spacer has the sequence set forth in any of SEQ ID NOs: 81-83, In some embodiments, the spacer has the sequence set forth in SEQ ID NO: 84. In some embodiments, the spacer has the sequence set forth in SEQ ID NO: 86. In some embodiments, the spacer has the sequence set forth in SEQ ID NO: 88.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4, 5, 31, 78, 80, 81, 82, 83, 84, 86, 88, or 89.

In some embodiments, the spacer has the sequence set forth in SEQ ID NOS: 157-165. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 157-165.

This antigen recognition domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the chimeric antigen receptor includes a transmembrane domain linking the extracellular domain and the intracellular signaling domain. In some embodiments, the transmembrane domain is fused to the extracellular domain, such as linked or fused between the extracellular domain (e.g. scFv) and intracellular signaling domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD8a, CD9, CD16, CD22, CD33, CD37, CD64, CD80, CD86, CD134, CD137 (4-1BB), CD154, CTLA-4 or PD-1. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28. Exemplary sequences of transmembrane domains are or comprise the sequences set forth in SEQ ID NOs: 8, 79, 85, 87, 142, or 143. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR CD3 chain that mediates T-cell stimulation and/or activation and cytotoxicity, e.g., CD3 zeta chain, CD3 gamma, CD3 delta, CD3 epsilon, FcR gamma, FcR beta, CDS, CD22, CD79a, CD79b and CD66d. In some examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell stimulation and/or activation is in some aspects described as being mediated by two classes of cytoplasmic signaling sequences: those that initiate antigen-dependent primary stimulation and/or activation through the TCR (primary cytoplasmic signaling regions, domains or sequences), and those that act in an antigen-independent manner to provide a secondary or co-stimulatory signal (secondary cytoplasmic signaling regions, domains or sequences). In some aspects, the CAR includes one or both of such signaling components.

In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1), or CD8a (Accession No. P01732.1), or variant thereof, such as a transmembrane domain that comprises the sequence of amino acids set forth in SEQ ID NO: 8, 79, 142, or 143 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 8, 79, 142, or 143. In some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the transmembrane domain is a transmembrane domain from CD8. In some embodiments, the transmembrane domain is any as described in Milone et al., Mol. Ther. (2009) 12(9):1453-64. In some embodiments, the transmembrane domain is or comprises the sequence set forth in SEQ ID NO: 143.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular domain comprises an intracellular costimulatory signaling domain of 4-1BB, In some embodiments, the 4-1BB co-stimulatory molecule is any as described in Milone et al., Mol. Ther. (2009) 12(9):1453-64. In some embodiments, the co-stimulatory molecular has the sequence set forth in SEQ ID NO: 12.

In some embodiments, the intracellular signaling domain of the recombinant receptor, e.g. the CAR, comprises a human CD3 zeta stimulatory signaling domain or functional variant thereof, such as a 112 AA cytoplasmic domain of isoform 3 of human CD3 ((Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15. In some embodiments, the CD3-zeta domain is any as described in Milone et al., Mol. Ther. (2009) 12(9):1453-64. In some embodiments, the CD3-zeta is or comprises the sequence set forth in SEQ ID NO: 13.

In some aspects, the spacer contains only a hinge region of an IgG, such as only a hinge of IgG4 or IgG1, such as the hinge only spacer set forth in SEQ ID NO: 1 or SEQ ID NO: 89. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains, such as set forth in SEQ ID NO: 4. In some embodiments, the spacer is an Ig hinge, e.g., an IgG4 hinge, linked to a CH3 domain only, such as set forth in SEQ ID NO: 3. In some embodiments, the spacer is or comprises a glycine-serine rich sequence or other flexible linker such as known flexible linkers. In some embodiments, the spacer is a CD8a hinge, such as set forth in any of SEQ ID NOs: 81-83, an FcγRIIIa hinge, such as set forth in SEQ ID NO: 88, a CTLA4 hinge, such as set forth in SEQ ID NO: 84, or a PD-1 hinge, such as set forth in SEQ ID NO: 86. In some embodiments the spacer is derived from CD8. In some embodiments, the spacer is a CD8□ hinge sequence. In some embodiments, the hinge sequence is any as described in Milone et al., Mol. Ther. (2009) 12(9):1453-64. In some embodiments, the hinge is or comprises the sequence set forth in SEQ ID NO:82.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. In some embodiments, the CAR includes an antibody or fragment, such as scFv, a spacer such as any of the Ig-hinge containing spacers, a CD8-derived transmembrane domain, a 4-1BB-derived intracellular signaling domain, and a CD3 zeta-derived signaling domain.

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. In some embodiments, such CAR constructs further includes a T2A ribosomal skip element and/or a tEGFR sequence, e.g., downstream of the CAR.

An exemplary polypeptide for a truncated EGFR (e.g. tEGFR) comprises the sequence of amino acids set forth in SEQ ID NO: 7 or 166 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166. An exemplary T2A linker sequence comprises the sequence of amino acids set forth in SEQ ID NO: 6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. Exemplary signal peptides include the sequences set forth in SEQ ID NOs: 39, 40 and 153. In some examples the signal peptide is derived from CD8. In some embodiments, the signal peptide is the sequence set forth in Accession No. NM_001768. In some embodiments, the signal peptide include the sequences set forth in SEQ ID NO: 39.

In some embodiments, the CAR includes an anti-BCMA antibody or fragment, such as any of the anti-human BCMA antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-BCMA antibody or fragment, such as any of the anti-human BCMA antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

In some embodiments, the CAR includes an anti-GPRC5D antibody or fragment, such as any of the anti-human GPRC5D antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-GPRC5D antibody or fragment, such as any of the anti-human GPRC5D antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

In some embodiments, the CAR includes an anti-CD19 antibody or fragment, such as any of the anti-human CD19 antibodies, including sdAbs and scFvs, described herein, a spacer such as any of the Ig-hinge containing spacers or other spacers described herein, a CD28 transmembrane domain, a CD28 intracellular signaling domain, and a CD3 zeta signaling domain. In some embodiments, the CAR includes an anti-CD19 antibody or fragment, such as any of the anti-human GPRC5D antibodies, including sdAbs and scFvs described herein, a spacer such as any of the Ig-hinge containing spacers or other spacers described herein, a CD28 transmembrane domain, a 4-1BB intracellular signaling domain, and a CD3 zeta signaling domain. In some embodiments, such CAR constructs further includes a T2A ribosomal skip element and/or a tEGFR sequence, e.g., downstream of the CAR.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition. In some embodiments, the CAR specifically binds to BCMA, such as human BCMA, and includes an anti-human BCMA antibody or fragment as described. Non-limiting exemplary CAR sequences, including anti-BCMA CAR sequences, are set forth in SEQ ID NOs: 90-141. In some embodiments, an anti-BCMA CAR includes the amino acid sequence set forth in any of SEQ ID NOS: 90-141 or an amino acid sequence that exhibits at least at or about 90%, at or about 91%, at or about 92%, at or about 93%, at or about 94%, at or about 95%, at or about 96%, at or about 97%, at or about 96%, at or about 97%, at or about 98%, at or about 99% sequence identity to any one of SEQ ID NOS: 90-141, and wherein the CAR specifically binds BCMA, e.g. human BCMA.

In some embodiments, the dose of genetically engineered T cells comprises idecabtagene vicleucel cells (e.g., such as ABECMA® cells); bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells, or GC012F cells. In some embodiments, the dose of genetically engineered T cells comprises idecabtagene vicleucel cells (e.g., such as ABECMA® cells).

In some embodiments, among such antibodies or antigen-binding domains in the provided CARs are antibodies capable of binding a target protein, such as human BCMA protein, with at least a certain affinity, as measured by any of a number of known methods. In some embodiments, the affinity is represented by an equilibrium dissociation constant (KD); in some embodiments, the affinity is represented by EC50.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for a target protein, e.g., BCMA. For example, in some embodiments, a BIAcore® instrument can be used to determine the binding kinetics and constants of a complex between two proteins (e.g., an antibody or fragment thereof, and an antigen, such as a BCMA cell surface protein, soluble BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In some embodiments, the binding molecule, e.g., antibody or fragment thereof or antigen-binding domain of a CAR, binds, such as specifically binds, to a target protein, e.g., a cell surface BCMA protein or soluble BCMA protein or an epitope therein, with an affinity or KA (i.e., an equilibrium association constant of a particular binding interaction with units of 1/M; equal to the ratio of the on-rate [kon or ka] to the off-rate [koff or kd] for this association reaction, assuming bimolecular interaction) equal to or greater than 105 M−1. In some embodiments, the antibody or fragment thereof or antigen-binding domain of a CAR exhibits a binding affinity for the peptide epitope with a KD (i.e., an equilibrium dissociation constant of a particular binding interaction with units of M; equal to the ratio of the off-rate [koff or kd] to the on-rate [kon or ka] for this association reaction, assuming bimolecular interaction) of equal to or less than 10−5 M. For example, the equilibrium dissociation constant KD ranges from 10−5 M to 10−13 M, such as 10−7 M to 10−11 M, 10−8 M to 10−10 M, or 10−9 M to 10−10 M. The on-rate (association rate constant; kon or ka; units of 1/Ms) and the off-rate (dissociation rate constant; koff or kd; units of 1/s) can be determined using any of the assay methods known in the art, for example, surface plasmon resonance (SPR).

In some embodiments, the binding affinity (EC50) and/or the dissociation constant of the antibody (e.g. antigen-binding fragment) or antigen-binding domain of a CAR to a target protein, such as human BCMA protein, is from or from about 0.01 nM to about 500 nM, from or from about 0.01 nM to about 400 nM, from or from about 0.01 nM to about 100 nM, from or from about 0.01 nM to about 50 nM, from or from about 0.01 nM to about 10 nM, from or from about 0.01 nM to about 1 nM, from or from about 0.01 nM to about 0.1 nM, is from or from about 0.1 nM to about 500 nM, from or from about 0.1 nM to about 400 nM, from or from about 0.1 nM to about 100 nM, from or from about 0.1 nM to about 50 nM, from or from about 0.1 nM to about 10 nM, from or from about 0.1 nM to about 1 nM, from or from about 0.5 nM to about 200 nM, from or from about 1 nM to about 500 nM, from or from about 1 nM to about 100 nM, from or from about 1 nM to about 50 nM, from or from about 1 nM to about 10 nM, from or from about 2 nM to about 50 nM, from or from about 10 nM to about 500 nM, from or from about 10 nM to about 100 nM, from or from about 10 nM to about 50 nM, from or from about 50 nM to about 500 nM, from or from about 50 nM to about 100 nM or from or from about 100 nM to about 500 nM. In certain embodiments, the binding affinity (EC50) and/or the equilibrium dissociation constant, KD, of the antibody to a target protein, such as human BCMA protein, is at or less than or about 400 nM, 300 nM, 200 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. In some embodiments, the antibodies bind to a target protein, such as human BCMA protein, with a sub-nanomolar binding affinity, for example, with a binding affinity less than about 1 nM, such as less than about 0.9 nM, about 0.8 nM, about 0.7 nM, about 0.6 nM, about 0.5 nM, about 0.4 nM, about 0.3 nM, about 0.2 nM or about 0.1 nM or less.

In some embodiments, the binding affinity may be classified as high affinity or as low affinity. In some cases, the binding molecule (e.g. antibody or fragment thereof) or antigen-binding domain of a CAR that exhibits low to moderate affinity binding exhibits a KA of up to 107 M−1, up to 106 M−1, up to 105 M−1. In some cases, a binding molecule (e.g. antibody or fragment thereof) that exhibits high affinity binding to a particular epitope interacts with such epitope with a KA of at least 107 M−1, at least 108 M−1, at least 109 M−1, at least 1010 M−1, at least 1011 M−1, at least 1012 M−1, or at least 1013 M−1. In some embodiments, the binding affinity (EC50) and/or the equilibrium dissociation constant, KD, of the binding molecule, e.g., anti-BCMA antibody or fragment thereof or antigen-binding domain of a CAR, to a target (e.g., BCMA) protein, is from or from about 0.01 nM to about 1 μM, 0.1 nM to 1 μM, 1 nM to 1 μM, 1 nM to 500 nM, 1 nM to 100 nM, 1 nM to 50 nM, 1 nM to 10 nM, 10 nM to 500 nM, 10 nM to 100 nM, 10 nM to 50 nM, 50 nM to 500 nM, 50 nM to 100 nM or 100 nM to 500 nM. In certain embodiments, the binding affinity (EC50) and/or the dissociation constant of the equilibrium dissociation constant, KD, of the binding molecule, e.g., anti-BCMA antibody or fragment thereof or antigen-binding domain of a CAR, to a target (e.g., BCMA) protein, is at or about or less than at or about 1 μM, 500 nM, 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or less. The degree of affinity of a particular antibody can be compared with the affinity of a known antibody, such as a reference antibody (e.g., anti-BCMA reference antibody).

In some embodiments, the binding affinity of the antibody or antigen-binding domain of a CAR, for different form or topological type of antigens, e.g., soluble or shed target protein compared to the binding affinity to a membrane-bound target protein, to determine the preferential binding or relative affinity for a particular form or topological type. For example, in some aspects, an anti-BCMA antibodies or antigen-binding domain of a CAR can exhibit preferential binding to membrane-bound BCMA as compared to soluble or shed BCMA and/or exhibit greater binding affinity for, membrane-bound BCMA compared to soluble or shed BCMA. In some embodiments, the equilibrium dissociation constant, KD, for different form or topological type of BCMA proteins, can be compared to determine preferential binding or relative binding affinity. In some embodiments, the preferential binding or relative affinity to a membrane-bound BCMA compared to soluble or shed BCMA can be high. For example, in some cases, the ratio of KD for soluble or shed BCMA and the KD for membrane-bound BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the ratio of KA for membrane-bound BCMA and the KA for soluble or shed BCMA is more than 10, 15, 20, 25, 30, 40, 50, 60, 70, 80, 90, 100, 200, 500, 1000, 2000 or more and the antibody or antigen-binding domain preferentially binds or has higher binding affinity for membrane-bound BCMA. In some cases, the antibody or antigen-binding domain of CAR binds soluble or shed BCMA and membrane-bound BCMA to a similar degree, e.g., the ratio of KD for soluble BCMA and KD for membrane-bound BCMA is or is about 1. In some cases, the antibody or antigen-binding domain of CAR binds soluble or shed BCMA and membrane-bound BCMA to a similar degree, e.g., the ratio of KA for soluble BCMA and KA for membrane-bound BCMA is or is about 1. The degree of preferential binding or relative affinity for membrane-bound BCMA or soluble or shed BCMA can be compared with that of a known antibody, such as a reference antibody (e.g., reference anti-BCMA CAR). In some embodiments, the reference antibody (e.g., reference anti-BCMA CAR) binds to membrane-bound and soluble or shed BCMA protein.

b. Cells and Preparation for Genetic Engineering

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., Blood. 1:72-82 (2012); Wang et al., J Immunother. 35(9):689-701 (2012). In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+T helper cells are sorted into naïve, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinity magnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, NJ).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in PCT Publication No. WO2009/072003 or US Publication No. US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al., J Immunother. 35(9): 651-660 (2012), Terakura et al., Blood. 1:72-82 (2012), and Wang et al., J Immunother. 35(9):689-701 (2012).

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al., Lab Chip 10, 1567-1573 (2010); and Godin et al., J Biophoton. 1(5):355-376 (2008). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL.

In some aspects, incubation is carried out in accordance with techniques such as those described in U.S. Pat. No. 6,040,177 to Riddell et al., Klebanoff et al., J Immunother. 35(9): 651-660 (2012), Terakura et al., Blood. 1:72-82 (2012), and/or Wang et al., J Immunother. 35(9):689-701 (2012).

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees Celsius, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4⁺ and/or CD8⁺ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen

c. Nucleic Acids, Vectors, and Methods for Genetic Engineering

In some embodiments, the cells, e.g., T cells, are genetically engineered to express a recombinant receptor. In some embodiments, the engineering is carried out by introducing nucleic acid molecules that encode the recombinant receptor. Also provided are nucleic acid molecules encoding a recombinant receptor, and vectors or constructs containing such nucleic acids and/or nucleic acid molecules.

In some cases, the nucleic acid sequence encoding the recombinant receptor, e.g., chimeric antigen receptor (CAR), contains a signal sequence that encodes a signal peptide. In some aspects, the signal sequence may encode a signal peptide derived from a native polypeptide. In other aspects, the signal sequence may encode a heterologous or non-native signal peptide. In some embodiments, the signal peptide is derived from a transmembrane protein. In some examples the signal peptide is derived from CD8a, CD33, or an IgG. Non-limiting exemplary examples of signal peptides include, for example, the CD33 signal peptide set forth in SEQ ID NO: 153, CD8a signal peptide set forth in SEQ ID NO: 154, or the signal peptide set forth in SEQ ID NO:39 or modified variant thereof. In some embodiments, the signal peptide is the CD8a signal peptide set forth in Accession No. NM_001768.

In some embodiments, the nucleic acid molecule encoding the recombinant receptor contains at least one promoter that is operatively linked to control expression of the recombinant receptor. In some examples, the nucleic acid molecule contains two, three, or more promoters operatively linked to control expression of the recombinant receptor. In some embodiments, nucleic acid molecule can contain regulatory sequences, such as transcription and translation initiation and termination codons, which are specific to the type of host (e.g., bacterium, fungus, plant, or animal) into which the nucleic acid molecule is to be introduced, as appropriate and taking into consideration whether the nucleic acid molecule is DNA- or RNA-based. In some embodiments, the nucleic acid molecule can contain regulatory/control elements, such as a promoter, an enhancer, an intron, a polyadenylation signal, a Kozak consensus sequence, and splice acceptor or donor. In some embodiments, the nucleic acid molecule can contain a nonnative promoter operably linked to the nucleotide sequence encoding the recombinant receptor and/or one or more additional polypeptide(s). In some embodiments, the promoter is selected from among an RNA pol I, pol II or pol III promoter. In some embodiments, the promoter is recognized by RNA polymerase II (e.g., a CMV, SV40 early region or adenovirus major late promoter). In another embodiment, the promoter is recognized by RNA polymerase III (e.g., a U6 or H1 promoter). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, the promoter is or comprises a constitutive promoter. Exemplary constitutive promoters include, e.g., simian virus 40 early promoter (SV40), cytomegalovirus immediate-early promoter (CMV), human Ubiquitin C promoter (UBC), human elongation factor 1α promoter (EF1α), mouse phosphoglycerate kinase 1 promoter (PGK), and chicken β-Actin promoter coupled with CMV early enhancer (CAGG). In some embodiments, the constitutive promoter is a synthetic or modified promoter. In some embodiments, the promoter is or comprises an MND promoter, a synthetic promoter that contains the U3 region of a modified MoMuLV LTR with myeloproliferative sarcoma virus enhancer (see Challita et al. (1995) J. Virol. 69(2):748-755). In some embodiments, the promoter is a tissue-specific promoter. In another embodiment, the promoter is a viral promoter. In another embodiment, the promoter is a non-viral promoter.

In another embodiment, the promoter is a regulated promoter (e.g., inducible promoter). In some embodiments, the promoter is an inducible promoter or a repressible promoter. In some embodiments, the promoter comprises a Lac operator sequence, a tetracycline operator sequence, a galactose operator sequence or a doxycycline operator sequence, or is an analog thereof or is capable of being bound by or recognized by a Lac repressor or a tetracycline repressor, or an analog thereof. In some embodiments, the nucleic acid molecule does not include a regulatory element, e.g. promoter.

In some embodiments, the nucleic acid molecule encoding the recombinant receptor, e.g., CAR or other antigen receptor, further includes nucleic acid sequences encoding a marker and/or cells expressing the CAR or other antigen receptor further includes a marker, e.g., a surrogate marker, such as a cell surface marker, which may be used to confirm transduction or engineering of the cell to express the receptor, such as a truncated version of a cell surface receptor, such as truncated EGFR (tEGFR). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the nucleic acid molecule, e.g., a nucleic acid molecule encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same nucleic acid molecule that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO:7 or 166) or a prostate-specific membrane antigen (PSMA) or modified form thereof tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Publication No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:7 or 166. An exemplary T2A linker sequence comprises the sequence of amino acids set forth in SEQ ID NO:6 or 167 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:6 or 167.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NO: 6 or 167, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 6 or 167. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NO: 7 or 166, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 166.

In some embodiments, a single promoter may direct expression of an RNA that contains, in a single open reading frame (ORF), two or three genes (e.g. encoding the molecule involved in modulating a metabolic pathway and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein, without limitation, 2A sequences from the foot-and-mouth disease virus (F2A; e.g. SEQ ID NO: 172), equine rhinitis A virus (E2A; e.g. SEQ ID NO: 170), Thosea asigna virus (T2A, e.g., SEQ ID NO: 6 or 167), and porcine teschovirus-1 (P2A; e.g. SEQ ID NO: 168 or 169) as described in U.S. Patent Publication No. 20070116690.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

Introduction of the nucleic acid molecules encoding the recombinant receptor in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle. bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

d. Dosage and Administration of a T Cell Therapy

In some embodiments of the methods, compositions, combinations, kits and uses provided herein, the treatment includes administering to a subject a T cell therapy (e.g. CAR T cells). For example, the T cell therapy is an anti-BCMA CAR T cell therapy.

In some embodiments, the cells for use in or administered in connection with the provided methods contain or are engineered to contain an engineered receptor, e.g., an engineered antigen receptor, such as a chimeric antigen receptor (CAR), or a T cell receptor (TCR). Among the compositions are pharmaceutical compositions and formulations for administration, such as for adoptive cell therapy. Also provided are therapeutic methods for administering the cells and compositions to subjects, e.g., patients, in accord with the provided methods, and/or with the provided articles of manufacture or compositions.

In some embodiments, the cell-based therapy is or comprises administration of cells, such as immune cells, for example T cell or NK cells, that target a molecule expressed on the surface of a lesion, such as a tumor or a cancer. In some embodiments, the cells express a recombinant receptor, e.g. CAR, that contains an extracellular ligand-binding domain that specifically binds to an antigen. In some embodiments, the recombinant receptor is a CAR that contains an extracellular antigen-recognition domain that specifically binds to BCMA. In some embodiments, the immune cells express a recombinant receptor, such as a chimeric antigen receptor (CAR). In some embodiments, the T cell therapy includes administering T cells engineered to express a chimeric antigen receptor (CAR). In particular embodiments, the cell therapy, e.g. anti-BCMA CAR T cell therapy, is for treating a multiple myeloma, such as a relapsed and refractory (R/R multiple myeloma). In some embodiments, the cells are autologous to the subject. In some embodiments, the cells are allogeneic to the subject. Exemplary engineered cells for administering as a cell therapy in the provided methods are described in Section I.C.1.b.

Methods for administration of cells for adoptive cell therapy are known and may be used in connection with the provided methods, compositions and articles of manufacture and kits. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells of the T cell therapy can be administered in a composition formulated for administration, or alternatively, in more than one composition (e.g., two compositions) formulated for separate administration. The dose(s) of the cells may include a particular number or relative number of cells or of the engineered cells, and/or a defined ratio or compositions of two or more sub-types within the composition, such as CD4+ vs CD8+ T cells.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the treatment of disease, the appropriate dosage may depend on the type of disease to be treated, the type of cells or recombinant receptors, the severity and course of the disease, previous therapy, the subject's clinical history and response to the cells, and the discretion of the attending physician. The compositions and cells are in some embodiments suitably administered to the subject at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In some embodiments, the dose includes fewer than about 5×10⁸ total recombinant receptor (e.g., CAR)-expressing cells, T cells, or peripheral blood mononuclear cells (PBMCs), e.g., in the range of about 1×10⁸ to 5×10⁸ such cells, such as 1.5×10⁸, 3×10⁸, or 4.5×10⁸ or total such cells, or the range between any two of the foregoing values.

The cells can be administered by any suitable means. The cells are administered in a dosing regimen to achieve a therapeutic effect, such as a reduction in tumor burden. Various dosing schedules of the T cell therapy include but are not limited to single or multiple administrations over various time-points, bolus administration, and pulse infusion.

Preconditioning subjects with immunodepleting (e.g., lymphodepleting) therapies, including any of those described in Section I.E, in some aspects can improve the effects of adoptive cell therapy (ACT).

Thus, in some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is administered a preconditioning agent (lymphodepleting treatment) as described in Section I.E.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In some embodiments, a dose of cells is administered to subjects in accord with the provided T cell therapy methods. In some embodiments, the size or timing of the doses is determined as a function of the particular disease or condition in the subject. One may empirically determine the size or timing of the doses for a particular disease in view of the provided description.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 150 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, nor more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In some embodiments, the cell therapy comprises administration of a dose comprising a number of cell from or from about 1×10⁵ to 2×10⁹ total recombinant receptor-expressing cells, total T cells, or total peripheral blood mononuclear cells (PBMCs), from or from about 5×10⁵ to 1×10⁹ total recombinant receptor-expressing cells, total T cells, or total peripheral blood mononuclear cells (PBMCs) or from or from about 1×10⁶ to 1×10⁹ total recombinant receptor-expressing cells, total T cells, or total peripheral blood mononuclear cells (PBMCs), each inclusive. In some embodiments, the cell therapy comprises administration of a dose of cells comprising a number of cells at least or about at least 1×10⁵ total recombinant receptor-expressing cells, total T cells, or total peripheral blood mononuclear cells (PBMCs), such at least or at least 1×10⁶, at least or about at least 1×10⁷, at least or about at least 1×10⁸, at least or about at least 1×10⁹ of such cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 1.0×10⁷ to at or about 1.2×10⁹ such cells, such as at or about 1.0×10⁷, 1.5×10⁷, 2.0×10⁷, 2.5×10⁷, 5×10⁷, 1.5×10⁸,3×10⁸, 4.5×10⁸, 6×10⁸, 8×10⁸ or 1.2×10⁹ total such cells, or the range between any two of the foregoing values. In some embodiments, for example, where the subject is a human, the dose includes more than at or about 1×10⁶ total recombinant receptor (e.g., CAR)-expressing (CAR+) cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1.5×10⁸, 3×10⁸, 4.5×10⁸, 6×10⁸, 8×10⁸ or 1.2×10⁹ total such cells, or the range between any two of the foregoing values. In some embodiments, for example, where the subject is a human, the dose includes at or about 1.0×10⁷ total recombinant receptor (e.g., CAR)-expressing cells, T cells, or peripheral blood mononuclear cells (PBMCs). In some embodiments, for example, where the subject is a human, the dose includes at or about 1.5×10⁷ total recombinant receptor (e.g., CAR)-expressing cells, T cells, or peripheral blood mononuclear cells (PBMCs). In some embodiments, for example, where the subject is a human, the dose includes at or about 2.0×10⁷ total recombinant receptor (e.g., CAR)-expressing cells, T cells, or peripheral blood mononuclear cells (PBMCs). In some embodiments, for example, where the subject is a human, the dose includes at or about 2.5×10⁷ total recombinant receptor (e.g., CAR)-expressing cells, T cells, or peripheral blood mononuclear cells (PBMCs). In some embodiments, for example, where the subject is a human, the dose includes at or about 5×10⁷ total recombinant receptor (e.g., CAR)-expressing cells, T cells, or peripheral blood mononuclear cells (PBMCs). In some embodiments, for example, where the subject is a human, the dose includes at or about 1.5×10⁸ total recombinant receptor (e.g., CAR)-expressing cells, T cells, or peripheral blood mononuclear cells (PBMCs). In some embodiments, for example, where the subject is a human, the dose includes at or about 3×10⁸ total recombinant receptor (e.g., CAR)-expressing cells, T cells, or peripheral blood mononuclear cells (PBMCs). In some embodiments, for example, where the subject is a human, the dose includes at or about 4.5×10⁸ total recombinant receptor (e.g., CAR)-expressing cells, T cells, or peripheral blood mononuclear cells (PBMCs). In some embodiments, for example, where the subject is a human, the dose includes at or about 6×10⁸ total recombinant receptor (e.g., CAR)-expressing cells, T cells, or peripheral blood mononuclear cells (PBMCs). In some embodiments, for example, where the subject is a human, the dose includes at or about 8×10⁸ total recombinant receptor (e.g., CAR)-expressing cells, T cells, or peripheral blood mononuclear cells (PBMCs). In some embodiments, for example, where the subject is a human, the dose includes at or about 1.2×10⁹ total recombinant receptor (e.g., CAR)-expressing cells, T cells, or peripheral blood mononuclear cells (PBMCs).

In some embodiments, the dose of genetically engineered cells comprises from at or about 1×10⁵ to at or about 2×10⁹ total CAR-expressing (CAR+) T cells, from at or about 1×10⁵ to at or about 5×10⁸ total CAR-expressing T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×10⁸ total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁸ total CAR-expressing T cells, from at or about 1×10⁸ to at or about 2.5×10⁸ total CAR-expressing T cells, from at or about or 2.5×10⁸ to at or about 5×10⁸ total CAR-expressing T cells. In some embodiments, the dose of genetically engineered cells comprises from at or about 1.0×10⁷ to at or about 8×10⁸ total CAR-expressing (CAR+) T cells, from at or about 1.0×10⁷ to at or about 6.5×10⁸ total CAR+ T cells, from at or about 1.5×10⁷ to at or about 6.5×10⁸ total CAR+ T cells, from at or about 1.5×10⁷ to at or about 6.0×10⁸ total CAR+ T cells, from at or about 2.5×10⁷ to at or about 6.0×10⁸ total CAR+ T cells, or from at or about 5.0×10⁷ to at or about 6.0×10⁸ total CAR+ T cells.

In some embodiments, the dose of genetically engineered cells comprises between at or about 2.5×10⁷ CAR-expressing (CAR+) T cells, total T cells, or total peripheral blood mononuclear cells (PBMCs) and at or about 1.2×10⁹ CAR-expressing T cells, total T cells, or total PBMCs, between at or about 5.0×10⁷ CAR-expressing T cells, total T cells, or total peripheral blood mononuclear cells (PBMCs) and at or about 6.0×10⁸ CAR-expressing T cells, total T cells, or total PBMCs, between at or about 5.0×10⁷ CAR-expressing T cells and at or about 4.5×10⁸ CAR-expressing T cells, total T cells, or total peripheral blood mononuclear cells (PBMCs), between at or about 1.5×10⁸ CAR-expressing T cells and at or about 3.0×10⁸ CAR-expressing T cells, total T cells, or total PBMCs, each inclusive. In some embodiments, the number is with reference to the total number of CD3+ or CD8+, in some cases also CAR-expressing (e.g. CAR+) cells. In some embodiments, the dose comprises a number of cell from or from about 2.5×10⁷ to or to about 1.2×10⁹ CD3+ or CD8+ total T cells or CD3+ or CD8+ CAR-expressing cells, from or from about 5.0×10⁷ to or to about 6.0×10⁸ CD3+ or CD8+ total T cells or CD3+ or CD8+ CAR-expressing cells, from or from about 5.0×10⁷ to or to about 4.5×10⁸ CD3+ or CD8+ total T cells or CD3+ or CD8+ CAR-expressing cells, or from or from about 1.5×10⁸ to or to about 3.0×10⁸ CD3+ or CD8+ total T cells or CD3+ or CD8+ CAR-expressing cells, each inclusive.

In some embodiments, the dose is at or about 1.0×10⁷ CD3+ CAR-expressing cells. In some embodiments, the dose is at or about 1.5×10⁷ CD3+ CAR-expressing cells. In some embodiments, the dose is at or about 2.0×10⁷ CD3+ CAR-expressing cells. In some embodiments, the dose is at or about 2.5×10⁷ CD3+ CAR-expressing cells. In some embodiments, the dose is at or about 5×10⁷ CD3+ CAR-expressing cells. In some embodiments, the dose is at or about 1.5×10⁸ CD3+ CAR-expressing cells. In some embodiments, the dose is at or about 3×10⁸ CD3+ CAR-expressing cells. In some embodiments, the dose is at or about 4.5×10⁸ CD3+ CAR-expressing cells. In some embodiments, the dose is at or about 6×10⁸ CD3+ CAR-expressing cells. In some embodiments, the dose is at or about 8×10⁸ CD3+ CAR-expressing cells. In some embodiments, the dose is at or about 1.2×10⁹ CD3+ CAR-expressing cells.

In some embodiments, the dose of genetically engineered cells is with reference to the total number of CD3+ CAR-expressing (CAR+) or CD4+/CD8+ CAR-expressing (CAR+) cells. In some embodiments, the dose comprises a number of genetically engineered cells from or from about 1.0×10⁷ to or to about 1.2×10⁹ CD3+ or CD4+/CD8+ total T cells or CD3+ CAR-expressing or CD4+/CD8+ CAR-expressing cells, from or from about 1.5×10⁷ to or to about 1.2×10⁹ CD3+ or CD4+/CD8+ total T cells or CD3+ CAR-expressing or CD4+/CD8+ CAR-expressing cells, from or from about 2.0×10⁷ to or to about 1.2×10⁹ CD3+ or CD4+/CD8+ total T cells or CD3+ CAR-expressing or CD4+/CD8+ CAR-expressing cells, from or from about 2.5×10⁷ to or to about 1.2×10⁹ CD3+ or CD4+/CD8+ total T cells or CD3+ CAR-expressing or CD4+/CD8+ CAR-expressing cells, from or from about 5.0×10⁷ to or to about 6.0×10⁸ CD3+ or CD4+/CD8+ total T cells or CD3+ CAR-expressing or CD4+/CD8+ CAR-expressing cells, from or from about 5.0×10⁷ to or to about 4.5×10⁸ CD3+ or CD4+/CD8+ total T cells or CD3+ CAR-expressing or CD4+/CD8+ CAR-expressing cells, or from or from about 1.5×10⁸ to or to about 3.0×10⁸ CD3+ or CD4+/CD8+ total T cells or CD3+ CAR-expressing or CD4+/CD8+ CAR-expressing cells, each inclusive. In some embodiments, the dose comprises at or about 1.0×10⁷, 1.5×10⁷, 2.0×10⁷, 2.5×10⁷, 5×10⁷, 1.5×10⁸,3×10⁸, 4.5×10⁸, 6×10⁸, 8×10⁸ or 1.2×10⁹ CD3+ or CD4+/CD8+ total T cells or CD3+ CAR-expressing or CD4+/CD8+ CAR-expressing cells. In some embodiments, the dose comprises at or about 2.5×10⁷, 5×10⁷, 1.5×10⁸, 3×10⁸, 4.5×10⁸, 6×10⁸, 8×10⁸ or 1.2×10⁹ CD3+ CAR-expressing cells. In some embodiments, the dose comprises at or about 1.0×10⁷, 1.5×10⁷, 2.0×10⁷, 2.5×10⁷, 5×10⁷, 1.5×10⁸,3×10⁸, 4.5×10⁸,6×10⁸, 8×10⁸ or 1.2×10⁹ CD4+/CD8+ CAR-expressing cells.

In some embodiments, the dose is at or about 1.0×10⁷ CD4+/CD8+ CAR-expressing cells. In some embodiments, the dose is at or about 1.5×10⁷ CD4+/CD8+ CAR-expressing cells. In some embodiments, the dose is at or about 2.0×10⁷ CD4+/CD8+ CAR-expressing cells. In some embodiments, the dose is at or about 2.5×10⁷ CD4+/CD8+ CAR-expressing cells. In some embodiments, the dose is at or about 5×10⁷ CD4+/CD8+ CAR-expressing cells. In some embodiments, the dose is at or about 1.5×10⁸ CD4+/CD8+ CAR-expressing cells. In some embodiments, the dose is at or about 3×10⁸ CD4+/CD8+ CAR-expressing cells. In some embodiments, the dose is at or about 4.5×10⁸ CD4+/CD8+ CAR-expressing cells. In some embodiments, the dose is at or about 6×10⁸ CD4+/CD8+ CAR-expressing cells. In some embodiments, the dose is at or about 8×10⁸ CD4+/CD8+ CAR-expressing cells. In some embodiments, the dose is at or about 1.2×10⁹ CD4+/CD8+ CAR-expressing cells. In some embodiments, the dose is at or about 2.5×10⁷ CD4+ or CD8+ CAR-expressing cells. In some embodiments, the dose is at or about 5×10⁷ CD4+ or CD8+ CAR-expressing cells. In some embodiments, the dose is at or about 1.5×10⁸ CD4+ or CD8+ CAR-expressing cells. In some embodiments, the dose is at or about 3×10⁸ CD4+ or CD8+ CAR-expressing cells. In some embodiments, the dose is at or about 4.5×10⁸ CD4+ or CD8+ CAR-expressing cells. In some embodiments, the dose is at or about 6×10⁸ CD4+ or CD8+ CAR-expressing cells. In some embodiments, the dose is at or about 6.5×10⁸ CD4+ or CD8+ CAR-expressing cells. In some embodiments, the dose is at or about 8×10⁸ CD4+ or CD8+ CAR-expressing cells. In some embodiments, the dose is at or about 1.2×10⁹ CD4+ or CD8+ CAR-expressing cells.

In some embodiments, the T cells of the dose include CD4+ T cells, CD8+ T cells or CD4+ and CD8+ T cells. In some embodiments, the T cells of the dose include CD4+ T cells. In some embodiments, the T cells of the dose include CD8+ T cells. In some embodiments, the T cells of the dose include CD4+ T cells or CD8+ T cells. In some embodiments, the T cells of the dose include CD4+ and CD8+ T cells.

In some embodiments, for example, where the subject is human, the total of CD4+ T cells and CD8+ T cells of the dose includes between at or about 1×10⁶ and at or about 2×10⁹ total CAR-expressing CD4+ cells and CAR-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 1.0×10⁷, at or about 2.5×10⁷, at or about 2.0×10⁷, at or about 2.5×10⁷, at or about 5×10⁷, at or about 1.5×10⁸, at or about 3×10⁸, at or about 4.5×10⁸, at or about 6×10⁸, at or about 6.5×10⁸, at or about 8×10⁸, or at or about 1.2×10⁹ total such cells, or the range between any two of the foregoing values. In some embodiments, for example, where the subject is human, the CD8+ T cells of the dose, including in a dose including CD4+ T cells and CD8+ T cells, includes between at or about 1×10⁶ and at or about 2×10⁹ total recombinant receptor (e.g., CAR)-expressing CD8+ cells, e.g., in the range of at or about 2.5×10⁷ to at or about 1.2×10⁹ such cells, for example, in the range of at or about 5×10⁷ to at or about 4.5×10⁸ such cells; such as at or about 2.5×10⁷, at or about 5×10⁷, at or about 1.5×10⁸, at or about 3×10⁸, at or about 4.5×10⁸, at or about 6×10⁸, at or about 8×10⁸, or at or about 1.2×10⁹ total such cells, or the range between any two of the foregoing values.

In some embodiments, the dose of cells, e.g., recombinant receptor-expressing T cells, is administered to the subject as a single dose or is administered only one time within a period of two weeks, one month, three months, six months, 1 year or more. In some embodiments, the patient is administered multiple doses, and each of the doses or the total dose can be within any of the foregoing values. In some embodiments, the engineered cells for administration or composition of engineered cells for administration, exhibits properties indicative of or consistent with cell health. In some embodiments, at or about or at least at or about 70, 75, 80, 85, or 90% CAR+ cells of such dose exhibit one or more properties or phenotypes indicative of cell health or biologically active CAR cell, such as absence expression of an apoptotic marker.

In particular embodiments, the phenotype is or includes an absence of apoptosis and/or an indication the cell is undergoing the apoptotic process. Apoptosis is a process of programmed cell death that includes a series of stereotyped morphological and biochemical events that lead to characteristic cell changes and death, including blebbing, cell shrinkage, nuclear fragmentation, chromatin condensation, chromosomal DNA fragmentation, and global mRNA decay. In some aspects, early stages of apoptosis can be indicated by activation of certain caspases, e.g., 2, 8, 9, and 10. In some aspects, middle to late stages of apoptosis are characterized by further loss of membrane integrity, chromatin condensation and DNA fragmentation, include biochemical events such as activation of caspases 3, 6, and 7.

In particular embodiments, the phenotype is negative expression of one or more factors associated with programmed cell death, for example pro-apoptotic factors known to initiate apoptosis, e.g., members of the death receptor pathway, activated members of the mitochondrial (intrinsic) pathway, such as Bcl-2 family members, e.g., Bax, Bad, and Bid, and caspases. In certain embodiments, the phenotype is the absence of an indicator, e.g., an Annexin V molecule or by TUNEL staining, that will preferentially bind to cells undergoing apoptosis when incubated with or contacted to a cell composition. In some embodiments, the phenotype is or includes the expression of one or more markers that are indicative of an apoptotic state in the cell. In some embodiments, the phenotype is lack of expression and/or activation of a caspase, such as Caspase 3. In some aspects, activation of Caspase 3 is indicative of an increase or revival of apoptosis. In certain embodiments, caspase activation can be detected by known methods. In some embodiments, an antibody that binds specifically to an activated caspase (i.e., binds specifically to the cleaved polypeptide) can be used to detect caspase activation. In particular embodiments, the phenotype is or includes active Caspase 3. In some embodiments, the marker of apoptosis is a reagent that detects a feature in a cell that is associated with apoptosis. In certain embodiments, the reagent is an Annexin V molecule.

In some embodiments, the compositions containing the engineered cells for administration contain a certain number or amount of cells that exhibit phenotypes indicative of or consistent with cell health. In some of any embodiments, less than about 25%, 20%, 15%, 10%, 9%, 8%, 7%, 6%, 5%, 4%, 3%, 2% or 1% of the CAR-expressing T cells in the dose of engineered T cells express a marker of apoptosis, optionally Annexin V or active Caspase 3. In some of any embodiments, less than 5%, 4%, 3%, 2% or 1% of the CAR-expressing T cells in the dose of engineered T cells express Annexin V or active Caspase 3.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1 or is between approximately 1:3 and approximately 3:1, such as approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is approximately 1:1. In some embodiments, the dose or composition of cells includes a defined or target ratio of CD4+ cells expressing a recombinant receptor to CD8+ cells expressing a recombinant receptor and/or of CD4+ cells to CD8+ cells that is between approximately 1:3 and approximately 3:1.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, for example, the dose contains between or between about 5.0×10⁶ and 2.25×10⁷, 5.0×10⁶ and 2.0×10⁷, 5.0×10⁶ and 1.5×10⁷, 5.0×10⁶ and 1.0×10⁷, 5.0×10⁶ and 7.5×10⁶, 7.5×10⁶ and 2.25×10⁷, 7.5×10⁶ and 2.0×10⁷, 7.5×10⁶ and 1.5×10⁷, 7.5×10⁶ and 1.0×10⁷, 1.0×10⁷ and 2.25×10⁷, 1.0×10⁷ and 2.0×10⁷, 1.0×10⁷ and 1.5×10⁷, 1.5×10⁷ and 2.25×10⁷, 1.5×10⁷ and 2.0×10⁷, 2.0×10⁷ and 2.25×10⁷ recombinant-receptor expressing cells. In some embodiments, the dose of cells contains a number of cells, that is about 1.5×10⁸ recombinant-receptor expressing cells, about 3.0×10⁸ recombinant-receptor expressing cells, or about 4.5×10⁸ recombinant-receptor expressing cells, such as recombinant-receptor expressing cells that are CD3+. In some embodiments, the dose of cells contains a number of cells, that is between at least or at least about 5×10⁶, 6×10⁶, 7×10⁶, 8×10⁶, 9×10⁶, 10×10⁶ and about 15×10⁶ recombinant-receptor expressing cells, such as recombinant-receptor expressing cells that are CD8+. In some embodiments, such dose, such as such target number of cells refers to the total recombinant-receptor expressing cells in the administered composition.

In some embodiments, for example, the lower dose contains less than about 5×10⁶ cells, recombinant receptor (e.g. CAR)-expressing cells, T cells, and/or PBMCs per kilogram body weight of the subject, such as less than about 4.5×10⁶, 4×10⁶, 3.5×10⁶, 3×10⁶, 2.5×10⁶, 2×10⁶, 1.5×10⁶, 1×10⁶, 5×10⁵, 2.5×10⁵, or 1×10⁵ such cells per kilogram body weight of the subject. In some embodiments, the lower dose contains less than about 1×10⁵, 2×10⁵, 5×10⁵, or 1×10⁶ of such cells per kilogram body weight of the subject, or a value within the range between any two of the foregoing values. In some embodiments, such values refer to numbers of recombinant receptor-expressing cells; in other embodiments, they refer to number of T cells or PBMCs or total cells administered.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses. In some embodiments, one or more subsequent dose of cells can be administered to the subject. In some embodiments, the subsequent dose of cells is administered greater than or greater than about 7 days, 14 days, 21 days, 28 days or 35 days after initiation of administration of the first dose of cells. The subsequent dose of cells can be more than, approximately the same as, or less than the first dose. In some embodiments, administration of the T cell therapy, such as administration of the first and/or second dose of cells, can be repeated

### 2. T Cell Engagers

In some embodiments, the T cell therapy is or comprises a T cell engager (TCE). that is or comprises a binding molecule capable of binding to a surface molecule expressed on a T cell. In some embodiments, the surface molecule is an activating component of a T cell, such as a component of the T cell receptor complex. In some embodiments, the surface molecule is CD3 or is CD2. In some embodiments, the TCE is or comprises an antibody or antigen-binding fragment.

In some embodiments, the TCE is selected from among the group consisting of a bispecific T cell engager (BiTE), a checkpoint-inhibitory T cell engager (CiTE), a simultaneous multiple interaction T cell engagers (SMITE), and BiTE-expressing CAR T cells (CART.BiTE cells).

In some embodiments, the TCE is a bispecific antibody containing at least one antigen-binding domain binding to an activating component of the T cell (e.g. a T cell surface molecule, e.g. CD3 or CD2) and at least one antigen-binding domain binding to a surface antigen on a target cell, such as a surface antigen on a tumor or cancer cell, for example any of the listed antigens as described herein, e.g. BCMA. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

In some embodiments, the TCE is a bi-specific T cell engager (BiTE). In some embodiments, BiTEs are used in connection with the provided methods, uses, articles of manufacture. In some embodiments, bi-specific T cell engagers have specificity toward two particular antigens (or markers or ligands). In some embodiments, the antigens are expressed on the surface of a particular type of cell. In particular embodiments, the first antigen is associated with an immune cell or an engineered immune cell, and the second antigen is associated with a target cell of the particular disease or condition, such as a cancer.

Numerous methods of producing bi-specific T cell engagers are known, including fusion of two different hybridomas (Milstein and Cuello, Nature 1983; 305:537-540), and chemical tethering though heterobifunctional cross linkers (Staerz et al. Nature 1985; 314:628-631). Among exemplary bi-specific antibody T cell-engaging molecules are those which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010).

In certain embodiments, the bi-specific T cell engager is a molecule encoded by a polypeptide construct. In certain embodiments, the polypeptide construct contains a first component comprising an antigen-binding domain binding to an activating portion of an immune cell or engineered immune cell, and a second component comprising an antigen-binding domain binding to a surface antigen (e.g. target or tumor associated antigen (TAA)) associated with a particular disease or condition (e.g. cancer). In some embodiments, the first and second components are coupled by a linker. In some embodiments, the first component is coupled to a leader sequence encoding a CD33 signal peptide.

In some embodiments, the polypeptide is a construct containing from N-terminus to C-terminus: a first component comprising an antigen-binding domain binding to an activating portion of the T cell, a peptide linker, and a second component comprising an antigen-binding domain binding to a surface antigen (e.g. target or tumor associated antigen (TAA)) associated with a disease or condition (e.g. cancer).

In some aspects, an activating component of the T cell is a T cell surface molecule, such as CD3 or CD2. In some embodiments, the surface antigen of the target cell is a tumor associated antigen (TAA, e.g. BCMA). In some aspects, the TAA contains one or more epitopes. In some embodiments, the peptide linker is or comprises a cleavable peptide linker.

In some embodiments, the antigen binding domain of the first component of the bi-specific T cell engager engages a receptor on an endogenous immune cell in the periphery of the tumor. In some embodiments, the endogenous immune cell is a T cell. In some aspects, the engagement of the endogenous T cell receptor redirects the endogenous T cells to the tumor. In some aspects, the engagement of the endogenous T cell receptor recruits tumor infiltrating lymphocytes (TILs) to the tumor. In some aspects, the engagement of the endogenous T cell receptor activates the endogenous immune repertoire.

In some embodiments, the simultaneous or near simultaneous binding of the bi-specific T cell engager to both of its targets (e.g. the immune cell and the TAA) can result in a temporary interaction between the target cell and T cell, thereby resulting in activation (e.g. cytotoxic activity, cytokine release), of the T cell and subsequent lysis of the target cell.

In some embodiments, the first component of the bi-specific T cell engager is or comprises an antigen binding domain that binds to an activating component of a T cell. In some embodiments, the activating component of the T cell is a surface molecule. In some embodiments, the surface molecule is or comprises a T-cell antigen. Exemplary T-cell antigens include but are not limited to CD2, CD3, CD4, CD5, CD6, CD8, CD25, CD28, CD30, CD40, CD44, CD45, CD69 and CD90. In some aspects, the binding of the bispecific T cell engaging molecule with the T cell antigen stimulates and/or activates the T cell.

In some embodiments, the anti-T cell binding domain includes an antibody or an antigen-binding fragment thereof selected from the group consisting of a Fab fragment, a F(ab′)₂ fragment, an Fv fragment, an scFv, a scAb, a dAb, a single domain heavy chain antibody, and a single domain light chain antibody.

In some embodiments, the T cell binding domain on the bi-specific T cell engager is an anti-CD3. In some aspects, the anti-CD3 domain is an scFv. In some embodiments, the anti-CD3 domain of the bi-specific T cell engager binds to a subunit of the CD3 complex on a receptor on a T cell. In some aspects, the receptor is on an endogenous T cell. In some embodiments, the receptor is on an engineered immune cell further expressing a recombinant receptor. The effects of CD3 engagement of T cells is well known in the art, and include but are not limited to T cell activation and other downstream cell signaling. Any of such bi-specific T cell engagers can be used in the provided disclosure herein.

In some embodiments, the second component of the bi-specific T cell engager comprising an antigen-binding domain binding to a surface antigen associated with a disease or condition is a tumor or cancer antigen. In some embodiments, among the antigens targeted by the bi-specific T cell engager are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the TCE is a checkpoint-inhibitory T cell engager (CiTE). CiTEs have been generated, including as described in Hermann et al., Blood (2018) 132(Suppl. 1):4069 and Zhou et al., Biomarker Res (2021) 9:38 (each incorporated herein by reference in its entirety). In some cases, the CiTE binds to an immune checkpoint protein, such as PD-1 or PD-L1. In some cases, the CiTE binds to PD-1. In some embodiments, the CiTE comprises a PD-1 binding domain with a BiTE targeting a tumor antigen (e.g., BCMA) and a surface molecule expressed by a T cell (e.g., CD3). In some cases, the CiTE binds to PD-L1. In some embodiments, the CiTE comprises a PD-L1 binding domain with a BiTE targeting a tumor antigen (e.g., BCMA) and a surface molecule expressed by a T cell (e.g., CD3).

In some embodiments, the TCE is a simultaneous multiple interaction T cell engager (SMITE). SMITES have been generated, including as described in Correnti et al., Leukemia (2018) 32(5):1239-43 and Zhou et al., Biomarker Res. (2021) 9:38 (each incorporated by reference herein in its entirety). In some embodiments, a SMITE comprises two BiTEs. In some embodiments, the SMITE binds to a surface molecule expressed by a T cell. In some embodiments, the SMITE binds to CD3. In some embodiments, the SMITE binds to CD28. In some embodiments, the SMITE binds to CD3 and CD28. In some embodiments, the SMITE binds two different tumor associated antigens (e.g., BCMA and GPRC5D). In some embodiments, the SMITE binds to one tumor associated antigen (TAA). In some embodiments, the SMITE binds to BCMA. In some embodiments, the SMITE binds to GPRC5D. In some embodiments, the SMITE binds to an immune checkpoint protein, such as PD-1 or PD-L1. In some embodiments, the SMITE binds to PD-1. In some embodiments, the SMITE binds to PD-L1. In some embodiments, the SMITE binds to CD3, CD28, an immune checkpoint protein, and a TAA.

In some embodiments, the TCE is or comprises BiTE-expressing CAR T cells (CART.BiTE cells). CART.BiTE cells have been generated as described in Xie and Gu, Nature Rev Canc (2022) 22:194 and Choi et al., Nat Biotechnol. (2019) 37(9):1049-58. In some embodiments, chimeric antigen receptor (CAR)-expressing T cells are engineered to secrete a BiTE, including any of those described herein. In some embodiments, the CAR binds a TAA. In some embodiments, the BiTE binds a TAA. In some embodiments, the BiTE binds to the same TAA as the CAR, or a variant thereof.

In some embodiments, the antigen includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes BCMA, GPRC5D, CD20, CD19, CD22, ROR1, CD45, CD21, CD5, CD33, Igkappa, Iglambda, CD79a, CD79b or CD30. In some embodiments, the antigen is BCMA. In some embodiments, the antigen is GPRC5D. In some embodiments, the antigen is CD19.

In some embodiments, both antigen binding domains, including the first antigen binding domain and the second antigen binding domain, comprise an antibody or an antigen-binding fragment.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv) or fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies (sdAb) are antibody fragments comprising all or a portion of the heavy chain variable domain or all or a portion of the light chain variable domain of an antibody. In certain embodiments, a single-domain antibody is a human single-domain antibody. In some embodiments, the bi-specific T cell engager comprises an antibody heavy chain domain that specifically binds the antigen, such as a cancer marker or cell surface antigen of a cell or disease to be targeted, such as a tumor cell or a cancer cell, such as any of the target antigens described herein or known. Exemplary single-domain antibodies include sdFv, nanobody, V_{H}H or V_{NAR}.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

In certain embodiments, the antigen binding domains are single chain variable fragments (scFv). In some embodiments, the scFv is a tandem scFv containing a heavy and a light chain. In some embodiments, the heavy and light chains are connected by peptide linkers. In some embodiments, the linker is composed primarily of serines and glycines. In some aspects, the linkage of the heavy chain and the light chain forms a single polypeptide antigen binding domain.

In certain embodiments, the first antigen binding domain of the bi-specific T cell engager is an anti-CD3 scFv. In certain embodiments, the second antigen binding domain of the bi-specific T cell engager is an anti-BCMA scFv. In certain embodiments, the second antigen binding domain of the bi-specific T cell engager is an anti-GPRC5D scFv. In certain embodiments, the second antigen binding domain of the bi-specific T cell engager is an anti-CD19 scFv.

In some aspects, the bi-specific T cell engager polypeptide constructs contain a linker that joins the first component comprising the antigen-binding domain that binds to an activating portion of the T cell, to the second component comprising an antigen-binding domain binding to a surface antigen (e.g. target or tumor associated antigen (TAA)) associated with a particular disease or condition. In some aspects, the linker is a short, medium or long linker.

In some embodiments, the linker is a peptide linker which is cleavable. In some aspects, the cleavable linker includes a sequence that is a substrate for a protease. In some embodiments, the sequence comprises a bond that can be broken under in vivo conditions. In some cases, the linker sequence is selectively cleaved by a protease present in a physiological environment. In some aspects, the environment is separate from the tumor microenvironment. In some embodiments, the protease is found in the periphery of the tumor.

In some embodiments, the selectively cleavable linker is cleaved by a protease produced by cells that do not co-localize with the tumor. In some embodiments, the selectively cleavable linker is not cleaved by proteases that are in the proximity of the tumor microenvironment. In some embodiments, the cleavage of the linker by the protease renders the bi-specific T cell engaging molecule inactive. In some embodiments, the protease is found in the circulating blood of a subject. In some embodiments, the protease is a part of the intrinsic or extrinsic coagulation pathway. In some aspects, the protease is a serine protease. In some aspects, the protease comprises but is not limited to a thrombin, factor X, factor XI, factor XII, and plasmin.

Among such exemplary bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). Any of such T cell-engagers can be used in used in the provided methods.

The immune system stimulator and/or the TCE can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, the TCE is administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In certain embodiments, one or more doses of a TCE are administered. In particular embodiments, between or between about 0.001 μg and about 5,000 μg, inclusive, of the TCE is administered. In particular embodiments, between or between about 0.001 μg and 1,000 μg, 0.001 μg to 1 μg, 0.01 μg to 1 μg, 0.1 μg to 10 μg, 0.01 μg to 1 μg, 0.1 μg and 5 μg, 0.1 μg and 50 μg, 1 μg and 100 μg, 10 μg and 100 μg, 50 μg and 500 μg, 100 μg and 1,000 μg, 1,000 μg and 2,000 μg, or 2,000 μg and 5,000 μg of the TCE is administered. In some embodiments, the dose of the TCE is or includes between or between about 0.01 μg/kg and 100 mg/kg, 0.1 μg/kg and 10 μg/kg, 10 μg/kg and 50 μg/kg, 50 μg/kg and 100 μg/kg, 0.1 mg/kg and 1 mg/kg, 1 mg/kg and 10 mg/kg, 10 mg/kg and 100 mg/kg, 100 mg/kg and 500 mg/kg, 200 mg/kg and 300 mg/kg, 100 mg/kg and 250 mg/kg, 200 mg/kg and 400 mg/kg, 250 mg/kg and 500 mg/kg, 250 mg/kg and 750 mg/kg, 50 mg/kg and 750 mg/kg, 1 mg/kg and 10 mg/kg, or 100 mg/kg and 1,000 mg/kg, each inclusive. In some embodiments, the dose of the TCE is at least or at least about or is or is about 0.1 μg/kg, 0.5 μg/kg, 1 μg/kg, 5 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.5 mg/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 200 mg/kg, 300 mg/kg, 400 mg/kg, 500 mg/kg, 600 mg/kg, 700 mg/kg, 800 mg/kg, 900 mg/kg, or 1,000 mg/kg. In particular embodiments, the TCE is administered orally, intravenously, intraperitoneally, transdermally, intrathecally, intramuscularly, intranasally, transmucosally, subcutaneously, or rectally.

### D. Subsequent Therapy

Provided herein are methods of selecting a subject for treatment with a subsequent therapy for treating the cancer. In some embodiments, the methods comprise selecting a subject for treatment with a subsequent therapy for treating a cancer, wherein the subject was previously treated with a T cell therapy for treating the cancer and a prior therapy for treating the cancer. In some embodiments, at the time of treatment with the T cell therapy, the subject had relapsed following treatment with, or was refractory to, the prior therapy. In some embodiments, the prior therapy and the subsequent therapy are of the same class of therapy. In some embodiments, the class of therapy is immunomodulatory drugs, proteasome inhibitors, anti-CD38 antibodies, BTK inhibitors, or BCL-2 inhibitors. In some embodiments, the class of therapy is immunomodulatory drugs. In some embodiments, the class of therapy is proteasome inhibitors. In some embodiments, the class of therapy is anti-CD38 antibodies. In some embodiments, the class of therapy is BTK inhibitors. In some embodiments, the class of therapy is BCL-2 inhibitors. In some embodiments, the methods comprise administering the subsequent therapy to the subject.

1. Selection of Subjects for Treatment with Subsequent Therapy

In some embodiments, the subject is selected for treatment with a subsequent therapy for treating the cancer. In some embodiments, the subsequent therapy is of the same class of therapy as the prior therapy for treating the cancer.

In some embodiments, the subject is selected for treatment with the subsequent therapy if the subject was administered the T cell therapy at a time when the subject had relapsed following treatment with, or was refractory to, the prior therapy. In some embodiments, the subject is selected for treatment with the subsequent therapy if, following administration of the T cell therapy, the subject achieves minimum residual disease (MRD) negative status; and after the subject achieving MRD negative status, the cancer progresses in the subject. In some embodiments, the subject is selected for treatment with the subsequent therapy if the subject was administered the T cell therapy at a time when the subject had relapsed following treatment with, or was refractory to, the prior therapy; following administration of the T cell therapy, the subject achieves minimum residual disease (MRD) negative status; and after the subject achieving MRD negative status, the cancer progresses in the subject.

In some embodiments, within about 1 month, about 2 months, about 3 months, about 6 months, or about 12 months of administration of the T cell therapy, the subject achieves MRD negative status. In some embodiments, within about 1 month of administration of the T cell therapy, the subject achieves MRD negative status. In some embodiments, within about 2 months of administration of the T cell therapy, the subject achieves MRD negative status. In some embodiments, within about 3 months of administration of the T cell therapy, the subject achieves MRD negative status. In some embodiments, within about 6 months of administration of the T cell therapy, the subject achieves MRD negative status. In some embodiments, within about 12 months of administration of the T cell therapy, the subject achieves MRD negative status.

In some embodiments, the subject is selected from treatment with the subsequent therapy if the subject was administered the T cell therapy at a time when the subject had relapsed following treatment with, or was refractory to, the prior therapy. In some embodiments, the subject is selected for treatment with the subsequent therapy if, prior to administration of the T cell therapy, cells of the cancer comprise one or more high risk feature(s) selected from among the group consisting of amplification of the long arm of chromosome 1 (amp1q), the MDMS8 gene signature, a cereblon (CRBN) mutation, biallelic p53 inactivation, high cancer clonal fraction del17p, and t(4,14); and following administration of the T cell therapy, cells of the cancer do not comprise at least one of the high risk features that the cells of the cancer comprised prior to administration of the T cell therapy. In some embodiments, the subject is selected from treatment with the subsequent therapy if, the subject was administered the T cell therapy at a time when the subject had relapsed following treatment with, or was refractory to, the prior therapy; prior to administration of the T cell therapy, cells of the cancer comprise one or more high risk feature(s) selected from among the group consisting of amplification of the long arm of chromosome 1 (amp1q), the MDMS8 gene signature, a cereblon (CRBN) mutation, biallelic p53 inactivation, high cancer clonal fraction del17p, and t(4,14); and following administration of the T cell therapy, cells of the cancer do not comprise at least one of the high risk features that the cells of the cancer comprised prior to administration of the T cell therapy.

In some embodiments, within about 1 month, about 2 months, about 3 months, about 6 months, or about 12 months of administration of the T cell therapy, the cells of the cancer do not comprise the at least one of the high risk feature(s) that the cells of the cancer comprised prior to administration of the T cell therapy. In some embodiments, within about 1 month of administration of the T cell therapy, the cells of the cancer do not comprise the at least one of the high risk feature(s) that the cells of the cancer comprised prior to administration of the T cell therapy. In some embodiments, within about 2 months of administration of the T cell therapy, the cells of the cancer do not comprise the at least one of the high risk feature(s) that the cells of the cancer comprised prior to administration of the T cell therapy. In some embodiments, within about 3 months of administration of the T cell therapy, the cells of the cancer do not comprise the at least one of the high risk feature(s) that the cells of the cancer comprised prior to administration of the T cell therapy. In some embodiments, within about 6 months of administration of the T cell therapy, the cells of the cancer do not comprise the at least one of the high risk feature(s) that the cells of the cancer comprised prior to administration of the T cell therapy. In some embodiments, within about 12 months of administration of the T cell therapy, the cells of the cancer do not comprise the at least one of the high risk feature(s) that the cells of the cancer comprised prior to administration of the T cell therapy.

In some embodiments, the subject is selected for treatment with the subsequent therapy if, prior to administration of the T cell therapy, cells of the cancer comprise a CRBN mutation; and following administration of the T cell therapy, cells of the cancer do not comprise a CRBN mutation. In some embodiments, within about 1 month, about 2 months, about 3 months, about 6 months, or about 12 months of administration of the T cell therapy, cells of the cancer do not comprise a CRBN mutation. In some embodiments, within about 1 month of administration of the T cell therapy, cells of the cancer do not comprise a CRBN mutation. In some embodiments, within about 2 months of administration of the T cell therapy, cells of the cancer do not comprise a CRBN mutation. In some embodiments, within about 3 months of administration of the T cell therapy, cells of the cancer do not comprise a CRBN mutation. In some embodiments, within about 6 months of administration of the T cell therapy, cells of the cancer do not comprise a CRBN mutation. In some embodiments, within about 12 months of administration of the T cell therapy, cells of the cancer do not comprise a CRBN mutation. In some embodiments, the subject is selected for treatment with the subsequent therapy if, prior to administration of the T cell therapy, cells of the cancer comprise a CRBN mutation; and within about 1 month, about 2 months, about 3 months, about 6 months, or about 12 months of administration of the T cell therapy, cells of the cancer do not comprise a CRBN mutation.

In some embodiments, the CRBN mutation is in exon 10 of the CRBN gene. In some embodiments, the CRBN mutation is a splice variant that results in deletion of exon 10 of the CRBN gene, or a portion thereof. In some embodiments, the CRBN mutation reduces or inhibits binding of thalidomide to the CRBN protein.

### 2. Subsequent Therapies

In some embodiments, the methods comprise selecting the subject for treatment with a subsequent therapy. In some embodiments, the methods comprise administering a subsequent therapy to a subject. In some embodiments, the subsequent therapy is administered to the subject at a time when the cancer has progressed following administration of the T cell therapy. In some embodiments, the subsequent therapy is administered to the subject about 1 month, about 2 months, about 3 months, about 6 months, about 12, about 15 months, about 18 months, or about 24 months after administration of the T cell therapy to the subject.

In some embodiments, the class of therapy is immunomodulatory drugs. In some embodiments, the subsequent therapy binds the cereblon (CRBN) protein. In some embodiments, the subsequent therapy induces degradation of Ailos. In some embodiments, the subsequent therapy induces degradation of Ikaros. In some embodiments, the subsequent therapy is selected from among the group consisting of: thalidomide, lenalidomide, pomalidomide, iberdomide, CC-92480, CC-99282, CC-91633, and CC-90009. In some embodiments, the subsequent therapy is thalidomide. In some embodiments, the subsequent therapy is lenlidomide. In some embodiments, the subsequent therapy is pomalidomide. In some embodiments, the subsequent therapy is iberdomide. In some embodiments, the subsequent therapy is CC-92480. In some embodiments, the subsequent therapy is CC-99282. In some embodiments, the subsequent therapy is CC-91633. In some embodiments, the subsequent therapy is CC-90009.

In some embodiments, the class of therapy is proteasome inhibitors. In some embodiments, the subsequent therapy is selected from among the group consisting of: bortezomib, carfilzomib and ixazomib. In some embodiments, the subsequent therapy is bortezomib. In some embodiments, the prior therapy is carfilzomib. In some embodiments, the prior therapy is ixazomib.

In some embodiments, the class of therapy is anti-CD38 antibodies. In some embodiments, the subsequent therapy is daratumumab or isatuximab. In some embodiments, the subsequent therapy is daratumumab. In some embodiments, the subsequent therapy is isatuximab.

In some embodiments, the the class of therapy is inhibitors of Bruton's tyrosine kinase (BTK). In some embodiments, the subsequent therapy is selected from among the among group consisting of ibrutinib, acalabrutinib, zanubrutinib, evobrutinib, tirabrutinib, and SNS-062. In some embodiments, the subsequent therapy is ibrutinib. In some embodiments, the subsequent therapy is acalabrutinib. In some embodiments, the subsequent therapy is zanubrutinib. In some embodiments, the subsequent therapy is evobrutinib. In some embodiments, the subsequent therapy is tirabrutinib. In some embodiments, the subsequent therapy is SNS-062.

In some embodiments, the class of therapy is inhibitors of BCL-2. In some embodiments, the subsequent therapy is selected from among the group consisting of venetoclax, navitoclax, ABT737, maritoclax, obatoclax, and clitocine. In some embodiments, the subsequent therapy is venetoclax. In some embodiments, the subsequent therapy is navitoclax. In some embodiments, the subsequent therapy is ABT737. In some embodiments, the subsequent therapy is maritoclax. In some embodiments, the subsequent therapy is obatoclax. In some embodiments, the subsequent therapy is clitocine.

In some embodiments, the subsequent therapy is a maintenance therapy. In some embodiments, the maintenance therapy is any of those as described in Section I.

In some embodiments, the subsequent therapy is of the same class of therapy as the prior therapy. In some embodiments, the prior therapy and the subsequent therapy are of any of the classes of therapies described in Section I.B.

In some embodiments, the prior therapy and the subsequent therapy are immunomodulatory drugs. In some embodiments, the prior therapy and the subsequent therapy bind to the CRBN protein. In some embodiments, the prior therapy and the subsequent therapy induce degradation of Ailos and/or Ikaros. In some embodiments, the prior therapy and the subsequent therapy are IMiDs®. In some embodiments, the prior therapy and the subsequent therapy are CELMoDs®. In some embodiments, one of the prior therapy and the subsequent therapy is an IMiD® and the other of the prior therapy and the subsequent therapy is a CELMoD®. In some embodiments, the prior therapy and the subsequent therapy are each separately selected from among the group consisting of thalidomide, lenalidomide, pomalidomide, iberdomide, CC-92480, CC-99282, CC-91633, and CC-90009.

In some embodiments, the prior therapy and the subsequent therapy are proteasome inhibitors. In some embodiments, the prior therapy and the subsequent therapy are reversible proteasome inhibitors. In some embodiments, the prior therapy and the subsequent therapy are irreversible proteasome inhibitors. In some embodiments, one of the prior therapy and the subsequent therapy is an irreversible proteasome inhibitor and the other of the prior therapy and the subsequent therapy is a reversible proteasome inhibitor. In some embodiments, the prior therapy and the subsequent therapy are each separately selected from among the group consisting of bortezomib, carfilzomib and ixazomib.

In some embodiments, the prior therapy and the subsequent therapy are anti-CD38 antibodies. In some embodiments, the prior therapy and the subsequent therapy are monoclonal antibodies. In some embodiments, the prior therapy and the subsequent therapy are fully human antibodies. In some embodiments, the prior therapy and the subsequent therapy are chimeric antibodies. In some embodiments, one of the prior therapy and the subsequent therapy is a fully human antibody and the other of the prior therapy and the subsequent therapy is a chimeric antibody. In some embodiments, the prior therapy and the subsequent therapy are each separately selected from among the group consisting of daratumumab and isatuximab.

In some embodiments, the prior therapy and the subsequent therapy are BTK inhibitors. In some embodiments, the prior therapy and the subsequent therapy are irreversible inhibitors of BTK. In some embodiments, the prior therapy and the subsequent therapy are reversible inhibitors of BTK. In some embodiments, one of the prior therapy and the subsequent therapy is an irreversible inhibitor of BTK, and the other of the prior therapy and the subsequent therapy is a reversible inhibitor of BTK. In some embodiments, the prior therapy and the subsequent therapy inhibit IL-2 inducible T-cell kinase (ITK). In some embodiments, the prior therapy and the subsequent therapy do not inhibit IL-2 inducible T-cell kinase (ITK). In some embodiments, one of the prior therapy and the subsequent therapy inhibits ITK, and the other of the prior therapy and the subsequent therapy does not inhibit ITK. In some embodiments, the prior therapy and the subsequent therapy are each separately selected from among the group consisting of ibrutinib, acalabrutinib, zanubrutinib, evobrutinib, tirabrutinib, and SNS-062.

In some embodiments, the prior therapy and the subsequent therapy are BCL-2 inhibitors. In some embodiments, the prior therapy and the subsequent therapy are selective BCL-2 inhibitors. In some embodiments, the prior therapy and the subsequent therapy are nonselective BCL-2 inhibitors. In some embodiments, one of the prior therapy and the subsequent therapy is a selective BCL-2 inhibitor, and the other of the prior therapy and the subsequent therapy is a nonselective BCL-2 inhibitor. In some embodiments, the prior therapy and the subsequent therapy are each separately selected from among the group consisting of venetoclax, navitoclax, ABT737, maritoclax, obatoclax, and clitocine.

### E. Lymphodepleting Treatment

In some aspects, the provided methods can further include administering one or more lymphodepleting therapies, such as prior to or simultaneous with initiation of administration of the T cell therapy. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the T cell therapy (e.g. dose of CAR T cells). In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the T cell therapy (e.g. dose of CAR T cells). For example, the subject may be administered a preconditioning agent at least 2 days prior, such as at least 3, 4, 5, 6, or 7 days prior, to the first dose of the T cell therapy (e.g. CAR T cells). In some embodiments, the subject is administered a preconditioning agent no more than 7 days prior, such as no more than 6, 5, 4, 3, or 2 days prior, to the administration of the T cell therapy (e.g., dose of CAR T cells).

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, the administration of the preconditioning agent prior to infusion of the dose of cells (e.g. CAR T cells) improves an outcome of the treatment. For example, in some aspects, preconditioning improves the efficacy of treatment with the dose or increases the persistence of recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells (e.g. CAR T cells). In some embodiments, the time to median disease-free survival is increased.

Once the T cell therapy (e.g. CART cells) is administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to administration of the T cell therapy (e.g., dose of CAR T cells) improves an outcome of the treatment such as by improving the efficacy of treatment with the T cell therapy or increases the persistence of T cell therapy (e.g., CAR-expressing cells, such as CAR-expressing T cells) in the subject.

### II. Exemplary Treatment Outcomes and Methods for Assessing Same

In some embodiments of the methods, uses, kits and articles of manufacture provided herein, the provided T cell therapy results in one or more treatment outcomes, such as a feature associated with any one or more of the parameters associated with the therapy or treatment, as described below. In some embodiments, the method includes assessment of one or more high risk (HR) tumor features following administration of the T cell therapy and/or following administration of the subsequent therapy. In some embodiments, the method includes assessment of the cytotoxicity of the T cells toward cancer cells, e.g., T cells administered for the T cell based therapy. In some embodiments, the method includes assessment of the exposure, persistence and proliferation of the T cells, e.g., T cells administered for the T cell based therapy. In some embodiments, the exposure, or prolonged expansion and/or persistence of the cells, and/or changes in cell phenotypes or functional activity of the cells, e.g., cells administered for immunotherapy, e.g. T cell therapy, in the methods provided herein, can be measured by assessing the characteristics of the T cells in vitro or ex vivo. In some embodiments, such assays can be used to determine or confirm the function of the T cells, e.g. T cell therapy, before, during, or after administering the T cell therapy provided herein.

In some embodiments, the T cell therapy can further include one or more screening steps to identify subjects for treatment with the T cell therapy and/or continuing the T cell therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the T cell therapy provided herein.

In some embodiments, the subsequent therapy can further include one or more screening steps to identify subjects for treatment with the subsequent therapy and/or continuing the subsequent therapy, and/or a step for assessment of treatment outcomes and/or monitoring treatment outcomes. In some embodiments, the step for assessment of treatment outcomes can include steps to evaluate and/or to monitor treatment and/or to identify subjects for administration of further or remaining steps of the therapy and/or for repeat therapy. In some embodiments, the screening step and/or assessment of treatment outcomes can be used to determine the dose, frequency, duration, timing and/or order of the T cell therapy provided herein.

In some embodiments, any of the screening steps and/or assessment of treatment of outcomes described herein can be used prior to, during, during the course of, or subsequent to administration of one or more steps of the provided T cell therapy (e.g. anti-BCMA CAR T cells). In some embodiments, any of the screening steps and/or assessment of treatment of outcomes described herein can be used prior to, during, during the course of, or subsequent to administration of one or more steps of the provided subsequent therapy (e.g. an immunomodulatory drug). In some embodiments, assessment is made prior to, during, during the course of, or after performing any of the methods provided herein. In some embodiments, the assessment is made prior to performing the methods provided herein. In some embodiments, assessment is made after performing one or more steps of the methods provided herein. In some embodiments, the assessment is performed prior to administration of one or more steps of the provided T cell therapy, for example, to screen and identify patients suitable and/or susceptible to receive the T cell therapy. In some embodiments, the assessment is performed during, during the course of, or subsequent to administration of one or more steps of the provided T cell therapy, for example, to assess the intermediate or final treatment outcome, e.g., to determine the efficacy of the treatment and/or to determine whether to continue or repeat the treatments and/or to determine whether to administer the remaining steps of the T cell therapy. In some embodiments, the assessment is performed during, during the course of, or subsequent to administration of one or more steps of the provided T cell therapy, for example, to determine whether to administer the subsequent therapy. In some embodiments, the assessment is performed prior to administration of the subsequent therapy, for example, to screen and identify patients suitable and/or susceptible to receive the subsequent therapy.

In some embodiments, treatment of outcomes includes improved immune function, e.g., immune function of the T cells administered for cell based therapy and/or of the endogenous T cells in the body. In some embodiments, exemplary treatment outcomes include, but are not limited to, enhanced T cell proliferation, enhanced T cell functional activity, changes in immune cell phenotypic marker expression, such as such features being associated with the engineered T cells, e.g. CAR-T cells, administered to the subject. In some embodiments, exemplary treatment outcomes include decreased disease burden, e.g., tumor burden, improved clinical outcomes and/or enhanced efficacy of therapy.

In some embodiments, the screening step and/or assessment of treatment of outcomes includes assessing the survival and/or function of the T cells administered for cell based therapy. In some embodiments, the screening step and/or assessment of treatment of outcomes includes assessing the levels of cytokines or growth factors. In some embodiments, the screening step and/or assessment of treatment of outcomes includes assessing disease burden and/or improvements, e.g., assessing tumor burden and/or clinical outcomes. In some embodiments, either of the screening step and/or assessment of treatment of outcomes can include any of the assessment methods and/or assays described herein and/or known in the art, and can be performed one or more times, e.g., prior to, during, during the course of, or subsequently to administration of one or more steps of the T cell therapy. Exemplary sets of parameters associated with a treatment outcome, which can be assessed in some embodiments of the methods provided herein, include peripheral blood immune cell population profile and/or tumor burden.

In some embodiments, the methods affect efficacy of the cell therapy in the subject. In some embodiments, the cytotoxicity of recombinant receptor-expressing, e.g., CAR-expressing, cells in the subject following administration of the dose of cells in the method with debulking, is greater as compared to that achieved via a method without debulking. In some embodiments, the cytotoxicity of recombinant receptor-expressing, e.g., CAR-expressing, cells in the subject following administration of the dose of cells in the method wherein a subject is selected for treatment if the subject achieves MRD negative status following administration of the T cell therapy and following achievement of MRD negative status, the cancer progresses (e.g., disease progression), is greater as compared to that achieved via a method without selecting the subject. In some embodiments, the cytotoxicity of recombinant receptor-expressing, e.g., CAR-expressing, cells in the subject following administration of the dose of cells in the method wherein, a subject is selected for treatment if, following administration of the T cell therapy, the cancer no longer exhibits at least one of the HR tumor features exhibits by the cancer prior to administration of the T cell therapy, is greater as compared to that achieved via a method without selecting the subject. In some embodiments, cytotoxicity in the subject of the administered T cell therapy, e.g., CAR-expressing T cells is assessed as compared to a method in which the T cell therapy is administered to a subject who is not selected for treatment. In some embodiments, the methods result in the administered T cells exhibiting increased or prolonged cytotoxicity in the subject as compared to a method in which the T cell therapy is administered to a subject who is not selected for treatment. In some embodiments, the methods result in the administered subsequent therapy exhibiting increased or prolonged cytotoxicity in the subject as compared to a method in which the subsequent therapy is administered to a subject who is not selected for treatment.

In some embodiments, the subject can be screened prior to the administration of one or more steps of the methods. For example, the subject can be screened for HR tumor features prior to administration of the subsequent therapy, to determine suitability, responsiveness and/or susceptibility to administering the subsequent therapy. For example, the subject can be screened for MRD negative status following administration of the T cell therapy, to determine suitability, responsiveness and/or susceptibility to administering the subsequent therapy. In some cases, the subject can be screened for characteristics of the disease prior to administration of the T cell therapy, to determine suitability, responsiveness and/or susceptibility to administering the T cell therapy. In some embodiments, the screening step and/or assessment of treatment outcomes can be used to determine the dose, frequency, duration, timing and/or order of the T cell therapy provided herein. The subject can be screened for characteristics of the disease prior to administration of the subsequent therapy, to determine suitability, responsiveness and/or susceptibility to administering the subsequent therapy. In some embodiments, the screening step and/or assessment of treatment outcomes can be used to determine the dose, frequency, duration, timing and/or order of the subsequent therapy provided herein.

In some embodiments, the subject can be screened after administration of one of the steps of the T cell therapy, to determine and identify subjects to receive the remaining steps of the T cell therapy and/or to monitor efficacy of the therapy. In some embodiments, the number, level or amount of administered T cells and/or proliferation and/or activity of the administered T cells is assessed after administration of the engineered T cells.

In some embodiments, the subject can be screened after administration of the T cell therapy, to determine and identify subjects to receive the subsequent therapy and/or to monitor efficacy of the therapy. In some embodiments, the MRD status of the subject is assessed after administration of the T cell therapy. In some embodiments, the presence of HR tumor features is assessed after administration of the T cell therapy.

In some embodiments, a change and/or an alteration, e.g., an increase, an elevation, a decrease or a reduction, in levels, values or measurements of a parameter or outcome compared to the levels, values or measurements of the same parameter or outcome in a different time point of assessment, a different condition, a reference point and/or a different subject is determined or assessed. In some embodiments, the levels, values or measurements of two or more parameters are determined, and relative levels are compared. In some embodiments, the determined levels, values or measurements of parameters are compared to the levels, values or measurements from a control sample or an untreated sample. In some embodiments, the determined levels, values or measurements of parameters are compared to the levels from a sample from the same subject but at a different time point. The values obtained in the quantification of individual parameter can be combined for the purpose of disease assessment, e.g., by forming an arithmetical or logical operation on the levels, values or measurements of parameters by using multi-parametric analysis. In some embodiments, a ratio of two or more specific parameters can be calculated.

Assessment and determination of parameters associated with T cell health, function, activity, and/or outcomes, such as response, efficacy and/or toxicity outcomes, can be assessed at various time points. In some aspects, the assessment can be performed multiple times, e.g., prior to, during, and/or after manufacturing of the cells, prior to, during, and/or after the initiation of administration of the T cell therapy.

In some embodiments, functional attributes of the administered cells and/or cell compositions include monitoring pharmacokinetic (PK) and pharmacodynamics parameters, expansion and persistence of the cells, cell functional assays (e.g., any described herein, such as cytotoxicity assay, cytokine secretion assay and in vivo assays), high-dimensional T cell signaling assessment, and assessment of exhaustion phenotypes and/or signatures of the T cells.

In some embodiments, parameters associated with therapy or a treatment outcome, which include parameters that can be assessed for the screening steps and/or assessment of treatment of outcomes and/or monitoring treatment outcomes, includes tumor or disease burden. The administration of the immunotherapy, such as a T cell therapy (e.g. CAR-expressing T cells) can reduce or prevent the expansion or burden of the disease or condition in the subject. For example, where the disease or condition is a tumor, the methods generally reduce tumor size, bulk, metastasis, percentage of blasts in the bone marrow or molecularly detectable cancer and/or improve prognosis or survival or other symptom associated with tumor burden.

In some embodiments, the provided methods result in a decreased tumor burden in treated subjects compared to alternative methods in which the subsequent therapy is given without providing the T cell therapy (e.g. anti-BCMA CAR T cells) prior to provision of the subsequent therapy. In some embodiments, the provided methods result in a decreased tumor burden in treated subjects compared to alternative methods in which the subsequent therapy is given to a subject without selecting the subject for treatment.

It is not necessary that the tumor burden actually be reduced in all subjects receiving the T cell therapy, but that tumor burden is reduced on average in subjects treated, such as based on clinical data, in which a majority of subjects treated with such a T cell therapy exhibit a reduced tumor burden, such as at least 50%, 60%, 70%, 80%, 90%, 95% or more of subjects treated with the T cell therapy, exhibit a reduced tumor burden.

It is not necessary that the tumor burden actually be reduced in all subjects receiving the subsequent therapy, but that tumor burden is reduced on average in subjects treated, such as based on clinical data, in which a majority of subjects treated with such a subsequent therapy exhibit a reduced tumor burden, such as at least 50%, 60%, 70%, 80%, 90%, 95% or more of subjects treated with the subsequent therapy, exhibit a reduced tumor burden.

Disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood, lymph or bone marrow in the context of certain hematological malignancies. Disease burden can include, in some embodiments, the mass of a tumor, the number or extent of metastases and/or the percentage of blast cells present in the bone marrow.

In the case of MM, exemplary parameters to assess the extent of disease burden include such parameters as number of clonal plasma cells (e.g., >10% on bone marrow biopsy or in any quantity in a biopsy from other tissues; plasmacytoma), presence of monoclonal protein (paraprotein) in either serum or urine, evidence of end-organ damage felt related to the plasma cell disorder (e.g., hypercalcemia (corrected calcium >2.75 mmol/l); renal insufficiency attributable to myeloma; anemia (hemoglobin <10 g/dl); and/or bone lesions (lytic lesions or osteoporosis with compression fractures)).

Exemplary methods for assessing disease status or disease burden include: measurement of M protein in biological fluids, such as blood and/or urine, by electrophoresis and immunofixation; quantification of sFLC (κ and λ) in blood; skeletal survey; and imaging by positron emission tomography (PET)/computed tomography (CT) in subjects with extramedullary disease. In some embodiments, disease status can be evaluated by bone marrow examination. In some examples, efficacy of the T cell therapy following its administration to the subject is determined by the expansion and persistence of the T cells (e.g. BCMA CAR T cells) in the blood and/or bone marrow. In some embodiments, efficacy of the T cell therapy is determined based on the antitumor activity of the administered cells (e.g. BCMA CAR T cells). In some embodiments antitumor activity is determined by the overall response rate (ORR) and/or International Myeloma Working Group (IMWG) Uniform Response Criteria (see Kumar et al. (2016) Lancet Oncol 17(8):e328-346). In some embodiments, response is evaluated using minimal residual disease (MRD) assessment. In some embodiments, MRD can be assessed by methods such as flow cytometry and high-throughput sequencing, e.g., deep sequencing. In some aspects, subjects that have a MRD-negative disease include those exhibiting absence of aberrant clonal plasma cells on bone marrow aspirate, ruled out by an assay with a minimum sensitivity of 1 in 105 nucleated cells or higher (i.e., 10−5 sensitivity), such as flow cytometry (next-generation flow cytometry; NGF) or high-throughput sequencing, e.g., deep sequencing or next-generation sequencing (NGS).

In some aspects, sustained MRD-negative includes subjects that exhibit MRD negativity in the marrow (NGF or NGS, or both) and by imaging as defined below, confirmed minimum of 1 year apart. Subsequent evaluations can be used to further specify the duration of negativity (e.g., MRD-negative at 5 years). In some aspects, flow MRD-negative includes subjects that exhibit an absence of phenotypically aberrant clonal plasma cells by NGF on bone marrow aspirates using the EuroFlow standard operation procedure for MRD detection in multiple myeloma (or validated equivalent method) with a minimum sensitivity of 1 in 105 nucleated cells or higher. In some aspects, sequencing MRD-negative includes subjects that exhibit an absence of clonal plasma cells by NGS on bone marrow aspirate in which presence of a clone is defined as less than two identical sequencing reads obtained after DNA sequencing of bone marrow aspirates using the LymphoSIGHT platform (or validated equivalent method) with a minimum sensitivity of 1 in 105 nucleated cells or higher. In some aspects, imaging plus MRD-negative includes subjects that exhibit MRD negativity as assessed by NGF or NGS plus disappearance of every area of increased tracer uptake found at baseline or a preceding PET/CT or decrease to less mediastinal blood pool SUV or decrease to less than that of surrounding normal tissue (see Kumar et al. (2016) Lancet Oncol 17(8):e328-346).

In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of tumor burden is specified. In some embodiments, exemplary parameters for determination include particular clinical outcomes indicative of amelioration or improvement in the tumor. Such parameters include: duration of disease control, including objective response (OR), complete response (CR), stringent complete response (sCR), very good partial response (VGPR), partial response (PR), minimal response (MR), Stable disease (SD), Progressive disease (PD) or relapse (see, e.g., International Myeloma Working Group (IMWG) Uniform Response Criteria; see Kumar et al. (2016) Lancet Oncol 17(8):e328-346), objective response rate (ORR), progression-free survival (PFS) and overall survival (OS). In some embodiments, response is evaluated using minimal residual disease (MRD) assessment. In some embodiments, response is evaluated using complete response (CR) or stringent CR (sCR) assessment. In some embodiments, response is evaluated using complete response (CR) assessment. In some embodiments, response is evaluated using stringent CR (sCR) assessment. Specific thresholds for the parameters can be set to determine the efficacy of the methods provided herein. In some embodiments, the disease or disorder to be treated is multiple myeloma. In some embodiments, measurable disease criteria for multiple myeloma can include (1) serum M-protein 1 g/dL or greater; (2) Urine M-protein 200 mg or greater/24 hour; (3) involved serum free light chain (sFLC) level 10 mg/dL or greater, with abnormal κ to λ ratio. In some cases, light chain disease is acceptable only for subjects without measurable disease in the serum or urine.

In some embodiments, response is evaluated based on the duration of response following administration of the T cell therapy, e.g. BCMA CAR T cells. In some aspects, the response to the therapy, e.g., according to the provided embodiments, can be measured at a designated time point after the initiation of administration of the T cell therapy. In some embodiments, the designated time point is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks months following initiation of the administration, or within a range defined by any of the foregoing. In some embodiments, the designated time point is at or about 1 month following initiation of the administration. In some embodiments, the designated time point is at or about 3 months following initiation of the administration. In some embodiments, the designated time point is at or about 6 months following initiation of the administration. In some embodiments, the designated time point is at or about 9 months following initiation of the administration. In some embodiments, the designated time point is at or about 12 months following initiation of the administration. In some embodiments, the response is a CR or a sCR. In some embodiments, the response is a CR. In some embodiments, the response is a sCR.

In some embodiments, response is evaluated based on the duration of response following administration of the subsequent therapy, e.g. an immunomodulatory drug. In some aspects, the response to the therapy, e.g., according to the provided embodiments, can be measured at a designated time point after the initiation of administration of the subsequent therapy. In some embodiments, the designated time point is at or about 1, 2, 3, 6, 9, 12, 18, 24, 30 or 36 months following initiation of the administration, or within a range defined by any of the foregoing. In some embodiments, the designated time point is 4, 8, 12, 16, 20, 24, 28, 32, 36, 48 or 52 weeks months following initiation of the administration, or within a range defined by any of the foregoing. In some embodiments, the designated time point is at or about 1 month following initiation of the administration. In some embodiments, the designated time point is at or about 3 months following initiation of the administration. In some embodiments, the designated time point is at or about 6 months following initiation of the administration. In some embodiments, the designated time point is at or about 9 months following initiation of the administration. In some embodiments, the designated time point is at or about 12 months following initiation of the administration. In some embodiments, the response is a CR or a sCR. In some embodiments, the response is a CR. In some embodiments, the response is a sCR.

In some embodiments, the response or outcome determined at or about 3, 6, 9 or 12 months after the designated time point is equal to or improved compared to the response or outcome determined at the initial designated time point. For example, in some aspects, if the response or outcome determined at the initial designated time point is stable disease (SD), Progressive disease (PD) or relapse, the subject treated according to the provided embodiments can show an equal or improved response or outcome (e.g., exhibiting a better response outcome according to the International Myeloma Working Group (IMWG) Uniform Response Criteria; see Kumar et al. (2016) Lancet Oncol 17(8):e328-346) at a subsequent time point, after at or about 3, 6, 9 or 12 months after the initial designated time point, that is equal to the response or outcome at the initial designated time point, or a response or outcome that is objective response (OR), complete response (CR), stringent complete response (sCR), very good partial response (VGPR) or partial response (PR). In some embodiments, the response is a CR or a sCR. In some embodiments, the response is a CR. In some embodiments, the response is a sCR. In some aspects, subjects treated according to the provided embodiments can show a response or outcome that is improved between two time point of determination. In some aspects, the subject can exhibit a PR or VGPR in the initial designated time point for assessment, e.g., at 4 weeks after the initiation of administration, then exhibit an improved response, such as a CR or an sCR, at a later time point, e.g., at 12 weeks after the initiation of administration. In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response or outcome is durable for greater than at or about 3, 6, 9 or 12 months.

In some embodiments, the Eastern Cooperative Oncology Group (ECOG) performance status indicator can be used to assess or select subjects for treatment, e.g., subjects who have had poor performance from prior therapies (see, e.g., Oken et al. (1982) Am J Clin Oncol. 5:649-655). The ECOG Scale of Performance Status describes a patient's level of functioning in terms of their ability to care for themselves, daily activity, and physical ability (e.g., walking, working, etc.). In some embodiments, an ECOG performance status of 0 indicates that a subject can perform normal activity. In some aspects, subjects with an ECOG performance status of 1 exhibit some restriction in physical activity but the subject is fully ambulatory. In some aspects, patients with an ECOG performance status of 2 is more than 50% ambulatory. In some cases, the subject with an ECOG performance status of 2 may also be capable of self-care; see e.g., Sorensen et al., (1993) Br J Cancer 67(4) 773-775. In some embodiments, the subject that are to be administered according to the methods or treatment regimen provided herein include those with an ECOG performance status of 0 or 1.

In some embodiments, the methods and/or administration of an a T cell therapy (e.g. BCMA CAR T cells) decrease(s) disease burden as compared with disease burden at a time immediately prior to the administration of the T cell therapy. In some embodiments, the methods and/or administration of an a T cell therapy (e.g. BCMA CAR T cells) changes the HR tumor feature(s) exhibited by the cancer as compared with a time immediately prior to the administration of the T cell therapy. In some embodiments, the methods and/or administration of an a T cell therapy (e.g. BCMA CAR T cells) changes the HR CRBN tumor feature(s) exhibited by the cancer as compared with a time immediately prior to the administration of the T cell therapy. In some embodiments, the methods and/or administration of an a T cell therapy (e.g. BCMA CAR T cells) results in the subject achieving MRD negative status, as compared with a time immediately prior to the administration of the T cell therapy.

In some aspects, administration of the T cell therapy may prevent an increase in disease burden, and this may be evidenced by no change in disease burden.

In some embodiments, the method reduces the burden of the disease or condition, e.g., number of tumor cells, size of tumor, duration of patient survival or event-free survival, to a greater degree and/or for a greater period of time as compared to the reduction that would be observed with a comparable method using an alternative therapy, such as one in which the subject receives a subsequent therapy in the absence of the subject receiving the T cell therapy therapy and/or in the absence of the subject being selected for treatment. In some embodiments, disease burden is reduced to a greater extent or for a greater duration following the of administration of the subsequent therapy, compared to the reduction that would be effected without provision of the T cell therapy prior to administration of the subsequent therapy and/or without selecting the subject for treatment.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). In some embodiments, the parameter is CR or sCR. In some embodiments, the parameter is CR. In some embodiments, the parameter is sCR. Specific thresholds for the parameters can be set to determine the efficacy of the method of T cell therapy provided herein.

In some embodiments, the subjects treated according to the method achieve a more durable response. In some cases, a measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate at approximately 1, 2, 3, 4, 5, 6, 7, 8, 9, 10, 11, 12, 18 or 24 months after initiation of therapy. In some embodiments, the response is durable for greater than 3 months, greater than 6 months, or great than 12 months. In some particular embodiments, the subjects treated according to the method achieve a more durable response after the subject previously relapsed following remission in response to the administration of the genetically engineered cells.

In some aspects, disease burden is measured or detected prior to administration of the prior to administration of the T cell therapy, and/or prior to administration of the subsequent therapy. In the context of multiple administration of one or more steps of the T cell therapy, disease burden in some embodiments may be measured prior to or following administration of any of the steps, doses and/or cycles of administration, or at a time between administration of any of the steps, doses and/or cycles of administration.

In some embodiments, the burden is decreased by or by at least at or about 10, 20, 30, 40, 50, 60, 70, 80, 90, or 100% by the provided methods compared to immediately prior to the administration of the T cell therapy. In some embodiments, disease burden, tumor size, tumor volume, tumor mass, and/or tumor load or bulk is reduced following administration of the T cell therapy, by at least at or about 10, 20, 30, 40, 50, 60, 70, 80, 90% or more compared to that immediately prior to the administration of the T cell therapy. In some embodiments, disease burden, tumor size, tumor volume, tumor mass, and/or tumor load or bulk is reduced following administration of the subsequent therapy, by at least at or about 10, 20, 30, 40, 50, 60, 70, 80, 90% or more compared to that immediately prior to the administration of the subsequent therapy.

In some embodiments, reduction of disease burden by the method comprises an induction in morphologic complete remission, for example, as assessed at 1 month, 2 months, 3 months, or more than 3 months, after administration of, e.g., initiation of, the T cell therapy. In some embodiments, reduction of disease burden by the method comprises an induction in morphologic complete remission, for example, as assessed at 1 month, 2 months, 3 months, or more than 3 months, after administration of, e.g., initiation of, the subsequent therapy.

In some aspects, an assay for minimal residual disease, for example, as measured by multiparametric flow cytometry, is negative, or the level of minimal residual disease is less than about 0.3%, less than about 0.2%, less than about 0.1%, or less than about 0.05%.

In some embodiments, the event-free survival rate or overall survival rate of the subject is improved by the methods, as compared with other methods. For example, in some embodiments, event-free survival rate or probability for subjects treated by the methods at 6 months following the method of subsequent therapy provided herein, is greater than about 40%, greater than about 50%, greater than about 60%, greater than about 70%, greater than about 80%, greater than about 90%, or greater than about 95%. In some aspects, overall survival rate is greater than about 40%, greater than about 50%, greater than about 60%, greater than about 70%, greater than about 80%, greater than about 90%, or greater than about 95%. In some embodiments, the subject treated with the methods exhibits event-free survival, relapse-free survival, or survival to at least 6 months, or at least 1, 2, 3, 4, 5, 6, 7, 8, 9, or 10 years. In some embodiments, the time to progression is improved, such as a time to progression of greater than at or about 6 months, or at least 1, 2, 3, 4, 5, 6, 7, 8, 9, or 10 years.

In some embodiments, following treatment by the method, the probability of relapse is reduced as compared to other methods. For example, in some embodiments, the probability of relapse at 6 months following the provided method is less than about 80%, less than about 70%, less than about 60%, less than about 50%, less than about 40%, less than about 30%, less than about 20%, or less than about 10%.

In some embodiments, the administration of the subsequent therapy can treat the subject despite the subject having become resistant to the prior therapy, which is of the same class of therapy as the subsequent therapy. In some embodiments, when administered to subjects according to the embodiments described herein, the dose or the composition is capable of achieving complete response (CR) or stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving complete response (CR), in at least 50%, at least 60%, at least 70%, at least 80%, at least 90%, or at least 95% of subjects that were administered. In some embodiments, when administered to subjects according to the embodiments described herein, the dose or the composition is capable of achieving stringent CR (sCR), in at least 50%, at least 60%, at least 70%, at least 80%, at least 90%, or at least 95% of subjects that were administered. In some embodiments, when administered to subjects according to the embodiments described herein, the dose or the composition is capable of achieving objective response (OR), in at least 50%, at least 60%, at least 70%, at least 80%, at least 90%, or at least 95% of subjects that were administered. In some embodiments, OR includes subjects who achieve stringent complete response (sCR), complete response (CR), very good partial response (VGPR), partial response (PR) and minimal response (MR). In some embodiments, when administered to subjects according to the embodiments described herein, the dose or the composition is capable of achieving stringent complete response (sCR), complete response (CR), very good partial response (VGPR) or partial response (PR), in at least 50%, 60%, 70%, 80%, or 85% of subjects that were administered. In some embodiments, when administered to subjects according to the embodiments described herein, the dose or the composition is capable of achieving stringent complete response (sCR) or complete response (CR) at least 20%, 30%, 40% 50%, 60% or 70% of subjects that were administered. In some embodiments, when administered to subjects according to the embodiments described herein, the dose or the composition is capable of achieving stringent complete response (sCR) at least 20%, 30%, 40% 50%, 60% or 70% of subjects that were administered. In some embodiments, when administered to subjects according to the embodiments described herein, the dose or the composition is capable of achieving complete response (CR) at least 20%, 30%, 40% 50%, 60% or 70% of subjects that were administered. In some embodiments, exemplary doses include about 1.0×10⁷, 1.5×10⁷, 2.0×10⁷, 2.5×10⁷, 5.0×10⁷, 1.5×10⁸, 3.0×10⁸, 4.5×10⁸, 6.0×10⁸ or 8.0×10⁸ CAR-expressing (CAR+) T cells. In some embodiments, exemplary doses include about 1.5×10⁸, 3.0×10⁸, or 4.5×10⁸, CAR-expressing (CAR+) T cells. In some aspects, particular response to the treatment, e.g., according to the methods provided herein, can be assessed based on the International Myeloma Working Group (IMWG) Uniform Response Criteria (see Kumar et al. (2016) Lancet Oncol 17(8):e328-346).

### III. Articles of Manufacture and Kits

Provided herein are articles of manufacture containing a T cell therapy, e.g. engineered cells or a T cell engager, and/or compositions thereof. Also provided herein are articles of manufacture containing a subsequent therapy, e.g. an immunomodulatory drug, and/or compositions thereof. Also provided herein are articles of manufacture containing (1) a T cell therapy, e.g. engineered cells or a T cell engager, and/or compositions thereof, and (2) a subsequent therapy, e.g. an immunomodulatory drug, and/or compositions thereof. The articles of manufacture may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition.

The article of manufacture may include (a) a first container with a composition contained therein, wherein the composition includes the T cell therapy, such as CAR T cells; and (b) a second container with a composition contained therein, wherein the composition includes the subsequent therapy, such as an immunomodulatory drug. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, reference to a “corresponding form” of an antibody means that when comparing a property or activity of two antibodies, the property is compared using the same form of the antibody. For example, if it is stated that an antibody has greater activity compared to the activity of the corresponding form of a first antibody, that means that a particular form, such as an scFv of that antibody, has greater activity compared to the scFv form of the first antibody.

“Human BCMA” refers to BCMA found in a human subject, and having, e.g., SEQ ID NO: 215.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The terms “full length antibody,” “intact antibody,” and “whole antibody” are used herein interchangeably to refer to an antibody having a structure substantially similar to a native antibody structure or having heavy chains that contain an Fc region as defined herein.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-BCMA antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The terms “polypeptide” and “protein” are used interchangeably to refer to a polymer of amino acid residues, and are not limited to a minimum length. Polypeptides, including the antibodies and antibody chains and other peptides, e.g., linkers and BCMA-binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways, for instance, using publicly available computer software such as BLAST, BLAST-2, ALIGN or Megalign (DNASTAR) software. Appropriate parameters for aligning sequences, including any algorithms needed to achieve maximal alignment over the full length of the sequences being compared, can be determined.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “package insert” is used to refer to instructions customarily included in commercial packages of therapeutic products, that contain information about the indications, usage, dosage, administration, T cell therapy, contraindications and/or warnings concerning the use of such therapeutic products.

The term “about” as used herein refers to the usual error range for the respective value readily known in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

### V. Exemplary Embodiments

Among the provided embodiments are:
1. A method of treating a cancer, comprising:
(a) selecting a subject having a cancer for treatment with a subsequent therapy for treating the cancer, wherein the subject was previously administered a T cell therapy for treating the cancer and a prior therapy for treating the cancer, and wherein:
   (i) the subject was administered the T cell therapy at a time when the subject had relapsed following treatment with, or was refractory to, the prior therapy;
   (ii) following administration of the T cell therapy, the subject achieves minimum residual disease (MRD) negative status; and
   (iii) after the subject achieving MRD negative status, the cancer progresses in the subject; and
(b) administering the subsequent therapy to the subject, wherein the prior therapy and the subsequent therapy are of the same class of therapy.
2. A method of selecting a subject having a cancer in which the cancer is re-sensitized to a class of therapy, comprising:
(a) administering a T cell therapy to a subject having a cancer at a time when the subject has relapsed following treatment with, or is refractory to, a prior therapy for treating the cancer; and
(b) selecting the subject for treatment with a subsequent therapy for treating the cancer, wherein the subject is selected for treatment with the subsequent therapy if:
   (i) following administration of the T cell therapy, the subject achieves minimum residual disease (MRD) negative status; and
   (ii) after the subject achieves MRD negative status, the cancer progresses in the subject, wherein the prior therapy and the subsequent therapy are of the same class of therapy.
3. The method of embodiment 2, further comprising (c) administering the subsequent therapy to the subject.
4. A method of treating a cancer, comprising:
(a) selecting a subject having a cancer for treatment with a subsequent therapy for treating the cancer, wherein the subject was previously administered a T cell therapy for treating the cancer and a prior therapy for treating the cancer, and wherein:
   (i) the subject was administered the T cell therapy at a time when the subject had relapsed following treatment with, or was refractory to, the prior therapy;
   (ii) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
   (iii) following administration of the T cell therapy, cells of the cancer do not comprise at least one of the high risk features that the cells of the cancer comprised prior to administration of the T cell therapy; and
(b) administering the subsequent therapy to the subject, wherein the prior therapy and the subsequent therapy are of the same class of therapy.
5. A method of selecting a subject having a cancer in which the cancer is re-sensitized to a class of therapy, comprising:
(a) administering a T cell therapy to a subject having a cancer at a time when the subject has relapsed following treatment with, or is refractory to, a prior therapy for treating the cancer; and
(b) selecting the subject for treatment with a subsequent therapy for treating the cancer, wherein the subject is selected for treatment with the subsequent therapy if:
   (i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
   (ii) following administration of the T cell therapy, cells of the cancer do not comprise at least one of the high risk features that the cells of the cancer comprised prior to administration of the T cell therapy,
wherein the prior therapy and the subsequent therapy are of the same class of therapy.
6. The method of embodiment 5, further comprising (c) administering the subsequent therapy to the subject.
7. A method of treating a cancer, comprising:
(a) administering to a subject having a cancer a T cell therapy for treating the cancer at a time when the subject has relapsed following treatment with, or is refractory to, a prior therapy for treating the cancer; and
(b) administering a subsequent therapy for treating the cancer to the subject, wherein the prior therapy and the subsequent therapy are of the same class of therapy.
8. A method of re-sensitizing a cancer in a subject, comprising:
(a) administering to a subject having a cancer a T cell therapy for treating the cancer at a time when the subject has relapsed following treatment with, or is refractory to, a prior therapy for treating the cancer; and
(b) administering a subsequent therapy for treating the cancer to the subject, wherein the prior therapy and the subsequent therapy are of the same class of therapy.
9. The method of embodiment 7 or embodiment 8, further comprising, prior to (b), selecting the subject for treatment with the subsequent therapy, wherein the subject is selected for treatment with the subsequent therapy if:
   (i) following administration of the T cell therapy, the subject achieves minimum residual disease (MRD) negative status; and
   (ii) subsequent to the subject achieving MRD negative status, the cancer progresses in the subject.
10. The method of any of embodiments 7-9, wherein:
   (i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
   (ii) following administration of the T cell therapy, cells of the cancer do not comprise at least one of the high risk feature(s) that the cells of the cancer comprised prior to administration of the T cell therapy.
11. The method of any of embodiments 1-3, 9, and 10, wherein, within about 1 month, about 2 months, about 3 months, about 6 months, or about 12 months of administration of the T cell therapy, the subject achieves MRD negative status.
12. The method of any of embodiments 4-6, 10, and 11, wherein, within about 1 month, about 2 months, about 3 months, about 6 months, or about 12 months of administration of the T cell therapy, the cells of the cancer do not comprise at least one of the high risk feature(s) that the cells of the cancer comprised prior to administration of the T cell therapy.
13. The method of any of embodiments 1-12, wherein, prior to administration of the T cell therapy, cells of the cancer comprise a CRBN mutation.
14. The method of any of embodiments 1-13, wherein, within about 1 month, about 2 months, about 3 months, about 6 months, or about 12 months of administration of the T cell therapy, cells of the cancer do not comprise a CRBN mutation.
15. The method of any of embodiments 4-6 and 12-14, wherein the CRBN mutation is in exon 10 of the CRBN gene.
16. The method of any of embodiments 4-6 and 12-15, wherein the CRBN mutation reduces or inhibits binding of thalidomide to the CRBN protein.
17. The method of any of embodiments 1-16, wherein the cancer is a B cell malignancy.
18. The method of any of embodiments 1-17, wherein the cancer is a multiple myeloma (MM).
19. The method of embodiment 18, wherein the MM is a relapsed/refractory (R/R) MM.
20. The method of any of embodiments 1-19, wherein the class of therapy is immunomodulatory drugs.
21. The method of any of embodiments 1-20, wherein the prior therapy and the subsequent therapy both bind the cereblon (CRBN) protein.
22. The method of any of embodiments 1-21, wherein the prior therapy and the subsequent therapy both induce degradation of Ailos and/or Ikaros.
23. The method of any of embodiments 1-22, wherein the prior therapy is selected from among the group consisting of: thalidomide, lenalidomide, pomalidomide, iberdomide, CC-92480, CC-99282, CC-91633, and CC-90009.
24. The method of any of embodiments 1-23, wherein the subsequent therapy is selected from among the group consisting of: thalidomide, lenalidomide, pomalidomide, iberdomide, CC-92480, CC-99282, CC-91633, and CC-90009.
25. The method of any of embodiments 1-19, wherein the class of therapy is proteasome inhibitors.
26. The method of any of embodiments 1-19 and 25, wherein the prior therapy is selected from among the group consisting of: bortezomib, carfilzomib and ixazomib.
27. The method of any of embodiments 1-19, 25, and 26, wherein the subsequent therapy is selected from among the group consisting of: bortezomib, carfilzomib and ixazomib.
28. The method of any of embodiments 1-19, wherein the class of therapy is anti-CD38 antibodies.
29. The method of any of embodiments 1-19 and 28, wherein the prior therapy is daratumumab or isatuximab.
30. The method of any of embodiments 1-19, 28, and 29, wherein the subsequent therapy is daratumumab or isatuximab.
31. The method of any of embodiments 1-17, wherein the cancer is a leukemia or a lymphoma.
32. The method of embodiment 31, wherein the leukemia or the lymphoma is selected from the group consisting of: acute lymphoblastic leukemia (ALL), adult ALL, chronic lymphoblastic leukemia (CLL), small lymphocytic lymphoma (SLL), non-Hodgkin lymphoma (NHL), and large B cell lymphoma (LBCL).
33 The method of any of embodiments 1-19, 31, and 32, wherein the class of therapy is inhibitors of Bruton's tyrosine kinase (BTK).
34. The method of any of embodiments 1-19 and 31-33, wherein the prior therapy is selected from among the group consisting of: ibrutinib, acalabrutinib, zanubrutinib, evobrutinib, tirabrutinib, and SNS-062.
35. The method of any of embodiments 1-19 and 31-34, wherein the subsequent therapy is selected from among the group consisting of: ibrutinib, acalabrutinib, zanubrutinib, evobrutinib, tirabrutinib, and SNS-062.
36. The method of any of embodiments 1-19, 31, and 32, wherein the class of therapy is inhibitors of BCL-2.
37. The method of any of embodiments 1-19, 31, 32, and 36, wherein the prior therapy is selected from among the group consisting of: venetoclax, navitoclax, ABT737, maritoclax, obatoclax, and clitocine.
38. The method of any of embodiments 1-19, 31, 32, 36, and 37, wherein the subsequent therapy is selected from among the group consisting of: venetoclax, navitoclax, ABT737, maritoclax, obatoclax, and clitocine.
39. The method of any of embodiments 1-38, wherein the subsequent therapy is a maintenance therapy.
40. The method of any of embodiments 1-39, wherein the T cell therapy comprises a dose of T cells expressing a recombinant receptor.
41. The method of embodiment 40, wherein the recombinant receptor is a T cell receptor (TCR) or a chimeric antigen receptor (CAR).
42. The method of embodiment 40 or embodiment 41, wherein the recombinant receptor is a CAR.
43. The method of embodiment 42, wherein the CAR comprises an extracellular antigen binding domain that binds to the antigen, a transmembrane domain, and an intracellular signaling region.
44. The method of embodiment 43, wherein the intracellular signaling region comprises an intracellular signaling domain of a CD3-zeta (CD3ζ) chain and a costimulatory signaling region.
45. The method of embodiment 44, wherein the costimulatory signaling region comprises an intracellular signaling domain of CD28, 4-1BB, or ICOS.
46. The method of embodiment 44 or embodiment 45, wherein the costimulatory signaling region comprises an intracellular signaling domain of 4-1BB.
47. The method of any one of embodiments 43-46, wherein the transmembrane domain is or comprises a transmembrane domain from CD28 or CD8, optionally human CD28 or CD8.
48. The method of any one of embodiments 43-47, wherein the CAR further comprises an extracellular spacer between the extracellular antigen binding domain and the transmembrane domain.
49. The method of embodiment 48, wherein the spacer is from CD8, optionally wherein the spacer is a CD8α hinge.
50. The method of embodiment 48 or embodiment 49, wherein the transmembrane domain and the spacer are from CD8.
51. The method of any of embodiments 43-50, wherein the extracellular antigen binding domain binds to B cell maturation antigen (BCMA).
52. The method of any of embodiments 43-51, wherein the extracellular antigen-binding domain comprises a variable heavy chain (V_{H}) region and, optionally, a variable light chain (V_{L}) region.
53. The method of embodiment 52, wherein:
the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively; or
the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively.
54. The method of embodiment 52 or embodiment 53, wherein:
the V_{H} region comprises an amino acid sequence set forth in SEQ ID NO: 18 and the V_{L} region comprises the amino acid sequence set forth in SEQ ID NO: 19;
or the V_{H} region comprises an amino acid sequence set forth in SEQ ID NO: 24, and the V_{L} region comprises the amino acid sequence set forth in SEQ ID NO: 25.
55. The method of any one of embodiments 43-54, wherein the extracellular antigen-binding domain is a single chain variable fragment (scFv).
56. The method of embodiment 55, wherein the scFv comprises the amino acid sequence set forth in SEQ ID NO: 213 or SEQ ID NO: 188.
57. The method of any one of embodiments 42-56, wherein the CAR comprises the amino acid sequence set forth in SEQ ID NO: 116 or SEQ ID NO: 124.
58. The method of any one of embodiments 42-57, wherein the CAR is encoded by the polynucleotide sequence set forth in SEQ ID NO: 214.
59. The method of any one of embodiments 40-58, wherein the dose of T cells comprises: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells.
60. The method of any one of embodiments 40-59, wherein the dose of T cells comprises idecabtagene vicleucel cells.
61. The method of any of embodiments 43-50, wherein the extracellular antigen binding domain binds to G protein-coupled receptor, class C group 5 member D (GPRCSD).
62. The method of any of embodiments 43-50, wherein the extracellular antigen binding domain binds to CD19.
63. The method of embodiment 62, wherein the extracellular antigen-binding domain comprises a variable heavy chain (V_{H}) region and, optionally, a variable light chain (V_{L}) region.
64. The method of embodiment 63, wherein:
the V_{H} region comprises a CDR-H1, a CDR-H2, and a CDR-H3 comprising the amino acid sequences set forth in SEQ ID NOS: 251, 252, and 253, respectively; and the V_{L} region comprises a CDR-L1, a CDR-L2, and a CDR-L3 comprising the amino acid sequences set forth in SEQ ID NOS: 248, 249, and 250, respectively; or
the V_{H} region comprises a CDR-H1, a CDR-H2, and a CDR-H3 comprising the amino acid sequences set forth in SEQ ID NOS: 260, 261, and 262, respectively; and the V_{L} region comprises a CDR-L1, a CDR-L2, and a CDR-L3 comprising the amino acid sequences set forth in SEQ ID NOS: 257, 258, and 259, respectively.
65. The method of embodiment 63 or embodiment 64, wherein:
the V_{H} region comprises an amino acid sequence set forth in SEQ ID NO: 254 and the V_{L} region comprises the amino acid sequence set forth in SEQ ID NO: 255; or
the V_{H} region comprises an amino acid sequence set forth in SEQ ID NO: 263 and the V_{L} region comprises the amino acid sequence set forth in SEQ ID NO: 264.
66. The method of any one of embodiments 62-65, wherein the extracellular antigen-binding domain is a single chain variable fragment (scFv).
67. The method of embodiment 66, wherein the scFv comprises the amino acid sequence set forth in SEQ ID NO: 256 or SEQ ID NO: 265.
68. The method of any one of embodiments 40-50 and 62-67, wherein the dose of T cells comprises: lisocabtagene maraleucel cells; tisagenlecleucel cells; axicabtagene ciloleucel cells; or brexucabtagene autoleucel cells.
69. The method of any of embodiments 40-68, wherein the dose of T cells comprises CD3⁺ CAR-expressing T cells.
70. The method of any of embodiments 40-69, wherein the dose of T cells comprises a combination of CD4⁺ CAR-expressing T cells and CD8⁺ CAR-expressing T cells.
71. The method of embodiment 70, wherein the ratio of CD4⁺ CAR-expressing T cells to CD8⁺ CAR-expressing T cells in the dose of T cells is approximately 1:1 or is between approximately 1:3 and approximately 3:1.
72. The method of any of embodiments 40-71, wherein, in the dose of T cells:
the percentage of naive-like T cells and/or central memory T cells is greater than or greater than about 60% of the total T cells in the dose, optionally greater than or greater than about 65%, 70%, 80%, 90% or 95%;
the percentage of naive-like T cells and/or central memory T cells is greater than or greater than about 40% of the total CD4+ T cells in the dose, optionally greater than or greater than about 50%, 60%, 70%, 80%, 90% or 95%; or
the percentage of naive-like T cells and/or central memory T cells is greater than or greater than about 40% of the total CD8+ T cells in the dose, optionally greater than or greater than about 50%, 60%, 70%, 80%, 90% or 95%.
73. The method of embodiment 72, wherein the naive-like T cells are CCR7+CD45RA+, CD27+CCR7+, or CD62L−CCR7+.
74. The method of any one of embodiments 40-72, wherein the dose of T cells comprises between about 0.5×10⁶ and about 6×10⁸ CAR-positive T cells.
75. The method of any one of embodiments 40-74, wherein the dose of T cells comprises between about 1×10⁸ and about 6×10⁸ CAR-positive T cells.
76. The method of any one of embodiments 40-75, wherein the dose of T cells comprises between about 1.5×10⁸ and about 4.5×10⁸ CAR-positive T cells.
77. The method of any one of embodiments 40-76, wherein the dose of T cells comprises about 1.5×10⁸, 3×10⁸, or about 4.5×10⁸ CAR-positive T cells.
78. The method of any one of embodiments 40-74, wherein the dose of T cells comprises between about 0.5×10⁶ and about 10×10⁶ CAR-positive T cells.
79. The method of any one of embodiments 40-78, wherein the cells of the dose of T cells were obtained from the subject.
80. The method of any one of embodiments 40-79, wherein the cells of the dose of T cells are autologous to the subject.
81. The method of any one of embodiments 40-78, wherein the cells of the dose of T cells are allogeneic to the subject.
82. The method of any of embodiments 1-39, wherein the T cell therapy comprises a T cell engager (TCE).
83. The method of embodiment 82, wherein the TCE is selected from among the group consisting of: a bispecific T cell engager (BiTE), a checkpoint-inhibitory T cell engager (CiTE), a simultaneous multiple interaction T cell engagers (SMITE), and BiTE-expressing CAR T cells (CART.BiTE cells).
84. The method of any of embodiments 1-83, wherein the method comprises, prior to administration of the T cell therapy, administering a lymphodepleting therapy to the subject.
85. The method of embodiment 84, wherein the lymphodepleting therapy is completed between 2 and 7 days before the initiation of administration of the T cell therapy.
86. The method of embodiment 84 or embodiment 85, wherein the lymphodepleting therapy comprises the administration of fludarabine and/or cyclophosphamide.
87. The method of any of embodiments 84-86, wherein the lymphodepleting therapy comprises administration of:
(i) cyclophosphamide at about 200-400 mg/m², optionally at or about 300 mg/m², inclusive, and/or fludarabine at about 20-40 mg/m², optionally 30 mg/m², daily for 2-4 days, optionally for 3 days; or
(ii) cyclophosphamide at about 500 mg/m².
88. The method of any one of embodiments 84-87, wherein:
the lymphodepleting therapy comprises administration of cyclophosphamide at or about 300 mg/m² and fludarabine at about 30 mg/m² daily for 3 days; or
the lymphodepleting therapy comprises administration of cyclophosphamide at or about 500 mg/m² and fludarabine at about 30 mg/m² daily for 3 days.

### VI. Examples

The following examples are included for illustrative purposes only and are not intended to limit the scope of the invention.

### Example 1: Transcriptional and Genomic Features in Relapsed/Refractory Multiple Myeloma

Chimeric antigen-receptor (CAR)-expressing T cell compositions containing autologous T cells expressing a CAR specific for B-cell maturation antigen (BCMA) were administered to human subjects with relapsed and refractory (R/R) multiple myeloma (MM).

### A. Subjects and Treatment

Compositions containing autologous T cells engineered to express an exemplary anti-BCMA CAR were administered to adult human subjects with R/R MM who had received 3 or more prior lines of therapy (the three or more prior lines of therapy including an immunomodulatory agent, a proteasome inhibitor (PI), and an anti-CD38 antibody) and were disease refractory to the previous line of therapy per International Myeloma Working Group (IMWG) criteria.

The administered T cell compositions were generated by obtaining peripheral-blood mononuclear cells (PBMCs) from leukapheresis samples from individual subjects with R/R MM, stimulating the PBMCs with anti-CD3 and anti-CD28 antibodies, transducing the cells with a lentiviral vector containing the exemplary anti-BCMA CAR, and expanding the cells for 10 days prior to cryopreservation. The exemplary CAR contained an anti-BCMA scFv, a hinge and transmembrane domain from a CD8α, and a CD137 (4-1BB) co-stimulatory domain followed by the intracellular signaling domain of a CD3ζ chain. The polynucleotide sequence encoding the exemplary BCMA CAR is set forth in SEQ ID NO: 116, and the polypeptide sequence of the exemplary BCMA CAR is set forth in SEQ ID NO: 214.

Subjects received a lymphodepleting chemotherapy (LDC) with fludarabine (flu, 30 mg/m2/day) and cyclophosphamide (cy, 300 mg/m2/day) for 3 consecutive days, the LDC being completed 2 days before CAR T cell infusion. Bridging therapy was allowed during CAR T cell manufacturing, but was stopped at least 14 days before LDC. The cryopreserved cell compositions were thawed at bedside prior to intravenous administration, and subjects were administered a dose of 150 to 450×10⁶ (e.g., 150, 300, or 450×10⁶) total CAR-expressing T cells.

### B. High Risk Tumor Features

Bone marrow aspirates were collected from subjects prior to treatment (“pretreatment”; n=97 RNA, n=68 DNA) and at disease progression (“PD”; n=64 RNA, n=33 DNA). CD138+ fractions were obtained from the bone marrow aspirates. 3′ poly-A libraries were generated from the pretreatment and PD CD138+ sorted fractions and subjected to RNA sequencing (RNAseq) and whole genome sequencing (WGS). Known molecular high-risk/resistance (HR) genomic (biallelic p53 inactivation, high cancer clonal fraction del17p, HR t(4;14), and cereblon dysregulation [i.e., mutation, copy number loss, translocation, or high expression of CRBNdel10]) and transcriptomic (MDMS8 gene signature) features were analyzed, and correlations with overall response (OR) and progression-free survival (PFS) were evaluated.

Differential gene expression and principal component analysis (PCA) explored novel transcriptomic signatures and response. CD138+ cell purity was regressed out, and PCA was performed on the residual matrix. Multivariate regression analysis was performed on the top 10 PCs to evaluate association with PFS. P values, or false discovery rates (FDR) if appropriate, of <0.01 were considered statistically significant.

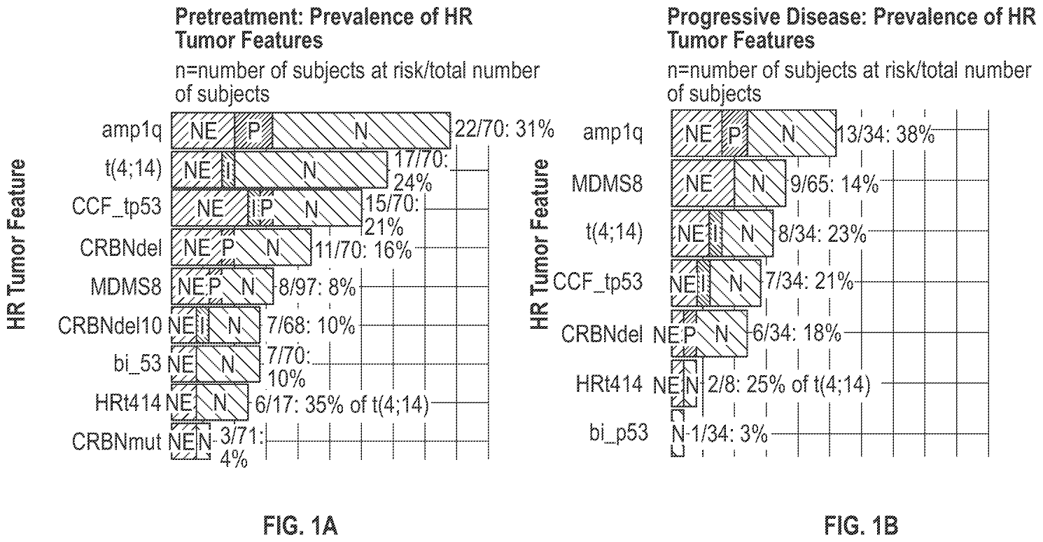

At least one molecular HR feature was identified in 44% (43/97) of subjects at pretreatment and in 48% (31/64) subjects at PD. Some tumors had multiple HR features at pretreatment, consistent with heterogeneity in the late-line R/R MM patient population. Gain and loss of HR tumor features, as well as changes in the number of HR tumor features per subject, was observed post-treatment across subjects. Cross-sectional analysis revealed increased prevalence of amp1q, MDMS8 gene signature, and CRBNdel HR tumor features at disease progression, and decreased prevalence of the other evaluated HR tumor features at disease progression (FIGS. 1A and 1B). 27% (17/62) of subjects exhibited fewer HR tumor features at PD relative to pretreatment, while 56% (35/62) of subjects exhibited no change in the frequency of HR tumor features at PD relative to pretreatment. Similar trends were observed in a subset of subjects achieving minimum residual disease (MRD) negative status (10-5) three months post-treatment.

While a similar proportion of subjects had HR tumor features at pretreatment and PD, paired sample analysis showed the evolution of the HR tumor feature landscape of tumors. Paired pretreatment and PD samples did not show dominant enrichment for one or more HR tumor features at disease progression. OR and PFS were similar in samples with and without each molecular HR tumor feature analyzed, suggesting the exemplary anti-BCMA CAR T cells had similar efficacy across the evaluated HR tumor features. These observations suggest that treatment with the exemplary anti-BCMA CAR T cells alters the heterogeneity of the HR tumor landscape without dominant pressure for increased heterogeneity in known HR tumor features at progression.

One principal component (PC4) was correlated with PFS (p=0.002). The top weighted genes in PC4 may be a novel signature associated with durable responses to treatment.

In summary, baseline multi-omic-based HR tumor features were not observed to correlate with OR or PFS, and a dominant selection for any one HR feature at PD was not noted. These data are consistent with a finding that activation and expansion of the administered anti-BCMA CAR T cells remain mediators of OR that may not be substantially influenced by tumor intrinsic features.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Sequences

| SEQ | | |
|---|---|---|
| ID | | |
| NO. | SEQUENCE | DESCRIPTION |
| 1 | ESKYGPPCPPCP | spacer |
| | | (IgG4hinge)(aa) |
| | | Homo sapiens |
| 2 | GAATCTAAGTACGGACCGCCCTGCCCCCCTTGCCCT | spacer |
| | | (IgG4hinge)(nt) |
| | | Homo sapiens |
| 3 | ESKYGPPCPPCPGQPREPQVYTLPPSQEEMTKNQVSLTCLVKGFYPSDIA | Hinge-CH3 |
| | VEWESNGQPENNYKTTPPVLDSDGSFFLYSRLTVDKSRWQEGNVFSCSVM | spacer |
| | HEALHNHYTQKSLSLSLGK | Homo sapiens |
| 4 | ESKYGPPCPPCPAPEFLGGPSVFLFPPKPKDTLMISRTPEVTCVVVDVSQ | Hinge-CH2-CH3 |
| | EDPEVQFNWYVDGVEVHNAKTKPREEQFNSTYRVVSVLTVLHQDWLNGKE | spacer |
| | YKCKVSNKGLPSSIEKTISKAKGQPREPQVYTLPPSQEEMTKNQVSLTCL | Homo sapiens |
| | VKGFYPSDIAVEWESNGQPENNYKTTPPVLDSDGSFFLYSRLTVDKSRWQ | |
| | EGNVFSCSVMHEALHNHYTQKSLSLSLGK | |
| 5 | RWPESPKAQASSVPTAQPQAEGSLAKATTAPATTRNTGRGGEEKKKEKEK | IgD-hinge-Fc |
| | EEQEERETKTPECPSHTQPLGVYLLTPAVQDLWLRDKATFTCFVVGSDLK | Homo sapiens |
| | DAHLTWEVAGKVPTGGVEEGLLERHSNGSQSQHSRLTLPRSLWNAGTSVT | |
| | CTLNHPSLPPQRLMALREPAAQAPVKLSLNLLASSDPPEAASWLLCEVSG | |
| | FSPPNILLMWLEDQREVNTSGFAPARPPPQPGSTTFWAWSVLRVPAPPSP | |
| | QPATYTCVVSHEDSRTLLNASRSLEVSYVTDH | |
| 6 | LEGGGEGRGSLLTCGDVEENPGPR | T2A |
| | | artificial |
| 7 | RKVCNGIGIGEFKDSLSINATNIKHFKNCTSISGDLHILPVAFRGDSFTH | tEGFR |
| | TPPLDPQELDILKTVKEITGFLLIQAWPENRTDLHAFENLEIIRGRTKQH | artificial |
| | GQFSLAVVSLNITSLGLRSLKEISDGDVIISGNKNLCYANTINWKKLFGT | |
| | SGQKTKIISNRGENSCKATGQVCHALCSPEGCWGPEPRDCVSCRNVSRGR | |
| | ECVDKCNLLEGEPREFVENSECIQCHPECLPQAMNITCTGRGPDNCIQCA | |
| | HYIDGPHCVKTCPAGVMGENNTLVWKYADAGHVCHLCHPNCTYGCTGPGL | |
| | EGCPTNGPKIPSIATGMVGALLLLLVVALGIGLFM | |
| 8 | FWVLVVVGGVLACYSLLVTVAFIIFWV | CD28 (amino |
| | | acids 153-179 of |
| | | Accession No. |
| | | P10747) |
| | | Homo sapiens |
| 9 | IEVMYPPPYLDNEKSNGTIIHVKGKHLCPSPLFPGPSKPFWVLVVVGGVL | CD28 (amino |
| | ACYSLLVTVAFIIFWV | acids 114-179 of |
| | | Accession No. |
| | | P10747) |
| | | Homo sapiens |
| 10 | RSKRSRLLHSDYMNMTPRRPGPTRKHYQPYAPPRDFAAYRS | CD28 (amino |
| | | acids 180-220 of |
| | | P10747) |
| | | Homo sapiens |
| 11 | RSKRSRGGHSDYMNMTPRRPGPTRKHYQPYAPPRDFAAYRS | CD28 (LL to |
| | | GG) |
| | | Homo sapiens |
| 12 | KRGRKKLLYIFKQPFMRPVQTTQEEDGCSCRFPEEEEGGCEL | 4-1BB (amino |
| | | acids 214-255 of |
| | | Q07011.1) |
| | | Homo sapiens |
| 13 | RVKFSRSADAPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | Homo sapiens |
| | YDALHMQALPPR | |
| 14 | RVKFSRSAEPPAYQQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | Homo sapiens |
| | YDALHMQALPPR | |
| 15 | RVKFSRSADAPAYKQGQNQLYNELNLGRREEYDVLDKRRGRDPEMGGKPR | CD3 zeta |
| | RKNPQEGLYNELQKDKMAEAYSEIGMKGERRRGKGHDGLYQGLSTATKDT | Homo sapiens |
| | YDALHMQALPPR | |
| 16 | PGGG-(SGGGG)5-P- wherein P is proline, G is | linker |
| | glycine and S is serine | |
| 17 | GSADDAKKDAAKKDGKS | Linker |
| 18 | QIQLVQSGPELKKPGETVKISCKASGYTFTDYSINWVKRAPGKGLKWMGW | Variable heavy |
| | INTETREPAYAYDFRGRFAFSLETSASTAYLQINNLKYEDTATYFCALDY | (VH) Anti- |
| | SYAMDYWGQGTSVTVSS | BCMA |
| 19 | DIVLTQSPPSLAMSLGKRATISCRASESVTILGSHLIHWYQQKPGQPPTL | Variable light |
| | LIQLASNVQTGVPARFSGSGSRTDFTLTIDPVEEDDVAVYYCLQSRTIPR | (VL) Anti- |
| | TFGGGTKLEIK | BCMA |
| 20 | QIQLVQSGPDLKKPGETVKLSCKASGYTFTNFGMNWVKQAPGKGFKWMAW | Variable heavy |
| | INTYTGESYFADDFKGRFAFSVETSATTAYLQINNLKTEDTATYFCARGE | (VH) Anti- |
| | IYYGYDGGFAYWGQGTLVTVSA | BCMA |
| 21 | DVVMTQSHRFMSTSVGDRVSITCRASQDVNTAVSWYQQKPGQSPKLLIFS | Variable light |
| | ASYRYTGVPDRFTGSGSGADFTLTISSVQAEDLAVYYCQQHYSTPWTFGG | (VL) Anti- |
| | GTKLDIK | BCMA |
| 22 | EVQLVQSGAEVKKPGESLKISCKGSGYSFTSYWIGWVRQMPGKGLEWMGI | Variable heavy |
| | IYPGDSDTRYSPSFQGHVTISADKSISTAYLQWSSLKASDTAMYYCARYS | (VH) Anti- |
| | GSFDNWGQGTLVTVSS | BCMA |
| 23 | SYELTQPPSASGTPGQRVTMSCSGTSSNIGSHSVNWYQQLPGTAPKLLIY | Variable light |
| | TNNQRPSGVPDRFSGSKSGTSASLAISGLQSEDEADYYCAAWDGSLNGLV | (VL) Anti- |
| | FGGGTKLTVLG | BCMA |
| 24 | EVQLVQSGAEMKKPGASLKLSCKASGYTFIDYYVYWMRQAPGQGLESMGW | Variable heavy |
| | INPNSGGTNYAQKFQGRVTMTRDTSISTAYMELSRLRSDDTAMYYCARSQ | (VH) Anti- |
| | RDGYMDYWGQGTLVTVSS | BCMA |
| 25 | QSALTQPASVSASPGQSIAISCTGTSSDVGWYQQHPGKAPKLMIYEDSKR | Variable light |
| | PSGVSNRFSGSKSGNTASLTISGLQAEDEADYYCSSNTRSSTLVFGGGTK | (VL) Anti- |
| | LTVLG | BCMA |
| 26 | GGGGS | Linker |
| 27 | GGGS | Linker |
| 28 | GGGGSGGGGSGGGGS | Linker |
| 29 | GSTSGSGKPGSGEGSTKG | Linker |
| 30 | SRGGGGSGGGGSGGGGSLEMA | Linker |
| 31 | ESKYGPPCPPCPAPPVAGPSVFLFPPKPKDTLMISRTPEVTCVVVDVSQE | Hinge-CH2-CH3 |
| | DPEVQFNWYVDGVEVHNAKTKPREEQFQSTYRVVSVLTVLHQDWLNGKEY | spacer |
| | KCKVSNKGLPSSIEKTISKAKGQPREPQVYTLPPSQEEMTKNQVSLTCLV | Homo sapiens |
| | KGFYPSDIAVEWESNGQPENNYKTTPPVLDSDGSFFLYSRLTVDKSRWQE | |
| | GNVFSCSVMHEALHNHYTQKSLSLSLGK | |
| 32 | EVQLVQSGAEVKKPGSSVKVSCKASGGTFSSYAISWVRQAPGQGLEWMGR | Variable heavy |
| | IIPILGIANYAQKFQGRVTMTEDTSTDTAYMELSSLRSEDTAVYYCARSG | (VH) Anti- |
| | YSKSIVSYMDYWGQGTLVTVSS | BCMA |
| 33 | LPVLTQPPSTSGTPGQRVTVSCSGSSSNIGSNVVFWYQQLPGTAPKLVIY | Variable light |
| | RNNQRPSGVPDRFSVSKSGTSASLAISGLRSEDEADYYCAAWDDSLSGYV | (VL) Anti- |
| | FGTGTKVTVLG | BCMA |
| 34 | QVQLVQSGAEVKKPGSSVKVSCKASGGTFSSYAISWVRQAPGQGLEWMGR | Variable heavy |
| | IIPILGTANYAQKFQGRVTITADESTSTAYMELSSLRSEDTAVYYCARSG | (VH) Anti- |
| | YGSYRWEDSWGQGTLVTVSS | BCMA |
| 35 | QAVLTQPPSASGTPGQRVTISCSGSSSNIGSNYVFWYQQLPGTAPKLLIY | Variable light |
| | SNNQRPSGVPDRFSGSKSGTSASLAISGLRSEDEADYYCAAWDDSLSASY | (VL) Anti- |
| | VFGTGTKVTVLG | BCMA |
| 36 | QVQLVQSGAEVKKPGASVKVSCKASGYTFTDYYMHWVRQAPGQRLEWMGW | Variable heavy |
| | INPNSGGTNYAQKFQDRITVTRDTSSNTGYMELTRLRSDDTAVYYCARSP | (VH) Anti- |
| | YSGVLDKWGQGTLVTVSS | BCMA |
| 37 | QSVLTQPPSVSGAPGQRVTISCTGSSSNIGAGFDVHWYQQLPGTAPKLLI | Variable light |
| | YGNSNRPSGVPDRFSGSKSGTSASLAITGLQAEDEADYYCQSYDSSLSGY | (VL) Anti- |
| | VFGTGTKVTVLG | BCMA |
| 38 | ASGGGGSGGRASGGGGS | Linker |
| 39 | MALPVTALLLPLALLLHAARP | CD8a signal |
| | | peptide |
| 40 | METDTLLLWVLLLWVPGSTG | signal peptide |
| 41 | EVQLLESGGGLVQPGGSLRLSCAASGFTFSSYAMSWVRQAPGKGLEWVSA | Variable heavy |
| | ISGSGGSTYYADSVKGRFTISRDNSKNTLYLQMNSLRAEDTAVYYCARAE | (VH) Anti- |
| | MGAVFDIWGQGTMVTVSS | BCMA |
| 42 | EIVLTQSPATLSLSPGERATLSCRASQSVSRYLAWYQQKPGQAPRLLIYD | Variable light |
| | ASNRATGIPARFSGSGSGTDFTLTISSLEPEDFAVYYCQQRISWPFTFGG | (VL) Anti- |
| | GTKVEIK | BCMA |
| 43 | QVQLVESGGGVVQPGRSLRLSCAASGFTFSSYGMHWVRQAPGKGLEWVAV | Variable heavy |
| | ISYDGSNKYYADSVKGRFTISRDNSKNTLYLQMNSLRAEDTAVYYCARDG | (VH) Anti- |
| | TYLGGLWYFDLWGRGTLVTVSS | BCMA |
| 44 | DIVMTQSPLSLPVTPGEPASISCRSSQSLLHSNGYNYLDWYLQKPGQSPQ | Variable light |
| | LLIYLGSNRASGVPDRFSGSGSGTDFTLKISRVEAEDVGVYYCMQGLGLP | (VL) Anti- |
| | LTFGGGTKVEIK | BCMA |
| 45 | QVQLVQSGAEVKKPGASVKVSCKASGYTFTSYYMHWVRQAPGOGLEWMGI | Variable heavy |
| | INPGGGSTSYAQKFQGRVTMTRDTSTSTVYMELSSLRSEDTAVYYCARES | (VH) Anti- |
| | WPMDVWGQGTTVTVSS | BCMA |
| 46 | EIVMTQSPATLSVSPGERATLSCRASQSVSSNLAWYQQKPGQAPRLLIYG | Variable light |
| | ASTRATGIPARFSGSGSGTEFTLTISSLQSEDFAVYYCQQYAAYPTFGGG | (VL) Anti- |
| | TKVEIK | BCMA |
| 47 | QLQLQESGPGLVKPSETLSLTCTVSGGSISSSSYYWGWIRQPPGKGLEWI | Variable heavy |
| | GSISYSGSTYYNPSLKSRVTISVDTSKNQFSLKLSSVTAADTAVYYCARG | (VH) Anti- |
| | RGYATSLAFDIWGQGTMVTVSS | BCMA |
| 48 | EIVLTQSPATLSLSPGERATLSCRASQSVSSYLAWYQQKPGQAPRLLIYD | Variable light |
| | ASNRATGIPARFSGSGSGTDFTLTISSLEPEDFAVYYCQQRHVWPPTFGG | (VL) Anti- |
| | GTKVEIK | BCMA |
| 49 | EVQLVESGGGLVQPGGSLRLSCAASGFTFSSYSMNWVRQAPGKGLEWVST | Variable heavy |
| | ISSSSSTIYYADSVKGRFTISRDNAKNSLYLQMNSLRAEDTAVYYCARGS | (VH) Anti- |
| | QEHLIFDYWGQGTLVTVSS | BCMA |
| 50 | EIVLTQSPATLSLSPGERATLSCRASQSVSRYLAWYQQKPGQAPRLLIYD | Variable light |
| | ASNRATGIPARFSGSGSGTDFTLTISSLEPEDFAVYYCQQRFYYPWTFGG | (VL) Anti- |
| | GTKVEIK | BCMA |
| 51 | QVQLVESGGGVVQPGRSLRLSCAASGFTFSSYGMHWVRQAPGKGLEWVAV | Variable heavy |
| | ISYDGSNKYYADSVKGRFTISRDNSKNTLYLQMNSLRAEDTAVYYCARTD | (VH) Anti- |
| | FWSGSPPGLDYWGQGTLVTVSS | BCMA |
| 52 | DIQLTQSPSSVSASVGDRVTITCRASQGISSWLAWYQQKPGKAPKLLIYG | Variable light |
| | ASSLQSGVPSRFSGSGSGTDFTLTISSLQPEDFATYYCQQIYTFPFTFGG | (VL) Anti- |
| | GTKVEIK | BCMA |
| 53 | QVQLVQSGAEVKKPGSSVKVSCKASGGTFSSYAISWVRQAPGQGLEWMGG | Variable heavy |
| | IIPIFGTANYAQKFQGRVTITADESTSTAYMELSSLRSEDTAVYYCARTP | (VH) Anti- |
| | EYSSSIWHYYYGMDVWGQGTTVTVSS | BCMA |
| 54 | DIVMTQSPDSLAVSLGERATINCKSSQSVLYSSNNKNYLAWYQQKPGQPP | Variable light |
| | KLLIYWASTRESGVPDRFSGSGSGTDFTLTISSLQAEDVAVYYCQQFAHT | (VL) Anti- |
| | PFTFGGGTKVEIK | BCMA |
| 55 | QVQLVESGGGVVQPGRSLRLSCAASGFTFSSYGMHWVRQAPGKGLEWVAV | Variable heavy |
| | ISYDGSNKYYADSVKGRFTISRDNSKNTLYLQMNSLRAEDTAVYYCVKGP | (VH) Anti- |
| | LQEPPYDYGMDVWGQGTTVTVSS | BCMA |
| 56 | EIVMTQSPATLSVSPGERATLSCRASQSVSSNLAWYQQKPGQAPRLLIYS | Variable light |
| | ASTRATGIPARFSGSGSGTEFTLTISSLQSEDFAVYYCQQHHVWPLTFGG | (VL) Anti- |
| | GTKVEIK | BCMA |
| 57 | QVQLVQSGAEVKKPGSSVKVSCKASGGTFSSYAISWVRQAPGQGLEWMGR | Variable heavy |
| | IIPILGIANYAQKFQGRVTITADKSTSTAYMELSSLRSEDTAVYYCARGG | (VH) Anti- |
| | YYSHDMWSEDWGQGTLVTVSS | BCMA |
| 58 | LPVLTQPPSASGTPGQRVTISCSGRSSNIGSNSVNWYRQLPGAAPKLLIY | Variable light |
| | SNNQRPPGVPVRFSGSKSGTSASLAISGLQSEDEATYYCATWDDNLNVHY | (VL) Anti- |
| | VFGTGTKVTVLG | BCMA |
| 59 | QVQLVQSGSELKKPGASVKVSCKASGYTFTDYSINWVRQAPGQGLEWMGW | Variable heavy |
| | INTETREPAYAYDFRGRFVFSLDTSVSTAYLQISSLKAEDTAVYYCARDY | (VH) Anti- |
| | SYAMDYWGQGTLVTVSS | BCMA |
| 60 | DIVLTQSPASLAVSLGERATINCRASESVSVIGAHLIHWYQQKPGQPPKL | Variable light |
| | LIYLASNLETGVPARFSGSGSGTDFTLTISSLQAEDAAIYYCLQSRIFPR | (VL) Anti- |
| | TFGQGTKLEIK | BCMA |
| 61 | EVQLVESGGGLVQPGGSLRLSCAVSGFALSNHGMSWVRRAPGKGLEWVSG | Variable heavy |
| | IVYSGSTYYAASVKGRFTISRDNSRNTLYLQMNSLRPEDTAIYYCSAHGG | (VH) Anti- |
| | ESDVWGQGTTVTVSS | BCMA |
| 62 | DIQLTQSPSSLSASVGDRVTITCRASQSISSYLNWYQQKPGKAPKLLIYA | Variable light |
| | ASSLQSGVPSRFSGSGSGTDFTLTISSLQPEDFATYYCQQSYSTPYTFGQ | (VL) Anti- |
| | GTKVEIK | BCMA |
| 63 | QVQLVESGGGLVQPGRSLRLSCAASGFTFSNYAMSWVRQAPGKGLGWVSG | Variable heavy |
| | ISRSGENTYYADSVKGRFTISRDNSKNTLYLQMNSLRDEDTAVYYCARSP | (VH) Anti- |
| | AHYYGGMDVWGQGTTVTVSS | BCMA |
| 64 | DIVLTQSPGTLSLSPGERATLSCRASQSISSSFLAWYQQKPGQAPRLLIY | Variable light |
| | GASRRATGIPDRFSGSGSGTDFTLTISRLEPEDSAVYYCQQYHSSPSWTF | (VL) Anti- |
| | GQGTKLEIK | BCMA |
| 65 | QVQLVESGGGLVQPGGSLRLSCAVSGFALSNHGMSWVRRAPGKGLEWVSG | Variable heavy |
| | IVYSGSTYYAASVKGRFTISRDNSRNTLYLQMNSLRPEDTAIYYCSAHGG | (VH) Anti- |
| | ESDVWGQGTTVTVSS | BCMA |
| 66 | DIRLTQSPSPLSASVGDRVTITCQASEDINKFLNWYHQTPGKAPKLLIYD | Variable light |
| | ASTLQTGVPSRFSGSGSGTDFTLTINSLQPEDIGTYYCQQYESLPLTFGG | (VL) Anti- |
| | GTKVEIK | BCMA |
| 67 | EVQLVESGGGLVQPGGSLRLSCAVSGFALSNHGMSWVRRAPGKGLEWVSG | Variable heavy |
| | IVYSGSTYYAASVKGRFTISRDNSRNTLYLQMNSLRPEDTAIYYCSAHGG | (VH) Anti- |
| | ESDVWGQGTTVTVSS | BCMA |
| 68 | EIVLTQSPGTLSLSPGERATLSCRASQSIGSSSLAWYQQKPGQAPRLLMY | Variable light |
| | GASSRASGIPDRFSGSGSGTDFTLTISRLEPEDFAVYYCQQYAGSPPFTF | (VL) Anti- |
| | GQGTKVEIK | BCMA |
| 69 | QIQLVQSGPELKKPGETVKISCKASGYTFRHYSMNWVKQAPGKGLKWMGR | Variable heavy |
| | INTESGVPIYADDFKGRFAFSVETSASTAYLVINNLKDEDTASYFCSNDY | (VH) Anti- |
| | LYSLDFWGQGTALTVSS | BCMA |
| 70 | DIVLTQSPPSLAMSLGKRATISCRASESVTILGSHLIYWYQQKPGQPPTL | Variable light |
| | LIQLASNVQTGVPARFSGSGSRTDFTLTIDPVEEDDVAVYYCLQSRTIPR | (VL) Anti- |
| | TFGGGTKLEIK | BCMA |
| 71 | QIQLVQSGPELKKPGETVKISCKASGYTFTHYSMNWVKQAPGKGLKWMGR | Variable heavy |
| | INTETGEPLYADDFKGRFAFSLETSASTAYLVINNLKNEDTATFFCSNDY | (VH) Anti- |
| | LYSCDYWGQGTTLTVSS | BCMA |
| 72 | DIVLTQSPASLAMSLGKRATISCRASESVSVIGAHLIHWYQQKPGQPPKL | Variable light |
| | LIYLASNLETGVPARFSGSGSGTDFTLTIDPVEEDDVAIYSCLQSRIFPR | (VL) Anti- |
| | TFGGGTKLEIK | BCMA |
| 73 | QVQLVQSGAEVKKPGASVKVSCKASGYSFPDYYINWVRQAPGQGLEWMGW | Variable heavy |
| | IYFASGNSEYNQKFTGRVTMTRDTSINTAYMELSSLTSEDTAVYFCASLY | (VH) Anti- |
| | DYDWYFDVWGQGTMVTVSS | BCMA |
| 74 | DIVMTQTPLSLSVTPGQPASISCKSSQSLVHSNGNTYLHWYLQKPGQSPQ | Variable light |
| | LLIYKVSNRFSGVPDRFSGSGSGTDFTLKISRVEAEDVGIYYCSQSSIYP | (VL) Anti- |
| | WTFGQGTKLEIK | BCMA |
| 75 | QVQLVQSGAEVKKPGASVKVSCKASGYSFPDYYINWVRQAPGQGLEWMGW | Variable heavy |
| | IYFASGNSEYNQKFTGRVTMTRDTSSSTAYMELSSLRSEDTAVYFCASLY | (VH) Anti- |
| | DYDWYFDVWGQGTMVTVSS | BCMA |
| 76 | DIVMTQTPLSLSVTPGEPASISCKSSQSLVHSNGNTYLHWYLQKPGQSPQ | Variable light |
| | LLIYKVSNRFSGVPDRFSGSGSGADFTLKISRVEAEDVGVYYCAETSHVP | (VL) Anti- |
| | WTFGQGTKLEIK | BCMA |
| 77 | QVQLVESGGGLVQPGGSLRLSCEASGFTLDYYAIGWFRQAPGKEREGVIC | Anti-BCMA |
| | ISRSDGSTYYADSVKGRFTISRDNAKKTVYLQMISLKPEDTAAYYCAAGA | sdAb |
| | DCSGYLRDYEFRGQGTQVTVSS | |
| 78 | IEVMYPPPYLDNEKSNGTIIHVKGKHLCPSPLFPGPSKP | CD28 spacer |
| 79 | IYIWAPLAGTCGVLLLSLVITLYCN | CD8a TM |
| 80 | LDNEKSNGTIIHVKGKHLCPSPLFPGPSKP | CD28 spacer |
| | | (truncated) |
| 81 | PTTTPAPRPPTPAPTIASQPLSLRPEACRPAAGGAVHTRGLDFACD | CD8a hinge |
| 82 | TTTPAPRPPTPAPTIASQPLSLRPEACRPAAGGAVHTRGLDFACD | CD8a hinge |
| 83 | FVPVFLPAKPTTTPAPRPPTPAPTIASQPLSLRPEACRPAAGGAVHTRGL | CD8a hinge |
| | DFACD | |
| 84 | DTGLYICKVELMYPPPYYLGIGNGTQIYVIDPEPCPDSD | CTLA4 hinge |
| 85 | FLLWILAAVSSGLFFYSFLLTAVS | CTLA4 TM |
| 86 | QIKESLRAELRVTERRAEVPTAHPSPSPRPAGQFQTLV | PD-1 hinge |
| 87 | VGVVGGLLGSLVLLVWVLAVI | PD-1 TM |
| 88 | GLAVSTISSFFPPGYQ | Fc(gamma)RIIIa |
| | | hinge |
| 89 | EPKSPDKTHTCPPCPAPPVAGPSVFLFPPKPKDTLMIARTPEVTCVVVDV | IgG1 hinge |
| | SHEDPEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSLT | |
| | CLVKGFYPSDIAVEWESNGQPENNYKTTPPVLDSDGSFFLYSKLTVDKSR | |
| | WQQGNVFSCSVMHEALHNHYTQKSLSLSPGK | |
| 90 | EVQLLESGGGLVQPGGSLRLSCAASGFTFSSYAMSWVRQAPGKGLEWVSA | anti-BCMA CAR |
| | ISGSGGSTYYADSVKGRFTISRDNSKNTLYLQMNSLRAEDTAVYYCARAE | |
| | MGAVFDIWGQGTMVTVSSGSTSGSGKPGSGEGSTKGEIVLTQSPATLSLS | |
| | PGERATLSCRASQSVSRYLAWYQQKPGQAPRLLIYDASNRATGIPARFSG | |
| | SGSGTDFTLTISSLEPEDFAVYYCQQRISWPFTFGGGTKVEIKRAAALDN | |
| | EKSNGTIIHVKGKHLCPSPLFPGPSKPFWVLVVVGGVLACYSLLVTVAFI | |
| | IFWVRSKRSRLLHSDYMNMTPRRPGPTRKHYQPYAPPRDFAAYRSRVKFS | |
| | RSADAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYDALH | |
| | MQALPPR | |
| 91 | EIVLTQSPATLSLSPGERATLSCRASQSVSRYLAWYQQKPGQAPRLLIYD | anti-BCMA CAR |
| | ASNRATGIPARFSGSGSGTDFTLTISSLEPEDFAVYYCQQRISWPFTFGG | |
| | GTKVEIKRGSTSGSGKPGSGEGSTKGEVQLLESGGGLVQPGGSLRLSCAA | |
| | SGFTFSSYAMSWVRQAPGKGLEWVSAISGSGGSTYYADSVKGRFTISRDN | |
| | SKNTLYLQMNSLRAEDTAVYYCARAEMGAVFDIWGQGTMVTVSSAAALDN | |
| | EKSNGTIIHVKGKHLCPSPLFPGPSKPFWVLVVVGGVLACYSLLVTVAFI | |
| | IFWVRSKRSRLLHSDYMNMTPRRPGPTRKHYQPYAPPRDFAAYRSRVKFS | |
| | RSADAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYDALH | |
| | MQALPPR | |
| 92 | QVQLVESGGGVVQPGRSLRLSCAASGFTFSSYGMHWVRQAPGKGLEWVAV | anti-BCMA CAR |
| | ISYDGSNKYYADSVKGRFTISRDNSKNTLYLQMNSLRAEDTAVYYCARDG | |
| | TYLGGLWYFDLWGRGTLVTVSSGSTSGSGKPGSGEGSTKGDIVMTQSPLS | |
| | LPVTPGEPASISCRSSQSLLHSNGYNYLDWYLQKPGQSPQLLIYLGSNRA | |
| | SGVPDRFSGSGSGTDFTLKISRVEAEDVGVYYCMQGLGLPLTFGGGTKVE | |
| | IKRAAALDNEKSNGTIIHVKGKHLCPSPLFPGPSKPFWVLVVVGGVLACY | |
| | SLLVTVAFIIFWVRSKRSRLLHSDYMNMTPRRPGPTRKHYQPYAPPRDFA | |
| | AYRSRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 93 | DIVMTQSPLSLPVTPGEPASISCRSSQSLLHSNGYNYLDWYLQKPGQSPQ | anti-BCMA CAR |
| | LLIYLGSNRASGVPDRFSGSGSGTDFTLKISRVEAEDVGVYYCMQGLGLP | |
| | LTFGGGTKVEIKRGSTSGSGKPGSGEGSTKGQVQLVESGGGVVQPGRSLR | |
| | LSCAASGFTFSSYGMHWVRQAPGKGLEWVAVISYDGSNKYYADSVKGRFT | |
| | ISRDNSKNTLYLQMNSLRAEDTAVYYCARDGTYLGGLWYFDLWGRGTLVT | |
| | VSSAAALDNEKSNGTIIHVKGKHLCPSPLFPGPSKPFWVLVVVGGVLACY | |
| | SLLVTVAFIIFWVRSKRSRLLHSDYMNMTPRRPGPTRKHYQPYAPPRDFA | |
| | AYRSRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 94 | QVQLVQSGAEVKKPGASVKVSCKASGYTFTSYYMHWVRQAPGQGLEWMGI | anti-BCMA CAR |
| | INPGGGSTSYAQKFQGRVTMTRDTSTSTVYMELSSLRSEDTAVYYCARES | |
| | WPMDVWGQGTTVTVSSGSTSGSGKPGSGEGSTKGEIVMTQSPATLSVSPG | |
| | ERATLSCRASQSVSSNLAWYQQKPGQAPRLLIYGASTRATGIPARFSGSG | |
| | SGTEFTLTISSLQSEDFAVYYCQQYAAYPTFGGGTKVEIKRAAALDNEKS | |
| | NGTIIHVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRVKFSRSA | |
| | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPR | |
| 95 | EIVMTQSPATLSVSPGERATLSCRASQSVSSNLAWYQQKPGQAPRLLIYG | anti-BCMA CAR |
| | ASTRATGIPARFSGSGSGTEFTLTISSLQSEDFAVYYCQQYAAYPTFGGG | |
| | TKVEIKRGSTSGSGKPGSGEGSTKGQVQLVQSGAEVKKPGASVKVSCKAS | |
| | GYTFTSYYMHWVRQAPGQGLEWMGIINPGGGSTSYAQKFQGRVTMTRDTS | |
| | TSTVYMELSSLRSEDTAVYYCARESWPMDVWGQGTTVTVSSAAALDNEKS | |
| | NGTIIHVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRVKFSRSA | |
| | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPR | |
| 96 | QLQLQESGPGLVKPSETLSLTCTVSGGSISSSSYYWGWIRQPPGKGLEWI | anti-BCMA CAR |
| | GSISYSGSTYYNPSLKSRVTISVDTSKNQFSLKLSSVTAADTAVYYCARG | |
| | RGYATSLAFDIWGQGTMVTVSSGSTSGSGKPGSGEGSTKGEIVLTQSPAT | |
| | LSLSPGERATLSCRASQSVSSYLAWYQQKPGQAPRLLIYDASNRATGIPA | |
| | RFSGSGSGTDFTLTISSLEPEDFAVYYCQQRHVWPPTFGGGTKVEIKRAA | |
| | ALDNEKSNGTIIHVKGKHLCPSPLFPGPSKPFWVLVVVGGVLACYSLLVT | |
| | VAFIIFWVRSKRSRLLHSDYMNMTPRRPGPTRKHYQPYAPPRDFAAYRSR | |
| | VKFSRSADAPAYQQGQNQLYNELNLGRREEYDVLDKRRGRDPEMGGKPRR | |
| | KNPQEGLYNELQKDKMAEAYSEIGMKGERRRGKGHDGLYQGLSTATKDTY | |
| | DALHMQALPPR | |
| 97 | EIVLTQSPATLSLSPGERATLSCRASQSVSSYLAWYQQKPGQAPRLLIYD | anti-BCMA CAR |
| | ASNRATGIPARFSGSGSGTDFTLTISSLEPEDFAVYYCQQRHVWPPTFGG | |
| | GTKVEIKRGSTSGSGKPGSGEGSTKGQLQLQESGPGLVKPSETLSLTCTV | |
| | SGGSISSSSYYWGWIRQPPGKGLEWIGSISYSGSTYYNPSLKSRVTISVD | |
| | TSKNQFSLKLSSVTAADTAVYYCARGRGYATSLAFDIWGQGTMVTVSSAA | |
| | ALDNEKSNGTIIHVKGKHLCPSPLFPGPSKPFWVLVVVGGVLACYSLLVT | |
| | VAFIIFWVRSKRSRLLHSDYMNMTPRRPGPTRKHYQPYAPPRDFAAYRSR | |
| | VKFSRSADAPAYQQGQNQLYNELNLGRREEYDVLDKRRGRDPEMGGKPRR | |
| | KNPQEGLYNELQKDKMAEAYSEIGMKGERRRGKGHDGLYQGLSTATKDTY | |
| | DALHMQALPPR | |
| 98 | EVQLVESGGGLVQPGGSLRLSCAASGFTFSSYSMNWVRQAPGKGLEWVST | anti-BCMA CAR |
| | ISSSSSTIYYADSVKGRFTISRDNAKNSLYLQMNSLRAEDTAVYYCARGS | |
| | QEHLIFDYWGQGTLVTVSSGSTSGSGKPGSGEGSTKGEIVLTQSPATLSL | |
| | SPGERATLSCRASQSVSRYLAWYQQKPGQAPRLLIYDASNRATGIPARFS | |
| | GSGSGTDFTLTISSLEPEDFAVYYCQQRFYYPWTFGGGTKVEIKRAAALD | |
| | NEKSNGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYRSRVKF | |
| | SRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDAL | |
| | HMQALPPR | |
| 99 | EIVLTQSPATLSLSPGERATLSCRASQSVSRYLAWYQQKPGQAPRLLIYD | anti-BCMA CAR |
| | ASNRATGIPARFSGSGSGTDFTLTISSLEPEDFAVYYCQQRFYYPWTFGG | |
| | GTKVEIKRGSTSGSGKPGSGEGSTKGEVQLVESGGGLVQPGGSLRLSCAA | |
| | SGFTFSSYSMNWVRQAPGKGLEWVSTISSSSSTIYYADSVKGRFTISRDN | |
| | AKNSLYLQMNSLRAEDTAVYYCARGSQEHLIFDYWGQGTLVTVSSAAALD | |
| | NEKSNGTIIHVKGKHLCPSPLFPGPSKPFWVLVVVGGVLACYSLLVTVAF | |
| | IIFWVRSKRSRLLHSDYMNMTPRRPGPTRKHYQPYAPPRDFAAYRSRVKF | |
| | SRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDAL | |
| | HMQALPPR | |
| 100 | QVQLVESGGGVVQPGRSLRLSCAASGFTFSSYGMHWVRQAPGKGLEWVAV | anti-BCMA CAR |
| | ISYDGSNKYYADSVKGRFTISRDNSKNTLYLQMNSLRAEDTAVYYCARTD | |
| | FWSGSPPGLDYWGQGTLVTVSSGSTSGSGKPGSGEGSTKGDIQLTQSPSS | |
| | VSASVGDRVTITCRASQGISSWLAWYQQKPGKAPKLLIYGASSLQSGVPS | |
| | RFSGSGSGTDFTLTISSLQPEDFATYYCQQIYTFPFTFGGGTKVEIKRAA | |
| | ALDNEKSNGTIIHVKGKHLCPSPLFPGPSKPFWVLVVVGGVLACYSLLVT | |
| | VAFIIFWVRSKRSRLLHSDYMNMTPRRPGPTRKHYQPYAPPRDFAAYRSR | |
| | VKFSRSADAPAYQQGQNQLYNELNLGRREEYDVLDKRRGRDPEMGGKPRR | |
| | KNPQEGLYNELQKDKMAEAYSEIGMKGERRRGKGHDGLYQGLSTATKDTY | |
| | DALHMQALPPR | |
| 101 | DIQLTQSPSSVSASVGDRVTITCRASQGISSWLAWYQQKPGKAPKLLIYG | anti-BCMA CAR |
| | ASSLQSGVPSRFSGSGSGTDFTLTISSLQPEDFATYYCQQIYTFPFTFGG | |
| | GTKVEIKRGSTSGSGKPGSGEGSTKGQVQLVESGGGVVQPGRSLRLSCAA | |
| | SGFTFSSYGMHWVRQAPGKGLEWVAVISYDGSNKYYADSVKGRFTISRDN | |
| | SKNTLYLQMNSLRAEDTAVYYCARTDFWSGSPPGLDYWGQGTLVTVSSAA | |
| | ALDNEKSNGTIIHVKGKHLCPSPLFPGPSKPFWVLVVVGGVLACYSLLVT | |
| | VAFIIFWVRSKRSRLLHSDYMNMTPRRPGPTRKHYQPYAPPRDFAAYRSR | |
| | VKFSRSADAPAYQQGQNQLYNELNLGRREEYDVLDKRRGRDPEMGGKPRR | |
| | KNPQEGLYNELQKDKMAEAYSEIGMKGERRRGKGHDGLYQGLSTATKDTY | |
| | DALHMQALPPR | |
| 102 | QVQLVQSGAEVKKPGSSVKVSCKASGGTFSSYAISWVRQAPGQGLEWMGG | anti-BCMA CAR |
| | IIPIFGTANYAQKFQGRVTITADESTSTAYMELSSLRSEDTAVYYCARTP | |
| | EYSSSIWHYYYGMDVWGQGTTVTVSSGSTSGSGKPGSGEGSTKGDIVMTQ | |
| | SPDSLAVSLGERATINCKSSQSVLYSSNNKNYLAWYQQKPGQPPKLLIYW | |
| | ASTRESGVPDRFSGSGSGTDFTLTISSLQAEDVAVYYCQQFAHTPFTFGG | |
| | GTKVEIKRAAALDNEKSNGTIIHVKGKHLCPSPLFPGPSKPFWVLVVVGG | |
| | VLACYSLLVTVAFIIFWVRSKRSRLLHSDYMNMTPRRPGPTRKHYQPYAP | |
| | PRDFAAYRSRVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQ | |
| | GLSTATKDTYDALHMQALPPR | |
| 103 | DIVMTQSPDSLAVSLGERATINCKSSQSVLYSSNNKNYLAWYQQKPGQPP | anti-BCMA CAR |
| | KLLIYWASTRESGVPDRFSGSGSGTDFTLTISSLQAEDVAVYYCQQFAHT | |
| | PFTFGGGTKVEIKRGSTSGSGKPGSGEGSTKGQVQLVQSGAEVKKPGSSV | |
| | KVSCKASGGTFSSYAISWVRQAPGQGLEWMGGIIPIFGTANYAQKFQGRV | |
| | TITADESTSTAYMELSSLRSEDTAVYYCARTPEYSSSIWHYYYGMDVWGQ | |
| | GTTVTVSSAAALDNEKSNGTIIHVKGKHLCPSPLFPGPSKPFWVLVVVGG | |
| | VLACYSLLVTVAFIIFWVRSKRSRLLHSDYMNMTPRRPGPTRKHYQPYAP | |
| | PRDFAAYRSRVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQ | |
| | GLSTATKDTYDALHMQALPPR | |
| 104 | QVQLVESGGGVVQPGRSLRLSCAASGFTFSSYGMHWVRQAPGKGLEWVAV | anti-BCMA CAR |
| | ISYDGSNKYYADSVKGRFTISRDNSKNTLYLQMNSLRAEDTAVYYCVKGP | |
| | LQEPPYDYGMDVWGQGTTVTVSSGSTSGSGKPGSGEGSTKGEIVMTQSPA | |
| | TLSVSPGERATLSCRASQSVSSNLAWYQQKPGQAPRLLIYSASTRATGIP | |
| | ARFSGSGSGTEFTLTISSLQSEDFAVYYCQQHHVWPLTFGGGTKVEIKRA | |
| | AALDNEKSNGTIIHVKGKHLCPSPLFPGPSKPFWVLVVVGGVLACYSLLV | |
| | TVAFIIFWVRSKRSRLLHSDYMNMTPRRPGPTRKHYQPYAPPRDFAAYRS | |
| | RVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 105 | EIVMTQSPATLSVSPGERATLSCRASQSVSSNLAWYQQKPGQAPRLLIYS | anti-BCMA CAR |
| | ASTRATGIPARFSGSGSGTEFTLTISSLQSEDFAVYYCQQHHVWPLTFGG | |
| | GTKVEIKRGSTSGSGKPGSGEGSTKGQVQLVESGGGVVQPGRSLRLSCAA | |
| | SGFTFSSYGMHWVRQAPGKGLEWVAVISYDGSNKYYADSVKGRFTISRDN | |
| | SKNTLYLQMNSLRAEDTAVYYCVKGPLQEPPYDYGMDVWGQGTTVTVSSA | |
| | AALDNEKSNGTIIHVKGKHLCPSPLFPGPSKPFWVLVVVGGVLACYSLLV | |
| | TVAFIIFWVRSKRSRLLHSDYMNMTPRRPGPTRKHYQPYAPPRDFAAYRS | |
| | RVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 106 | QSALTQPASVSASPGQSIAISCTGTSSDVGWYQQHPGKAPKLMIYEDSKR | anti-BCMA CAR |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSSAAAIEV | |
| | MYPPPYLDNEKSNGTIIHVKGKHLCPSPLFPGPSKPFWVLVVVGGVLACY | |
| | SLLVTVAFIIFWVRSKRSRLLHSDYMNMTPRRPGPTRKHYQPYAPPRDFA | |
| | AYRSRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 107 | QSVLTQPPSVSGAPGQRVTISCTGSSSNIGAGFDVHWYQQLPGTAPKLLI | anti-BCMA CAR |
| | YGNSNRPSGVPDRFSGSKSGTSASLAITGLQAEDEADYYCQSYDSSLSGY | |
| | VFGTGTKVTVLGSRGGGGSGGGGSGGGGSLEMAQVQLVQSGAEVKKPGAS | |
| | VKVSCKASGYTFTDYYMHWVRQAPGQRLEWMGWINPNSGGTNYAQKFQDR | |
| | ITVTRDTSSNTGYMELTRLRSDDTAVYYCARSPYSGVLDKWGQGTLVTVS | |
| | SAAAIEVMYPPPYLDNEKSNGTIIHVKGKHLCPSPLFPGPSKPFWVLVVV | |
| | GGVLACYSLLVTVAFIIFWVRSKRSRLLHSDYMNMTPRRPGPTRKHYQPY | |
| | APPRDFAAYRSRVKFSRSADAPAYQQGQNQLYNELNLGRREEYDVLDKRR | |
| | GRDPEMGGKPRRKNPQEGLYNELQKDKMAEAYSEIGMKGERRRGKGHDGL | |
| | YQGLSTATKDTYDALHMQALPPR | |
| 108 | SYELTQPPSASGTPGQRVTMSCSGTSSNIGSHSVNWYQQLPGTAPKLLIY | anti-BCMA CAR |
| | TNNQRPSGVPDRFSGSKSGTSASLAISGLQSEDEADYYCAAWDGSLNGLV | |
| | FGGGTKLTVLGSRGGGGSGGGGSGGGGSLEMAEVQLVQSGAEVKKPGESL | |
| | KISCKGSGYSFTSYWIGWVRQMPGKGLEWMGIIYPGDSDTRYSPSFQGHV | |
| | TISADKSISTAYLQWSSLKASDTAMYYCARYSGSFDNWGQGTLVTVSSAA | |
| | AIEVMYPPPYLDNEKSNGTIIHVKGKHLCPSPLFPGPSKPFWVLVVVGGV | |
| | LACYSLLVTVAFIIFWVRSKRSRLLHSDYMNMTPRRPGPTRKHYQPYAPP | |
| | RDFAAYRSRVKFSRSADAPAYQQGQNQLYNELNLGRREEYDVLDKRRGRD | |
| | PEMGGKPRRKNPQEGLYNELQKDKMAEAYSEIGMKGERRRGKGHDGLYQG | |
| | LSTATKDTYDALHMQALPPR | |
| 109 | LPVLTQPPSASGTPGQRVTISCSGRSSNIGSNSVNWYRQLPGAAPKLLIY | anti-BCMA CAR |
| | SNNQRPPGVPVRFSGSKSGTSASLAISGLQSEDEATYYCATWDDNLNVHY | |
| | VFGTGTKVTVLGSRGGGGSGGGGSGGGGSLEMAQVQLVQSGAEVKKPGSS | |
| | VKVSCKASGGTFSSYAISWVRQAPGQGLEWMGRIIPILGIANYAQKFQGR | |
| | VTITADKSTSTAYMELSSLRSEDTAVYYCARGGYYSHDMWSEDWGQGTLV | |
| | TVSSAAAIEVMYPPPYLDNEKSNGTIIHVKGKHLCPSPLFPGPSKPFWVL | |
| | VVVGGVLACYSLLVTVAFIIFWVRSKRSRLLHSDYMNMTPRRPGPTRKHY | |
| | QPYAPPRDFAAYRSRVKFSRSADAPAYQQGQNQLYNELNLGRREEYDVLD | |
| | KRRGRDPEMGGKPRRKNPQEGLYNELQKDKMAEAYSEIGMKGERRRGKGH | |
| | DGLYQGLSTATKDTYDALHMQALPPR | |
| 110 | QAVLTQPPSASGTPGQRVTISCSGSSSNIGSNYVFWYQQLPGTAPKLLIY | anti-BCMA CAR |
| | SNNQRPSGVPDRFSGSKSGTSASLAISGLRSEDEADYYCAAWDDSLSASY | |
| | VFGTGTKVTVLGSRGGGGSGGGGSGGGGSLEMAQVQLVQSGAEVKKPGSS | |
| | VKVSCKASGGTFSSYAISWVRQAPGQGLEWMGRIIPILGTANYAQKFQGR | |
| | VTITADESTSTAYMELSSLRSEDTAVYYCARSGYGSYRWEDSWGQGTLVT | |
| | VSSAAAIEVMYPPPYLDNEKSNGTIIHVKGKHLCPSPLFPGPSKPFWVLV | |
| | VVGGVLACYSLLVTVAFIIFWVRSKRSRLLHSDYMNMTPRRPGPTRKHYQ | |
| | PYAPPRDFAAYRSRVKFSRSADAPAYQQGQNQLYNELNLGRREEYDVLDK | |
| | RRGRDPEMGGKPRRKNPQEGLYNELQKDKMAEAYSEIGMKGERRRGKGHD | |
| | GLYQGLSTATKDTYDALHMQALPPR | |
| 111 | LPVLTQPPSASGTPGQRVTISCSGRSSNIGSNSVNWYRQLPGAAPKLLIY | anti-BCMA CAR |
| | SNNQRPPGVPVRFSGSKSGTSASLAISGLQSEDEATYYCATWDDNLNVHY | |
| | VFGTGTKVTVLGSRGGGGSGGGGSGGGGSLEMAQVQLVQSGAEVKKPGSS | |
| | VKVSCKASGGTFSSYAISWVRQAPGQGLEWMGRIIPILGIANYAQKFQGR | |
| | VTITADKSTSTAYMELSSLRSEDTAVYYCARGGYYSHDMWSEDWGQGTLV | |
| | TVSSAAAPTTTPAPRPPTPAPTIASQPLSLRPEACRPAAGGAVHTRGLDF | |
| | ACDIYIWAPLAGTCGVLLLSLVITLYCNKRGRKKLLYIFKQPFMRPVQTT | |
| | QEEDGCSCRFPEEEEGGCELRVKFSRSAEPPAYQQGQNQLYNELNLGRRE | |
| | EYDVLDKRRGRDPEMGGKPRRKNPQEGLYNELQKDKMAEAYSEIGMKGER | |
| | RRGKGHDGLYQGLSTATKDTYDALHMQALPPR | |
| 112 | SYELTQPPSASGTPGQRVTMSCSGTSSNIGSHSVNWYQQLPGTAPKLLIY | anti-BCMA CAR |
| | TNNQRPSGVPDRFSGSKSGTSASLAISGLQSEDEADYYCAAWDGSLNGLV | |
| | FGGGTKLTVLGSRGGGGSGGGGSGGGGSLEMAEVQLVQSGAEVKKPGESL | |
| | KISCKGSGYSFTSYWIGWVRQMPGKGLEWMGIIYPGDSDTRYSPSFQGHV | |
| | TISADKSISTAYLQWSSLKASDTAMYYCARYSGSFDNWGQGTLVTVSSAA | |
| | APTTTPAPRPPTPAPTIASQPLSLRPEACRPAAGGAVHTRGLDFACDIYI | |
| | WAPLAGTCGVLLLSLVITLYCNKRGRKKLLYIFKQPFMRPVQTTQEEDGC | |
| | SCRFPEEEEGGCELRVKFSRSAEPPAYQQGQNQLYNELNLGRREEYDVLD | |
| | KRRGRDPEMGGKPRRKNPQEGLYNELQKDKMAEAYSEIGMKGERRRGKGH | |
| | DGLYQGLSTATKDTYDALHMQALPPR | |
| 113 | QAVLTQPPSASGTPGQRVTISCSGSSSNIGSNYVFWYQQLPGTAPKLLIY | anti-BCMA CAR |
| | SNNQRPSGVPDRFSGSKSGTSASLAISGLRSEDEADYYCAAWDDSLSASY | |
| | VFGTGTKVTVLGSRGGGGSGGGGSGGGGSLEMAQVQLVQSGAEVKKPGSS | |
| | VKVSCKASGGTFSSYAISWVRQAPGQGLEWMGRIIPILGTANYAQKFQGR | |
| | VTITADESTSTAYMELSSLRSEDTAVYYCARSGYGSYRWEDSWGQGTLVT | |
| | VSSAAAPTTTPAPRPPTPAPTIASQPLSLRPEACRPAAGGAVHTRGLDFA | |
| | CDIYIWAPLAGTCGVLLLSLVITLYCNKRGRKKLLYIFKQPFMRPVQTTQ | |
| | EEDGCSCRFPEEEEGGCELRVKFSRSAEPPAYQQGQNQLYNELNLGRREE | |
| | YDVLDKRRGRDPEMGGKPRRKNPQEGLYNELQKDKMAEAYSEIGMKGERR | |
| | RGKGHDGLYQGLSTATKDTYDALHMQALPPR | |
| 114 | QSVLTQPPSVSGAPGQRVTISCTGSSSNIGAGFDVHWYQQLPGTAPKLLI | anti-BCMA CAR |
| | YGNSNRPSGVPDRFSGSKSGTSASLAITGLQAEDEADYYCQSYDSSLSGY | |
| | VFGTGTKVTVLGSRGGGGSGGGGSGGGGSLEMAQVQLVQSGAEVKKPGAS | |
| | VKVSCKASGYTFTDYYMHWVRQAPGQRLEWMGWINPNSGGTNYAQKFQDR | |
| | ITVTRDTSSNTGYMELTRLRSDDTAVYYCARSPYSGVLDKWGQGTLVTVS | |
| | SAAAPTTTPAPRPPTPAPTIASQPLSLRPEACRPAAGGAVHTRGLDFACD | |
| | IYIWAPLAGTCGVLLLSLVITLYCNKRGRKKLLYIFKQPFMRPVQTTQEE | |
| | DGCSCRFPEEEEGGCELRVKFSRSAEPPAYQQGQNQLYNELNLGRREEYD | |
| | VLDKRRGRDPEMGGKPRRKNPQEGLYNELQKDKMAEAYSEIGMKGERRRG | |
| | KGHDGLYQGLSTATKDTYDALHMQALPPR | |
| 115 | QSALTQPASVSASPGQSIAISCTGTSSDVGWYQQHPGKAPKLMIYEDSKR | anti-BCMA CAR |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSSAAAPTT | |
| | TPAPRPPTPAPTIASQPLSLRPEACRPAAGGAVHTRGLDFACDIYIWAPL | |
| | AGTCGVLLLSLVITLYCNKRGRKKLLYIFKQPFMRPVQTTQEEDGCSCRF | |
| | PEEEEGGCELRVKFSRSAEPPAYQQGQNQLYNELNLGRREEYDVLDKRRG | |
| | RDPEMGGKPRRKNPQEGLYNELQKDKMAEAYSEIGMKGERRRGKGHDGLY | |
| | QGLSTATKDTYDALHMQALPPR | |
| 116 | DIVLTQSPPSLAMSLGKRATISCRASESVTILGSHLIHWYQQKPGQPPTL | anti-BCMA CAR |
| | LIQLASNVQTGVPARFSGSGSRTDFTLTIDPVEEDDVAVYYCLQSRTIPR | |
| | TFGGGTKLEIKGSTSGSGKPGSGEGSTKGQIQLVQSGPELKKPGETVKIS | |
| | CKASGYTFTDYSINWVKRAPGKGLKWMGWINTETREPAYAYDFRGRFAFS | |
| | LETSASTAYLQINNLKYEDTATYFCALDYSYAMDYWGQGTSVTVSSAAAT | |
| | TTPAPRPPTPAPTIASQPLSLRPEACRPAAGGAVHTRGLDFACDIYIWAP | |
| | LAGTCGVLLLSLVITLYCKRGRKKLLYIFKQPEMRPVQTTQEEDGCSCRF | |
| | PEEEEGGCELRVKFSRSADAPAYQQGQNQLYNELNLGRREEYDVLDKRRG | |
| | RDPEMGGKPRRKNPQEGLYNELQKDKMAEAYSEIGMKGERRRGKGHDGLY | |
| | QGLSTATKDTYDALHMQALPPR | |
| 117 | DIVLTQSPASLAVSLGERATINCRASESVSVIGAHLIHWYQQKPGQPPKL | anti-BCMA CAR |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSSAAAT | |
| | TTPAPRPPTPAPTIASQPLSLRPEACRPAAGGAVHTRGLDFACDIYIWAP | |
| | LAGTCGVLLLSLVITLYCKRGRKKLLYIFKQPFMRPVQTTQEEDGCSCRF | |
| | PEEEEGGCELRVKFSRSADAPAYQQGQNQLYNELNLGRREEYDVLDKRRG | |
| | RDPEMGGKPRRKNPQEGLYNELQKDKMAEAYSEIGMKGERRRGKGHDGLY | |
| | QGLSTATKDTYDALHMQALPPR | |
| 118 | DIVLTQSPASLAVSLGERATINCRASESVSVIGAHLIHWYQQKPGQPPKL | anti-BCMA CAR |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSSAAAD | |
| | TGLYICKVELMYPPPYYLGIGNGTQIYVIDPEPCPDSDFLLWILAAVSSG | |
| | LFFYSFLLTAVSKRGRKKLLYIFKQPFMRPVQTTQEEDGCSCRFPEEEEG | |
| | GCELRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 119 | DIVLTQSPASLAVSLGERATINCRASESVSVIGAHLIHWYQQKPGQPPKL | anti-BCMA CAR |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSSAAAQ | |
| | IKESLRAELRVTERRAEVPTAHPSPSPRPAGQFQTLVVGVVGGLLGSLVL | |
| | LVWVLAVICSKRGRKKLLYIFKQPFMRPVQTTQEEDGCSCRFPEEEEGGC | |
| | ELRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATK | |
| | DTYDALHMQALPPR | |
| 120 | EVQLVESGGGLVKPGGSLRLSCAASGFTFSDYYMSWIRQAPGKGLEWVSY | anti-BCMA CAR |
| | ISSSGSTIYYADSVKGRFTISRDNAKNSLYLQMNSLRAEDTAVYYCAKVD | |
| | GDYTEDYWGQGTLVTVSSGGGGSGGGGSGGGGSQSALTQPASVSGSPGQS | |
| | ITISCTGSSSDVGKYNLVSWYQQPPGKAPKLIIYDVNKRPSGVSNRFSGS | |
| | KSGNTATLTISGLQGDDEADYYCSSYGGSRSYVFGTGTKVTVLESKYGPP | |
| | CPPCPAPPVAGPSVFLFPPKPKDTLMISRTPEVTCVVVDVSQEDPEVQFN | |
| | WYVDGVEVHNAKTKPREEQFQSTYRVVSVLTVLHQDWLNGKEYKCKVSNK | |
| | GLPSSIEKTISKAKGQPREPQVYTLPPSQEEMTKNQVSLTCLVKGFYPSD | |
| | IAVEWESNGQPENNYKTTPPVLDSDGSFFLYSRLTVDKSRWQEGNVFSCS | |
| | VMHEALHNHYTQKSLSLSLGKMFWVLVVVGGVLACYSLLVTVAFIIFWVK | |
| | RGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQAL | |
| | PPR | |
| 121 | EVQLVQSGGGLVQPGRSLRLSCTASGFTFGDYAMSWFKQAPGKGLEWVGF | anti-BCMA CAR |
| | IRSKAYGGTTEYAASVKGRFTISRDDSKSIAYLQMNSLKTEDTAVYYCAA | |
| | WSAPTDYWGQGTLVTVSSGGGGSGGGGSGGGGSDIQMTQSPAFLSASVGD | |
| | RVTVTCRASQGISNYLAWYQQKPGNAPRLLIYSASTLQSGVPSRFRGTGY | |
| | GTEFSLTIDSLQPEDFATYYCQQSYTSRQTFGPGTRLDIKESKYGPPCPP | |
| | CPAPPVAGPSVFLFPPKPKDTLMISRTPEVTCVVVDVSQEDPEVQFNWYV | |
| | DGVEVHNAKTKPREEQFQSTYRVVSVLTVLHQDWLNGKEYKCKVSNKGLP | |
| | SSIEKTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFSCSVMH | |
| | EALHNHYTQKSLSLSLGKMFWVLVVVGGVLACYSLLVTVAFIIFWVKRGR | |
| | KKLLYIFKQPFMRPVQTTQEEDGCSCRFPEEEEGGCELRVKFSRSADAPA | |
| | YQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| 122 | EVQLVESGGGLVKPGGSLRLSCAASGFTFSDYYMSWIRQAPGKGLEWVSY | anti-BCMA CAR |
| | ISSSGSTIYYADSVKGRFTISRDNAKNSLYLQMNSLRAEDTAVYYCAKVD | |
| | GPPSFDIWGQGTMVTVSSGGGGSGGGGSGGGGSSYVLTQPPSVSVAPGQT | |
| | ARITCGANNIGSKSVHWYQQKPGQAPMLVVYDDDDRPSGIPERFSGSNSG | |
| | NTATLTISGVEAGDEADYFCHLWDRSRDHYVFGTGTKLTVLESKYGPPCP | |
| | PCPAPPVAGPSVFLFPPKPKDTLMISRTPEVTCVVVDVSQEDPEVQFNWY | |
| | VDGVEVHNAKTKPREEQFQSTYRVVSVLTVLHQDWLNGKEYKCKVSNKGL | |
| | PSSIEKTISKAKGQPREPQVYTLPPSQEEMTKNQVSLTCLVKGFYPSDIA | |
| | VEWESNGQPENNYKTTPPVLDSDGSFFLYSRLTVDKSRWQEGNVFSCSVM | |
| | HEALHNHYTQKSLSLSLGKMFWVLVVVGGVLACYSLLVTVAFIIFWVKRG | |
| | RKKLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLYNE | |
| | LQKDKMAEAYSEIGMKGERRRGKGHDGLYQGLSTATKDTYDALHMQALPP | |
| | R | |
| 123 | SYELTQPPSASGTPGQRVTMSCSGTSSNIGSHSVNWYQQLPGTAPKLLIY | anti-BCMA CAR |
| | TNNQRPSGVPDRFSGSKSGTSASLAISGLQSEDEADYYCAAWDGSLNGLV | |
| | FGGGTKLTVLGSRGGGGSGGGGSGGGGSLEMAEVQLVQSGAEVKKPGESL | |
| | KISCKGSGYSFTSYWIGWVRQMPGKGLEWMGIIYPGDSDTRYSPSFQGHV | |
| | TISADKSISTAYLQWSSLKASDTAMYYCARYSGSFDNWGQGTLVTVSSES | |
| | KYGPPCPPCPAPPVAGPSVFLFPPKPKDTLMISRTPEVTCVVVDVSQEDP | |
| | EVQFNWYVDGVEVHNAKTKPREEQFQSTYRVVSVLTVLHQDWLNGKEYKC | |
| | KVSNKGLPSSIEKTISKAKGQPREPQVYTLPPSQEEMTKNQVSLTCLVKG | |
| | FYPSDIAVEWESNGQPENNYKTTPPVLDSDGSFFLYSRLTVDKSRWQEGN | |
| | VFSCSVMHEALHNHYTQKSLSLSLGKMFWVLVVVGGVLACYSLLVTVAFI | |
| | IFWVKRGRKKLLYIFKQPFMRPVQTTQEEDGCSCRFPEEEEGGCELRVKF | |
| | SRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDAL | |
| | HMQALPPR | |
| 124 | QSALTQPASVSASPGQSIAISCTGTSSDVGWYQQHPGKAPKLMIYEDSKR | anti-BCMA CAR |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSSESKYGP | |
| | PCPPCPAPPVAGPSVFLFPPKPKDTLMISRTPEVTCVVVDVSQEDPEVQF | |
| | NWYVDGVEVHNAKTKPREEQFQSTYRVVSVLTVLHQDWLNGKEYKCKVSN | |
| | KGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSC | |
| | SVMHEALHNHYTQKSLSLSLGKMFWVLVVVGGVLACYSLLVTVAFIIFWV | |
| | KRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPR | |
| 125 | QSALTQPASVSASPGQSIAISCTGTSSDVGWYQQHPGKAPKLMIYEDSKR | anti-BCMA CAR |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSSESKYGP | |
| | PCPPCPAPPVAGPSVFLFPPKPKDTLMISRTPEVTCVVVDVSQEDPEVQF | |
| | NWYVDGVEVHNAKTKPREEQFQSTYRVVSVLTVLHQDWLNGKEYKCKVSN | |
| | KGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSC | |
| | SVMHEALHNHYTQKSLSLSLGKMFWVLVVVGGVLACYSLLVTVAFIIFWV | |
| | RSKRSRLLHSDYMNMTPRRPGPTRKHYQPYAPPRDFAAYRSRVKFSRSAD | |
| | APAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQAL | |
| | PPR | |
| 126 | EVQLVESGGGLVQPGGSLRLSCAVSGFALSNHGMSWVRRAPGKGLEWVSG | anti-BCMA CAR |
| | IVYSGSTYYAASVKGRFTISRDNSRNTLYLQMNSLRPEDTAIYYCSAHGG | |
| | ESDVWGQGTTVTVSSASGGGGSGGRASGGGGSDIQLTQSPSSLSASVGDR | |
| | VTITCRASQSISSYLNWYQQKPGKAPKLLIYAASSLQSGVPSRFSGSGSG | |
| | TDFTLTISSLQPEDFATYYCQQSYSTPYTFGQGTKVEIKTTTPAPRPPTP | |
| | APTIASQPLSLRPEACRPAAGGAVHTRGLDFACDIYIWAPLAGTCGVLLL | |
| | SLVITLYCKRGRKKLLYIFKQPFMRPVQTTQEEDGCSCRFPEEEEGGCEL | |
| | RVKFSRSADAPAYKQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 127 | QVQLVESGGGLVQPGRSLRLSCAASGFTFSNYAMSWVRQAPGKGLGWVSG | anti-BCMA CAR |
| | ISRSGENTYYADSVKGRFTISRDNSKNTLYLQMNSLRDEDTAVYYCARSP | |
| | AHYYGGMDVWGQGTTVTVSSASGGGGSGGRASGGGGSDIVLTQSPGTLSL | |
| | SPGERATLSCRASQSISSSFLAWYQQKPGQAPRLLIYGASRRATGIPDRF | |
| | SGSGSGTDFTLTISRLEPEDSAVYYCQQYHSSPSWTFGQGTKLEIKTTTP | |
| | APRPPTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCRFPEE | |
| | EEGGCELRVKFSRSADAPAYKQGQNQLYNELNLGRREEYDVLDKRRGRDP | |
| | EMGGKPRRKNPQEGLYNELQKDKMAEAYSEIGMKGERRRGKGHDGLYQGL | |
| | STATKDTYDALHMQALPPR | |
| 128 | QVQLVESGGGLVQPGGSLRLSCAVSGFALSNHGMSWVRRAPGKGLEWVSG | anti-BCMA CAR |
| | IVYSGSTYYAASVKGRFTISRDNSRNTLYLQMNSLRPEDTAIYYCSAHGG | |
| | ESDVWGQGTTVTVSSASGGGGSGGRASGGGGSDIRLTQSPSPLSASVGDR | |
| | VTITCQASEDINKFLNWYHQTPGKAPKLLIYDASTLQTGVPSRFSGSGSG | |
| | TDFTLTINSLQPEDIGTYYCQQYESLPLTFGGGTKVEIKTTTPAPRPPTP | |
| | APTIASQPLSLRPEACRPAAGGAVHTRGLDFACDIYIWAPLAGTCGVLLL | |
| | SLVITLYCKRGRKKLLYIFKQPFMRPVQTTQEEDGCSCRFPEEEEGGCEL | |
| | RVKFSRSADAPAYKQGQNQLYNELNLGRREEYDVLDKRRGRDPEMGGKPR | |
| | RKNPQEGLYNELQKDKMAEAYSEIGMKGERRRGKGHDGLYQGLSTATKDT | |
| | YDALHMQALPPR | |
| 129 | EVQLVESGGGLVQPGGSLRLSCAVSGFALSNHGMSWVRRAPGKGLEWVSG | anti-BCMA CAR |
| | IVYSGSTYYAASVKGRFTISRDNSRNTLYLQMNSLRPEDTAIYYCSAHGG | |
| | ESDVWGQGTTVTVSSASGGGGSGGRASGGGGSEIVLTQSPGTLSLSPGER | |
| | ATLSCRASQSIGSSSLAWYQQKPGQAPRLLMYGASSRASGIPDRFSGSGS | |
| | GTDFTLTISRLEPEDFAVYYCQQYAGSPPFTFGQGTKVEIKTTTPAPRPP | |
| | TPAPTIASQPLSLRPEACRPAAGGAVHTRGLDFACDIYIWAPLAGTCGVL | |
| | LLSLVITLYCKRGRKKLLYIFKQPFMRPVQTTQEEDGCSCRFPEEEEGGC | |
| | ELRVKFSRSADAPAYKQGQNQLYNELNLGRREEYDVLDKRRGRDPEMGGK | |
| | PRRKNPQEGLYNELQKDKMAEAYSEIGMKGERRRGKGHDGLYQGLSTATK | |
| | DTYDALHMQALPPR | |
| 130 | QIQLVQSGPDLKKPGETVKLSCKASGYTFTNFGMNWVKQAPGKGFKWMAW | anti-BCMA CAR |
| | INTYTGESYFADDFKGRFAFSVETSATTAYLQINNLKTEDTATYFCARGE | |
| | IYYGYDGGFAYWGQGTLVTVSAGGGGSGGGGSGGGGSDVVMTQSHRFMST | |
| | SVGDRVSITCRASQDVNTAVSWYQQKPGQSPKLLIFSASYRYTGVPDRFT | |
| | GSGSGADFTLTISSVQAEDLAVYYCQQHYSTPWTFGGGTKLDIKTTTPAP | |
| | RPPTPAPTIASQPLSLRPEACRPAAGGAVHTRGLDFACDIYIWAPLAGTC | |
| | GVLLLSLVITLYCKRGRKKLLYIFKQPFMRPVQTTQEEDGCSCRFPEEEE | |
| | GGCELRVKFSRSADAPAYKQGQNQLYNELNLGRREEYDVLDKRRGRDPEM | |
| | GGKPRRKNPQEGLYNELQKDKMAEAYSEIGMKGERRRGKGHDGLYQGLST | |
| | ATKDTYDALHMQALPPR | |
| 131 | QIQLVQSGPELKKPGETVKISCKASGYTFTDYSINWVKRAPGKGLKWMGW | anti-BCMA CAR |
| | INTETREPAYAYDFRGRFAFSLETSASTAYLQINNLKYEDTATYFCALDY | |
| | SYAMDYWGQGTSVTVSSGGGGSGGGGSGGGGSQIQLVQSGPELKKPGETV | |
| | KISCKASGYTFTDYSINWVKRAPGKGLKWMGWINTETREPAYAYDFRGRF | |
| | AFSLETSASTAYLQINNLKYEDTATYFCALDYSYAMDYWGQGTSVTVSST | |
| | TTPAPRPPTPAPTIASQPLSLRPEACRPAAGGAVHTRGLDFACDIYIWAP | |
| | LAGTCGVLLLSLVITLYCKRGRKKLLYIFKQPFMRPVQTTQEEDGCSCRF | |
| | PEEEEGGCELRVKFSRSADAPAYKQGQNQLYNELNLGRREEYDVLDKRRG | |
| | RDPEMGGKPRRKNPQEGLYNELQKDKMAEAYSEIGMKGERRRGKGHDGLY | |
| | QGLSTATKDTYDALHMQALPPR | |
| 132 | QIQLVQSGPELKKPGETVKISCKASGYTFTDYSINWVKRAPGKGLKWMGW | anti-BCMA CAR |
| | INTETREPAYAYDFRGRFAFSLETSASTAYLQINNLKYEDTATYFCALDY | |
| | SYAMDYWGQGTSVTVSSGGGGSGGGGSGGGGSDIVLTQSPASLAMSLGKR | |
| | ATISCRASESVSVIGAHLIHWYQQKPGQPPKLLIYLASNLETGVPARFSG | |
| | SGSGTDFTLTIDPVEEDDVAIYSCLQSRIFPRTFGGGTKLEIKTTTPAPR | |
| | PPTPAPTIASQPLSLRPEACRPAAGGAVHTRGLDFACDIYIWAPLAGTCG | |
| | VLLLSLVITLYCKRGRKKLLYIFKQPFMRPVQTTQEEDGCSCRFPEEEEG | |
| | GCELRVKFSRSADAPAYKQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 133 | QIQLVQSGPELKKPGETVKISCKASGYTFRHYSMNWVKQAPGKGLKWMGR | anti-BCMA CAR |
| | INTESGVPIYADDFKGRFAFSVETSASTAYLVINNLKDEDTASYFCSNDY | |
| | LYSLDFWGQGTALTVSSGGGGSGGGGSGGGGSDIVLTQSPPSLAMSLGKR | |
| | ATISCRASESVTILGSHLIYWYQQKPGQPPTLLIQLASNVQTGVPARFSG | |
| | SGSRTDFTLTIDPVEEDDVAVYYCLQSRTIPRTFGGGTKLEIKTTTPAPR | |
| | PPTPAPTIASQPLSLRPEACRPAAGGAVHTRGLDFACDIYIWAPLAGTCG | |
| | VLLLSLVITLYCKRGRKKLLYIFKQPFMRPVQTTQEEDGCSCRFPEEEEG | |
| | GCELRVKFSRSADAPAYKQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 134 | QIQLVQSGPELKKPGETVKISCKASGYTFTHYSMNWVKQAPGKGLKWMGR | anti-BCMA CAR |
| | INTETGEPLYADDFKGRFAFSLETSASTAYLVINNLKNEDTATFFCSNDY | |
| | LYSCDYWGQGTTLTVSSGGGGSGGGGSGGGGSDIVLTQSPPSLAMSLGKR | |
| | ATISCRASESVTILGSHLIYWYQQKPGQPPTLLIQLASNVQTGVPARFSG | |
| | SGSRTDFTLTIDPVEEDDVAVYYCLQSRTIPRTFGGGTKLEIKTTTPAPR | |
| | PPTPAPTIASQPLSLRPEACRPAAGGAVHTRGLDFACDIYIWAPLAGTCG | |
| | VLLLSLVITLYCKRGRKKLLYIFKQPFMRPVQTTQEEDGCSCRFPEEEEG | |
| | GCELRVKFSRSADAPAYKQGQNQLYNELNLGRREEYDVLDKRRGRDPEMG | |
| | GKPRRKNPQEGLYNELQKDKMAEAYSEIGMKGERRRGKGHDGLYQGLSTA | |
| | TKDTYDALHMQALPPR | |
| 135 | DIVLTQSPPSLAMSLGKRATISCRASESVTILGSHLIHWYQQKPGQPPTL | anti-BCMA CAR |
| | LIQLASNVQTGVPARFSGSGSRTDFTLTIDPVEEDDVAVYYCLQSRTIPR | |
| | TFGGGTKLEIKGSTSGSGKPGSGEGSTKGQIQLVQSGPELKKPGETVKIS | |
| | CKASGYTFTDYSINWVKRAPGKGLKWMGWINTETREPAYAYDFRGRFAFS | |
| | LETSASTAYLQINNLKYEDTATYFCALDYSYAMDYWGQGTSVTVSSFVPV | |
| | FLPAKPTTTPAPRPPTPAPTIASQPLSLRPEACRPAAGGAVHTRGLDFAC | |
| | DIYIWAPLAGTCGVLLLSLVITLYCNHRNRSKRSRLLHSDYMNMTPRRPG | |
| | PTRKHYQPYAPPRDFAAYRSRVKFSRSADAPAYQQGQNQLYNELNLGRRE | |
| | EYDVLDKRRGRDPEMGGKPRRKNPQEGLYNELQKDKMAEAYSEIGMKGER | |
| | RRGKGHDGLYQGLSTATKDTYDALHMQALPPR | |
| 136 | QVQLVQSGAEVKKPGASVKVSCKASGYSFPDYYINWVRQAPGQGLEWMGW | anti-BCMA CAR |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIKGLAV | |
| | STISSFFPPGYQIYIWAPLAGTCGVLLLSLVITLYCKRGRKKLLYIFKQP | |
| | FMRPVQTTQEEDGCSCRFPEEEEGGCELRVKfSRSADAPAYQQGQNQLYN | |
| | ELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| 137 | QVQLVQSGAEVKKPGASVKVSCKASGYSFPDYYINWVRQAPGQGLEWMGW | anti-BCMA CAR |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIKTTTP | |
| | APRPPTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCRFPEE | |
| | EEGGCELRVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGL | |
| | STATKDTYDALHMQALPPR | |
| 138 | QVQLVQSGAEVKKPGASVKVSCKASGYSFPDYYINWVRQAPGQGLEWMGW | anti-BCMA CAR |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIKEPKS | |
| | PDKTHTCPPCPAPPVAGPSVFLFPPKPKDTLMIARTPEVTCVVVDVSHED | |
| | PEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQG | |
| | NVFSCSVMHEALHNHYTQKSLSLSPGKIYIWAPLAGTCGVLLLSLVITLY | |
| | CKRGRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQ | |
| | ALPPR | |
| 139 | QVQLVQSGAEVKKPGASVKVSCKASGYSFPDYYINWVRQAPGQGLEWMGW | anti-BCMA CAR |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIKGLAV | |
| | STISSFFPPGYQIYIWAPLAGTCGVLLLSLVITLYCKRGRKKLLYIFKQP | |
| | FMRPVQTTQEEDGCSCRFPEEEEGGCELRVKFSRSADAPAYQQGQNQLYN | |
| | ELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| 140 | QVQLVQSGAEVKKPGASVKVSCKASGYSFPDYYINWVRQAPGQGLEWMGW | anti-BCMA CAR |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIKTTTP | |
| | APRPPTPAPTIASQPLSLRPEACRPAAGGAVHTRGLDFACDIYIWAPLAG | |
| | TCGVLLLSLVITLYCKRGRKKLLYIFKQPFMRPVQTTQEEDGCSCRFPEE | |
| | EEGGCELRVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGL | |
| | STATKDTYDALHMQALPPR | |
| 141 | QVQLVQSGAEVKKPGASVKVSCKASGYSFPDYYINWVRQAPGQGLEWMGW | anti-BCMA CAR |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIKEPKS | |
| | PDKTHTCPPCPAPPVAGPSVFLFPPKPKDTLMIARTPEVTCVVVDVSHED | |
| | PEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQG | |
| | NVFSCSVMHEALHNHYTQKSLSLSPGKIYIWAPLAGTCGVLLLSLVITLY | |
| | CKRGRKKLLYIFKQPFMRPVQTTQEEDGCSCRFPEEEEGGCELRVKFSRS | |
| | ADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQ | |
| | ALPPR | |
| 142 | IYIWAPLAGTCGVLLLSLVITLYCNHRN | CD8a TM |
| 143 | IYIWAPLAGTCGVLLLSLVIT | CD8a TM |
| 144 | RAAA | linking peptide |
| 145 | EVQLVESGGGLVKPGGSLRLSCAASGFTFSDYYMSWIRQAPGKGLEWVSY | Variable heavy |
| | ISSSGSTIYYADSVKGRFTISRDNAKNSLYLQMNSLRAEDTAVYYCAKVD | (VH) Anti- |
| | GDYTEDYWGQGTLVTVSS | BCMA |
| 146 | QSALTQPASVSGSPGQSITISCTGSSSDVGKYNLVSWYQQPPGKAPKLII | Variable light |
| | YDVNKRPSGVSNRFSGSKSGNTATLTISGLQGDDEADYYCSSYGGSRSYV | (VL) Anti- |
| | FGTGTKVTVL | BCMA |
| 147 | EVQLVQSGGGLVQPGRSLRLSCTASGFTFGDYAMSWFRQAPGKGLEWVGF | Variable heavy |
| | IRSKAYGGTTEYAASVKGRFTISRDDSKSIAYLQMNSLKTEDTAVYYCAA | (VH) Anti- |
| | WSAPTDYWGQGTLVTVSS | BCMA |
| 148 | DIQMTQSPAFLSASVGDRVTVTCRASQGISNYLAWYQQKPGNAPRLLIYS | Variable light |
| | ASTLQSGVPSRFRGTGYGTEFSLTIDSLQPEDFATYYCQQSYTSRQTFGP | (VL) Anti- |
| | GTRLDIK | BCMA |
| 149 | EVQLVESGGGLVKPGGSLRLSCAASGFTFSDYYMSWIRQAPGKGLEWVSY | Variable heavy |
| | ISSSGSTIYYADSVKGRFTISRDNAKNSLYLQMNSLRAEDTAVYYCAKVD | (VH) Anti- |
| | GPPSFDIWGQGTMVTVSS | BCMA |
| 150 | SYVLTQPPSVSVAPGQTARITCGANNIGSKSVHWYQQKPGQAPMLVVYDD | Variable light |
| | DDRPSGIPERFSGSNSGNTATLTISGVEAGDEADYFCHLWDRSRDHYVFG | (VL) Anti- |
| | TGTKLTVL | BCMA |
| 151 | EVQLVQSGAEVKKPGSSVKVSCKASGGTFSSYAISWVRQAPGQGLEWMGR | Variable heavy |
| | IIPILGIANYAQKFQGRVTMTEDTSTDTAYMELSSLRSEDTAVYYCARSG | (VH) Anti- |
| | YSKSIVSYMDYWGQGTLVTVSS | BCMA |
| 152 | LPVLTQPPSTSGTPGQRVTVSCSGSSSNIGSNVVFWYQQLPGTAPKLVIY | Variable light |
| | RNNQRPSGVPDRFSVSKSGTSASLAISGLRSEDEADYYCAAWDDSLSGYV | (VL) Anti- |
| | FGTGTKVTVLG | BCMA |
| 153 | MPLLLLLPLLWAGALA | CD33 Signal |
| | | peptide |
| 154 | MALPVTALLLPLALLLHA | CD8 alpha signal |
| | | peptide |
| 155 | atgcttctcctggtgacaagccttctgctctgtgagttaccacacccagc | GMCSFR alpha |
| | attcctcctgatccca | chain signal |
| | | sequence |
| 156 | MLLLVTSLLLCELPHPAFLLIP | GMCSFR alpha |
| | | chain signal |
| | | sequence |
| 157 | Glu Val Val Val Lys Tyr Gly Pro Pro Cys Pro Pro | Exemplary IgG |
| | Cys Pro | Hinge |
| 158 | X1PPX2P | Exemplary IgG |
| | X1 is glycine, cysteine or arginine | Hinge |
| | X2 is cysteine or threonine | |
| 159 | Glu Pro Lys Ser Cys Asp Lys Thr His Thr Cys Pro | Exemplary IgG |
| | Pro Cys Pro | Hinge |
| 160 | Glu Arg Lys Cys Cys Val Glu Cys Pro Pro Cys Pro | Exemplary IgG |
| | | Hinge |
| 161 | ELKTPLGDTHTCPRCPEPKSCDTPPPCPRCPEPKSCDTPPPCPRCPEPKS | Exemplary IgG |
| | CDTPPPCPRCP | Hinge |
| 162 | Glu Ser Lys Tyr Gly Pro Pro Cys Pro Ser Cys Pro | Exemplary IgG |
| | | Hinge |
| 163 | Glu Ser Lys Tyr Gly Pro Pro Cys Pro Pro Cys Pro | Exemplary IgG |
| | | Hinge |
| 164 | Tyr Gly Pro Pro Cys Pro Pro Cys Pro | Exemplary IgG |
| | | Hinge |
| 165 | Lys Tyr Gly Pro Pro Cys Pro Pro Cys Pro | Exemplary IgG |
| | | Hinge |
| 166 | MLLLVTSLLLCELPHPAFLLIPRKVCNGIGIGEFKDSLSINATNIKHFKN | tEGFR |
| | CTSISGDLHILPVAFRGDSFTHTPPLDPQELDILKTVKEITGFLLIQAWP | artificial |
| | ENRTDLHAFENLEIIRGRTKQHGQFSLAVVSLNITSLGLRSLKEISDGDV | |
| | IISGNKNLCYANTINWKKLFGTSGQKTKIISNRGENSCKATGQVCHALCS | |
| | PEGCWGPEPRDCVSCRNVSRGRECVDKCNLLEGEPREFVENSECIQCHPE | |
| | CLPQAMNITCTGRGPDNCIQCAHYIDGPHCVKTCPAGVMGENNTLVWKYA | |
| | DAGHVCHLCHPNCTYGCTGPGLEGCPTNGPKIPSIATGMVGALLLLLVVA | |
| | LGIGLFM | |
| 167 | EGRGSLLTCGDVEENPGP | T2A artificial |
| 168 | GSGATNFSLLKQAGDVEENPGP | P2A |
| 169 | ATNFSLLKQAGDVEENPGP | P2A |
| 170 | QCTNYALLKLAGDVESNPGP | E2A |
| 171 | VKQTLNFDLLKLAGDVESNPGP | F2A |
| 172 | MLQMAGQCSQNEYFDSLLHACIPCQLRCSSNTPPLTCQRYCNASVTNSVK | BCMA-Fc fusion |
| | GTNAGGGGSPKSSDKTHTCPPCPAPEAEGAPSVFLFPPKPKDTLMISRTP | polypeptide |
| | EVTCVVVDVSHEDPEVKFNWYVDGVEVHNAKTKPREEQYNSTYRVVSVLT | |
| | VLHQDWLNGKEYKCKVSNKALPSSIEKTISKAKGQPREPQVYTLPPSRDE | |
| | LTKNQVSLTCLVKGFYPSDIAVEWESNGQPENNYKTTPPVLDSDGSFFLY | |
| | SKLTVDKSRWQQGNVFSCSVMHEALHNHYTQKSLSLSPGK | |
| 173 | DYYVY | CDR-H1 |
| 174 | WINPNSGGTNYAQKFQG | CDR-H2 |
| 175 | SQRDGYMDY | CDR-H3 |
| 176 | GYTFIDY | CDR-H1 |
| 177 | NPNSGG | CDR-H2 |
| 178 | GYTFIDYYVY | CDR-H1 |
| 179 | WINPNSGGTN | CDR-H2 |
| 180 | GYTFIDYY | CDR-H1 |
| 181 | INPNSGGT | CDR-H2 |
| 182 | ARSQRDGYMDY | CDR-H3 |
| 183 | TGTSSDVG | CDR-L1 |
| 184 | EDSKRPS | CDR-L2 |
| 185 | SSNTRSSTLV | CDR-L3 |
| 186 | ISCTGTSSD | CDR-L1 |
| 187 | EDS | CDR-L2 |
| 188 | QSALTQPASVSASPGQSIAISCTGTSSDVGWYQQHPGKAPKLMIYEDSKR | anti-BCMA scFv |
| | PSGVSNRFSGSKSGNTASLTISGLQAEDEADYYCSSNTRSSTLVFGGGTK | |
| | LTVLGSRGGGGSGGGGSGGGGSLEMAEVQLVQSGAEMKKPGASLKLSCKA | |
| | SGYTFIDYYVYWMRQAPGQGLESMGWINPNSGGTNYAQKFQGRVTMTRDT | |
| | SISTAYMELSRLRSDDTAMYYCARSQRDGYMDYWGQGTLVTVSS | |
| 189 | DYSIN | CDR-H1 |
| 190 | WINTETREPAYAYDFRG | CDR-H2 |
| 191 | DYSYAMDY | CDR-H3 |
| 192 | RASESVTILGSHLIH | CDR-L1 |
| 193 | LASNVQT | CDR-L2 |
| 194 | LQSRTIPRT | CDR-L3 |
| 195 | GYTFTDY | CDR-H1 |
| 196 | NTETRE | CDR-H2 |
| 197 | DYSYAMDY | CDR-H3 |
| 198 | RASESVTILGSHLIH | CDR-L1 |
| 199 | LASNVQT | CDR-L2 |
| 200 | LQSRTIPRT | CDR-L3 |
| 201 | GYTFTDYSIN | CDR-H1 |
| 202 | WINTETREPA | CDR-H2 |
| 203 | DYSYAMDY | CDR-H3 |
| 204 | RASESVTILGSHLIH | CDR-L1 |
| 205 | LASNVQT | CDR-L2 |
| 206 | LQSRTIPRT | CDR-L3 |
| 207 | GYTFTDYS | CDR-H1 |
| 208 | INTETREP | CDR-H2 |
| 209 | ALDYSYAMDY | CDR-H3 |
| 210 | ESVTILGSHL | CDR-L1 |
| 211 | LA | CDR-L2 |
| 212 | LQSRTIPRT | CDR-L3 |
| 213 | DIVLTQSPPSLAMSLGKRATISCRASESVTILGSHLIHWYQQKPGQPPTL | scFv |
| | LIQLASNVQTGVPARFSGSGSRTDFTLTIDPVEEDDVAVYYCLQSRTIPR | |
| | TFGGGTKLEIKGSTSGSGKPGSGEGSTKGQIQLVQSGPELKKPGETVKIS | |
| | CKASGYTFTDYSINWVKRAPGKGLKWMGWINTETREPAYAYDFRGRFAFS | |
| | LETSASTAYLQINNLKYEDTATYFCALDYSYAMDYWGQGTSVTVSS | |
| 214 | atggcactcc ccgtcaccgc ccttctcttg cccctcgccc | BCMA CAR (nt) |
| | tgctgctgca tgctgccagg cccgacattg tgctcactca | |
| | gtcacctccc agcctggcca tgagcctggg aaaaagggcc | |
| | accatctcct gtagagccag tgagtccgtc acaatcttgg | |
| | ggagccatct tattcactgg tatcagcaga agcccgggca | |
| | gcctccaacc cttcttattc agctcgcgtc aaacgtccag | |
| | acgggtgtac ctgccagatt ttctggtagc gggtcccgca | |
| | ctgattttac actgaccata | |
| | gatccagtgg aagaagacga tgtggccgtg tattattgtc | |
| | tgcagagcag aacgattcct cgcacatttg gtgggggtac | |
| | taagctggag attaagggaa gcacgtccgg ctcagggaag | |
| | ccgggctccg gcgagggaag cacgaagggg caaattcagc | |
| | tggtccagag cggacctgag ctgaaaaaac ccggcgagac | |
| | tgttaagatc agttgtaaag catctggcta taccttcacc | |
| | gactacagca taaattgggt gaaacgggcc cctggaaagg | |
| | gcctcaaatg gatgggttgg | |
| | atcaataccg aaactaggga gcctgcttat gcatatgact | |
| | tccgcgggag attcgccttt tcactcgaga catctgcctc | |
| | tactgcttac ctccaaataa acaacctcaa gtatgaagat | |
| | acagccactt acttttgcgc cctcgactat agttacgcca | |
| | tggactactg gggacaggga acctccgtta ccgtcagttc | |
| | cgcggccgca accacaacac ctgctccaag gccccccaca | |
| | cccgctccaa ctatagccag ccaaccattg agcctcagac | |
| | ctgaagcttg caggcccgca | |
| | gcaggaggcg ccgtccatac gcgaggcctg gacttcgcgt | |
| | gtgatattta tatttgggcc cctttggccg gaacatgtgg | |
| | ggtgttgctt ctctcccttg tgatcactct gtattgtaag | |
| | cgcgggagaa agaagctcct gtacatcttc aagcagcctt | |
| | ttatgcgacc tgtgcaaacc actcaggaag aagatgggtg | |
| | ttcatgccgc ttccccgagg aggaagaagg agggtgtgaa | |
| | ctgagggtga aattttctag aagcgccgat gctcccgcat | |
| | atcagcaggg tcagaatcag | |
| | ctctacaatg aattgaatct cggcaggcga gaagagtacg | |
| | atgttctgga caagagacgg ggcagggatc ccgagatggg | |
| | gggaaagccc cggagaaaaa atcctcagga ggggttgtac | |
| | aatgagctgc agaaggacaa gatggctgaa gcctatagcg | |
| | agatcggaat gaaaggcgaa agacgcagag gcaaggggca | |
| | tgacggtctg taccagggtc tctctacagc caccaaggac | |
| | acttatgatg ccttgcatat gcaagccttg ccaccccgct | |
| | aatga | |
| 215 | MLQMAGQCSQNEYFDSLLHACIPCQLRCSSNTPPLTCQRYCNASVTNSVK | Human BCMA |
| | GTNAILWTCLGLSLIISLAVFVLMFLLRKINSEPLKDEFKNTGSGLLGMA | |
| | NIDLEKSRTGDEIILPRGLEYTVEECTCEDCIKSKPKVDSDHCFPLPAME | |
| | EGATILVTTKTNDYCKSLPAALSATEIEKSISAR | |
| 216 | EVQLLESGGGLVQPGGSLRLSCAASGFTFSSYAMSWVRQAPGKGLEWVSA | Anti-BCMA scFv |
| | ISGSGGSTYYADSVKGRFTISRDNSKNTLYLQMNSLRAEDTAVYYCARAE | |
| | MGAVFDIWGQGTMVTVSSGSTSGSGKPGSGEGSTKGEIVLTQSPATLSLS | |
| | PGERATLSCRASQSVSRYLAWYQQKPGQAPRLLIYDASNRATGIPARFSG | |
| | SGSGTDFTLTISSLEPEDFAVYYCQQRISWPFTFGGGTKVEIK | |
| 217 | EIVLTQSPATLSLSPGERATLSCRASQSVSRYLAWYQQKPGQAPRLLIYD | Anti-BCMA scFv |
| | ASNRATGIPARFSGSGSGTDFTLTISSLEPEDFAVYYCQQRISWPFTFGG | |
| | GTKVEIKRGSTSGSGKPGSGEGSTKGEVQLLESGGGLVQPGGSLRLSCAA | |
| | SGFTFSSYAMSWVRQAPGKGLEWVSAISGSGGSTYYADSVKGRFTISRDN | |
| | SKNTLYLQMNSLRAEDTAVYYCARAEMGAVFDIWGQGTMVTVSS | |
| 218 | QVQLVESGGGVVQPGRSLRLSCAASGFTFSSYGMHWVRQAPGKGLEWVAV | Anti-BCMA scFv |
| | ISYDGSNKYYADSVKGRFTISRDNSKNTLYLQMNSLRAEDTAVYYCARDG | |
| | TYLGGLWYFDLWGRGTLVTVSSGSTSGSGKPGSGEGSTKGDIVMTQSPLS | |
| | LPVTPGEPASISCRSSQSLLHSNGYNYLDWYLQKPGQSPQLLIYLGSNRA | |
| | SGVPDRFSGSGSGTDFTLKISRVEAEDVGVYYCMQGLGLPLTFGGGTKVE | |
| | IK | |
| 219 | DIVMTQSPLSLPVTPGEPASISCRSSQSLLHSNGYNYLDWYLQKPGQSPQ | Anti-BCMA scFv |
| | LLIYLGSNRASGVPDRFSGSGSGTDFTLKISRVEAEDVGVYYCMQGLGLP | |
| | LTFGGGTKVEIKRGSTSGSGKPGSGEGSTKGQVQLVESGGGVVQPGRSLR | |
| | LSCAASGFTFSSYGMHWVRQAPGKGLEWVAVISYDGSNKYYADSVKGRFT | |
| | ISRDNSKNTLYLQMNSLRAEDTAVYYCARDGTYLGGLWYFDLWGRGTLVT | |
| | VSS | |
| 220 | QVQLVQSGAEVKKPGASVKVSCKASGYTFTSYYMHWVRQAPGQGLEWMGI | Anti-BCMA scFv |
| | INPGGGSTSYAQKFQGRVTMTRDTSTSTVYMELSSLRSEDTAVYYCARES | |
| | WPMDVWGQGTTVTVSSGSTSGSGKPGSGEGSTKGEIVMTQSPATLSVSPG | |
| | ERATLSCRASQSVSSNLAWYQQKPGQAPRLLIYGASTRATGIPARFSGSG | |
| | SGTEFTLTISSLQSEDFAVYYCQQYAAYPTFGGGTKVEIK | |
| 221 | EIVMTQSPATLSVSPGERATLSCRASQSVSSNLAWYQQKPGQAPRLLIYG | Anti-BCMA scFv |
| | ASTRATGIPARFSGSGSGTEFTLTISSLQSEDFAVYYCQQYAAYPTFGGG | |
| | TKVEIKRGSTSGSGKPGSGEGSTKGQVQLVQSGAEVKKPGASVKVSCKAS | |
| | GYTFTSYYMHWVRQAPGQGLEWMGIINPGGGSTSYAQKFQGRVTMTRDTS | |
| | TSTVYMELSSLRSEDTAVYYCARESWPMDVWGQGTTVTVSS | |
| 222 | QLQLQESGPGLVKPSETLSLTCTVSGGSISSSSYYWGWIRQPPGKGLEWI | Anti-BCMA scFv |
| | GSISYSGSTYYNPSLKSRVTISVDTSKNQFSLKLSSVTAADTAVYYCARG | |
| | RGYATSLAFDIWGQGTMVTVSSGSTSGSGKPGSGEGSTKGEIVLTQSPAT | |
| | LSLSPGERATLSCRASQSVSSYLAWYQQKPGQAPRLLIYDASNRATGIPA | |
| | RFSGSGSGTDFTLTISSLEPEDFAVYYCQQRHVWPPTFGGGTKVEIK | |
| 223 | EIVLTQSPATLSLSPGERATLSCRASQSVSSYLAWYQQKPGQAPRLLIYD | Anti-BCMA scFv |
| | ASNRATGIPARFSGSGSGTDFTLTISSLEPEDFAVYYCQQRHVWPPTFGG | |
| | GTKVEIKRGSTSGSGKPGSGEGSTKGQLQLQESGPGLVKPSETLSLTCTV | |
| | SGGSISSSSYYWGWIRQPPGKGLEWIGSISYSGSTYYNPSLKSRVTISVD | |
| | TSKNQFSLKLSSVTAADTAVYYCARGRGYATSLAFDIWGQGTMVTVSS | |
| 224 | EVQLVESGGGLVQPGGSLRLSCAASGFTFSSYSMNWVRQAPGKGLEWVST | Anti-BCMA scFv |
| | ISSSSSTIYYADSVKGRFTISRDNAKNSLYLQMNSLRAEDTAVYYCARGS | |
| | QEHLIFDYWGQGTLVTVSSGSTSGSGKPGSGEGSTKGEIVLTQSPATLSL | |
| | SPGERATLSCRASQSVSRYLAWYQQKPGQAPRLLIYDASNRATGIPARFS | |
| | GSGSGTDFTLTISSLEPEDFAVYYCQQRFYYPWTFGGGTKVEIK | |
| 225 | EIVLTQSPATLSLSPGERATLSCRASQSVSRYLAWYQQKPGQAPRLLIYD | Anti-BCMA scFv |
| | ASNRATGIPARFSGSGSGTDFTLTISSLEPEDFAVYYCQQRFYYPWTFGG | |
| | GTKVEIKRGSTSGSGKPGSGEGSTKGEVQLVESGGGLVQPGGSLRLSCAA | |
| | SGFTFSSYSMNWVRQAPGKGLEWVSTISSSSSTIYYADSVKGRFTISRDN | |
| | AKNSLYLQMNSLRAEDTAVYYCARGSQEHLIFDYWGQGTLVTVSS | |
| 226 | QVQLVESGGGVVQPGRSLRLSCAASGFTFSSYGMHWVRQAPGKGLEWVAV | Anti-BCMA scFv |
| | ISYDGSNKYYADSVKGRFTISRDNSKNTLYLQMNSLRAEDTAVYYCARTD | |
| | FWSGSPPGLDYWGQGTLVTVSSGSTSGSGKPGSGEGSTKGDIQLTQSPSS | |
| | VSASVGDRVTITCRASQGISSWLAWYQQKPGKAPKLLIYGASSLQSGVPS | |
| | RFSGSGSGTDFTLTISSLQPEDFATYYCQQIYTFPFTFGGGTKVEIK | |
| 227 | DIQLTQSPSSVSASVGDRVTITCRASQGISSWLAWYQQKPGKAPKLLIYG | Anti-BCMA scFv |
| | ASSLQSGVPSRFSGSGSGTDFTLTISSLQPEDFATYYCQQIYTFPFTFGG | |
| | GTKVEIKRGSTSGSGKPGSGEGSTKGQVQLVESGGGVVQPGRSLRLSCAA | |
| | SGFTFSSYGMHWVRQAPGKGLEWVAVISYDGSNKYYADSVKGRFTISRDN | |
| | SKNTLYLQMNSLRAEDTAVYYCARTDFWSGSPPGLDYWGQGTLVTVSS | |
| 228 | QVQLVQSGAEVKKPGSSVKVSCKASGGTFSSYAISWVRQAPGQGLEWMGG | Anti-BCMA scFv |
| | IIPIFGTANYAQKFQGRVTITADESTSTAYMELSSLRSEDTAVYYCARTP | |
| | EYSSSIWHYYYGMDVWGQGTTVTVSSGSTSGSGKPGSGEGSTKGDIVMTQ | |
| | SPDSLAVSLGERATINCKSSQSVLYSSNNKNYLAWYQQKPGQPPKLLIYW | |
| | ASTRESGVPDRFSGSGSGTDFTLTISSLQAEDVAVYYCQQFAHTPFTFGG | |
| | GTKVEIK | |
| 229 | DIVMTQSPDSLAVSLGERATINCKSSQSVLYSSNNKNYLAWYQQKPGQPP | Anti-BCMA scFv |
| | KLLIYWASTRESGVPDRFSGSGSGTDFTLTISSLQAEDVAVYYCQQFAHT | |
| | PFTFGGGTKVEIKRGSTSGSGKPGSGEGSTKGQVQLVQSGAEVKKPGSSV | |
| | KVSCKASGGTFSSYAISWVRQAPGQGLEWMGGIIPIFGTANYAQKFQGRV | |
| | TITADESTSTAYMELSSLRSEDTAVYYCARTPEYSSSIWHYYYGMDVWGQ | |
| | GTTVTVSS | |
| 230 | QVQLVESGGGVVQPGRSLRLSCAASGFTFSSYGMHWVRQAPGKGLEWVAV | Anti-BCMA scFv |
| | ISYDGSNKYYADSVKGRFTISRDNSKNTLYLQMNSLRAEDTAVYYCVKGP | |
| | LQEPPYDYGMDVWGQGTTVTVSSGSTSGSGKPGSGEGSTKGEIVMTQSPA | |
| | TLSVSPGERATLSCRASQSVSSNLAWYQQKPGQAPRLLIYSASTRATGIP | |
| | ARFSGSGSGTEFTLTISSLQSEDFAVYYCQQHHVWPLTFGGGTKVEIK | |
| 231 | EIVMTQSPATLSVSPGERATLSCRASQSVSSNLAWYQQKPGQAPRLLIYS | Anti-BCMA scFv |
| | ASTRATGIPARFSGSGSGTEFTLTISSLQSEDFAVYYCQQHHVWPLTFGG | |
| | GTKVEIKRGSTSGSGKPGSGEGSTKGQVQLVESGGGVVQPGRSLRLSCAA | |
| | SGFTFSSYGMHWVRQAPGKGLEWVAVISYDGSNKYYADSVKGRFTISRDN | |
| | SKNTLYLQMNSLRAEDTAVYYCVKGPLQEPPYDYGMDVWGQGTTVTVSS | |
| 232 | DIVLTQSPASLAVSLGERATINCRASESVSVIGAHLIHWYQQKPGQPPKL | Anti-BCMA scFv |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSS | |
| 233 | DIVLTQSPASLAVSLGERATINCRASESVSVIGAHLIHWYQQKPGQPPKL | Anti-BCMA scFv |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSS | |
| 234 | DIVLTQSPASLAVSLGERATINCRASESVSVIGAHLIHWYQQKPGQPPKL | Anti-BCMA scFv |
| | LIYLASNLETGVPARFSGSGSGTDFTLTISSLQAEDAAIYYCLQSRIFPR | |
| | TFGQGTKLEIKGSTSGSGKPGSGEGSTKGQVQLVQSGSELKKPGASVKVS | |
| | CKASGYTFTDYSINWVRQAPGQGLEWMGWINTETREPAYAYDFRGRFVFS | |
| | LDTSVSTAYLQISSLKAEDTAVYYCARDYSYAMDYWGQGTLVTVSS | |
| 235 | EVQLVESGGGLVQPGGSLRLSCAVSGFALSNHGMSWVRRAPGKGLEWVSG | Anti-BCMA scFv |
| | IVYSGSTYYAASVKGRFTISRDNSRNTLYLQMNSLRPEDTAIYYCSAHGG | |
| | ESDVWGQGTTVTVSSASGGGGSGGRASGGGGSDIQLTQSPSSLSASVGDR | |
| | VTITCRASQSISSYLNWYQQKPGKAPKLLIYAASSLQSGVPSRFSGSGSG | |
| | TDFTLTISSLQPEDFATYYCQQSYSTPYTFGQGTKVEIK | |
| 236 | QVQLVESGGGLVQPGRSLRLSCAASGFTFSNYAMSWVRQAPGKGLGWVSG | Anti-BCMA scFv |
| | ISRSGENTYYADSVKGRFTISRDNSKNTLYLQMNSLRDEDTAVYYCARSP | |
| | AHYYGGMDVWGQGTTVTVSSASGGGGSGGRASGGGGSDIVLTQSPGTLSL | |
| | SPGERATLSCRASQSISSSFLAWYQQKPGQAPRLLIYGASRRATGIPDRF | |
| | SGSGSGTDFTLTISRLEPEDSAVYYCQQYHSSPSWTFGQGTKLEIK | |
| 237 | QVQLVESGGGLVQPGGSLRLSCAVSGFALSNHGMSWVRRAPGKGLEWVSG | Anti-BCMA scFv |
| | IVYSGSTYYAASVKGRFTISRDNSRNTLYLQMNSLRPEDTAIYYCSAHGG | |
| | ESDVWGQGTTVTVSSASGGGGSGGRASGGGGSDIRLTQSPSPLSASVGDR | |
| | VTITCQASEDINKFLNWYHQTPGKAPKLLIYDASTLQTGVPSRFSGSGSG | |
| | TDFTLTINSLQPEDIGTYYCQQYESLPLTFGGGTKVEIK | |
| 238 | EVQLVESGGGLVQPGGSLRLSCAVSGFALSNHGMSWVRRAPGKGLEWVSG | Anti-BCMA scFv |
| | IVYSGSTYYAASVKGRFTISRDNSRNTLYLQMNSLRPEDTAIYYCSAHGG | |
| | ESDVWGQGTTVTVSSASGGGGSGGRASGGGGSEIVLTQSPGTLSLSPGER | |
| | ATLSCRASQSIGSSSLAWYQQKPGQAPRLLMYGASSRASGIPDRFSGSGS | |
| | GTDFTLTISRLEPEDFAVYYCQQYAGSPPFTFGQGTKVEIK | |
| 239 | QIQLVQSGPELKKPGETVKISCKASGYTFTDYSINWVKRAPGKGLKWMGW | Anti-BCMA scFv |
| | INTETREPAYAYDFRGRFAFSLETSASTAYLQINNLKYEDTATYFCALDY | |
| | SYAMDYWGQGTSVTVSSGGGGSGGGGSGGGGSDIVLTQSPASLAMSLGKR | |
| | ATISCRASESVSVIGAHLIHWYQQKPGQPPKLLIYLASNLETGVPARFSG | |
| | SGSGTDFTLTIDPVEEDDVAIYSCLQSRIFPRTFGGGTKLEIK | |
| 240 | QIQLVQSGPELKKPGETVKISCKASGYTFRHYSMNWVKQAPGKGLKWMGR | Anti-BCMA scFv |
| | INTESGVPIYADDFKGRFAFSVETSASTAYLVINNLKDEDTASYFCSNDY | |
| | LYSLDFWGQGTALTVSSGGGGSGGGGSGGGGSDIVLTQSPPSLAMSLGKR | |
| | ATISCRASESVTILGSHLIYWYQQKPGQPPTLLIQLASNVQTGVPARFSG | |
| | SGSRTDFTLTIDPVEEDDVAVYYCLQSRTIPRTFGGGTKLEIK | |
| 241 | QIQLVQSGPELKKPGETVKISCKASGYTFTHYSMNWVKQAPGKGLKWMGR | Anti-BCMA scFv |
| | INTETGEPLYADDFKGRFAFSLETSASTAYLVINNLKNEDTATFFCSNDY | |
| | LYSCDYWGQGTTLTVSSGGGGSGGGGSGGGGSDIVLTQSPPSLAMSLGKR | |
| | ATISCRASESVTILGSHLIYWYQQKPGQPPTLLIQLASNVQTGVPARFSG | |
| | SGSRTDFTLTIDPVEEDDVAVYYCLQSRTIPRTFGGGTKLEIK | |
| 242 | QVQLVQSGAEVKKPGASVKVSCKASGYSFPDYYINWVRQAPGQGLEWMGW | Anti-BCMA scFv |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIK | |
| 243 | QVQLVQSGAEVKKPGASVKVSCKASGYSFPDYYINWVRQAPGQGLEWMGW | Anti-BCMA scFv |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIK | |
| 244 | QVQLVQSGAEVKKPGASVKVSCKASGYSFPDYYINWVRQAPGQGLEWMGW | Anti-BCMA scFv |
| | IYFASGNSEYNQKFTGRVTMTRDTSINTAYMELSSLTSEDTAVYFCASLY | |
| | DYDWYFDVWGQGTMVTVSSGGGGSGGGGSGGGGSDIVMTQTPLSLSVTPG | |
| | QPASISCKSSQSLVHSNGNTYLHWYLQKPGQSPQLLIYKVSNRFSGVPDR | |
| | FSGSGSGTDFTLKISRVEAEDVGIYYCSQSSIYPWTFGQGTKLEIK | |
| 245 | QVQLVQSGAEVKKPGASVKVSCKASGYSFPDYYINWVRQAPGQGLEWMGW | Anti-BCMA scFv |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIK | |
| 246 | QVQLVQSGAEVKKPGASVKVSCKASGYSFPDYYINWVRQAPGQGLEWMGW | Anti-BCMA scFv |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIK | |
| 247 | QVQLVQSGAEVKKPGASVKVSCKASGYSFPDYYINWVRQAPGQGLEWMGW | Anti-BCMA scFv |
| | IYFASGNSEYNQKFTGRVTMTRDTSSSTAYMELSSLRSEDTAVYFCASLY | |
| | DYDWYFDVWGQGTMVTVSSGGGGSGGGGSGGGGSDIVMTQTPLSLSVTPG | |
| | EPASISCKSSQSLVHSNGNTYLHWYLQKPGQSPQLLIYKVSNRFSGVPDR | |
| | FSGSGSGADFTLKISRVEAEDVGVYYCAETSHVPWTFGQGTKLEIK | |
| 248 | RASQDISKYLN | CDR L1 |
| 249 | SRLHSGV | CDR L2 |
| 250 | GNTLPYTFG | CDR L3 |
| 251 | DYGVS | CDR H1 |
| 252 | VIWGSETTYYNSALKS | CDR H2 |
| 253 | YAMDYWG | CDR H3 |
| 254 | EVKLQESGPGLVAPSQSLSVTCTVSGVSLPDYGVSWIRQPPRKGLEWLGV | VH |
| | IWGSETTYYNSALKSRLTIIKDNSKSQVFLKMNSLQTDDTAIYYCAKHYY | |
| | YGGSYAMDYWGQGTSVTVSS | |
| 255 | DIQMTQTTSSLSASLGDRVTISCRASQDISKYLNWYQQKPDGTVKLLIYH | VL |
| | TSRLHSGVPSRFSGSGSGTDYSLTISNLEQEDIATYFCQQGNTLPYTFGG | |
| | GTKLEIT | |
| 256 | DIQMTQTTSSLSASLGDRVTISCRASQDISKYLNWYQQKPDGTVKLLIYH | scFv |
| | TSRLHSGVPSRFSGSGSGTDYSLTISNLEQEDIATYFCQQGNTLPYTFGG | |
| | GTKLEITGSTSGSGKPGSGEGSTKGEVKLQESGPGLVAPSQSLSVTCTVS | |
| | GVSLPDYGVSWIRQPPRKGLEWLGVIWGSETTYYNSALKSRLTIIKDNSK | |
| | SQVFLKMNSLQTDDTAIYYCAKHYYYGGSYAMDYWGQGTSVTVSS | |
| 257 | KASQNVGTNVA | CDR L1 |
| 258 | SATYRNS | CDR L2 |
| 259 | QQYNRYPYT | CDR L3 |
| 260 | SYWMN | CDR H1 |
| 261 | QIYPGDGDTNYNGKFKG | CDR H2 |
| 262 | KTISSVVDFYFDY | CDR H3 |
| 263 | EVKLQQSGAELVRPGSSVKISCKASGYAFSSYWMNWVKQRPGQGLEWIGQ | VH |
| | IYPGDGDTNYNGKFKGQATLTADKSSSTAYMQLSGLTSEDSAVYFCARKT | |
| | ISSVVDFYFDYWGQGTTVTVSS | |
| 264 | DIELTQSPKFMSTSVGDRVSVTCKASQNVGTNVAWYQQKPGQSPKPLIYS | VL |
| | ATYRNSGVPDRFTGSGSGTDFTLTITNVQSKDLADYFCQQYNRYPYTSGG | |
| | GTKLEIKR | |
| 265 | EVKLQQSGAELVRPGSSVKISCKASGYAFSSYWMNWVKQRPGQGLEWIGQ | scFv |
| | IYPGDGDTNYNGKFKGQATLTADKSSSTAYMQLSGLTSEDSAVYFCARKT | |
| | ISSVVDFYFDYWGQGTTVTVSSGGGGSGGGGSGGGGSDIELTQSPKFMST | |
| | SVGDRVSVTCKASQNVGTNVAWYQQKPGQSPKPLIYSATYRNSGVPDRFT | |
| | GSGSGTDFTLTITNVQSKDLADYFCQQYNRYPYTSGGGTKLEIKR | |
| 266 | HYYYGGSYAMDY | HC-CDR3 |
| 267 | HTSRLHS | LC-CDR2 |
| 268 | QQGNTLPYT | LC-CDR3 |
| 269 | gacatccagatgacccagaccacctccagcctgagcgccagcctgggcga | Sequence |
| | ccgggtgaccatcagctgccgggccagccaggacatcagcaagtacctga | encoding scFv |
| | actggtatcagcagaagcccgacggcaccgtcaagctgctgatctaccac | |
| | accagccggctgcacagcggcgtgcccagccggtttagcggcagcggctc | |
| | cggcaccgactacagcctgaccatctccaacctggaacaggaagatatcg | |
| | ccacctacttttgccagcagggcaacacactgccctacacctttggcggc | |
| | ggaacaaagctggaaatcaccggcagcacctccggcagcggcaagcctgg | |
| | cagcggcgagggcagcaccaagggcgaggtgaagctgcaggaaagcggcc | |
| | ctggcctggtggcccccagccagagcctgagcgtgacctgcaccgtgagc | |
| | ggcgtgagcctgcccgactacggcgtgagctggatccggcagccccccag | |
| | gaagggcctggaatggctgggcgtgatctggggcagcgagaccacctact | |
| | acaacagcgccctgaagagccggctgaccatcatcaaggacaacagcaag | |
| | agccaggtgttcctgaagatgaacagcctgcagaccgacgacaccgccat | |
| | ctactactgcgccaagcactactactacggcggcagctacgccatggact | |
| | actggggccagggcaccagcgtgaccgtgagcagc | |
| 270 | SFAMS | CDR-H1 |
| 271 | AISGSGGGTYYADSVKG | CDR-H2 |
| 272 | DKILWFGEPVFDY | CDR-H3 |
| 273 | RASQSVSSYLA | CDR-L1 |
| 274 | DASNRAT | CDR-L2 |
| 275 | FGQGTKVEIKRTV | CDR-L3 |
| 276 | EVQLLESGGGLVQPGGSLRLSCAVSGFTFNSFAMSWVRQAPGKGLEWVSA | VH |
| | ISGSGGGTYYADSVKGRFTISRDNSKNTLYLQMNSLRAEDTAVYFCAKDK | |
| | ILWFGEPVFDYWGQGTLVTVSS | |
| 277 | EIVLTQSPATLSLSPGERATLSCRASQSVSSYLAWYQQKPGQAPRLLIYD | VL |
| | ASNRATGIPARFSGSGSGTDFTLTISSLEPEDFAVYYCQQRSNWPPTFGQ | |
| | GTKVEIKRTV | |
| 278 | DYWMQ | CDR-H1 |
| 279 | TIYPGDGDTGYAQKFQG | CDR-H2 |
| 280 | GDYYGSNSLDY | CDR-H3 |
| 281 | KASQDVSTVVA | CDR-L1 |
| 282 | SASYRYI | CDR-L2 |
| 282 | QQHYSPPYT | CDR-L3 |
| 284 | QVQLVQSGAEVAKPGTSVKLSCKASGYTFTDYWMQWVKQRPGQGLEWIG | VH |
| | TIYPGDGDTGYAQKFQGKATLTADKSSKTVYMHLSSLASEDSAVYYCAR | |
| | GDYYGSNSLDYWGQGTSVTVSS | |
| 285 | DIVMTQSHLSMSTSLGDPVSITCKASQDVSTVVAWYQQKPGQSPRRLIY | VL |
| | SASYRYIGVPDRFTGSGAGTDFTFTISSVQAEDLAVYYCQQHYSPPYTFG | |
| | GGTKLEIKRT | |

What is claimed:

1. A method of treating a cancer, comprising:
(a) selecting a subject having a cancer for treatment with a subsequent therapy for treating the cancer, wherein the subject was previously administered a T cell therapy for treating the cancer and a prior therapy for treating the cancer, and wherein:
(i) the subject was administered the T cell therapy at a time when the subject had relapsed following treatment with, or was refractory to, the prior therapy;
(ii) following administration of the T cell therapy, the subject achieves minimum residual disease (MRD) negative status; and
(iii) after the subject achieving MRD negative status, the cancer progresses in the subject; and
(b) administering the subsequent therapy to the subject, wherein the prior therapy and the subsequent therapy are of the same class of therapy.

2. A method of selecting a subject having a cancer in which the cancer is re-sensitized to a class of therapy, comprising:
(a) administering a T cell therapy to a subject having a cancer at a time when the subject has relapsed following treatment with, or is refractory to, a prior therapy for treating the cancer; and
(b) selecting the subject for treatment with a subsequent therapy for treating the cancer, wherein the subject is selected for treatment with the subsequent therapy if:
(i) following administration of the T cell therapy, the subject achieves minimum residual disease (MRD) negative status; and
(ii) after the subject achieves MRD negative status, the cancer progresses in the subject, wherein the prior therapy and the subsequent therapy are of the same class of therapy.

3. The method of claim 2, further comprising (c) administering the subsequent therapy to the subject.

4. A method of treating a cancer, comprising:
(a) selecting a subject having a cancer for treatment with a subsequent therapy for treating the cancer, wherein the subject was previously administered a T cell therapy for treating the cancer and a prior therapy for treating the cancer, and wherein:
(i) the subject was administered the T cell therapy at a time when the subject had relapsed following treatment with, or was refractory to, the prior therapy;
(ii) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
(iii) following administration of the T cell therapy, cells of the cancer do not comprise at least one of the high risk features that the cells of the cancer comprised prior to administration of the T cell therapy; and
(b) administering the subsequent therapy to the subject, wherein the prior therapy and the subsequent therapy are of the same class of therapy.

5. A method of selecting a subject having a cancer in which the cancer is re-sensitized to a class of therapy, comprising:
(a) administering a T cell therapy to a subject having a cancer at a time when the subject has relapsed following treatment with, or is refractory to, a prior therapy for treating the cancer; and
(b) selecting the subject for treatment with a subsequent therapy for treating the cancer, wherein the subject is selected for treatment with the subsequent therapy if:
(i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
(ii) following administration of the T cell therapy, cells of the cancer do not comprise at least one of the high risk features that the cells of the cancer comprised prior to administration of the T cell therapy,
wherein the prior therapy and the subsequent therapy are of the same class of therapy.

6. The method of claim 5, further comprising (c) administering the subsequent therapy to the subject.

7. A method of treating a cancer, comprising:
(a) administering to a subject having a cancer a T cell therapy for treating the cancer at a time when the subject has relapsed following treatment with, or is refractory to, a prior therapy for treating the cancer; and
(b) administering a subsequent therapy for treating the cancer to the subject, wherein the prior therapy and the subsequent therapy are of the same class of therapy.

8. A method of re-sensitizing a cancer in a subject, comprising:
(a) administering to a subject having a cancer a T cell therapy for treating the cancer at a time when the subject has relapsed following treatment with, or is refractory to, a prior therapy for treating the cancer; and
(b) administering a subsequent therapy for treating the cancer to the subject, wherein the prior therapy and the subsequent therapy are of the same class of therapy.

9. The method of claim 7 or claim 8, further comprising, prior to (b), selecting the subject for treatment with the subsequent therapy, wherein the subject is selected for treatment with the subsequent therapy if:
(i) following administration of the T cell therapy, the subject achieves minimum residual disease (MRD) negative status; and
(ii) subsequent to the subject achieving MRD negative status, the cancer progresses in the subject.

10. The method of any of claims 7-9, wherein:
(i) prior to administration of the T cell therapy, cells of the cancer comprise one or more high risk feature(s) selected from among the group consisting of amplification of the long arm of chromosome 1 (amp1q), MDMS8 gene signature, a cereblon (CRBN) mutation, biallelic p53 inactivation, high cancer clonal fraction del17p, and t(4,14); and
(ii) following administration of the T cell therapy, cells of the cancer do not comprise at least one of the high risk feature(s) that the cells of the cancer comprised prior to administration of the T cell therapy.

11. The method of any of claims 1-3, 9, and 10, wherein, within about 1 month, about 2 months, about 3 months, about 6 months, or about 12 months of administration of the T cell therapy, the subject achieves MRD negative status.

12. The method of any of claims 4-6, 10, and 11, wherein, within about 1 month, about 2 months, about 3 months, about 6 months, or about 12 months of administration of the T cell therapy, the cells of the cancer do not comprise at least one of the high risk feature(s) that the cells of the cancer comprised prior to administration of the T cell therapy.

13. The method of any of claims 1-12, wherein, prior to administration of the T cell therapy, cells of the cancer comprise a CRBN mutation.

14. The method of any of claims 1-13, wherein, within about 1 month, about 2 months, about 3 months, about 6 months, or about 12 months of administration of the T cell therapy, cells of the cancer do not comprise a CRBN mutation.

15. The method of any of claims 4-6 and 12-14, wherein the CRBN mutation is in exon 10 of the CRBN gene.

16. The method of any of claims 4-6 and 12-15, wherein the CRBN mutation reduces or inhibits binding of thalidomide to the CRBN protein.

17. The method of any of claims 1-16, wherein the cancer is a B cell malignancy.

18. The method of any of claims 1-17, wherein the cancer is a multiple myeloma (MM).

19. The method of claim 18, wherein the MM is a relapsed/refractory (R/R) MM.

20. The method of any of claims 1-19, wherein the class of therapy is immunomodulatory drugs.

21. The method of any of claims 1-20, wherein the prior therapy and the subsequent therapy both bind the cereblon (CRBN) protein.

22. The method of any of claims 1-21, wherein the prior therapy and the subsequent therapy both induce degradation of Ailos and/or Ikaros.

23. The method of any of claims 1-22, wherein the prior therapy is selected from among the group consisting of: thalidomide, lenalidomide, pomalidomide, iberdomide, CC-92480, CC-99282, CC-91633, and CC-90009.

24. The method of any of claims 1-23, wherein the subsequent therapy is selected from among the group consisting of: thalidomide, lenalidomide, pomalidomide, iberdomide, CC-92480, CC-99282, CC-91633, and CC-90009.

25. The method of any of claims 1-19, wherein the class of therapy is proteasome inhibitors.

26. The method of any of claims 1-19 and 25, wherein the prior therapy is selected from among the group consisting of: bortezomib, carfilzomib and ixazomib.

27. The method of any of claims 1-19, 25, and 26, wherein the subsequent therapy is selected from among the group consisting of: bortezomib, carfilzomib and ixazomib.

28. The method of any of claims 1-19, wherein the class of therapy is anti-CD38 antibodies.

29. The method of any of claims 1-19 and 28, wherein the prior therapy is daratumumab or isatuximab.

30. The method of any of claims 1-19, 28, and 29, wherein the subsequent therapy is daratumumab or isatuximab.

31. The method of any of claims 1-17, wherein the cancer is a leukemia or a lymphoma.

32. The method of claim 31, wherein the leukemia or the lymphoma is selected from the group consisting of: acute lymphoblastic leukemia (ALL), adult ALL, chronic lymphoblastic leukemia (CLL), small lymphocytic lymphoma (SLL), non-Hodgkin lymphoma (NHL), and large B cell lymphoma (LBCL).

33. The method of any of claims 1-19, 31, and 32, wherein the class of therapy is inhibitors of Bruton's tyrosine kinase (BTK).

34. The method of any of claims 1-19 and 31-33, wherein the prior therapy is selected from among the group consisting of: ibrutinib, acalabrutinib, zanubrutinib, evobrutinib, tirabrutinib, and SNS-062.

35. The method of any of claims 1-19 and 31-34, wherein the subsequent therapy is selected from among the group consisting of: ibrutinib, acalabrutinib, zanubrutinib, evobrutinib, tirabrutinib, and SNS-062.

36. The method of any of claims 1-19, 31, and 32, wherein the class of therapy is inhibitors of BCL-2.

37. The method of any of claims 1-19, 31, 32, and 36, wherein the prior therapy is selected from among the group consisting of: venetoclax, navitoclax, ABT737, maritoclax, obatoclax, and clitocine.

38. The method of any of claims 1-19, 31, 32, 36, and 37, wherein the subsequent therapy is selected from among the group consisting of: venetoclax, navitoclax, ABT737, maritoclax, obatoclax, and clitocine.

39. The method of any of claims 1-38, wherein the subsequent therapy is a maintenance therapy.

40. The method of any of claims 1-39, wherein the T cell therapy comprises a dose of T cells expressing a recombinant receptor.

41. The method of claim 40, wherein the recombinant receptor is a T cell receptor (TCR) or a chimeric antigen receptor (CAR).

42. The method of claim 40 or claim 41, wherein the recombinant receptor is a CAR.

43. The method of claim 42, wherein the CAR comprises an extracellular antigen binding domain that binds to the antigen, a transmembrane domain, and an intracellular signaling region.

44. The method of claim 43, wherein the intracellular signaling region comprises an intracellular signaling domain of a CD3-zeta (CD3ζ) chain and a costimulatory signaling region.

45. The method of claim 44, wherein the costimulatory signaling region comprises an intracellular signaling domain of CD28, 4-1BB, or ICOS.

46. The method of claim 44 or claim 45, wherein the costimulatory signaling region comprises an intracellular signaling domain of 4-1BB.

47. The method of any one of claims 43-46, wherein the transmembrane domain is or comprises a transmembrane domain from CD28 or CD8, optionally human CD28 or CD8.

48. The method of any one of claims 43-47, wherein the CAR further comprises an extracellular spacer between the extracellular antigen binding domain and the transmembrane domain.

49. The method of claim 48, wherein the spacer is from CD8, optionally wherein the spacer is a CD8α hinge.

50. The method of claim 48 or claim 49, wherein the transmembrane domain and the spacer are from CD8.

51. The method of any of claims 43-50, wherein the extracellular antigen binding domain binds to B cell maturation antigen (BCMA).

52. The method of any of claims 43-51, wherein the extracellular antigen-binding domain comprises a variable heavy chain (V_{H}) region and, optionally, a variable light chain (V_{L}) region.

53. The method of claim 52, wherein:
the V_{H} region comprises a CDR-H1, a CDR-H2, and a CDR-H3 comprising the amino acid sequences set forth in SEQ ID NOS: 189, 190, and 191, respectively; and the V_{L} region comprises a CDR-L1, a CDR-L2, and a CDR-L3 comprising the amino acid sequences set forth in SEQ ID NOS: 192, 193, and 194, respectively; or
the V_{H} region comprises a CDR-H1, a CDR-H2, and a CDR-H3 comprising the amino acid sequences set forth in SEQ ID NOS: 173, 174 and 175, respectively; and the V_{L} region comprises a CDR-L1, a CDR-L2, and a CDR-L3 comprising the amino acid sequences set forth in SEQ ID NOS: 183, 184 and 185, respectively.

54. The method of claim 52 or claim 53, wherein:
the V_{H} region comprises an amino acid sequence set forth in SEQ ID NO: 18 and the V_{L} region comprises the amino acid sequence set forth in SEQ ID NO: 19;
or the V_{H} region comprises an amino acid sequence set forth in SEQ ID NO: 24, and the V_{L} region comprises the amino acid sequence set forth in SEQ ID NO: 25.

55. The method of any one of claims 43-54, wherein the extracellular antigen-binding domain is a single chain variable fragment (scFv).

56. The method of claim 55, wherein the scFv comprises the amino acid sequence set forth in SEQ ID NO: 213 or SEQ ID NO: 188.

57. The method of any one of claims 42-56, wherein the CAR comprises the amino acid sequence set forth in SEQ ID NO: 116 or SEQ ID NO: 124.

58. The method of any one of claims 42-57, wherein the CAR is encoded by the polynucleotide sequence set forth in SEQ ID NO: 214.

59. The method of any one of claims 40-58, wherein the dose of T cells comprises: idecabtagene vicleucel cells; bb21217 cells; orvacabtagene autoleucel cells; CT103A cells; ciltacabtagene autoleucel cells; KITE585 cells; CT053 cells; BCMA-CS1 cCAR (BC1cCAR) cells; P-BCMA-101 cells; P-BCMA-ALLO1 cells; C-CAR088 cells; Descartes-08 cells; PBCAR269A cells; ALLO-715 cells; PHE885 cells; AUTO8 cells; CTX120 cells; CB-011 cells; ALLO-605 (TuboCAR/MM) cells; pCDCAR1 (TriCAR-Z136) cells; or GC012F cells.

60. The method of any one of claims 40-59, wherein the dose of T cells comprises idecabtagene vicleucel cells.

61. The method of any of claims 43-50, wherein the extracellular antigen binding domain binds to G protein-coupled receptor, class C group 5 member D (GPRC5D).

62. The method of any of claims 43-50, wherein the extracellular antigen binding domain binds to CD19.

63. The method of claim 62, wherein the extracellular antigen-binding domain comprises a variable heavy chain (V_{H}) region and, optionally, a variable light chain (V_{L}) region.

64. The method of claim 63, wherein:
the V_{H} region comprises a CDR-H1, a CDR-H2, and a CDR-H3 comprising the amino acid sequences set forth in SEQ ID NOS: 251, 252, and 253, respectively; and the V_{L} region comprises a CDR-L1, a CDR-L2, and a CDR-L3 comprising the amino acid sequences set forth in SEQ ID NOS: 248, 249, and 250, respectively; or
the V_{H} region comprises a CDR-H1, a CDR-H2, and a CDR-H3 comprising the amino acid sequences set forth in SEQ ID NOS: 260, 261, and 262, respectively; and the V_{L} region comprises a CDR-L1, a CDR-L2, and a CDR-L3 comprising the amino acid sequences set forth in SEQ ID NOS: 257, 258, and 259, respectively.

65. The method of claim 63 or claim 64, wherein:
the V_{H} region comprises an amino acid sequence set forth in SEQ ID NO: 254 and the V_{L} region comprises the amino acid sequence set forth in SEQ ID NO: 255; or
the V_{H} region comprises an amino acid sequence set forth in SEQ ID NO: 263 and the V_{L} region comprises the amino acid sequence set forth in SEQ ID NO: 264.

66. The method of any one of claims 62-65, wherein the extracellular antigen-binding domain is a single chain variable fragment (scFv).

67. The method of claim 66, wherein the scFv comprises the amino acid sequence set forth in SEQ ID NO: 256 or SEQ ID NO: 265.

68. The method of any one of claims 40-50 and 62-67, wherein the dose of T cells comprises: lisocabtagene maraleucel cells; tisagenlecleucel cells; axicabtagene ciloleucel cells; or brexucabtagene autoleucel cells.

69. The method of any of claims 40-68, wherein the dose of T cells comprises CD3⁺ CAR-expressing T cells.

70. The method of any of claims 40-69, wherein the dose of T cells comprises a combination of CD4⁺ CAR-expressing T cells and CD8⁺ CAR-expressing T cells.

71. The method of claim 70, wherein the ratio of CD4⁺ CAR-expressing T cells to CD8⁺ CAR-expressing T cells in the dose of T cells is approximately 1:1 or is between approximately 1:3 and approximately 3:1.

72. The method of any of claims 40-71, wherein, in the dose of T cells:
the percentage of naive-like T cells and/or central memory T cells is greater than or greater than about 60% of the total T cells in the dose, optionally greater than or greater than about 65%, 70%, 80%, 90% or 95%;
the percentage of naive-like T cells and/or central memory T cells is greater than or greater than about 40% of the total CD4+ T cells in the dose, optionally greater than or greater than about 50%, 60%, 70%, 80%, 90% or 95%; or
the percentage of naive-like T cells and/or central memory T cells is greater than or greater than about 40% of the total CD8+ T cells in the dose, optionally greater than or greater than about 50%, 60%, 70%, 80%, 90% or 95%.

73. The method of claim 72, wherein the naive-like T cells are CCR7+CD45RA+, CD27+CCR7+, or CD62L−CCR7+.

74. The method of any one of claims 40-72, wherein the dose of T cells comprises between about 0.5×10⁶ and about 6×10⁸ CAR-positive T cells.

75. The method of any one of claims 40-74, wherein the dose of T cells comprises between about 1×10⁸ and about 6×10⁸ CAR-positive T cells.

76. The method of any one of claims 40-75, wherein the dose of T cells comprises between about 1.5×10⁸ and about 4.5×10⁸ CAR-positive T cells.

77. The method of any one of claims 40-76, wherein the dose of T cells comprises about 1.5×10⁸, 3×10⁸, or about 4.5×10⁸ CAR-positive T cells.

78. The method of any one of claims 40-74, wherein the dose of T cells comprises between about 0.5×10⁶ and about 10×10⁶ CAR-positive T cells.

79. The method of any one of claims 40-78, wherein the cells of the dose of T cells were obtained from the subject.

80. The method of any one of claims 40-79, wherein the cells of the dose of T cells are autologous to the subject.

81. The method of any one of claims 40-78, wherein the cells of the dose of T cells are allogeneic to the subject.

82. The method of any of claims 1-39, wherein the T cell therapy comprises a T cell engager (TCE).

83. The method of claim 82, wherein the TCE is selected from among the group consisting of: a bispecific T cell engager (BiTE), a checkpoint-inhibitory T cell engager (CiTE), a simultaneous multiple interaction T cell engagers (SMITE), and BiTE-expressing CAR T cells (CART.BiTE cells).

84. The method of any of claims 1-83, wherein the method comprises, prior to administration of the T cell therapy, administering a lymphodepleting therapy to the subject.

85. The method of claim 84, wherein the lymphodepleting therapy is completed between 2 and 7 days before the initiation of administration of the T cell therapy.

86. The method of claim 84 or claim 85, wherein the lymphodepleting therapy comprises the administration of fludarabine and/or cyclophosphamide.

87. The method of any of claims 84-86, wherein the lymphodepleting therapy comprises administration of:
(i) cyclophosphamide at about 200-400 mg/m², optionally at or about 300 mg/m², inclusive, and/or fludarabine at about 20-40 mg/m², optionally 30 mg/m², daily for 2-4 days, optionally for 3 days; or
(ii) cyclophosphamide at about 500 mg/m².

88. The method of any one of claims 84-87, wherein:
the lymphodepleting therapy comprises administration of cyclophosphamide at or about 300 mg/m² and fludarabine at about 30 mg/m² daily for 3 days; or
the lymphodepleting therapy comprises administration of cyclophosphamide at or about 500 mg/m² and fludarabine at about 30 mg/m² daily for 3 days.


SEQUENCE LISTING (285 sequences)

SEQ ID NO: 1 - protein, 12 aa, Homo sapiens
  REGION 1..12: note = spacer (IgG4hinge) (aa)
ESKYGPPCPPCP

SEQ ID NO: 2 - protein, 36 aa, Homo sapiens
  REGION 1..36: note = spacer (IgG4hinge) (nt)
GAATCTAAGTACGGACCGCCCTGCCCCCCTTGCCCT

SEQ ID NO: 3 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, synthetic construct
  REGION 1..24: note = T2A
LEGGGEGRGSLLTCGDVEENPGPR

SEQ ID NO: 7 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  REGION 1..27: note = CD28 (amino acids 153-179 of Accession No. P10747)
FWVLVVVGGVLACYSLLVTVAFIIFWV

SEQ ID NO: 9 - protein, 66 aa, Homo sapiens
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  REGION 1..41: note = CD28 (amino acids 180-220 of P10747)
RSKRSRLLHSDYMNMTPRRPGPTRKHYQPYAPPRDFAAYRS

SEQ ID NO: 11 - protein, 41 aa, Homo sapiens
  REGION 1..41: note = CD28 (LL to GG)
RSKRSRGGHSDYMNMTPRRPGPTRKHYQPYAPPRDFAAYRS

SEQ ID NO: 12 - protein, 42 aa, Homo sapiens
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 13 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 29 aa, synthetic construct
  REGION 1..29: note = linker
PGGGSGGGGSGGGGSGGGGSGGGGSGGGG

SEQ ID NO: 17 - protein, 17 aa, synthetic construct
  REGION 1..17: note = linker
GSADDAKKDAAKKDGKS

SEQ ID NO: 18 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 19 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 20 - protein, 122 aa, synthetic construct
  REGION 1..122: note = Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 21 - protein, 107 aa, synthetic construct
  REGION 1..107: note = Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 22 - protein, 116 aa, synthetic construct
  REGION 1..116: note = Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 23 - protein, 111 aa, synthetic construct
  REGION 1..111: note = Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 24 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 25 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 26 - protein, 5 aa, synthetic construct
  REGION 1..5: note = Linker
GGGGS

SEQ ID NO: 27 - protein, 4 aa, synthetic construct
  REGION 1..4: note = Linker
GGGS

SEQ ID NO: 28 - protein, 15 aa, synthetic construct
  REGION 1..15: note = Linker
GGGGSGGGGSGGGGS

SEQ ID NO: 29 - protein, 18 aa, synthetic construct
  REGION 1..18: note = Linker
GSTSGSGKPGSGEGSTKG

SEQ ID NO: 30 - protein, 21 aa, synthetic construct
  REGION 1..21: note = Linker
SRGGGGSGGGGSGGGGSLEMA

SEQ ID NO: 31 - protein, 228 aa, synthetic construct
  REGION 1..228: note = Hinge-CH2-CH3 spacer Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 122 aa, synthetic construct
  REGION 1..122: note = Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 33 - protein, 111 aa, synthetic construct
  REGION 1..111: note = Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 34 - protein, 120 aa, synthetic construct
  REGION 1..120: note = Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 35 - protein, 112 aa, synthetic construct
  REGION 1..112: note = Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 36 - protein, 118 aa, synthetic construct
  REGION 1..118: note = Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 37 - protein, 112 aa, synthetic construct
  REGION 1..112: note = Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 38 - protein, 17 aa, synthetic construct
  REGION 1..17: note = Linker
ASGGGGSGGRASGGGGS

SEQ ID NO: 39 - protein, 21 aa, synthetic construct
  REGION 1..21: note = CD8a signal peptide
MALPVTALLLPLALLLHAARP

SEQ ID NO: 40 - protein, 20 aa, synthetic construct
  REGION 1..20: note = signal peptide
METDTLLLWVLLLWVPGSTG

SEQ ID NO: 41 - protein, 118 aa, synthetic construct
  REGION 1..118: note = Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 42 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 43 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 44 - protein, 112 aa, synthetic construct
  REGION 1..112: note = Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 45 - protein, 116 aa, synthetic construct
  REGION 1..116: note = Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 46 - protein, 106 aa, synthetic construct
  REGION 1..106: note = Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 47 - protein, 122 aa, synthetic construct
  REGION 1..122: note = Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 48 - protein, 107 aa, synthetic construct
  REGION 1..107: note = Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 49 - protein, 119 aa, synthetic construct
  REGION 1..119: note = Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 50 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 51 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 52 - protein, 107 aa, synthetic construct
  REGION 1..107: note = Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 53 - protein, 126 aa, synthetic construct
  REGION 1..126: note = Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 54 - protein, 113 aa, synthetic construct
  REGION 1..113: note = Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 55 - protein, 123 aa, synthetic construct
  REGION 1..123: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 56 - protein, 107 aa, synthetic construct
  REGION 1..107: note = Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 57 - protein, 121 aa, synthetic construct
  REGION 1..121: note = Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 58 - protein, 112 aa, synthetic construct
  REGION 1..112: note = Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 59 - protein, 117 aa, synthetic construct
  REGION 1..117: note = Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 60 - protein, 111 aa, synthetic construct
  REGION 1..111: note = Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 61 - protein, 115 aa, synthetic construct
  REGION 1..115: note = Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 62 - protein, 107 aa, synthetic construct
  REGION 1..107: note = Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 63 - protein, 120 aa, synthetic construct
  REGION 1..120: note = Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 64 - protein, 109 aa, synthetic construct
  REGION 1..109: note = Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 65 - protein, 115 aa, synthetic construct
  REGION 1..115: note = Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 66 - protein, 107 aa, synthetic construct
  REGION 1..107: note = Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 67 - protein, 115 aa, synthetic construct; identical to SEQ ID NO: 61
  REGION 1..115: note = Variable heavy (VH) Anti-BCMA

SEQ ID NO: 68 - protein, 109 aa, synthetic construct
  REGION 1..109: note = Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 69 - protein, 117 aa, synthetic construct
  REGION 1..117: note = Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 70 - protein, 111 aa, synthetic construct
  REGION 1..111: note = Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 71 - protein, 117 aa, synthetic construct
  REGION 1..117: note = Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 72 - protein, 111 aa, synthetic construct
  REGION 1..111: note = Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 73 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 74 - protein, 112 aa, synthetic construct
  REGION 1..112: note = Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 75 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 76 - protein, 112 aa, synthetic construct
  REGION 1..112: note = Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 77 - protein, 122 aa, synthetic construct
  REGION 1..122: note = Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 78 - protein, 39 aa, synthetic construct
  REGION 1..39: note = CD28 spacer
IEVMYPPPYLDNEKSNGTIIHVKGKHLCPSPLFPGPSKP

SEQ ID NO: 79 - protein, 25 aa, synthetic construct
  REGION 1..25: note = CD8a TM
IYIWAPLAGTCGVLLLSLVITLYCN

SEQ ID NO: 80 - protein, 30 aa, synthetic construct
  REGION 1..30: note = CD28 spacer (truncated)
LDNEKSNGTIIHVKGKHLCPSPLFPGPSKP

SEQ ID NO: 81 - protein, 46 aa, synthetic construct
  REGION 1..46: note = CD8a hinge
PTTTPAPRPPTPAPTIASQPLSLRPEACRPAAGGAVHTRGLDFACD

SEQ ID NO: 82 - protein, 45 aa, synthetic construct
  REGION 1..45: note = CD8a hinge
TTTPAPRPPTPAPTIASQPLSLRPEACRPAAGGAVHTRGLDFACD

SEQ ID NO: 83 - protein, 55 aa, synthetic construct
  REGION 1..55: note = CD8a hinge
FVPVFLPAKPTTTPAPRPPTPAPTIASQPLSLRPEACRPAAGGAVHTRGLDFACD

SEQ ID NO: 84 - protein, 39 aa, synthetic construct
  REGION 1..39: note = CTLA4 hinge
DTGLYICKVELMYPPPYYLGIGNGTQIYVIDPEPCPDSD

SEQ ID NO: 85 - protein, 24 aa, synthetic construct
  REGION 1..24: note = CTLA4 TM
FLLWILAAVSSGLFFYSFLLTAVS

SEQ ID NO: 86 - protein, 38 aa, synthetic construct
  REGION 1..38: note = PD-1 hinge
QIKESLRAELRVTERRAEVPTAHPSPSPRPAGQFQTLV

SEQ ID NO: 87 - protein, 21 aa, synthetic construct
  REGION 1..21: note = PD-1 TM
VGVVGGLLGSLVLLVWVLAVI

SEQ ID NO: 88 - protein, 16 aa, synthetic construct
  REGION 1..16: note = Fc(gamma)RIIIa hinge
GLAVSTISSFFPPGYQ

SEQ ID NO: 89 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 457 aa, synthetic construct
  REGION 1..457: note = anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 457 aa, synthetic construct
  REGION 1..457: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 466 aa, synthetic construct
  REGION 1..466: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 466 aa, synthetic construct
  REGION 1..466: note = anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 454 aa, synthetic construct
  REGION 1..454: note = anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 454 aa, synthetic construct
  REGION 1..454: note = anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 461 aa, synthetic construct
  REGION 1..461: note = anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 461 aa, synthetic construct
  REGION 1..461: note = anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 458 aa, synthetic construct
  REGION 1..458: note = anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 458 aa, synthetic construct
  REGION 1..458: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - protein, 461 aa, synthetic construct
  REGION 1..461: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 461 aa, synthetic construct
  REGION 1..461: note = anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - protein, 471 aa, synthetic construct
  REGION 1..471: note = anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 471 aa, synthetic construct
  REGION 1..471: note = anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 462 aa, synthetic construct
  REGION 1..462: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - protein, 462 aa, synthetic construct
  REGION 1..462: note = anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 466 aa, synthetic construct
  REGION 1..466: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 473 aa, synthetic construct
  REGION 1..473: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 470 aa, synthetic construct
  REGION 1..470: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 476 aa, synthetic construct
  REGION 1..476: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 475 aa, synthetic construct
  REGION 1..475: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 482 aa, synthetic construct
  REGION 1..482: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 476 aa, synthetic construct
  REGION 1..476: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - protein, 481 aa, synthetic construct
  REGION 1..481: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 114 - protein, 479 aa, synthetic construct
  REGION 1..479: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 472 aa, synthetic construct
  REGION 1..472: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 472 aa, synthetic construct
  REGION 1..472: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - protein, 472 aa, synthetic construct
  REGION 1..472: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 466 aa, synthetic construct
  REGION 1..466: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - protein, 464 aa, synthetic construct
  REGION 1..464: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 653 aa, synthetic construct
  REGION 1..653: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 650 aa, synthetic construct
  REGION 1..650: note = anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 651 aa, synthetic construct
  REGION 1..651: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 658 aa, synthetic construct
  REGION 1..658: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 653 aa, synthetic construct
  REGION 1..653: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 462 aa, synthetic construct
  REGION 1..462: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 469 aa, synthetic construct
  REGION 1..469: note = anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 462 aa, synthetic construct
  REGION 1..462: note = anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 464 aa, synthetic construct
  REGION 1..464: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 467 aa, synthetic construct
  REGION 1..467: note = anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 472 aa, synthetic construct
  REGION 1..472: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 466 aa, synthetic construct
  REGION 1..466: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 466 aa, synthetic construct
  REGION 1..466: note = anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 466 aa, synthetic construct
  REGION 1..466: note = anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 482 aa, synthetic construct
  REGION 1..482: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 28 aa, synthetic construct
  REGION 1..28: note = CD8a TM
IYIWAPLAGTCGVLLLSLVITLYCNHRN

SEQ ID NO: 143 - protein, 21 aa, synthetic construct
  REGION 1..21: note = CD8a TM
IYIWAPLAGTCGVLLLSLVIT

SEQ ID NO: 144 - protein, 4 aa, synthetic construct
  REGION 1..4: note = linking peptide
RAAA

SEQ ID NO: 145 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 146 - protein, 110 aa, synthetic construct
  REGION 1..110: note = Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 147 - protein, 118 aa, synthetic construct
  REGION 1..118: note = Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 148 - protein, 107 aa, synthetic construct
  REGION 1..107: note = Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 149 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 150 - protein, 108 aa, synthetic construct
  REGION 1..108: note = Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 151 - protein, 122 aa, synthetic construct; identical to SEQ ID NO: 32
  REGION 1..122: note = Variable heavy (VH) Anti-BCMA

SEQ ID NO: 152 - protein, 111 aa, synthetic construct; identical to SEQ ID NO: 33
  REGION 1..111: note = Variable light (VL) Anti-BCMA

SEQ ID NO: 153 - protein, 16 aa, synthetic construct
  REGION 1..16: note = CD33 Signal peptide
MPLLLLLPLLWAGALA

SEQ ID NO: 154 - protein, 18 aa, synthetic construct
  REGION 1..18: note = CD8 alpha signal peptide
MALPVTALLLPLALLLHA

SEQ ID NO: 155 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 156 - protein, 22 aa, synthetic construct
  REGION 1..22: note = GMCSFR alpha chain signal sequence
MLLLVTSLLLCELPHPAFLLIP

SEQ ID NO: 157 - protein, 14 aa, synthetic construct
  REGION 1..14: note = Exemplary IgG Hinge
EVVVKYGPPCPPCP

SEQ ID NO: 158 - skipped

SEQ ID NO: 159 - protein, 15 aa, synthetic construct
  REGION 1..15: note = Exemplary IgG Hinge
EPKSCDKTHTCPPCP

SEQ ID NO: 160 - protein, 12 aa, synthetic construct
  REGION 1..12: note = Exemplary IgG Hinge
ERKCCVECPPCP

SEQ ID NO: 161 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 162 - protein, 12 aa, synthetic construct
  REGION 1..12: note = Exemplary IgG Hinge
ESKYGPPCPSCP

SEQ ID NO: 163 - protein, 12 aa, synthetic construct
  REGION 1..12: note = Exemplary IgG Hinge
ESKYGPPCPPCP

SEQ ID NO: 164 - protein, 9 aa, synthetic construct
  REGION 1..9: note = Exemplary IgG Hinge
YGPPCPPCP

SEQ ID NO: 165 - protein, 10 aa, synthetic construct
  REGION 1..10: note = Exemplary IgG Hinge
KYGPPCPPCP

SEQ ID NO: 166 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 167 - protein, 18 aa, synthetic construct
  REGION 1..18: note = T2A
EGRGSLLTCGDVEENPGP

SEQ ID NO: 168 - protein, 22 aa, synthetic construct
  REGION 1..22: note = P2A
GSGATNFSLLKQAGDVEENPGP

SEQ ID NO: 169 - protein, 19 aa, synthetic construct
  REGION 1..19: note = P2A
ATNFSLLKQAGDVEENPGP

SEQ ID NO: 170 - protein, 20 aa, synthetic construct
  REGION 1..20: note = E2A
QCTNYALLKLAGDVESNPGP

SEQ ID NO: 171 - protein, 22 aa, synthetic construct
  REGION 1..22: note = E2A
VKQTLNFDLLKLAGDVESNPGP

SEQ ID NO: 172 - protein, 290 aa, synthetic construct
  REGION 1..290: note = BCMA-Fc fusion polypeptide
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 5 aa, synthetic construct
  REGION 1..5: note = CDR-H1
DYYVY

SEQ ID NO: 174 - protein, 17 aa, synthetic construct
  REGION 1..17: note = CDR-H2
WINPNSGGTNYAQKFQG

SEQ ID NO: 175 - protein, 9 aa, synthetic construct
  REGION 1..9: note = CDR-H3
SQRDGYMDY

SEQ ID NO: 176 - protein, 7 aa, synthetic construct
  REGION 1..7: note = CDR-H1
GYTFIDY

SEQ ID NO: 177 - protein, 6 aa, synthetic construct
  REGION 1..6: note = CDR-H2
NPNSGG

SEQ ID NO: 178 - protein, 10 aa, synthetic construct
  REGION 1..10: note = CDR-H1
GYTFIDYYVY

SEQ ID NO: 179 - protein, 10 aa, synthetic construct
  REGION 1..10: note = CDR-H2
WINPNSGGTN

SEQ ID NO: 180 - protein, 8 aa, synthetic construct
  REGION 1..8: note = CDR-H1
GYTFIDYY

SEQ ID NO: 181 - protein, 8 aa, synthetic construct
  REGION 1..8: note = CDR-H2
INPNSGGT

SEQ ID NO: 182 - protein, 11 aa, synthetic construct
  REGION 1..11: note = CDR-H3
ARSQRDGYMDY

SEQ ID NO: 183 - protein, 8 aa, synthetic construct
  REGION 1..8: note = CDR-L1
TGTSSDVG

SEQ ID NO: 184 - protein, 7 aa, synthetic construct
  REGION 1..7: note = CDR-L2
EDSKRPS

SEQ ID NO: 185 - protein, 10 aa, synthetic construct
  REGION 1..10: note = CDR-L3
SSNTRSSTLV

SEQ ID NO: 186 - protein, 9 aa, synthetic construct
  REGION 1..9: note = CDR-L1
ISCTGTSSD

SEQ ID NO: 187 - skipped

SEQ ID NO: 188 - protein, 244 aa, synthetic construct
  REGION 1..244: note = anti-BCMA scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 189 - protein, 5 aa, synthetic construct
  REGION 1..5: note = CDR-H1
DYSIN

SEQ ID NO: 190 - protein, 17 aa, synthetic construct
  REGION 1..17: note = CDR-H2
WINTETREPAYAYDFRG

SEQ ID NO: 191 - protein, 8 aa, synthetic construct
  REGION 1..8: note = CDR-H3
DYSYAMDY

SEQ ID NO: 192 - protein, 15 aa, synthetic construct
  REGION 1..15: note = CDR-L1
RASESVTILGSHLIH

SEQ ID NO: 193 - protein, 7 aa, synthetic construct
  REGION 1..7: note = CDR-L2
LASNVQT

SEQ ID NO: 194 - protein, 9 aa, synthetic construct
  REGION 1..9: note = CDR-L3
LQSRTIPRT

SEQ ID NO: 195 - protein, 7 aa, synthetic construct
  REGION 1..7: note = CDR-H1
GYTFTDY

SEQ ID NO: 196 - protein, 6 aa, synthetic construct
  REGION 1..6: note = CDR-H2
NTETRE

SEQ ID NO: 197 - protein, 8 aa, synthetic construct
  REGION 1..8: note = CDR-H3
DYSYAMDY

SEQ ID NO: 198 - protein, 15 aa, synthetic construct
  REGION 1..15: note = CDR-L1
RASESVTILGSHLIH

SEQ ID NO: 199 - protein, 7 aa, synthetic construct
  REGION 1..7: note = CDR-L2
LASNVQT

SEQ ID NO: 200 - protein, 9 aa, synthetic construct
  REGION 1..9: note = CDR-L3
LQSRTIPRT

SEQ ID NO: 201 - protein, 10 aa, synthetic construct
  REGION 1..10: note = CDR-H1
GYTFTDYSIN

SEQ ID NO: 202 - protein, 10 aa, synthetic construct
  REGION 1..10: note = CDR-H2
WINTETREPA

SEQ ID NO: 203 - protein, 8 aa, synthetic construct
  REGION 1..8: note = CDR-H3
DYSYAMDY

SEQ ID NO: 204 - protein, 15 aa, synthetic construct
  REGION 1..15: note = CDR-L1
RASESVTILGSHLIH

SEQ ID NO: 205 - protein, 7 aa, synthetic construct
  REGION 1..7: note = CDR-L2
LASNVQT

SEQ ID NO: 206 - protein, 9 aa, synthetic construct
  REGION 1..9: note = CDR-L3
LQSRTIPRT

SEQ ID NO: 207 - protein, 8 aa, synthetic construct
  REGION 1..8: note = CDR-H1
GYTFTDYS

SEQ ID NO: 208 - protein, 8 aa, synthetic construct
  REGION 1..8: note = CDR-H2
INTETREP

SEQ ID NO: 209 - protein, 10 aa, synthetic construct
  REGION 1..10: note = CDR-H3
ALDYSYAMDY

SEQ ID NO: 210 - protein, 10 aa, synthetic construct
  REGION 1..10: note = CDR-L1
ESVTILGSHL

SEQ ID NO: 211 - skipped

SEQ ID NO: 212 - protein, 9 aa, synthetic construct
  REGION 1..9: note = CDR-L3
LQSRTIPRT

SEQ ID NO: 213 - protein, 246 aa, synthetic construct
  REGION 1..246: note = scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 214 - DNA, 1485 bp, synthetic construct
  misc_feature 1..1485: note = BCMA CAR (nt)
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 215 - protein, 184 aa, Homo sapiens
  REGION 1..184: note = Human BCMA
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 216 - protein, 243 aa, synthetic construct
  REGION 1..243: note = Anti-BCMA scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 217 - protein, 244 aa, synthetic construct
  REGION 1..244: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 218 - protein, 252 aa, synthetic construct
  REGION 1..252: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 219 - protein, 253 aa, synthetic construct
  REGION 1..253: note = Anti-BCMA scFv
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 220 - protein, 240 aa, synthetic construct
  REGION 1..240: note = Anti-BCMA scFv
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 221 - protein, 241 aa, synthetic construct
  REGION 1..241: note = Anti-BCMA scFv
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 222 - protein, 247 aa, synthetic construct
  REGION 1..247: note = Anti-BCMA scFv
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 223 - protein, 248 aa, synthetic construct
  REGION 1..248: note = Anti-BCMA scFv
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 224 - protein, 244 aa, synthetic construct
  REGION 1..244: note = Anti-BCMA scFv
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 225 - protein, 245 aa, synthetic construct
  REGION 1..245: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 226 - protein, 247 aa, synthetic construct
  REGION 1..247: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 227 - protein, 248 aa, synthetic construct
  REGION 1..248: note = Anti-BCMA scFv
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 228 - protein, 257 aa, synthetic construct
  REGION 1..257: note = Anti-BCMA scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 229 - protein, 258 aa, synthetic construct
  REGION 1..258: note = Anti-BCMA scFv
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 230 - protein, 248 aa, synthetic construct
  REGION 1..248: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 231 - protein, 249 aa, synthetic construct
  REGION 1..249: note = Anti-BCMA scFv
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 232 - protein, 246 aa, synthetic construct
  REGION 1..246: note = Anti-BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 233 - protein, 246 aa, synthetic construct; identical to SEQ ID NO: 232
  REGION 1..246: note = Anti-BCMA scFv

SEQ ID NO: 234 - protein, 246 aa, synthetic construct; identical to SEQ ID NO: 232
  REGION 1..246: note = Anti-BCMA scFv

SEQ ID NO: 235 - protein, 239 aa, synthetic construct
  REGION 1..239: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 236 - protein, 246 aa, synthetic construct
  REGION 1..246: note = Anti-BCMA scFv
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 237 - protein, 239 aa, synthetic construct
  REGION 1..239: note = Anti-BCMA scFv
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 238 - protein, 241 aa, synthetic construct
  REGION 1..241: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 239 - protein, 243 aa, synthetic construct
  REGION 1..243: note = Anti-BCMA scFv
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 240 - protein, 243 aa, synthetic construct
  REGION 1..243: note = Anti-BCMA scFv
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 241 - protein, 243 aa, synthetic construct
  REGION 1..243: note = Anti-BCMA scFv
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 242 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 243 - protein, 246 aa, synthetic construct; identical to SEQ ID NO: 242
  REGION 1..246: note = Anti-BCMA scFv

SEQ ID NO: 244 - protein, 246 aa, synthetic construct; identical to SEQ ID NO: 242
  REGION 1..246: note = Anti-BCMA scFv

SEQ ID NO: 245 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 246 - protein, 246 aa, synthetic construct; identical to SEQ ID NO: 245
  REGION 1..246: note = Anti-BCMA scFv

SEQ ID NO: 247 - protein, 246 aa, synthetic construct; identical to SEQ ID NO: 245
  REGION 1..246: note = Anti-BCMA scFv

SEQ ID NO: 248 - protein, 11 aa, synthetic construct
  REGION 1..11: note = CDR L1
RASQDISKYLN

SEQ ID NO: 249 - protein, 7 aa, synthetic construct
  REGION 1..7: note = CDR L2
SRLHSGV

SEQ ID NO: 250 - protein, 9 aa, synthetic construct
  REGION 1..9: note = CDR L3
GNTLPYTFG

SEQ ID NO: 251 - protein, 5 aa, synthetic construct
  REGION 1..5: note = CDR H1
DYGVS

SEQ ID NO: 252 - protein, 16 aa, synthetic construct
  REGION 1..16: note = CDR H2
VIWGSETTYYNSALKS

SEQ ID NO: 253 - protein, 7 aa, synthetic construct
  REGION 1..7: note = CDR H3
YAMDYWG

SEQ ID NO: 254 - protein, 120 aa, synthetic construct
  REGION 1..120: note = VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 255 - protein, 107 aa, synthetic construct
  REGION 1..107: note = VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 256 - protein, 245 aa, synthetic construct
  REGION 1..245: note = scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 257 - protein, 11 aa, synthetic construct
  REGION 1..11: note = CDR L1
KASQNVGTNVA

SEQ ID NO: 258 - protein, 7 aa, synthetic construct
  REGION 1..7: note = CDR L2
SATYRNS

SEQ ID NO: 259 - protein, 9 aa, synthetic construct
  REGION 1..9: note = CDR L3
QQYNRYPYT

SEQ ID NO: 260 - protein, 5 aa, synthetic construct
  REGION 1..5: note = CDR H1
SYWMN

SEQ ID NO: 261 - protein, 17 aa, synthetic construct
  REGION 1..17: note = CDR H2
QIYPGDGDTNYNGKFKG

SEQ ID NO: 262 - protein, 13 aa, synthetic construct
  REGION 1..13: note = CDR H3
KTISSVVDFYFDY

SEQ ID NO: 263 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 264 - protein, 108 aa, synthetic construct
  REGION 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 265 - protein, 245 aa, synthetic construct
  REGION 1..245: note = scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 266 - protein, 12 aa, synthetic construct
  REGION 1..12: note = HC-CDR3
HYYYGGSYAMDY

SEQ ID NO: 267 - protein, 7 aa, synthetic construct
  REGION 1..7: note = LC-CDR2
HTSRLHS

SEQ ID NO: 268 - protein, 9 aa, synthetic construct
  REGION 1..9: note = LC-CDR3
QQGNTLPYT

SEQ ID NO: 269 - DNA, 735 bp, synthetic construct
  misc_feature 1..735: note = Sequence encoding scFv
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 270 - protein, 5 aa, synthetic construct
  REGION 1..5: note = CDR-H1
SFAMS

SEQ ID NO: 271 - protein, 17 aa, synthetic construct
  REGION 1..17: note = CDR-H2
AISGSGGGTYYADSVKG

SEQ ID NO: 272 - protein, 13 aa, synthetic construct
  REGION 1..13: note = CDR-H3
DKILWFGEPVFDY

SEQ ID NO: 273 - protein, 11 aa, synthetic construct
  REGION 1..11: note = CDR-L1
RASQSVSSYLA

SEQ ID NO: 274 - protein, 7 aa, synthetic construct
  REGION 1..7: note = CDR-L2
DASNRAT

SEQ ID NO: 275 - protein, 13 aa, synthetic construct
  REGION 1..13: note = CDR-L3
FGQGTKVEIKRTV

SEQ ID NO: 276 - protein, 122 aa, synthetic construct [sequence omitted]

SEQ ID NO: 277 - protein, 110 aa, synthetic construct
  REGION 1..110: note = VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTV

SEQ ID NO: 278 - protein, 5 aa, synthetic construct
  REGION 1..5: note = CDR-H1
DYWMQ

SEQ ID NO: 279 - protein, 17 aa, synthetic construct
  REGION 1..17: note = CDR-H2
TIYPGDGDTGYAQKFQG

SEQ ID NO: 280 - protein, 11 aa, synthetic construct
  REGION 1..11: note = CDR-H3
GDYYGSNSLDY

SEQ ID NO: 281 - protein, 11 aa, synthetic construct
  REGION 1..11: note = CDR-L1
KASQDVSTVVA

SEQ ID NO: 282 - protein, 7 aa, synthetic construct
  REGION 1..7: note = CDR-L2
SASYRYI

SEQ ID NO: 283 - protein, 9 aa, synthetic construct
  REGION 1..9: note = CDR-L3
QQHYSPPYT

SEQ ID NO: 284 - protein, 120 aa, synthetic construct [sequence omitted]

SEQ ID NO: 285 - protein, 109 aa, synthetic construct
  REGION 1..109: note = VL
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRT